SEQUENCE LISTING (14 sequences)

SEQ ID NO: 1 - DNA, 11766 bp, synthetic construct
  misc_feature 1..11766: note = depicts the nucleic acid sequence encoding amino acid sequence of PKSTE protein
  source 1..11766: mol_type = other DNA
atggtctctgccgaacatccgatcgagccgaccccgagcttcgcaatcatcggctacgccgcacgttttcccggcgcggcgagcgccgaggaatactgggcgttgttgcgggacgggcgtgaagcgatatccgatgttccgaaagaccgctgggacatcgaggagttcttcgacccggacccgtcgacaccgggcaaggtcgtcacgcgccgcgcggggttcgtcgatgacgtaacgggtttcgacgcgccgttcttcggtatgtccgcccgcgaggtacgtctgatggacccgcagcaccggattctcatggaaaccgcgtggcgcgcggtcgaacactcgggaatcgcgccgacgtcgctggccaacagcaacacgggcgtcttcgtcggcctggccacccacgactacctcggtatggcctccgacgaactcacctaccccgagatcgaggcctacatggccatcggaacctccaatgccgctgcggccggacggatcagttaccgcctgggattgcagggaccggcggtggcggtcgacaccgcgtgcagttcgtcgctggtggcgattcaccaggcgtgtcaggcacttcgcctgaacgagtgcgatctcgcgttggccggcggggcgaacgtcctgctcaccccggccaccatgatcacgttctccaacgcgcacatgctggctccggacggccggtgcaagaccttcgacgcggcggccgacggctacgtgcgcggcgagggctgcggtgtcgtcgtggtcaagcgcctcgaggacgcggtgcgcgacggcgaccggatccgcgcggtgatccgcggaagcgcaatcaaccaggacggggcgtcgggcgggctgaccgtgccgaacggcgttgctcagcaacgggttatcgccgacgcgttgaagcgcgccggcctgcgaccggccgatgtcggttatctcgaggcgcacggcaccggaacctcacttggcgaccccatcgaggcccaggccgcgggcgaggtgctcggggccggacgcgcacctgaagaaccgctgctgatcggctcggcgaagaccaacatcggccatctggaagcggccgcgggcatcgccggtgtcatcaaggtggtcatggcgctcgagaacgagacgttgcccaagcacctcaatttcgagactccgtcgccccacatcccgtgggagcgtctacccatcgaggtggtgagagagaccgttccctggcagcgcaacggcaagccgcggatcgccggggtgagctcattcgggtttgccgggactaacgcgcacgtcatcctcgaagaggcgcccgccgtcggaacaccggcgacgacagagcctgtcgaggatccgaggcgctacagcgtccttccgctgtcggcacgcacgcccgaggcgttggtccgactcgccggtgagtaccgcgactggctgcgcgcccacccgcaggcccgcctggccgacgtgtcccacaccgcgggaaccgcacgtgcccacctggagcagcgtgccgcgttggtggtcaactcgcgggaatcggccatcgaactgctcggcgcgctcgccgacgaccgtccggcgcccggcctggtccgcggtgaatcccacgaggcacccaagaccgcgtggttgttcaccggccagggcagccagtaccccggaatggcgcgtgagttgttcgacaccgaaccggtgttcgccgaaaccgtgaggaaatgcgccgaggccgtcgcagatatcctcgaaaagcctttgctcgacgtcattttcgacgccgacgagttggatggtgacaccgaatcgccgctgcggcacacgacctatgcgcaacccgcgctgttcgccgtcgaattgggcctggcccggctctggcagtcctggggcttcgaacccgacgtggtggtcggacacagtgtcgggcagtactcggccgcctgtgtggcgggtgtgttcgacgtcgaggacggcgcacgactgatcgccgagcgcggccgattgttcggcagcctgcccgcgggtggacgcatggcggcggttttcaccgcccccgagcgtgcagagcgcctcaccgacgagtaccccagcctgtcggttgccgcgtacaacggtgccaacaccgtattgtccggtcccgcaaaggatctcgaatccgccgtggccacattggtggccgacggtgtgcggtgcgactggctggacaccagccacgccttccactcggcactgctcgacccgattctcgacgatttcgagtcgttcgcccagaagctcgaataccgtgaaccgcaacgtatcctgatcgacaaccgcaccgggtccgcgctcgggcggagcaccaggctcgacggtgcctactggcggcggcacgcacgccagcctgtcgagttcgccaagagcgtccgcaccctcgccgacatgaactgcagggtgctgctggagatagggcccaggcccgtgctcaccgccaccgcgctggcagcatggcccgacccggccaccacgccgcaggtgctcgcgtcgctgcggcccaccacggccgatcaccggcagatcaccgaggccgtcgccgccgcctatgccgcgggacatctgcccgatttccgcgcgttccggcaaccggacgcgcgcaaactcgacctgcccacatatcccttcgagcatcgccagttctggttctcggacaaccgtgcgatcgaccgtgacacccagaccgcgagcgccggttccgtcacgcctcaccgcacacaggccgtccgacttctcgaagacggccagatcgaggaactcgccgccctcatcgacggcgagaccgtcgacgagcagaccctgcgcgtgctgaacaagctcgcggcgcgccacaaccagcagcggtccacacacgtcgaccctgatgcgcggtacgagatccgttgggacgcgatcgcttcggccgccgcgagcacgggcgcggcagccgactggatcctggtcggcgacgacaccgctgccgtcctggagttcgccgatgcgctcaccgcaggcggtcatcgccaccagatcgtcgcgctgcccggctccgacgccgacgaggcccagctggtcgatacgctgcgcgctgcgtcggccggtgagctgtacgtcgtgcacatcgcggcaggagacggtgccgctgccccgatgcgggacctgctgcgcgtgcagcaccgaacccttggtgggctgcggcgcctgttccgcgccgcgaacgacgcggaactgcgcagccccatctggttggtgacacgtgacgggcagcgggtcaccgacaccgacaccgtggtaccggaacagagttgcctgtggggattcggccgcgcggcggccctggaactcccgcacatctggggtggcctcgccgacctctccggtgcggacaccgagtggccgcggctcatcgcgcggatcacgtcaccgcgcgacggtgaggaccagatcgcgctgcgcggggacgccgtgtacgcaccccgactggtccgccggaccggcgagccgagcgacacccccctgccggtgcgggaggaccgcacctatctggtgacgggcggtctcggggcgatcgggctcgaggtcgccggctacctggcctcccacggtgccggtcatgtggtgctcaccagccgacgggaaccgggcgacgacgcgcgccggcgcctcgacggtctccgcgagcagtacgcatgtgacgtccgggtgatcaccgccgatgtcgccgacgcgcacgacgtcgcgcgcctcatgaccaccgtcgccgccgaactcccgccgctggccggcatcgtgcacgccgcgggcgagatcggcaccacgccgctgagcggcctcgacgacgccgaggtggatcgcgtcttcgccgggaaggtctggggcgcctggcatctgagcgaggccctgacagatcttcgcaccgatctcgacttcttcgtcagcacatcgtcgatcgcgtcggtgtggggcggattcgggcagaccgcctacggcgcggcgaacgcgtttctcgacggtctggcgtggcgactgcgcgagcggggtgtcaccgctgtcagcgtgaacttcgggccgtgggccgcgggcatggccgacgccgaatcacgtgcccgcctcgagcagcgaggcgtgcggacgttgtcgcccgccgaggccctcgcaggcatggccgacgtcgtggccggccccgttcagggagtcgtcgccaagatcgactggtcacgtttcctgccgctgtaccagcaggcaggccggcgcgcgttcctgtcggaactcgaaagcgaattgcccgcagcggcaaccggcgctgctgcgcccgcgacggtgcccgggaaaccaccgctcgtcgagcagttgaccaaagcgcctgtgcagcaacgcaaaagcctcatcacgaactacctgcgcaacgcggtcgccgaggtcacacgggtcgacgccgacgagatccgcgatgaggccgggttcttcgacctcgggatggactccctgatggccgtcgaactgcggcggcgcatcgagcagggtgtcggcaaggacattcccgtcacgctcgtgatggatcatccgcggttgtccgacgcggccgactacctgctggtcgaggtactcgggttgggcgagcagaccaacgtgcggcaggcgtcgaccgtgaccgcgcgaaccgacgatcccatcgcggtcgtcgcggtgtcctgccgcttccccggcgcacccgacccggagtcgttctgggagctgctcgccggtggtgtcgatgccatccgagaggtccccgaggaccgctgggacatcgacgagttctacgaccccgatccggacaccccgggcaagacctacacgcggttcggcggatttctagacggcatcgacggattcgatcccgagttcttcggcatctcaccgcgtgaggccgtgtggatcgagccgcagcagcgcctcatgctcgagacggtgtgggagggtctcgagcgggccggctacgcaccttcggacctccggggcagccgcacgggtgtcttcaccggtgttgccgccaacgagtacgcgcatctgctgtcggccgagtcgatcgacaagatcgagccgcacttcatcacgggcaatgcgctcaacgcgatctccggtcgcgtggcgttcgcgctgggcttcgaaggtcctgccgtcgcggtcgacaccgcgtgcagctcggctctggtcgcggtccatcaggcatgtcaggcactgcagtccggggactgcgatctggccctcgcgggaggcgtcaacgtgctgctgagccccgtcaccgtggtcgcggcctcacgcgcgcggatgctgtcgccggtgggccggtgcaagacgttcgacgcgtccgccgacgggtatgtgcgcagtgagggctgcggcgtcctggtgctcaagcgcctcagcgacgccgtgcgtgacggcgaccgggtgtgcgcggtgatccctgcgagctcggtgaaccaggacggcgcctccagcgggttgaccgtccccaacggcggtgcgcagcaacggctcatcgaggcgacgctggcccgcgccggactgagcggcgccgatgtggactacctcgaggcacacggcacgggtacgcccctcggtgacccgatcgaggtgcaggccgccgcggccgcgtacggcgccgggcgtgacgccgaccggccgctgctgatgggatcggtgaagaccaacatcgggcacaccgaatccgcctcgggtgcagcgggtctgatcaaggtggtgctgtcactgcaacacggggtgctgccgcagagcctgcacttcgagaatccgtcaccgcacatcccgtgggacgcattgccggtgcgggtgatcgacgaaccggtgccgtggcagaccaacggccgggtgcgccgtgccggtgtcagttcgttcgggttcaccggcaccaacgcgcacgtgctcgtcgaggaggccccgccgcaactgcacaccgccgacgctgcgcccgtggaaccggccgccaccgcggaggccgggaccgacgactcgccgcaggtgctggcgctgtcggcgcggtcgcccgaggcgctcgtggagttggcgcggcgctacgacgactggttgagcgcgacgcccggcctcgacatcgccgacgtgtgccacaccgcgggcgtgggccgttcgcacttcgaataccgcgcagccctggtggtcgactcggtcgagcgggcacgcgagggtctcgccgcgctcgccgaggggcgcactcctgcgggcgtggtgcgcggcgagcatgtgcaccgtccgacgacggcatggctgttcaccggacagggcagccagtacccgggcatggcccgcgaattgttcgacacgcaaccggttttcgcccagaccgttcggcagtgcgccgaggcggtcgccgacatgctgccgcgaccactgctggacgtgctgttcgcgaccgaccgcgaaaccgcggacctgctgcagcacacgtcgttcgcgcagcccgcgctgttcgcggtcgagatggggctagcgcggctgtggcagtcatgggccatcgaacccgatgtggtgctggggcacagcgtcggccagtacgccgcggcctgcgtggcgggcgtgttcagccttgacgacggcgcacgactgatggccgagcgcggccgcatgttcggcagccttcccgaaggcggccggatggtggccgtgttcgccgacgccgaacacgtcgagcaggtggccggtgagttcccccgggtgtcggtcggcgcctacaacggccccaacaccgtgctctcgggtcccggcgaggacctcgaagagatcgtcgagaggttcgaggacgaggggatccgctgcacgtggctggcgaccagccacgcgttccactccgaactgctcgatcccgtgctcgacgagttcgaggcctacgcggcccagttccagttcgcagcgccgacactgcctttggtgtgcaaccggaccggggccgtgctcaccgggcagaccccgctcgacgcgcagtactggcgcaggcattcccgccagcccgtgcagttcgccgagagtgtgcgcaccgtcgccgcgctcggctgctcggtgctgatggagatcggcccgcaacctgtgctgaccggggccgcggtacaggtgtggcccgaacacatggctgccccgcgggccgtcgcctcgctgcgcaagggcgtcgccgatcgccgacagatcgccgatgcgctggcgtcggcctacgtcggcggccaccgccccgacttcgccgcggtgcaccggcagccacgccgcccggtcgagttgcccacctatccgttccagcgccgcaggttctggccgaaggcctcgagcatcgccgtcgacggtgggaccgcggcgtcgggaatcctgggcagcggcaaggatctggcgtccggcgacaccatctacacgagccggctgtccgtcaaatcgcagccgtggctggccgatcacgtcatctacggcaccgtcgtcgtccccggcgcgacgtatgcggcgatggcgctcgccgcggtcggcacaccggcacgcctgaaggacgtgttcttctacgagccgatcatcctgcccgagaaggcctctcgcgaggtccagctgacgttgcaccctgccgacggtggcagcgtgctcagcttccagatccacagccgcccgtacggtgaacgtggtgccgactggtcgctgaacgccgagggcacggtcgacacggccggcagcgacccggacgccgccgcagacgagcagtcggatccggtcgacgaggtgatcgaacgcctcgagcgcatgcgtccgcaggacctcttcgagaccttcgccgacctggaactggcgtggggtcccacatggtcgggctcgctgaaatcgttgtggctcggcgacggtgaggccgtcggcgacatcctcgtcggcgccgaactcgccgagcaactcggctccgagcccatgcacccggtgctgatggacctgtgcacgggcgtcgcgttccccgcgttcccggcgctgctcgcggccgaacagggcgtcaacgacctgttcctgccgctgcgctacgggcaggtgacgctcgcggagaagatgccgcggcggttctactgccgggcgaggtggcaccgtagcgcgctcgacagcgaaacgcaggtgttcgaactcgaattcgtcgacctcggtgggcgagcgctcggcggcatccgcgagttcaccgtcaaacgtgcgccgcgcgaggcactgctgcgcggcctcgggggtgacgcgacccgcctgctctacacgctcgggtggcacgaggtgccgccgccaccatcggccgacgacgcggtcggcacgtggctgatcgccgggttcgacgagctggccgcgaaggtgcccggctgtgtccccatcgaccgcaacaccgatctgcaggtcctcggtgaggtgctgtcgcaggcgcacgctcgcggcgcgggattctcgggcgtcgtgtggcgttgcgcgacgcctggccggaaggaatcgggttctgcctccgccgaatccgcccgtctggaggccgagatcaccaatctgctcagcgcggtgcacgccgcgcaggggccgagcgtgaaactgcccggcggactctggatcgtcaccgaacacggcgtggcatgtgaatccggcgagccggtggacccggtgcaggccgcgctgtggggcttcggccgaacgacgatcaacgaggaaccggcgctgcactgccggctcgtcgactgcgacggatcttccgaggccgtcgacctgctggccgctctgctggccgcaccgggcatccaggaaccggaaatcgccgtgcggcagggcaaactgctggcgtcacggttgttgccgtgggcccgcagtggtcatctcacgctgccgcgcgggggcgacttcgtccttgcgcccaccgagcgcggcgcgatcgacaacctgcgcctcacggagacggacgtgccggagccggacgagggctacgtgcaggttcgggtcgaggccgcgggcctcaacttccgcgacgtgctcaacgtgctcggcctgtaccccggtgatccgggaccgatcggcggcgacttcgcaggcaccgtgacccaactgggggcaggtgtgagtggactcgaaatcggccaacgcgtgtacgggtcgatgcagggtgccttcgcgagccgcttcaacgtgcccgcccagttcctggcgccgattcccgacggcatcggcgcggtcgaggccgccacgatcccggccgcggcgctgacggtccggctcgcgttcgactgggcgcaactcaaacccggcgacaaggtgctcatccacgccgccagcggtggcgtggggctcgcggccatccagatggcgcagcagtccggcgccgaggtcttcgccaccgcgagcaccttcaagcgttcgaccctgcgcaggctgggcgtgaagtacgtctacgactctcggacgacggacttcgccgaccagatcctcgcggacaccaacggtgccggcgtggacgtggtgctcaacagcctcactagtgcgggtttcgtcgaggcgacgctgaaggccaccgcgcagggcggacgcttcgccgagatcgccaagcgcgacatctggtcgcatgagcagatggccgaggcccgccccgacatcgcctacgagatcgtcgcgctggacaccgtgatgttcaccgagcccgatcgcatccgcgacctgctcaccgaggtgtccgacgggatggcgcgcggcgagtggacaccactacccgccgagatctacccgatcaccgaggcccggtcggcgttccggcgcatgcaacaggcacggcacatcggcaagattgtgctgcaggtccccaaaccccttcagccgcagcaggaccggagctacctgatcaccggcggactcggggcgatcggcctgcacacggcggcgtatctggcgcagctcggcgcgggcgacatcgtgctgaccagccggcgcgaccccgacgagtcggcccagcgggcgatcgacgagatcaccgagcgccacaagtgccgcatccacacctatgcggccgatgtgggtgacgaggcacaggtggccgatctgctggcgcggatccgggccgagttgcctccactggccggaatcgcccatctggcgggcgtgctcgacgacgcgctgctgtcggcgcagaaccccgagcgcttccggaccacgttggcgcccaaggcctatggcgcgagccacctggaccgcctgacgcgcgacgacgacctggacttcttcatcgtgtcctcgtcggtgtcgagcctgttcggatcaccggggcaggccaactacgcaaccgccaatgcgatgctcgacggtctggtcgccaagcgccgtgcgcagggcctggtggccacgggcatcaacttcgggccgtggggtcagggcggcatggcgtcgtcggaggccgcgaccgcgaacatcaccgcccaaggtctgatcccgttggatccgtcggccgcgttgcacgcgctggccgaggtcatcgcgaacggctccgggcaggcgacggtgctcaaggccaactggcagcgcgcggcgaaggttctcggctcggcgcgcccgccgatcctggacctcgtcctgccgagcgcggccggggaggccacgggtgacagcgagctgctcaagcagttgatggagatcccgataccgcagcgcgccgggttcgtcaccgagttcctgcaacgcgaagtgcagaacttcctgcgtctcgcgtcgccgcccgcggcgtcgagccggttcctggacctcggcacggattcgctgatggccatcgagctgcgcaaccggctgcacagccagttcgggggcaagttcaccatcaacgcgaccgcggtgttcgactacccgaccatcggcgggctcgcggagtacctcactagtcagctcgacagcgggactcccgcccgggaagcgagcagcgctcttcgcgacggctaccggcaggcgggcgtgtcgggcagggtccggtcctacctcgacctgctggcggggctgtcggacttccgcgagcacttcgacggctccgacgggttctccctcgatctcgtggacatggccgacggtcccggagaggtcacggtgatctgctgcgcgggaacggcggcgatctccggtccgcacgagttcacccggctcgccggggcgctgcgcggaatcgctccggttcgggccgtgccccagcccggctacgaggagggcgaacctctgccgtcgtcgatggcggcggtggcggcggtgcaggccgatgcggtcatcaggacacagggggacaagccgttcgtggtggccggtcactccgcgggggcactgatggcctacgcgctggcgaccgaactgctcgatcgcgggcacccgccacgcggtgtcgtcctgatcgacgtctacccgcccggtcaccaggacgcgatgaacgcctggctggaggagctgaccgccacgctgttcgaccgcgagacggtgcggatggacgacaccaggctcaccgccctgggcgcctacgaccgcctcaccggtcagtggcgaccccgggaaaccgggctgccgacgctgctggtcagcgccggcgagccgatgggtccgtggcccgacgacagctggaagccgacgtggcccttcgagcacgacaccgtcgccgtccccggcgaccacttcacgatggtgcaggaacacgccgacgcgatcgcgcggcacatcgacgcctggctgggcggagggaattcatga

SEQ ID NO: 2 - protein, 3921 aa, synthetic construct
  REGION 1..3921: note = depicts the amino acid sequence of PKSTE protein
MVSAEHPIEPTPSFAIIGYAARFPGAASAEEYWALLRDGREAISDVPKDRWDIEEFFDPDPSTPGKVVTRRAGFVDDVTGFDAPFFGMSAREVRLMDPQHRILMETAWRAVEHSGIAPTSLANSNTGVFVGLATHDYLGMASDELTYPEIEAYMAIGTSNAAAAGRISYRLGLQGPAVAVDTACSSSLVAIHQACQALRLNECDLALAGGANVLLTPATMITFSNAHMLAPDGRCKTFDAAADGYVRGEGCGVVVVKRLEDAVRDGDRIRAVIRGSAINQDGASGGLTVPNGVAQQRVIADALKRAGLRPADVGYLEAHGTGTSLGDPIEAQAAGEVLGAGRAPEEPLLIGSAKTNIGHLEAAAGIAGVIKVVMALENETLPKHLNFETPSPHIPWERLPIEVVRETVPWQRNGKPRIAGVSSFGFAGTNAHVILEEAPAVGTPATTEPVEDPRRYSVLPLSARTPEALVRLAGEYRDWLRAHPQARLADVSHTAGTARAHLEQRAALVVNSRESAIELLGALADDRPAPGLVRGESHEAPKTAWLFTGQGSQYPGMARELFDTEPVFAETVRKCAEAVADILEKPLLDVIFDADELDGDTESPLRHTTYAQPALFAVELGLARLWQSWGFEPDVVVGHSVGQYSAACVAGVFDVEDGARLIAERGRLFGSLPAGGRMAAVFTAPERAERLTDEYPSLSVAAYNGANTVLSGPAKDLESAVATLVADGVRCDWLDTSHAFHSALLDPILDDFESFAQKLEYREPQRILIDNRTGSALGRSTRLDGAYWRRHARQPVEFAKSVRTLADMNCRVLLEIGPRPVLTATALAAWPDPATTPQVLASLRPTTADHRQITEAVAAAYAAGHLPDFRAFRQPDARKLDLPTYPFEHRQFWFSDNRAIDRDTQTASAGSVTPHRTQAVRLLEDGQIEELAALIDGETVDEQTLRVLNKLAARHNQQRSTHVDPDARYEIRWDAIASAAASTGAAADWILVGDDTAAVLEFADALTAGGHRHQIVALPGSDADEAQLVDTLRAASAGELYVVHIAAGDGAAAPMRDLLRVQHRTLGGLRRLFRAANDAELRSPIWLVTRDGQRVTDTDTVVPEQSCLWGFGRAAALELPHIWGGLADLSGADTEWPRLIARITSPRDGEDQIALRGDAVYAPRLVRRTGEPSDTPLPVREDRTYLVTGGLGAIGLEVAGYLASHGAGHVVLTSRREPGDDARRRLDGLREQYACDVRVITADVADAHDVARLMTTVAAELPPLAGIVHAAGEIGTTPLSGLDDAEVDRVFAGKVWGAWHLSEALTDLRTDLDFFVSTSSIASVWGGFGQTAYGAANAFLDGLAWRLRERGVTAVSVNFGPWAAGMADAESRARLEQRGVRTLSPAEALAGMADVVAGPVQGVVAKIDWSRFLPLYQQAGRRAFLSELESELPAAATGAAAPATVPGKPPLVEQLTKAPVQQRKSLITNYLRNAVAEVTRVDADEIRDEAGFFDLGMDSLMAVELRRRIEQGVGKDIPVTLVMDHPRLSDAADYLLVEVLGLGEQTNVRQASTVTARTDDPIAVVAVSCRFPGAPDPESFWELLAGGVDAIREVPEDRWDIDEFYDPDPDTPGKTYTRFGGFLDGIDGFDPEFFGISPREAVWIEPQQRLMLETVWEGLERAGYAPSDLRGSRTGVFTGVAANEYAHLLSAESIDKIEPHFITGNALNAISGRVAFALGFEGPAVAVDTACSSALVAVHQACQALQSGDCDLALAGGVNVLLSPVTVVAASRARMLSPVGRCKTFDASADGYVRSEGCGVLVLKRLSDAVRDGDRVCAVIPASSVNQDGASSGLTVPNGGAQQRLIEATLARAGLSGADVDYLEAHGTGTPLGDPIEVQAAAAAYGAGRDADRPLLMGSVKTNIGHTESASGAAGLIKVVLSLQHGVLPQSLHFENPSPHIPWDALPVRVIDEPVPWQTNGRVRRAGVSSFGFTGTNAHVLVEEAPPQLHTADAAPVEPAATAEAGTDDSPQVLALSARSPEALVELARRYDDWLSATPGLDIADVCHTAGVGRSHFEYRAALVVDSVERAREGLAALAEGRTPAGVVRGEHVHRPTTAWLFTGQGSQYPGMARELFDTQPVFAQTVRQCAEAVADMLPRPLLDVLFATDRETADLLQHTSFAQPALFAVEMGLARLWQSWAIEPDVVLGHSVGQYAAACVAGVFSLDDGARLMAERGRMFGSLPEGGRMVAVFADAEHVEQVAGEFPRVSVGAYNGPNTVLSGPGEDLEEIVERFEDEGIRCTWLATSHAFHSELLDPVLDEFEAYAAQFQFAAPTLPLVCNRTGAVLTGQTPLDAQYWRRHSRQPVQFAESVRTVAALGCSVLMEIGPQPVLTGAAVQVWPEHMAAPRAVASLRKGVADRRQIADALASAYVGGHRPDFAAVHRQPRRPVELPTYPFQRRRFWPKASSIAVDGGTAASGILGSGKDLASGDTIYTSRLSVKSQPWLADHVIYGTVVVPGATYAAMALAAVGTPARLKDVFFYEPIILPEKASREVQLTLHPADGGSVLSFQIHSRPYGERGADWSLNAEGTVDTAGSDPDAAADEQSDPVDEVIERLERMRPQDLFETFADLELAWGPTWSGSLKSLWLGDGEAVGDILVGAELAEQLGSEPMHPVLMDLCTGVAFPAFPALLAAEQGVNDLFLPLRYGQVTLAEKMPRRFYCRARWHRSALDSETQVFELEFVDLGGRALGGIREFTVKRAPREALLRGLGGDATRLLYTLGWHEVPPPPSADDAVGTWLIAGFDELAAKVPGCVPIDRNTDLQVLGEVLSQAHARGAGFSGVVWRCATPGRKESGSASAESARLEAEITNLLSAVHAAQGPSVKLPGGLWIVTEHGVACESGEPVDPVQAALWGFGRTTINEEPALHCRLVDCDGSSEAVDLLAALLAAPGIQEPEIAVRQGKLLASRLLPWARSGHLTLPRGGDFVLAPTERGAIDNLRLTETDVPEPDEGYVQVRVEAAGLNFRDVLNVLGLYPGDPGPIGGDFAGTVTQLGAGVSGLEIGQRVYGSMQGAFASRFNVPAQFLAPIPDGIGAVEAATIPAAALTVRLAFDWAQLKPGDKVLIHAASGGVGLAAIQMAQQSGAEVFATASTFKRSTLRRLGVKYVYDSRTTDFADQILADTNGAGVDVVLNSLTSAGFVEATLKATAQGGRFAEIAKRDIWSHEQMAEARPDIAYEIVALDTVMFTEPDRIRDLLTEVSDGMARGEWTPLPAEIYPITEARSAFRRMQQARHIGKIVLQVPKPLQPQQDRSYLITGGLGAIGLHTAAYLAQLGAGDIVLTSRRDPDESAQRAIDEITERHKCRIHTYAADVGDEAQVADLLARIRAELPPLAGIAHLAGVLDDALLSAQNPERFRTTLAPKAYGASHLDRLTRDDDLDFFIVSSSVSSLFGSPGQANYATANAMLDGLVAKRRAQGLVATGINFGPWGQGGMASSEAATANITAQGLIPLDPSAALHALAEVIANGSGQATVLKANWQRAAKVLGSARPPILDLVLPSAAGEATGDSELLKQLMEIPIPQRAGFVTEFLQREVQNFLRLASPPAASSRFLDLGTDSLMAIELRNRLHSQFGGKFTINATAVFDYPTIGGLAEYLTSQLDSGTPAREASSALRDGYRQAGVSGRVRSYLDLLAGLSDFREHFDGSDGFSLDLVDMADGPGEVTVICCAGTAAISGPHEFTRLAGALRGIAPVRAVPQPGYEEGEPLPSSMAAVAAVQADAVIRTQGDKPFVVAGHSAGALMAYALATELLDRGHPPRGVVLIDVYPPGHQDAMNAWLEELTATLFDRETVRMDDTRLTALGAYDRLTGQWRPRETGLPTLLVSAGEPMGPWPDDSWKPTWPFEHDTVAVPGDHFTMVQEHADAIARHIDAWLGGGNS

SEQ ID NO: 3 - DNA, 11805 bp, synthetic construct
  misc_feature 1..11805: note = depicts the nucleic acid sequence encoding PKS14TE protein
  source 1..11805: mol_type = other DNA
atggtctctgccgaacatccgatcgagccgaccccgagcttcgcaatcatcggctacgccgcacgttttcccggcgcggcgagcgccgaggaatactgggcgttgttgcgggacgggcgtgaagcgatatccgatgttccgaaagaccgctgggacatcgaggagttcttcgacccggacccgtcgacaccgggcaaggtcgtcacgcgccgcgcggggttcgtcgatgacgtaacgggtttcgacgcgccgttcttcggtatgtccgcccgcgaggtacgtctgatggacccgcagcaccggattctcatggaaaccgcgtggcgcgcggtcgaacactcgggaatcgcgccgacgtcgctggccaacagcaacacgggcgtcttcgtcggcctggccacccacgactacctcggtatggcctccgacgaactcacctaccccgagatcgaggcctacatggccatcggaacctccaatgccgctgcggccggacggatcagttaccgcctgggattgcagggaccggcggtggcggtcgacaccgcgtgcagttcgtcgctggtggcgattcaccaggcgtgtcaggcacttcgcctgaacgagtgcgatctcgcgttggccggcggggcgaacgtcctgctcaccccggccaccatgatcacgttctccaacgcgcacatgctggctccggacggccggtgcaagaccttcgacgcggcggccgacggctacgtgcgcggcgagggctgcggtgtcgtcgtggtcaagcgcctcgaggacgcggtgcgcgacggcgaccggatccgcgcggtgatccgcggaagcgcaatcaaccaggacggggcgtcgggcgggctgaccgtgccgaacggcgttgctcagcaacgggttatcgccgacgcgttgaagcgcgccggcctgcgaccggccgatgtcggttatctcgaggcgcacggcaccggaacctcacttggcgaccccatcgaggcccaggccgcgggcgaggtgctcggggccggacgcgcacctgaagaaccgctgctgatcggctcggcgaagaccaacatcggccatctggaagcggccgcgggcatcgccggtgtcatcaaggtggtcatggcgctcgagaacgagacgttgcccaagcacctcaatttcgagactccgtcgccccacatcccgtgggagcgtctacccatcgaggtggtgagagagaccgttccctggcagcgcaacggcaagccgcggatcgccggggtgagctcattcgggtttgccgggactaacgcgcacgtcatcctcgaagaggcgcccgccgtcggaacaccggcgacgacagagcctgtcgaggatccgaggcgctacagcgtccttccgctgtcggcacgcacgcccgaggcgttggtccgactcgccggtgagtaccgcgactggctgcgcgcccacccgcaggcccgcctggccgacgtgtcccacaccgcgggaaccgcacgtgcccacctggagcagcgtgccgcgttggtggtcaactcgcgggaatcggccatcgaactgctcggcgcgctcgccgacgaccgtccggcgcccggcctggtccgcggtgaatcccacgaggcacccaagaccgcgtggttgttcaccggccagggcagccagtaccccggaatggcgcgtgagttgttcgacaccgaaccggtgttcgccgaaaccgtgaggaaatgcgccgaggccgtcgcagatatcctcgaaaagcctttgctcgacgtcattttcgacgccgacgagttggatggtgacaccgaatcgccgctgcggcacacgacctatgcgcaacccgcgctgttcgccgtcgaattgggcctggcccggctctggcagtcctggggcttcgaacccgacgtggtggtcggacacagtgtcgggcagtactcggccgcctgtgtggcgggtgtgttcgacgtcgaggacggcgcacgactgatcgccgagcgcggccgattgttcggcagcctgcccgcgggtggacgcatggcggcggttttcaccgcccccgagcgtgcagagcgcctcaccgacgagtaccccagcctgtcggttgccgcgtacaacggtgccaacaccgtattgtccggtcccgcaaaggatctcgaatccgccgtggccacattggtggccgacggtgtgcggtgcgactggctggacaccagccacgccttccactcggcactgctcgacccgattctcgacgatttcgagtcgttcgcccagaagctcgaataccgtgaaccgcaacgtatcctgatcgacaaccgcaccgggtccgcgctcgggcggagcaccaggctcgacggtgcctactggcggcggcacgcacgccagcctgtcgagttcgccaagagcgtccgcaccctcgccgacatgaactgcagggtgctgctggagatagggcccaggcccgtgctcaccgccaccgcgctggcagcatggcccgacccggccaccacgccgcaggtgctcgcgtcgctgcggcccaccacggccgatcaccggcagatcaccgaggccgtcgccgccgcctatgccgcgggacatctgcccgatttccgcgcgttccggcaaccggacgcgcgcaaactcgacctgcccacatatcccttcgagcatcgccagttctggttctcggacaaccgtgcgatcgaccgtgacacccagaccgcgagcgccggttccgtcacgcctcaccgcacacaggccgtccgacttctcgaagacggccagatcgaggaactcgccgccctcatcgacggcgagaccgtcgacgagcagaccctgcgcgtgctgaacaagctcgcggcgcgccacaaccagcagcggtccacacacgtcgaccctgatgcgcggtacgagatccgttgggacgcgatcgcttcggccgccgcgagcacgggcgcggcagccgactggatcctggtcggcgacgacaccgctgccgtcctggagttcgccgatgcgctcaccgcaggcggtcatcgccaccagatcgtcgcgctgcccggctccgacgccgacgaggcccagctggtcgatacgctgcgcgctgcgtcggccggtgagctgtacgtcgtgcacatcgcggcaggagacggtgccgctgccccgatgcgggacctgctgcgcgtgcagcaccgaacccttggtgggctgcggcgcctgttccgcgccgcgaacgacgcggaactgcgcagccccatctggttggtgacacgtgacgggcagcgggtcaccgacaccgacaccgtggtaccggaacagagttgcctgtggggattcggccgcgcggcggccctggaactcccgcacatctggggtggcctcgccgacctctccggtgcggacaccgagtggccgcggctcatcgcgcggatcacgtcaccgcgcgacggtgaggaccagatcgcgctgcgcggggacgccgtgtacgcaccccgactggtccgccggaccggcgagccgagcgacacccccctgccggtgcgggaggaccgcacctatctggtgacgggcggtctcggggcgatcgggctcgaggtcgccggctacctggcctcccacggtgccggtcatgtggtgctcaccagccgacgggaaccgggcgacgacgcgcgccggcgcctcgacggtctccgcgagcagtacgcatgtgacgtccgggtgatcaccgccgatgtcgccgacgcgcacgacgtcgcgcgcctcatgaccaccgtcgccgccgaactcccgccgctggccggcatcgtgcacgccgcgggcgagatcggcaccacgccgctgagcggcctcgacgacgccgaggtggatcgcgtcttcgccgggaaggtctggggcgcctggcatctgagcgaggccctgacagatcttcgcaccgatctcgacttcttcgtcagcacatcgtcgatcgcgtcggtgtggggcggattcgggcagaccgcctacggcgcggcgaacgcgtttctcgacggtctggcgtggcgactgcgcgagcggggtgtcaccgctgtcagcgtgaacttcgggccgtgggccgcgggcatggccgacgccgaatcacgtgcccgcctcgagcagcgaggcgtgcggacgttgtcgcccgccgaggccctcgcaggcatggccgacgtcgtggccggccccgttcagggagtcgtcgccaagatcgactggtcacgtttcctgccgctgtaccagcaggcaggccggcgcgcgttcctgtcggaactcgaaagcgaattgcccgcagcggcaaccggcgctgctgcgcccgcgacggtgcccgggaaaccaccgctcgtcgagcagttgaccaaagcgcctgtgcagcaacgcaaaagcctcatcacgaactacctgcgcaacgcggtcgccgaggtcacacgggtcgacgccgacgagatccgcgatgaggccgggttcttcgacctcgggatggactccctgatggccgtcgaactgcggcggcgcatcgagcagggtgtcggcaaggacattcccgtcacgctcgtgatggatcatccgcggttgtccgacgcggccgactacctgctggtcgaggtactcgggttgggcgagcagaccaacgtgcggcaggcgtcgaccgtgaccgcgcgaaccgacgatcccatcgcggtcgtcgcggtgtcctgccgcttccccggcgcacccgacccggagtcgttctgggagctgctcgccggtggtgtcgatgccatccgagaggtccccgaggaccgctgggacatcgacgagttctacgaccccgatccggacaccccgggcaagacctacacgcggttcggcggatttctagacggcatcgacggattcgatcccgagttcttcggcatctcaccgcgtgaggccgtgtggatcgagccgcagcagcgcctcatgctcgagacggtgtgggagggtctcgagcgggccggctacgcaccttcggacctccggggcagccgcacgggtgtcttcaccggtgttgccgccaacgagtacgcgcatctgctgtcggccgagtcgatcgacaagatcgagccgcacttcatcacgggcaatgcgctcaacgcgatctccggtcgcgtggcgttcgcgctgggcttcgaaggtcctgccgtcgcggtcgacaccgcgtgcagctcggctctggtcgcggtccatcaggcatgtcaggcactgcagtccggggactgcgatctggccctcgcgggaggcgtcaacgtgctgctgagccccgtcaccgtggtcgcggcctcacgcgcgcggatgctgtcgccggtgggccggtgcaagacgttcgacgcgtccgccgacgggtatgtgcgcagtgagggctgcggcgtcctggtgctcaagcgcctcagcgacgccgtgcgtgacggcgaccgggtgtgcgcggtgatccctgcgagctcggtgaaccaggacggcgcctccagcgggttgaccgtccccaacggcggtgcgcagcaacggctcatcgaggcgacgctggcccgcgccggactgagcggcgccgatgtggactacctcgaggcacacggcacgggtacgcccctcggtgacccgatcgaggtgcaggccgccgcggccgcgtacggcgccgggcgtgacgccgaccggccgctgctgatgggatcggtgaagaccaacatcgggcacaccgaatccgcctcgggtgcagcgggtctgatcaaggtggtgctgtcactgcaacacggggtgctgccgcagagcctgcacttcgagaatccgtcaccgcacatcccgtgggacgcattgccggtgcgggtgatcgacgaaccggtgccgtggcagaccaacggccgggtgcgccgtgccggtgtcagttcgttcgggttcaccggcaccaacgcgcacgtgctcgtcgaggaggccccgccgcaactgcacaccgccgacgctgcgcccgtggaaccggccgccaccgcggaggccgggaccgacgactcgccgcaggtgctggcgctgtcggcgcggtcgcccgaggcgctcgtggagttggcgcggcgctacgacgactggttgagcgcgacgcccggcctcgacatcgccgacgtgtgccacaccgcgggcgtgggccgttcgcacttcgaataccgcgcagccctggtggtcgactcggtcgagcgggcacgcgagggtctcgccgcgctcgccgaggggcgcactcctgcgggcgtggtgcgcggcgagcatgtgcaccgtccgacgacggcatggctgttcaccggacagggcagccagtacccgggcatggcccgcgaattgttcgacacgcaaccggttttcgcccagaccgttcggcagtgcgccgaggcggtcgccgacatgctgccgcgaccactgctggacgtgctgttcgcgaccgaccgcgaaaccgcggacctgctgcagcacacgtcgttcgcgcagcccgcgctgttcgcggtcgagatggggctagcgcggctgtggcagtcatgggccatcgaacccgatgtggtgctggggcacagcgtcggccagtacgccgcggcctgcgtggcgggcgtgttcagccttgacgacggcgcacgactgatggccgagcgcggccgcatgttcggcagccttcccgaaggcggccggatggtggccgtgttcgccgacgccgaacacgtcgagcaggtggccggtgagttcccccgggtgtcggtcggcgcctacaacggccccaacaccgtgctctcgggtcccggcgaggacctcgaagagatcgtcgagaggttcgaggacgaggggatccgctgcacgtggctggcgaccagccacgcgttccactccgaactgctcgatcccgtgctcgacgagttcgaggcctacgcggcccagttccagttcgcagcgccgacactgcctttggtgtgcaaccggaccggggccgtgctcaccgggcagaccccgctcgacgcgcagtactggcgcaggcattcccgccagcccgtgcagttcgccgagagtgtgcgcaccgtcgccgcgctcggctgctcggtgctgatggagatcggcccgcaacctgtgctgaccggggccgcggtacaggtgtggcccgaacacatggctgccccgcgggccgtcgcctcgctgcgcaagggcgtcgccgatcgccgacagatcgccgatgcgctggcgtcggcctacgtcggcggccaccgccccgacttcgccgcggtgcaccggcagccacgccgcccggtcgagttgcccacctatccgttccagcgccgcaggttctggccgaaggcctcgagcatcgccgtcgacggtgggaccgcggcgtcgggaatcctgggcagcggcaaggatctggcgtccggcgacaccatctacacgagccggctgtccgtcaaatcgcagccgtggctggccgatcacgtcatctacggcaccgtcgtcgtccccggcgcgacgtatgcggcgatggcgctcgccgcggtcggcacaccggcacgcctgaaggacgtgttcttctacgagccgatcatcctgcccgagaaggcctctcgcgaggtccagctgacgttgcaccctgccgacggtggcagcgtgctcagcttccagatccacagccgcccgtacggtgaacgtggtgccgactggtcgctgaacgccgagggcacggtcgacacggccggcagcgacccggacgccgccgcagacgagcagtcggatccggtcgacgaggtgatcgaacgcctcgagcgcatgcgtccgcaggacctcttcgagaccttcgccgacctggaactggcgtggggtcccacatggtcgggctcgctgaaatcgttgtggctcggcgacggtgaggccgtcggcgacatcctcgtcggcgccgaactcgccgagcaactcggctccgagcccatgcacccggtgctgatggacctgtgcacgggcgtcgcgttccccgcgttcccggcgctgctcgcggccgaacagggcgtcaacgacctgttcctgccgctgcgctacgggcaggtgacgctcgcggagaagatgccgcggcggttctactgccgggcgaggtggcaccgtagcgcgctcgacagcgaaacgcaggtgttcgaactcgaattcgtcgacctcggtgggcgagcgctcggcggcatccgcgagttcaccgtcaaacgtgcgccgcgcgaggcactgctgcgcggcctcgggggtgacgcgacccgcctgctctacacgctcgggtggcacgaggtgccgccgccaccatcggccgacgacgcggtcggcacgtggctgatcgccgggttcgacgagctggccgcgaaggtgcccggctgtgtccccatcgaccgcaacaccgatctgcaggtcctcggtgaggtgctgtcgcaggcgcacgctcgcggcgcgggattctcgggcgtcgtgtggcgttgcgcgacgcctggccggaaggaatcgggttctgcctccgccgaatccgcccgtctggaggccgagatcaccaatctgctcagcgcggtgcacgccgcgcaggggccgagcgtgaaactgcccggcggactctggatcgtcaccgaacacggcgtggcatgtgaatccggcgagccggtggacccggtgcaggccgcgctgtggggcttcggccgaacgacgatcaacgaggaaccggcgctgcactgccggctcgtcgactgcgacggatcttccgaggccgtcgacctgctggccgctctgctggccgcaccgggcatccaggaaccggaaatcgccgtgcggcagggcaaactgctggcgtcacggttgttgccgtgggcccgcagtggtcatctcacgctgccgcgcgggggcgacttcgtccttgcgcccaccgagcgcggcgcgatcgacaacctgcgcctcacggagacggacgtgccggagccggacgagggctacgtgcaggttcgggtcgaggccgcgggcctcaacttccgcgacgtgctcaacgtgctcggcctgtaccccggtgatccgggaccgatcggcggcgacttcgcaggcaccgtgacccaactgggggcaggtgtgagtggactcgaaatcggccaacgcgtgtacgggtcgatgcagggtgccttcgcgagccgcttcaacgtgcccgcccagttcctggcgccgattcccgacggcatcggcgcggtcgaggccgccacgatcccggccgcggcgctgacggtccggctcgcgttcgactgggcgcaactcaaacccggcgacaaggtgctcatccacgccgccagcggtggcgtggggctcgcggccatccagatggcgcagcagtccggcgccgaggtcttcgccaccgcgagcaccttcaagcgttcgaccctgcgcaggctgggcgtgaagtacgtctacgactctcggacgacggacttcgccgaccagatcctcgcggacaccaacggtgccggcgtggacgtggtgctcaacagcctcactagtgcgggtttcgtcgaggcgacgctgaaggccaccgcgcagggcggacgcttcgccgagatcgccaagcgcgacatctggtcgcatgagcagatggccgaggcccgccccgacatcgcctacgagatcgtcgcgctggacaccgtgatgttcaccgagcccgatcgcatccgcgacctgctcaccgaggtgtccgacgggatggcgcgcggcgagtggacaccactacccgccgagatctacccgatcaccgaggcccggtcggcgttccggcgcatgcaacaggcacggcacatcggcaagattgtgctgcaggtccccaaaccccttcagccgcagcaggaccggagctacctgatcaccggcggactcggggcgatcggcctgcacacggcggcgtatctggcgcagctcggcgcgggcgacatcgtgctgaccagccggcgcgaccccgacgagtcggcccagcgggcgatcgacgagatcaccgagcgccacaagtgccgcatccacacctatgcggccgatgtgggtgacgaggcacaggtggccgatctgctggcgcggatccgggccgagttgcctccactggccggaatcgcccatctggcgggcgtgctcgacgacgcgctgctgtcggcgcagaaccccgagcgcttccggaccacgttggcgcccaaggcctatggcgcgagccacctggaccgcctgacgcgcgacgacgacctggacttcttcatcgtgtcctcgtcggtgtcgagcctgttcggatcaccggggcaggccaactacgcaaccgccaatgcgatgctcgacggtctggtcgccaagcgccgtgcgcagggcctggtggccacgggcatcaacttcgggccgtggggtcagggcggcatggcgtcgtcggaggccgcgaccgcgaacatcaccgcccaaggtctgatcccgttggatccgtcggccgcgttgcacgcgctggccgaggtcatcgcgaacggctccgggcaggcgacggtgctcaaggccaactggcagcgcgcggcgaaggttctcggctcggcgcgcccgccgatcctggacctcgtcctgccgagcgcggccggggaggccacgggtgacagcgagctgctcaagcagttgatggagatcccgataccgcagcgcgccgggttcgtcaccgagttcctgcaacgcgaagtgcagaacttcctgcgtctcgcgtcgccgcccgcggcgtcgagccggttcctggacctcggcacggattcgctgatggccatcgagctgcgcaaccggctgcacagccagttcgggggcaagttcaccatcaacgcgaccgcggtgttcgactacccgaccatcggcgggctcgcggagtacctcgtgggtcagctgcccgacgccgaggcgcccgaaacggccactagtcagctcgacagcgggactcccgcccgggaagcgagcagcgctcttcgcgacggctaccggcaggcgggcgtgtcgggcagggtccggtcctacctcgacctgctggcggggctgtcggacttccgcgagcacttcgacggctccgacgggttctccctcgatctcgtggacatggccgacggtcccggagaggtcacggtgatctgctgcgcgggaacggcggcgatctccggtccgcacgagttcacccggctcgccggggcgctgcgcggaatcgctccggttcgggccgtgccccagcccggctacgaggagggcgaacctctgccgtcgtcgatggcggcggtggcggcggtgcaggccgatgcggtcatcaggacacagggggacaagccgttcgtggtggccggtcactccgcgggggcactgatggcctacgcgctggcgaccgaactgctcgatcgcgggcacccgccacgcggtgtcgtcctgatcgacgtctacccgcccggtcaccaggacgcgatgaacgcctggctggaggagctgaccgccacgctgttcgaccgcgagacggtgcggatggacgacaccaggctcaccgccctgggcgcctacgaccgcctcaccggtcagtggcgaccccgggaaaccgggctgccgacgctgctggtcagcgccggcgagccgatgggtccgtggcccgacgacagctggaagccgacgtggcccttcgagcacgacaccgtcgccgtccccggcgaccacttcacgatggtgcaggaacacgccgacgcgatcgcgcggcacatcgacgcctggctgggcggagggaattcatga

SEQ ID NO: 4 - protein, 3934 aa, synthetic construct
  REGION 1..3934: note = depicts the amino acid sequence encoding PKS14TE protein
MVSAEHPIEPTPSFAIIGYAARFPGAASAEEYWALLRDGREAISDVPKDRWDIEEFFDPDPSTPGKVVTRRAGFVDDVTGFDAPFFGMSAREVRLMDPQHRILMETAWRAVEHSGIAPTSLANSNTGVFVGLATHDYLGMASDELTYPEIEAYMAIGTSNAAAAGRISYRLGLQGPAVAVDTACSSSLVAIHQACQALRLNECDLALAGGANVLLTPATMITFSNAHMLAPDGRCKTFDAAADGYVRGEGCGVVVVKRLEDAVRDGDRIRAVIRGSAINQDGASGGLTVPNGVAQQRVIADALKRAGLRPADVGYLEAHGTGTSLGDPIEAQAAGEVLGAGRAPEEPLLIGSAKTNIGHLEAAAGIAGVIKVVMALENETLPKHLNFETPSPHIPWERLPIEVVRETVPWQRNGKPRIAGVSSFGFAGTNAHVILEEAPAVGTPATTEPVEDPRRYSVLPLSARTPEALVRLAGEYRDWLRAHPQARLADVSHTAGTARAHLEQRAALVVNSRESAIELLGALADDRPAPGLVRGESHEAPKTAWLFTGQGSQYPGMARELFDTEPVFAETVRKCAEAVADILEKPLLDVIFDADELDGDTESPLRHTTYAQPALFAVELGLARLWQSWGFEPDVVVGHSVGQYSAACVAGVFDVEDGARLIAERGRLFGSLPAGGRMAAVFTAPERAERLTDEYPSLSVAAYNGANTVLSGPAKDLESAVATLVADGVRCDWLDTSHAFHSALLDPILDDFESFAQKLEYREPQRILIDNRTGSALGRSTRLDGAYWRRHARQPVEFAKSVRTLADMNCRVLLEIGPRPVLTATALAAWPDPATTPQVLASLRPTTADHRQITEAVAAAYAAGHLPDFRAFRQPDARKLDLPTYPFEHRQFWFSDNRAIDRDTQTASAGSVTPHRTQAVRLLEDGQIEELAALIDGETVDEQTLRVLNKLAARHNQQRSTHVDPDARYEIRWDAIASAAASTGAAADWILVGDDTAAVLEFADALTAGGHRHQIVALPGSDADEAQLVDTLRAASAGELYVVHIAAGDGAAAPMRDLLRVQHRTLGGLRRLFRAANDAELRSPIWLVTRDGQRVTDTDTVVPEQSCLWGFGRAAALELPHIWGGLADLSGADTEWPRLIARITSPRDGEDQIALRGDAVYAPRLVRRTGEPSDTPLPVREDRTYLVTGGLGAIGLEVAGYLASHGAGHVVLTSRREPGDDARRRLDGLREQYACDVRVITADVADAHDVARLMTTVAAELPPLAGIVHAAGEIGTTPLSGLDDAEVDRVFAGKVWGAWHLSEALTDLRTDLDFFVSTSSIASVWGGFGQTAYGAANAFLDGLAWRLRERGVTAVSVNFGPWAAGMADAESRARLEQRGVRTLSPAEALAGMADVVAGPVQGVVAKIDWSRFLPLYQQAGRRAFLSELESELPAAATGAAAPATVPGKPPLVEQLTKAPVQQRKSLITNYLRNAVAEVTRVDADEIRDEAGFFDLGMDSLMAVELRRRIEQGVGKDIPVTLVMDHPRLSDAADYLLVEVLGLGEQTNVRQASTVTARTDDPIAVVAVSCRFPGAPDPESFWELLAGGVDAIREVPEDRWDIDEFYDPDPDTPGKTYTRFGGFLDGIDGFDPEFFGISPREAVWIEPQQRLMLETVWEGLERAGYAPSDLRGSRTGVFTGVAANEYAHLLSAESIDKIEPHFITGNALNAISGRVAFALGFEGPAVAVDTACSSALVAVHQACQALQSGDCDLALAGGVNVLLSPVTVVAASRARMLSPVGRCKTFDASADGYVRSEGCGVLVLKRLSDAVRDGDRVCAVIPASSVNQDGASSGLTVPNGGAQQRLIEATLARAGLSGADVDYLEAHGTGTPLGDPIEVQAAAAAYGAGRDADRPLLMGSVKTNIGHTESASGAAGLIKVVLSLQHGVLPQSLHFENPSPHIPWDALPVRVIDEPVPWQTNGRVRRAGVSSFGFTGTNAHVLVEEAPPQLHTADAAPVEPAATAEAGTDDSPQVLALSARSPEALVELARRYDDWLSATPGLDIADVCHTAGVGRSHFEYRAALVVDSVERAREGLAALAEGRTPAGVVRGEHVHRPTTAWLFTGQGSQYPGMARELFDTQPVFAQTVRQCAEAVADMLPRPLLDVLFATDRETADLLQHTSFAQPALFAVEMGLARLWQSWAIEPDVVLGHSVGQYAAACVAGVFSLDDGARLMAERGRMFGSLPEGGRMVAVFADAEHVEQVAGEFPRVSVGAYNGPNTVLSGPGEDLEEIVERFEDEGIRCTWLATSHAFHSELLDPVLDEFEAYAAQFQFAAPTLPLVCNRTGAVLTGQTPLDAQYWRRHSRQPVQFAESVRTVAALGCSVLMEIGPQPVLTGAAVQVWPEHMAAPRAVASLRKGVADRRQIADALASAYVGGHRPDFAAVHRQPRRPVELPTYPFQRRRFWPKASSIAVDGGTAASGILGSGKDLASGDTIYTSRLSVKSQPWLADHVIYGTVVVPGATYAAMALAAVGTPARLKDVFFYEPIILPEKASREVQLTLHPADGGSVLSFQIHSRPYGERGADWSLNAEGTVDTAGSDPDAAADEQSDPVDEVIERLERMRPQDLFETFADLELAWGPTWSGSLKSLWLGDGEAVGDILVGAELAEQLGSEPMHPVLMDLCTGVAFPAFPALLAAEQGVNDLFLPLRYGQVTLAEKMPRRFYCRARWHRSALDSETQVFELEFVDLGGRALGGIREFTVKRAPREALLRGLGGDATRLLYTLGWHEVPPPPSADDAVGTWLIAGFDELAAKVPGCVPIDRNTDLQVLGEVLSQAHARGAGFSGVVWRCATPGRKESGSASAESARLEAEITNLLSAVHAAQGPSVKLPGGLWIVTEHGVACESGEPVDPVQAALWGFGRTTINEEPALHCRLVDCDGSSEAVDLLAALLAAPGIQEPEIAVRQGKLLASRLLPWARSGHLTLPRGGDFVLAPTERGAIDNLRLTETDVPEPDEGYVQVRVEAAGLNFRDVLNVLGLYPGDPGPIGGDFAGTVTQLGAGVSGLEIGQRVYGSMQGAFASRFNVPAQFLAPIPDGIGAVEAATIPAAALTVRLAFDWAQLKPGDKVLIHAASGGVGLAAIQMAQQSGAEVFATASTFKRSTLRRLGVKYVYDSRTTDFADQILADTNGAGVDVVLNSLTSAGFVEATLKATAQGGRFAEIAKRDIWSHEQMAEARPDIAYEIVALDTVMFTEPDRIRDLLTEVSDGMARGEWTPLPAEIYPITEARSAFRRMQQARHIGKIVLQVPKPLQPQQDRSYLITGGLGAIGLHTAAYLAQLGAGDIVLTSRRDPDESAQRAIDEITERHKCRIHTYAADVGDEAQVADLLARIRAELPPLAGIAHLAGVLDDALLSAQNPERFRTTLAPKAYGASHLDRLTRDDDLDFFIVSSSVSSLFGSPGQANYATANAMLDGLVAKRRAQGLVATGINFGPWGQGGMASSEAATANITAQGLIPLDPSAALHALAEVIANGSGQATVLKANWQRAAKVLGSARPPILDLVLPSAAGEATGDSELLKQLMEIPIPQRAGFVTEFLQREVQNFLRLASPPAASSRFLDLGTDSLMAIELRNRLHSQFGGKFTINATAVFDYPTIGGLAEYLVGQLPDAEAPETATSQLDSGTPAREASSALRDGYRQAGVSGRVRSYLDLLAGLSDFREHFDGSDGFSLDLVDMADGPGEVTVICCAGTAAISGPHEFTRLAGALRGIAPVRAVPQPGYEEGEPLPSSMAAVAAVQADAVIRTQGDKPFVVAGHSAGALMAYALATELLDRGHPPRGVVLIDVYPPGHQDAMNAWLEELTATLFDRETVRMDDTRLTALGAYDRLTGQWRPRETGLPTLLVSAGEPMGPWPDDSWKPTWPFEHDTVAVPGDHFTMVQEHADAIARHIDAWLGGGNS

SEQ ID NO: 5 - DNA, 17148 bp, synthetic construct
  misc_feature 1..17148: note = depicts the nucleic acid sequence of recombinant vector pSSRi11
  source 1..17148: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatggtctctgccgaacatccgatcgagccgaccccgagcttcgcaatcatcggctacgccgcacgttttcccggcgcggcgagcgccgaggaatactgggcgttgttgcgggacgggcgtgaagcgatatccgatgttccgaaagaccgctgggacatcgaggagttcttcgacccggacccgtcgacaccgggcaaggtcgtcacgcgccgcgcggggttcgtcgatgacgtaacgggtttcgacgcgccgttcttcggtatgtccgcccgcgaggtacgtctgatggacccgcagcaccggattctcatggaaaccgcgtggcgcgcggtcgaacactcgggaatcgcgccgacgtcgctggccaacagcaacacgggcgtcttcgtcggcctggccacccacgactacctcggtatggcctccgacgaactcacctaccccgagatcgaggcctacatggccatcggaacctccaatgccgctgcggccggacggatcagttaccgcctgggattgcagggaccggcggtggcggtcgacaccgcgtgcagttcgtcgctggtggcgattcaccaggcgtgtcaggcacttcgcctgaacgagtgcgatctcgcgttggccggcggggcgaacgtcctgctcaccccggccaccatgatcacgttctccaacgcgcacatgctggctccggacggccggtgcaagaccttcgacgcggcggccgacggctacgtgcgcggcgagggctgcggtgtcgtcgtggtcaagcgcctcgaggacgcggtgcgcgacggcgaccggatccgcgcggtgatccgcggaagcgcaatcaaccaggacggggcgtcgggcgggctgaccgtgccgaacggcgttgctcagcaacgggttatcgccgacgcgttgaagcgcgccggcctgcgaccggccgatgtcggttatctcgaggcgcacggcaccggaacctcacttggcgaccccatcgaggcccaggccgcgggcgaggtgctcggggccggacgcgcacctgaagaaccgctgctgatcggctcggcgaagaccaacatcggccatctggaagcggccgcgggcatcgccggtgtcatcaaggtggtcatggcgctcgagaacgagacgttgcccaagcacctcaatttcgagactccgtcgccccacatcccgtgggagcgtctacccatcgaggtggtgagagagaccgttccctggcagcgcaacggcaagccgcggatcgccggggtgagctcattcgggtttgccgggactaacgcgcacgtcatcctcgaagaggcgcccgccgtcggaacaccggcgacgacagagcctgtcgaggatccgaggcgctacagcgtccttccgctgtcggcacgcacgcccgaggcgttggtccgactcgccggtgagtaccgcgactggctgcgcgcccacccgcaggcccgcctggccgacgtgtcccacaccgcgggaaccgcacgtgcccacctggagcagcgtgccgcgttggtggtcaactcgcgggaatcggccatcgaactgctcggcgcgctcgccgacgaccgtccggcgcccggcctggtccgcggtgaatcccacgaggcacccaagaccgcgtggttgttcaccggccagggcagccagtaccccggaatggcgcgtgagttgttcgacaccgaaccggtgttcgccgaaaccgtgaggaaatgcgccgaggccgtcgcagatatcctcgaaaagcctttgctcgacgtcattttcgacgccgacgagttggatggtgacaccgaatcgccgctgcggcacacgacctatgcgcaacccgcgctgttcgccgtcgaattgggcctggcccggctctggcagtcctggggcttcgaacccgacgtggtggtcggacacagtgtcgggcagtactcggccgcctgtgtggcgggtgtgttcgacgtcgaggacggcgcacgactgatcgccgagcgcggccgattgttcggcagcctgcccgcgggtggacgcatggcggcggttttcaccgcccccgagcgtgcagagcgcctcaccgacgagtaccccagcctgtcggttgccgcgtacaacggtgccaacaccgtattgtccggtcccgcaaaggatctcgaatccgccgtggccacattggtggccgacggtgtgcggtgcgactggctggacaccagccacgccttccactcggcactgctcgacccgattctcgacgatttcgagtcgttcgcccagaagctcgaataccgtgaaccgcaacgtatcctgatcgacaaccgcaccgggtccgcgctcgggcggagcaccaggctcgacggtgcctactggcggcggcacgcacgccagcctgtcgagttcgccaagagcgtccgcaccctcgccgacatgaactgcagggtgctgctggagatagggcccaggcccgtgctcaccgccaccgcgctggcagcatggcccgacccggccaccacgccgcaggtgctcgcgtcgctgcggcccaccacggccgatcaccggcagatcaccgaggccgtcgccgccgcctatgccgcgggacatctgcccgatttccgcgcgttccggcaaccggacgcgcgcaaactcgacctgcccacatatcccttcgagcatcgccagttctggttctcggacaaccgtgcgatcgaccgtgacacccagaccgcgagcgccggttccgtcacgcctcaccgcacacaggccgtccgacttctcgaagacggccagatcgaggaactcgccgccctcatcgacggcgagaccgtcgacgagcagaccctgcgcgtgctgaacaagctcgcggcgcgccacaaccagcagcggtccacacacgtcgaccctgatgcgcggtacgagatccgttgggacgcgatcgcttcggccgccgcgagcacgggcgcggcagccgactggatcctggtcggcgacgacaccgctgccgtcctggagttcgccgatgcgctcaccgcaggcggtcatcgccaccagatcgtcgcgctgcccggctccgacgccgacgaggcccagctggtcgatacgctgcgcgctgcgtcggccggtgagctgtacgtcgtgcacatcgcggcaggagacggtgccgctgccccgatgcgggacctgctgcgcgtgcagcaccgaacccttggtgggctgcggcgcctgttccgcgccgcgaacgacgcggaactgcgcagccccatctggttggtgacacgtgacgggcagcgggtcaccgacaccgacaccgtggtaccggaacagagttgcctgtggggattcggccgcgcggcggccctggaactcccgcacatctggggtggcctcgccgacctctccggtgcggacaccgagtggccgcggctcatcgcgcggatcacgtcaccgcgcgacggtgaggaccagatcgcgctgcgcggggacgccgtgtacgcaccccgactggtccgccggaccggcgagccgagcgacacccccctgccggtgcgggaggaccgcacctatctggtgacgggcggtctcggggcgatcgggctcgaggtcgccggctacctggcctcccacggtgccggtcatgtggtgctcaccagccgacgggaaccgggcgacgacgcgcgccggcgcctcgacggtctccgcgagcagtacgcatgtgacgtccgggtgatcaccgccgatgtcgccgacgcgcacgacgtcgcgcgcctcatgaccaccgtcgccgccgaactcccgccgctggccggcatcgtgcacgccgcgggcgagatcggcaccacgccgctgagcggcctcgacgacgccgaggtggatcgcgtcttcgccgggaaggtctggggcgcctggcatctgagcgaggccctgacagatcttcgcaccgatctcgacttcttcgtcagcacatcgtcgatcgcgtcggtgtggggcggattcgggcagaccgcctacggcgcggcgaacgcgtttctcgacggtctggcgtggcgactgcgcgagcggggtgtcaccgctgtcagcgtgaacttcgggccgtgggccgcgggcatggccgacgccgaatcacgtgcccgcctcgagcagcgaggcgtgcggacgttgtcgcccgccgaggccctcgcaggcatggccgacgtcgtggccggccccgttcagggagtcgtcgccaagatcgactggtcacgtttcctgccgctgtaccagcaggcaggccggcgcgcgttcctgtcggaactcgaaagcgaattgcccgcagcggcaaccggcgctgctgcgcccgcgacggtgcccgggaaaccaccgctcgtcgagcagttgaccaaagcgcctgtgcagcaacgcaaaagcctcatcacgaactacctgcgcaacgcggtcgccgaggtcacacgggtcgacgccgacgagatccgcgatgaggccgggttcttcgacctcgggatggactccctgatggccgtcgaactgcggcggcgcatcgagcagggtgtcggcaaggacattcccgtcacgctcgtgatggatcatccgcggttgtccgacgcggccgactacctgctggtcgaggtactcgggttgggcgagcagaccaacgtgcggcaggcgtcgaccgtgaccgcgcgaaccgacgatcccatcgcggtcgtcgcggtgtcctgccgcttccccggcgcacccgacccggagtcgttctgggagctgctcgccggtggtgtcgatgccatccgagaggtccccgaggaccgctgggacatcgacgagttctacgaccccgatccggacaccccgggcaagacctacacgcggttcggcggatttctagacggcatcgacggattcgatcccgagttcttcggcatctcaccgcgtgaggccgtgtggatcgagccgcagcagcgcctcatgctcgagacggtgtgggagggtctcgagcgggccggctacgcaccttcggacctccggggcagccgcacgggtgtcttcaccggtgttgccgccaacgagtacgcgcatctgctgtcggccgagtcgatcgacaagatcgagccgcacttcatcacgggcaatgcgctcaacgcgatctccggtcgcgtggcgttcgcgctgggcttcgaaggtcctgccgtcgcggtcgacaccgcgtgcagctcggctctggtcgcggtccatcaggcatgtcaggcactgcagtccggggactgcgatctggccctcgcgggaggcgtcaacgtgctgctgagccccgtcaccgtggtcgcggcctcacgcgcgcggatgctgtcgccggtgggccggtgcaagacgttcgacgcgtccgccgacgggtatgtgcgcagtgagggctgcggcgtcctggtgctcaagcgcctcagcgacgccgtgcgtgacggcgaccgggtgtgcgcggtgatccctgcgagctcggtgaaccaggacggcgcctccagcgggttgaccgtccccaacggcggtgcgcagcaacggctcatcgaggcgacgctggcccgcgccggactgagcggcgccgatgtggactacctcgaggcacacggcacgggtacgcccctcggtgacccgatcgaggtgcaggccgccgcggccgcgtacggcgccgggcgtgacgccgaccggccgctgctgatgggatcggtgaagaccaacatcgggcacaccgaatccgcctcgggtgcagcgggtctgatcaaggtggtgctgtcactgcaacacggggtgctgccgcagagcctgcacttcgagaatccgtcaccgcacatcccgtgggacgcattgccggtgcgggtgatcgacgaaccggtgccgtggcagaccaacggccgggtgcgccgtgccggtgtcagttcgttcgggttcaccggcaccaacgcgcacgtgctcgtcgaggaggccccgccgcaactgcacaccgccgacgctgcgcccgtggaaccggccgccaccgcggaggccgggaccgacgactcgccgcaggtgctggcgctgtcggcgcggtcgcccgaggcgctcgtggagttggcgcggcgctacgacgactggttgagcgcgacgcccggcctcgacatcgccgacgtgtgccacaccgcgggcgtgggccgttcgcacttcgaataccgcgcagccctggtggtcgactcggtcgagcgggcacgcgagggtctcgccgcgctcgccgaggggcgcactcctgcgggcgtggtgcgcggcgagcatgtgcaccgtccgacgacggcatggctgttcaccggacagggcagccagtacccgggcatggcccgcgaattgttcgacacgcaaccggttttcgcccagaccgttcggcagtgcgccgaggcggtcgccgacatgctgccgcgaccactgctggacgtgctgttcgcgaccgaccgcgaaaccgcggacctgctgcagcacacgtcgttcgcgcagcccgcgctgttcgcggtcgagatggggctagcgcggctgtggcagtcatgggccatcgaacccgatgtggtgctggggcacagcgtcggccagtacgccgcggcctgcgtggcgggcgtgttcagccttgacgacggcgcacgactgatggccgagcgcggccgcatgttcggcagccttcccgaaggcggccggatggtggccgtgttcgccgacgccgaacacgtcgagcaggtggccggtgagttcccccgggtgtcggtcggcgcctacaacggccccaacaccgtgctctcgggtcccggcgaggacctcgaagagatcgtcgagaggttcgaggacgaggggatccgctgcacgtggctggcgaccagccacgcgttccactccgaactgctcgatcccgtgctcgacgagttcgaggcctacgcggcccagttccagttcgcagcgccgacactgcctttggtgtgcaaccggaccggggccgtgctcaccgggcagaccccgctcgacgcgcagtactggcgcaggcattcccgccagcccgtgcagttcgccgagagtgtgcgcaccgtcgccgcgctcggctgctcggtgctgatggagatcggcccgcaacctgtgctgaccggggccgcggtacaggtgtggcccgaacacatggctgccccgcgggccgtcgcctcgctgcgcaagggcgtcgccgatcgccgacagatcgccgatgcgctggcgtcggcctacgtcggcggccaccgccccgacttcgccgcggtgcaccggcagccacgccgcccggtcgagttgcccacctatccgttccagcgccgcaggttctggccgaaggcctcgagcatcgccgtcgacggtgggaccgcggcgtcgggaatcctgggcagcggcaaggatctggcgtccggcgacaccatctacacgagccggctgtccgtcaaatcgcagccgtggctggccgatcacgtcatctacggcaccgtcgtcgtccccggcgcgacgtatgcggcgatggcgctcgccgcggtcggcacaccggcacgcctgaaggacgtgttcttctacgagccgatcatcctgcccgagaaggcctctcgcgaggtccagctgacgttgcaccctgccgacggtggcagcgtgctcagcttccagatccacagccgcccgtacggtgaacgtggtgccgactggtcgctgaacgccgagggcacggtcgacacggccggcagcgacccggacgccgccgcagacgagcagtcggatccggtcgacgaggtgatcgaacgcctcgagcgcatgcgtccgcaggacctcttcgagaccttcgccgacctggaactggcgtggggtcccacatggtcgggctcgctgaaatcgttgtggctcggcgacggtgaggccgtcggcgacatcctcgtcggcgccgaactcgccgagcaactcggctccgagcccatgcacccggtgctgatggacctgtgcacgggcgtcgcgttccccgcgttcccggcgctgctcgcggccgaacagggcgtcaacgacctgttcctgccgctgcgctacgggcaggtgacgctcgcggagaagatgccgcggcggttctactgccgggcgaggtggcaccgtagcgcgctcgacagcgaaacgcaggtgttcgaactcgaattcgtcgacctcggtgggcgagcgctcggcggcatccgcgagttcaccgtcaaacgtgcgccgcgcgaggcactgctgcgcggcctcgggggtgacgcgacccgcctgctctacacgctcgggtggcacgaggtgccgccgccaccatcggccgacgacgcggtcggcacgtggctgatcgccgggttcgacgagctggccgcgaaggtgcccggctgtgtccccatcgaccgcaacaccgatctgcaggtcctcggtgaggtgctgtcgcaggcgcacgctcgcggcgcgggattctcgggcgtcgtgtggcgttgcgcgacgcctggccggaaggaatcgggttctgcctccgccgaatccgcccgtctggaggccgagatcaccaatctgctcagcgcggtgcacgccgcgcaggggccgagcgtgaaactgcccggcggactctggatcgtcaccgaacacggcgtggcatgtgaatccggcgagccggtggacccggtgcaggccgcgctgtggggcttcggccgaacgacgatcaacgaggaaccggcgctgcactgccggctcgtcgactgcgacggatcttccgaggccgtcgacctgctggccgctctgctggccgcaccgggcatccaggaaccggaaatcgccgtgcggcagggcaaactgctggcgtcacggttgttgccgtgggcccgcagtggtcatctcacgctgccgcgcgggggcgacttcgtccttgcgcccaccgagcgcggcgcgatcgacaacctgcgcctcacggagacggacgtgccggagccggacgagggctacgtgcaggttcgggtcgaggccgcgggcctcaacttccgcgacgtgctcaacgtgctcggcctgtaccccggtgatccgggaccgatcggcggcgacttcgcaggcaccgtgacccaactgggggcaggtgtgagtggactcgaaatcggccaacgcgtgtacgggtcgatgcagggtgccttcgcgagccgcttcaacgtgcccgcccagttcctggcgccgattcccgacggcatcggcgcggtcgaggccgccacgatcccggccgcggcgctgacggtccggctcgcgttcgactgggcgcaactcaaacccggcgacaaggtgctcatccacgccgccagcggtggcgtggggctcgcggccatccagatggcgcagcagtccggcgccgaggtcttcgccaccgcgagcaccttcaagcgttcgaccctgcgcaggctgggcgtgaagtacgtctacgactctcggacgacggacttcgccgaccagatcctcgcggacaccaacggtgccggcgtggacgtggtgctcaacagcctcactagtgcgggtttcgtcgaggcgacgctgaaggccaccgcgcagggcggacgcttcgccgagatcgccaagcgcgacatctggtcgcatgagcagatggccgaggcccgccccgacatcgcctacgagatcgtcgcgctggacaccgtgatgttcaccgagcccgatcgcatccgcgacctgctcaccgaggtgtccgacgggatggcgcgcggcgagtggacaccactacccgccgagatctacccgatcaccgaggcccggtcggcgttccggcgcatgcaacaggcacggcacatcggcaagattgtgctgcaggtccccaaaccccttcagccgcagcaggaccggagctacctgatcaccggcggactcggggcgatcggcctgcacacggcggcgtatctggcgcagctcggcgcgggcgacatcgtgctgaccagccggcgcgaccccgacgagtcggcccagcgggcgatcgacgagatcaccgagcgccacaagtgccgcatccacacctatgcggccgatgtgggtgacgaggcacaggtggccgatctgctggcgcggatccgggccgagttgcctccactggccggaatcgcccatctggcgggcgtgctcgacgacgcgctgctgtcggcgcagaaccccgagcgcttccggaccacgttggcgcccaaggcctatggcgcgagccacctggaccgcctgacgcgcgacgacgacctggacttcttcatcgtgtcctcgtcggtgtcgagcctgttcggatcaccggggcaggccaactacgcaaccgccaatgcgatgctcgacggtctggtcgccaagcgccgtgcgcagggcctggtggccacgggcatcaacttcgggccgtggggtcagggcggcatggcgtcgtcggaggccgcgaccgcgaacatcaccgcccaaggtctgatcccgttggatccgtcggccgcgttgcacgcgctggccgaggtcatcgcgaacggctccgggcaggcgacggtgctcaaggccaactggcagcgcgcggcgaaggttctcggctcggcgcgcccgccgatcctggacctcgtcctgccgagcgcggccggggaggccacgggtgacagcgagctgctcaagcagttgatggagatcccgataccgcagcgcgccgggttcgtcaccgagttcctgcaacgcgaagtgcagaacttcctgcgtctcgcgtcgccgcccgcggcgtcgagccggttcctggacctcggcacggattcgctgatggccatcgagctgcgcaaccggctgcacagccagttcgggggcaagttcaccatcaacgcgaccgcggtgttcgactacccgaccatcggcgggctcgcggagtacctcactagtcagctcgacagcgggactcccgcccgggaagcgagcagcgctcttcgcgacggctaccggcaggcgggcgtgtcgggcagggtccggtcctacctcgacctgctggcggggctgtcggacttccgcgagcacttcgacggctccgacgggttctccctcgatctcgtggacatggccgacggtcccggagaggtcacggtgatctgctgcgcgggaacggcggcgatctccggtccgcacgagttcacccggctcgccggggcgctgcgcggaatcgctccggttcgggccgtgccccagcccggctacgaggagggcgaacctctgccgtcgtcgatggcggcggtggcggcggtgcaggccgatgcggtcatcaggacacagggggacaagccgttcgtggtggccggtcactccgcgggggcactgatggcctacgcgctggcgaccgaactgctcgatcgcgggcacccgccacgcggtgtcgtcctgatcgacgtctacccgcccggtcaccaggacgcgatgaacgcctggctggaggagctgaccgccacgctgttcgaccgcgagacggtgcggatggacgacaccaggctcaccgccctgggcgcctacgaccgcctcaccggtcagtggcgaccccgggaaaccgggctgccgacgctgctggtcagcgccggcgagccgatgggtccgtggcccgacgacagctggaagccgacgtggcccttcgagcacgacaccgtcgccgtccccggcgaccacttcacgatggtgcaggaacacgccgacgcgatcgcgcggcacatcgacgcctggctgggcggagggaattcatgaaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 6 - DNA, 17187 bp, synthetic construct
  misc_feature 1..17187: note = depicts the nucleic acid sequence of recombinant vector pSSRi15
  source 1..17187: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatggtctctgccgaacatccgatcgagccgaccccgagcttcgcaatcatcggctacgccgcacgttttcccggcgcggcgagcgccgaggaatactgggcgttgttgcgggacgggcgtgaagcgatatccgatgttccgaaagaccgctgggacatcgaggagttcttcgacccggacccgtcgacaccgggcaaggtcgtcacgcgccgcgcggggttcgtcgatgacgtaacgggtttcgacgcgccgttcttcggtatgtccgcccgcgaggtacgtctgatggacccgcagcaccggattctcatggaaaccgcgtggcgcgcggtcgaacactcgggaatcgcgccgacgtcgctggccaacagcaacacgggcgtcttcgtcggcctggccacccacgactacctcggtatggcctccgacgaactcacctaccccgagatcgaggcctacatggccatcggaacctccaatgccgctgcggccggacggatcagttaccgcctgggattgcagggaccggcggtggcggtcgacaccgcgtgcagttcgtcgctggtggcgattcaccaggcgtgtcaggcacttcgcctgaacgagtgcgatctcgcgttggccggcggggcgaacgtcctgctcaccccggccaccatgatcacgttctccaacgcgcacatgctggctccggacggccggtgcaagaccttcgacgcggcggccgacggctacgtgcgcggcgagggctgcggtgtcgtcgtggtcaagcgcctcgaggacgcggtgcgcgacggcgaccggatccgcgcggtgatccgcggaagcgcaatcaaccaggacggggcgtcgggcgggctgaccgtgccgaacggcgttgctcagcaacgggttatcgccgacgcgttgaagcgcgccggcctgcgaccggccgatgtcggttatctcgaggcgcacggcaccggaacctcacttggcgaccccatcgaggcccaggccgcgggcgaggtgctcggggccggacgcgcacctgaagaaccgctgctgatcggctcggcgaagaccaacatcggccatctggaagcggccgcgggcatcgccggtgtcatcaaggtggtcatggcgctcgagaacgagacgttgcccaagcacctcaatttcgagactccgtcgccccacatcccgtgggagcgtctacccatcgaggtggtgagagagaccgttccctggcagcgcaacggcaagccgcggatcgccggggtgagctcattcgggtttgccgggactaacgcgcacgtcatcctcgaagaggcgcccgccgtcggaacaccggcgacgacagagcctgtcgaggatccgaggcgctacagcgtccttccgctgtcggcacgcacgcccgaggcgttggtccgactcgccggtgagtaccgcgactggctgcgcgcccacccgcaggcccgcctggccgacgtgtcccacaccgcgggaaccgcacgtgcccacctggagcagcgtgccgcgttggtggtcaactcgcgggaatcggccatcgaactgctcggcgcgctcgccgacgaccgtccggcgcccggcctggtccgcggtgaatcccacgaggcacccaagaccgcgtggttgttcaccggccagggcagccagtaccccggaatggcgcgtgagttgttcgacaccgaaccggtgttcgccgaaaccgtgaggaaatgcgccgaggccgtcgcagatatcctcgaaaagcctttgctcgacgtcattttcgacgccgacgagttggatggtgacaccgaatcgccgctgcggcacacgacctatgcgcaacccgcgctgttcgccgtcgaattgggcctggcccggctctggcagtcctggggcttcgaacccgacgtggtggtcggacacagtgtcgggcagtactcggccgcctgtgtggcgggtgtgttcgacgtcgaggacggcgcacgactgatcgccgagcgcggccgattgttcggcagcctgcccgcgggtggacgcatggcggcggttttcaccgcccccgagcgtgcagagcgcctcaccgacgagtaccccagcctgtcggttgccgcgtacaacggtgccaacaccgtattgtccggtcccgcaaaggatctcgaatccgccgtggccacattggtggccgacggtgtgcggtgcgactggctggacaccagccacgccttccactcggcactgctcgacccgattctcgacgatttcgagtcgttcgcccagaagctcgaataccgtgaaccgcaacgtatcctgatcgacaaccgcaccgggtccgcgctcgggcggagcaccaggctcgacggtgcctactggcggcggcacgcacgccagcctgtcgagttcgccaagagcgtccgcaccctcgccgacatgaactgcagggtgctgctggagatagggcccaggcccgtgctcaccgccaccgcgctggcagcatggcccgacccggccaccacgccgcaggtgctcgcgtcgctgcggcccaccacggccgatcaccggcagatcaccgaggccgtcgccgccgcctatgccgcgggacatctgcccgatttccgcgcgttccggcaaccggacgcgcgcaaactcgacctgcccacatatcccttcgagcatcgccagttctggttctcggacaaccgtgcgatcgaccgtgacacccagaccgcgagcgccggttccgtcacgcctcaccgcacacaggccgtccgacttctcgaagacggccagatcgaggaactcgccgccctcatcgacggcgagaccgtcgacgagcagaccctgcgcgtgctgaacaagctcgcggcgcgccacaaccagcagcggtccacacacgtcgaccctgatgcgcggtacgagatccgttgggacgcgatcgcttcggccgccgcgagcacgggcgcggcagccgactggatcctggtcggcgacgacaccgctgccgtcctggagttcgccgatgcgctcaccgcaggcggtcatcgccaccagatcgtcgcgctgcccggctccgacgccgacgaggcccagctggtcgatacgctgcgcgctgcgtcggccggtgagctgtacgtcgtgcacatcgcggcaggagacggtgccgctgccccgatgcgggacctgctgcgcgtgcagcaccgaacccttggtgggctgcggcgcctgttccgcgccgcgaacgacgcggaactgcgcagccccatctggttggtgacacgtgacgggcagcgggtcaccgacaccgacaccgtggtaccggaacagagttgcctgtggggattcggccgcgcggcggccctggaactcccgcacatctggggtggcctcgccgacctctccggtgcggacaccgagtggccgcggctcatcgcgcggatcacgtcaccgcgcgacggtgaggaccagatcgcgctgcgcggggacgccgtgtacgcaccccgactggtccgccggaccggcgagccgagcgacacccccctgccggtgcgggaggaccgcacctatctggtgacgggcggtctcggggcgatcgggctcgaggtcgccggctacctggcctcccacggtgccggtcatgtggtgctcaccagccgacgggaaccgggcgacgacgcgcgccggcgcctcgacggtctccgcgagcagtacgcatgtgacgtccgggtgatcaccgccgatgtcgccgacgcgcacgacgtcgcgcgcctcatgaccaccgtcgccgccgaactcccgccgctggccggcatcgtgcacgccgcgggcgagatcggcaccacgccgctgagcggcctcgacgacgccgaggtggatcgcgtcttcgccgggaaggtctggggcgcctggcatctgagcgaggccctgacagatcttcgcaccgatctcgacttcttcgtcagcacatcgtcgatcgcgtcggtgtggggcggattcgggcagaccgcctacggcgcggcgaacgcgtttctcgacggtctggcgtggcgactgcgcgagcggggtgtcaccgctgtcagcgtgaacttcgggccgtgggccgcgggcatggccgacgccgaatcacgtgcccgcctcgagcagcgaggcgtgcggacgttgtcgcccgccgaggccctcgcaggcatggccgacgtcgtggccggccccgttcagggagtcgtcgccaagatcgactggtcacgtttcctgccgctgtaccagcaggcaggccggcgcgcgttcctgtcggaactcgaaagcgaattgcccgcagcggcaaccggcgctgctgcgcccgcgacggtgcccgggaaaccaccgctcgtcgagcagttgaccaaagcgcctgtgcagcaacgcaaaagcctcatcacgaactacctgcgcaacgcggtcgccgaggtcacacgggtcgacgccgacgagatccgcgatgaggccgggttcttcgacctcgggatggactccctgatggccgtcgaactgcggcggcgcatcgagcagggtgtcggcaaggacattcccgtcacgctcgtgatggatcatccgcggttgtccgacgcggccgactacctgctggtcgaggtactcgggttgggcgagcagaccaacgtgcggcaggcgtcgaccgtgaccgcgcgaaccgacgatcccatcgcggtcgtcgcggtgtcctgccgcttccccggcgcacccgacccggagtcgttctgggagctgctcgccggtggtgtcgatgccatccgagaggtccccgaggaccgctgggacatcgacgagttctacgaccccgatccggacaccccgggcaagacctacacgcggttcggcggatttctagacggcatcgacggattcgatcccgagttcttcggcatctcaccgcgtgaggccgtgtggatcgagccgcagcagcgcctcatgctcgagacggtgtgggagggtctcgagcgggccggctacgcaccttcggacctccggggcagccgcacgggtgtcttcaccggtgttgccgccaacgagtacgcgcatctgctgtcggccgagtcgatcgacaagatcgagccgcacttcatcacgggcaatgcgctcaacgcgatctccggtcgcgtggcgttcgcgctgggcttcgaaggtcctgccgtcgcggtcgacaccgcgtgcagctcggctctggtcgcggtccatcaggcatgtcaggcactgcagtccggggactgcgatctggccctcgcgggaggcgtcaacgtgctgctgagccccgtcaccgtggtcgcggcctcacgcgcgcggatgctgtcgccggtgggccggtgcaagacgttcgacgcgtccgccgacgggtatgtgcgcagtgagggctgcggcgtcctggtgctcaagcgcctcagcgacgccgtgcgtgacggcgaccgggtgtgcgcggtgatccctgcgagctcggtgaaccaggacggcgcctccagcgggttgaccgtccccaacggcggtgcgcagcaacggctcatcgaggcgacgctggcccgcgccggactgagcggcgccgatgtggactacctcgaggcacacggcacgggtacgcccctcggtgacccgatcgaggtgcaggccgccgcggccgcgtacggcgccgggcgtgacgccgaccggccgctgctgatgggatcggtgaagaccaacatcgggcacaccgaatccgcctcgggtgcagcgggtctgatcaaggtggtgctgtcactgcaacacggggtgctgccgcagagcctgcacttcgagaatccgtcaccgcacatcccgtgggacgcattgccggtgcgggtgatcgacgaaccggtgccgtggcagaccaacggccgggtgcgccgtgccggtgtcagttcgttcgggttcaccggcaccaacgcgcacgtgctcgtcgaggaggccccgccgcaactgcacaccgccgacgctgcgcccgtggaaccggccgccaccgcggaggccgggaccgacgactcgccgcaggtgctggcgctgtcggcgcggtcgcccgaggcgctcgtggagttggcgcggcgctacgacgactggttgagcgcgacgcccggcctcgacatcgccgacgtgtgccacaccgcgggcgtgggccgttcgcacttcgaataccgcgcagccctggtggtcgactcggtcgagcgggcacgcgagggtctcgccgcgctcgccgaggggcgcactcctgcgggcgtggtgcgcggcgagcatgtgcaccgtccgacgacggcatggctgttcaccggacagggcagccagtacccgggcatggcccgcgaattgttcgacacgcaaccggttttcgcccagaccgttcggcagtgcgccgaggcggtcgccgacatgctgccgcgaccactgctggacgtgctgttcgcgaccgaccgcgaaaccgcggacctgctgcagcacacgtcgttcgcgcagcccgcgctgttcgcggtcgagatggggctagcgcggctgtggcagtcatgggccatcgaacccgatgtggtgctggggcacagcgtcggccagtacgccgcggcctgcgtggcgggcgtgttcagccttgacgacggcgcacgactgatggccgagcgcggccgcatgttcggcagccttcccgaaggcggccggatggtggccgtgttcgccgacgccgaacacgtcgagcaggtggccggtgagttcccccgggtgtcggtcggcgcctacaacggccccaacaccgtgctctcgggtcccggcgaggacctcgaagagatcgtcgagaggttcgaggacgaggggatccgctgcacgtggctggcgaccagccacgcgttccactccgaactgctcgatcccgtgctcgacgagttcgaggcctacgcggcccagttccagttcgcagcgccgacactgcctttggtgtgcaaccggaccggggccgtgctcaccgggcagaccccgctcgacgcgcagtactggcgcaggcattcccgccagcccgtgcagttcgccgagagtgtgcgcaccgtcgccgcgctcggctgctcggtgctgatggagatcggcccgcaacctgtgctgaccggggccgcggtacaggtgtggcccgaacacatggctgccccgcgggccgtcgcctcgctgcgcaagggcgtcgccgatcgccgacagatcgccgatgcgctggcgtcggcctacgtcggcggccaccgccccgacttcgccgcggtgcaccggcagccacgccgcccggtcgagttgcccacctatccgttccagcgccgcaggttctggccgaaggcctcgagcatcgccgtcgacggtgggaccgcggcgtcgggaatcctgggcagcggcaaggatctggcgtccggcgacaccatctacacgagccggctgtccgtcaaatcgcagccgtggctggccgatcacgtcatctacggcaccgtcgtcgtccccggcgcgacgtatgcggcgatggcgctcgccgcggtcggcacaccggcacgcctgaaggacgtgttcttctacgagccgatcatcctgcccgagaaggcctctcgcgaggtccagctgacgttgcaccctgccgacggtggcagcgtgctcagcttccagatccacagccgcccgtacggtgaacgtggtgccgactggtcgctgaacgccgagggcacggtcgacacggccggcagcgacccggacgccgccgcagacgagcagtcggatccggtcgacgaggtgatcgaacgcctcgagcgcatgcgtccgcaggacctcttcgagaccttcgccgacctggaactggcgtggggtcccacatggtcgggctcgctgaaatcgttgtggctcggcgacggtgaggccgtcggcgacatcctcgtcggcgccgaactcgccgagcaactcggctccgagcccatgcacccggtgctgatggacctgtgcacgggcgtcgcgttccccgcgttcccggcgctgctcgcggccgaacagggcgtcaacgacctgttcctgccgctgcgctacgggcaggtgacgctcgcggagaagatgccgcggcggttctactgccgggcgaggtggcaccgtagcgcgctcgacagcgaaacgcaggtgttcgaactcgaattcgtcgacctcggtgggcgagcgctcggcggcatccgcgagttcaccgtcaaacgtgcgccgcgcgaggcactgctgcgcggcctcgggggtgacgcgacccgcctgctctacacgctcgggtggcacgaggtgccgccgccaccatcggccgacgacgcggtcggcacgtggctgatcgccgggttcgacgagctggccgcgaaggtgcccggctgtgtccccatcgaccgcaacaccgatctgcaggtcctcggtgaggtgctgtcgcaggcgcacgctcgcggcgcgggattctcgggcgtcgtgtggcgttgcgcgacgcctggccggaaggaatcgggttctgcctccgccgaatccgcccgtctggaggccgagatcaccaatctgctcagcgcggtgcacgccgcgcaggggccgagcgtgaaactgcccggcggactctggatcgtcaccgaacacggcgtggcatgtgaatccggcgagccggtggacccggtgcaggccgcgctgtggggcttcggccgaacgacgatcaacgaggaaccggcgctgcactgccggctcgtcgactgcgacggatcttccgaggccgtcgacctgctggccgctctgctggccgcaccgggcatccaggaaccggaaatcgccgtgcggcagggcaaactgctggcgtcacggttgttgccgtgggcccgcagtggtcatctcacgctgccgcgcgggggcgacttcgtccttgcgcccaccgagcgcggcgcgatcgacaacctgcgcctcacggagacggacgtgccggagccggacgagggctacgtgcaggttcgggtcgaggccgcgggcctcaacttccgcgacgtgctcaacgtgctcggcctgtaccccggtgatccgggaccgatcggcggcgacttcgcaggcaccgtgacccaactgggggcaggtgtgagtggactcgaaatcggccaacgcgtgtacgggtcgatgcagggtgccttcgcgagccgcttcaacgtgcccgcccagttcctggcgccgattcccgacggcatcggcgcggtcgaggccgccacgatcccggccgcggcgctgacggtccggctcgcgttcgactgggcgcaactcaaacccggcgacaaggtgctcatccacgccgccagcggtggcgtggggctcgcggccatccagatggcgcagcagtccggcgccgaggtcttcgccaccgcgagcaccttcaagcgttcgaccctgcgcaggctgggcgtgaagtacgtctacgactctcggacgacggacttcgccgaccagatcctcgcggacaccaacggtgccggcgtggacgtggtgctcaacagcctcactagtgcgggtttcgtcgaggcgacgctgaaggccaccgcgcagggcggacgcttcgccgagatcgccaagcgcgacatctggtcgcatgagcagatggccgaggcccgccccgacatcgcctacgagatcgtcgcgctggacaccgtgatgttcaccgagcccgatcgcatccgcgacctgctcaccgaggtgtccgacgggatggcgcgcggcgagtggacaccactacccgccgagatctacccgatcaccgaggcccggtcggcgttccggcgcatgcaacaggcacggcacatcggcaagattgtgctgcaggtccccaaaccccttcagccgcagcaggaccggagctacctgatcaccggcggactcggggcgatcggcctgcacacggcggcgtatctggcgcagctcggcgcgggcgacatcgtgctgaccagccggcgcgaccccgacgagtcggcccagcgggcgatcgacgagatcaccgagcgccacaagtgccgcatccacacctatgcggccgatgtgggtgacgaggcacaggtggccgatctgctggcgcggatccgggccgagttgcctccactggccggaatcgcccatctggcgggcgtgctcgacgacgcgctgctgtcggcgcagaaccccgagcgcttccggaccacgttggcgcccaaggcctatggcgcgagccacctggaccgcctgacgcgcgacgacgacctggacttcttcatcgtgtcctcgtcggtgtcgagcctgttcggatcaccggggcaggccaactacgcaaccgccaatgcgatgctcgacggtctggtcgccaagcgccgtgcgcagggcctggtggccacgggcatcaacttcgggccgtggggtcagggcggcatggcgtcgtcggaggccgcgaccgcgaacatcaccgcccaaggtctgatcccgttggatccgtcggccgcgttgcacgcgctggccgaggtcatcgcgaacggctccgggcaggcgacggtgctcaaggccaactggcagcgcgcggcgaaggttctcggctcggcgcgcccgccgatcctggacctcgtcctgccgagcgcggccggggaggccacgggtgacagcgagctgctcaagcagttgatggagatcccgataccgcagcgcgccgggttcgtcaccgagttcctgcaacgcgaagtgcagaacttcctgcgtctcgcgtcgccgcccgcggcgtcgagccggttcctggacctcggcacggattcgctgatggccatcgagctgcgcaaccggctgcacagccagttcgggggcaagttcaccatcaacgcgaccgcggtgttcgactacccgaccatcggcgggctcgcggagtacctcgtgggtcagctgcccgacgccgaggcgcccgaaacggccactagtcagctcgacagcgggactcccgcccgggaagcgagcagcgctcttcgcgacggctaccggcaggcgggcgtgtcgggcagggtccggtcctacctcgacctgctggcggggctgtcggacttccgcgagcacttcgacggctccgacgggttctccctcgatctcgtggacatggccgacggtcccggagaggtcacggtgatctgctgcgcgggaacggcggcgatctccggtccgcacgagttcacccggctcgccggggcgctgcgcggaatcgctccggttcgggccgtgccccagcccggctacgaggagggcgaacctctgccgtcgtcgatggcggcggtggcggcggtgcaggccgatgcggtcatcaggacacagggggacaagccgttcgtggtggccggtcactccgcgggggcactgatggcctacgcgctggcgaccgaactgctcgatcgcgggcacccgccacgcggtgtcgtcctgatcgacgtctacccgcccggtcaccaggacgcgatgaacgcctggctggaggagctgaccgccacgctgttcgaccgcgagacggtgcggatggacgacaccaggctcaccgccctgggcgcctacgaccgcctcaccggtcagtggcgaccccgggaaaccgggctgccgacgctgctggtcagcgccggcgagccgatgggtccgtggcccgacgacagctggaagccgacgtggcccttcgagcacgacaccgtcgccgtccccggcgaccacttcacgatggtgcaggaacacgccgacgcgatcgcgcggcacatcgacgcctggctgggcggagggaattcatgaaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 7 - DNA, 345 bp, synthetic construct
  misc_feature 1..345: note = depicts the sequence of the recombinant vector pSSRi11 confirmed by sanger Sequencing
  source 1..345: mol_type = other DNA
cgcgaagtgcagaacttcctgcgtctcgcgtcgccgcccgcggcgtcgagccggttcctggacctcggcacggattcgctgatggccatcgagctgcgcaaccggctgcacagccagttcgggggcaagttcaccatcaacgcgaccgcggtgttcgactacccgaccatcggcgggctcgcggagtacctcactagtcagctcgacagcgggactcccgcccgggaagcgagcagcgctcttcgcgacggctaccggcaggcgggcgtgtcgggcagggtccggtcctacctcgacctgctggcggggctgtcggacttccgcgagcacttcgacggctccgac

SEQ ID NO: 8 - DNA, 375 bp, synthetic construct
  misc_feature 1..375: note = depicts the sequence of the recombinant vector pSSRi15 confirmed by sanger Sequencing
  source 1..375: mol_type = other DNA
tcgctgatggccatcgagctgcgcaaccggctgcacagccagttcgggggcaagttcaccatcaacgcgaccgcggtgttcgactacccgaccatcggcgggctcgcggagtacctcgtgggtcagctgcccgacgccgaggcgcccgaaacggccactagtcagctcgacagcgggactcccgcccgggaagcgagcagcgctcttcgcgacggctaccggcaggcgggcgtgtcgggcagggtccggtcctacctcgacctgctggcggggctgtcggacttccgcgagcacttcgacggctccgacgggttctccctcgatctcgtggacatggccgacggtcccggagaggtcacggtgatctgctgcgcg

SEQ ID NO: 9 - DNA, 27 bp, synthetic construct
  misc_feature 1..27: note = depicts the nucleotide sequence of forward primer IML578
  source 1..27: mol_type = other DNA
ctcaacagcctcactagtgcgggtttc

SEQ ID NO: 10 - DNA, 30 bp, synthetic construct
  misc_feature 1..30: note = depicts the nucleotide sequence of reverse primer IML579
  source 1..30: mol_type = other DNA
ctgctgactagtgaggtactccgcgagccc

SEQ ID NO: 11 - DNA, 27 bp, synthetic construct
  misc_feature 1..27: note = depicts the nucleotide sequence of IML808
  source 1..27: mol_type = other DNA
ctgctgactagtggccgtttcgggcgc

SEQ ID NO: 12 - DNA, 11787 bp, synthetic construct
  misc_feature 1..11787: note = Depicts the nucleic acid sequence encoding PKS7TE protein
  source 1..11787: mol_type = other DNA
atggtctctgccgaacatccgatcgagccgaccccgagcttcgcaatcatcggctacgccgcacgttttcccggcgcggcgagcgccgaggaatactgggcgttgttgcgggacgggcgtgaagcgatatccgatgttccgaaagaccgctgggacatcgaggagttcttcgacccggacccgtcgacaccgggcaaggtcgtcacgcgccgcgcggggttcgtcgatgacgtaacgggtttcgacgcgccgttcttcggtatgtccgcccgcgaggtacgtctgatggacccgcagcaccggattctcatggaaaccgcgtggcgcgcggtcgaacactcgggaatcgcgccgacgtcgctggccaacagcaacacgggcgtcttcgtcggcctggccacccacgactacctcggtatggcctccgacgaactcacctaccccgagatcgaggcctacatggccatcggaacctccaatgccgctgcggccggacggatcagttaccgcctgggattgcagggaccggcggtggcggtcgacaccgcgtgcagttcgtcgctggtggcgattcaccaggcgtgtcaggcacttcgcctgaacgagtgcgatctcgcgttggccggcggggcgaacgtcctgctcaccccggccaccatgatcacgttctccaacgcgcacatgctggctccggacggccggtgcaagaccttcgacgcggcggccgacggctacgtgcgcggcgagggctgcggtgtcgtcgtggtcaagcgcctcgaggacgcggtgcgcgacggcgaccggatccgcgcggtgatccgcggaagcgcaatcaaccaggacggggcgtcgggcgggctgaccgtgccgaacggcgttgctcagcaacgggttatcgccgacgcgttgaagcgcgccggcctgcgaccggccgatgtcggttatctcgaggcgcacggcaccggaacctcacttggcgaccccatcgaggcccaggccgcgggcgaggtgctcggggccggacgcgcacctgaagaaccgctgctgatcggctcggcgaagaccaacatcggccatctggaagcggccgcgggcatcgccggtgtcatcaaggtggtcatggcgctcgagaacgagacgttgcccaagcacctcaatttcgagactccgtcgccccacatcccgtgggagcgtctacccatcgaggtggtgagagagaccgttccctggcagcgcaacggcaagccgcggatcgccggggtgagctcattcgggtttgccgggactaacgcgcacgtcatcctcgaagaggcgcccgccgtcggaacaccggcgacgacagagcctgtcgaggatccgaggcgctacagcgtccttccgctgtcggcacgcacgcccgaggcgttggtccgactcgccggtgagtaccgcgactggctgcgcgcccacccgcaggcccgcctggccgacgtgtcccacaccgcgggaaccgcacgtgcccacctggagcagcgtgccgcgttggtggtcaactcgcgggaatcggccatcgaactgctcggcgcgctcgccgacgaccgtccggcgcccggcctggtccgcggtgaatcccacgaggcacccaagaccgcgtggttgttcaccggccagggcagccagtaccccggaatggcgcgtgagttgttcgacaccgaaccggtgttcgccgaaaccgtgaggaaatgcgccgaggccgtcgcagatatcctcgaaaagcctttgctcgacgtcattttcgacgccgacgagttggatggtgacaccgaatcgccgctgcggcacacgacctatgcgcaacccgcgctgttcgccgtcgaattgggcctggcccggctctggcagtcctggggcttcgaacccgacgtggtggtcggacacagtgtcgggcagtactcggccgcctgtgtggcgggtgtgttcgacgtcgaggacggcgcacgactgatcgccgagcgcggccgattgttcggcagcctgcccgcgggtggacgcatggcggcggttttcaccgcccccgagcgtgcagagcgcctcaccgacgagtaccccagcctgtcggttgccgcgtacaacggtgccaacaccgtattgtccggtcccgcaaaggatctcgaatccgccgtggccacattggtggccgacggtgtgcggtgcgactggctggacaccagccacgccttccactcggcactgctcgacccgattctcgacgatttcgagtcgttcgcccagaagctcgaataccgtgaaccgcaacgtatcctgatcgacaaccgcaccgggtccgcgctcgggcggagcaccaggctcgacggtgcctactggcggcggcacgcacgccagcctgtcgagttcgccaagagcgtccgcaccctcgccgacatgaactgcagggtgctgctggagatagggcccaggcccgtgctcaccgccaccgcgctggcagcatggcccgacccggccaccacgccgcaggtgctcgcgtcgctgcggcccaccacggccgatcaccggcagatcaccgaggccgtcgccgccgcctatgccgcgggacatctgcccgatttccgcgcgttccggcaaccggacgcgcgcaaactcgacctgcccacatatcccttcgagcatcgccagttctggttctcggacaaccgtgcgatcgaccgtgacacccagaccgcgagcgccggttccgtcacgcctcaccgcacacaggccgtccgacttctcgaagacggccagatcgaggaactcgccgccctcatcgacggcgagaccgtcgacgagcagaccctgcgcgtgctgaacaagctcgcggcgcgccacaaccagcagcggtccacacacgtcgaccctgatgcgcggtacgagatccgttgggacgcgatcgcttcggccgccgcgagcacgggcgcggcagccgactggatcctggtcggcgacgacaccgctgccgtcctggagttcgccgatgcgctcaccgcaggcggtcatcgccaccagatcgtcgcgctgcccggctccgacgccgacgaggcccagctggtcgatacgctgcgcgctgcgtcggccggtgagctgtacgtcgtgcacatcgcggcaggagacggtgccgctgccccgatgcgggacctgctgcgcgtgcagcaccgaacccttggtgggctgcggcgcctgttccgcgccgcgaacgacgcggaactgcgcagccccatctggttggtgacacgtgacgggcagcgggtcaccgacaccgacaccgtggtaccggaacagagttgcctgtggggattcggccgcgcggcggccctggaactcccgcacatctggggtggcctcgccgacctctccggtgcggacaccgagtggccgcggctcatcgcgcggatcacgtcaccgcgcgacggtgaggaccagatcgcgctgcgcggggacgccgtgtacgcaccccgactggtccgccggaccggcgagccgagcgacacccccctgccggtgcgggaggaccgcacctatctggtgacgggcggtctcggggcgatcgggctcgaggtcgccggctacctggcctcccacggtgccggtcatgtggtgctcaccagccgacgggaaccgggcgacgacgcgcgccggcgcctcgacggtctccgcgagcagtacgcatgtgacgtccgggtgatcaccgccgatgtcgccgacgcgcacgacgtcgcgcgcctcatgaccaccgtcgccgccgaactcccgccgctggccggcatcgtgcacgccgcgggcgagatcggcaccacgccgctgagcggcctcgacgacgccgaggtggatcgcgtcttcgccgggaaggtctggggcgcctggcatctgagcgaggccctgacagatcttcgcaccgatctcgacttcttcgtcagcacatcgtcgatcgcgtcggtgtggggcggattcgggcagaccgcctacggcgcggcgaacgcgtttctcgacggtctggcgtggcgactgcgcgagcggggtgtcaccgctgtcagcgtgaacttcgggccgtgggccgcgggcatggccgacgccgaatcacgtgcccgcctcgagcagcgaggcgtgcggacgttgtcgcccgccgaggccctcgcaggcatggccgacgtcgtggccggccccgttcagggagtcgtcgccaagatcgactggtcacgtttcctgccgctgtaccagcaggcaggccggcgcgcgttcctgtcggaactcgaaagcgaattgcccgcagcggcaaccggcgctgctgcgcccgcgacggtgcccgggaaaccaccgctcgtcgagcagttgaccaaagcgcctgtgcagcaacgcaaaagcctcatcacgaactacctgcgcaacgcggtcgccgaggtcacacgggtcgacgccgacgagatccgcgatgaggccgggttcttcgacctcgggatggactccctgatggccgtcgaactgcggcggcgcatcgagcagggtgtcggcaaggacattcccgtcacgctcgtgatggatcatccgcggttgtccgacgcggccgactacctgctggtcgaggtactcgggttgggcgagcagaccaacgtgcggcaggcgtcgaccgtgaccgcgcgaaccgacgatcccatcgcggtcgtcgcggtgtcctgccgcttccccggcgcacccgacccggagtcgttctgggagctgctcgccggtggtgtcgatgccatccgagaggtccccgaggaccgctgggacatcgacgagttctacgaccccgatccggacaccccgggcaagacctacacgcggttcggcggatttctagacggcatcgacggattcgatcccgagttcttcggcatctcaccgcgtgaggccgtgtggatcgagccgcagcagcgcctcatgctcgagacggtgtgggagggtctcgagcgggccggctacgcaccttcggacctccggggcagccgcacgggtgtcttcaccggtgttgccgccaacgagtacgcgcatctgctgtcggccgagtcgatcgacaagatcgagccgcacttcatcacgggcaatgcgctcaacgcgatctccggtcgcgtggcgttcgcgctgggcttcgaaggtcctgccgtcgcggtcgacaccgcgtgcagctcggctctggtcgcggtccatcaggcatgtcaggcactgcagtccggggactgcgatctggccctcgcgggaggcgtcaacgtgctgctgagccccgtcaccgtggtcgcggcctcacgcgcgcggatgctgtcgccggtgggccggtgcaagacgttcgacgcgtccgccgacgggtatgtgcgcagtgagggctgcggcgtcctggtgctcaagcgcctcagcgacgccgtgcgtgacggcgaccgggtgtgcgcggtgatccctgcgagctcggtgaaccaggacggcgcctccagcgggttgaccgtccccaacggcggtgcgcagcaacggctcatcgaggcgacgctggcccgcgccggactgagcggcgccgatgtggactacctcgaggcacacggcacgggtacgcccctcggtgacccgatcgaggtgcaggccgccgcggccgcgtacggcgccgggcgtgacgccgaccggccgctgctgatgggatcggtgaagaccaacatcgggcacaccgaatccgcctcgggtgcagcgggtctgatcaaggtggtgctgtcactgcaacacggggtgctgccgcagagcctgcacttcgagaatccgtcaccgcacatcccgtgggacgcattgccggtgcgggtgatcgacgaaccggtgccgtggcagaccaacggccgggtgcgccgtgccggtgtcagttcgttcgggttcaccggcaccaacgcgcacgtgctcgtcgaggaggccccgccgcaactgcacaccgccgacgctgcgcccgtggaaccggccgccaccgcggaggccgggaccgacgactcgccgcaggtgctggcgctgtcggcgcggtcgcccgaggcgctcgtggagttggcgcggcgctacgacgactggttgagcgcgacgcccggcctcgacatcgccgacgtgtgccacaccgcgggcgtgggccgttcgcacttcgaataccgcgcagccctggtggtcgactcggtcgagcgggcacgcgagggtctcgccgcgctcgccgaggggcgcactcctgcgggcgtggtgcgcggcgagcatgtgcaccgtccgacgacggcatggctgttcaccggacagggcagccagtacccgggcatggcccgcgaattgttcgacacgcaaccggttttcgcccagaccgttcggcagtgcgccgaggcggtcgccgacatgctgccgcgaccactgctggacgtgctgttcgcgaccgaccgcgaaaccgcggacctgctgcagcacacgtcgttcgcgcagcccgcgctgttcgcggtcgagatggggctagcgcggctgtggcagtcatgggccatcgaacccgatgtggtgctggggcacagcgtcggccagtacgccgcggcctgcgtggcgggcgtgttcagccttgacgacggcgcacgactgatggccgagcgcggccgcatgttcggcagccttcccgaaggcggccggatggtggccgtgttcgccgacgccgaacacgtcgagcaggtggccggtgagttcccccgggtgtcggtcggcgcctacaacggccccaacaccgtgctctcgggtcccggcgaggacctcgaagagatcgtcgagaggttcgaggacgaggggatccgctgcacgtggctggcgaccagccacgcgttccactccgaactgctcgatcccgtgctcgacgagttcgaggcctacgcggcccagttccagttcgcagcgccgacactgcctttggtgtgcaaccggaccggggccgtgctcaccgggcagaccccgctcgacgcgcagtactggcgcaggcattcccgccagcccgtgcagttcgccgagagtgtgcgcaccgtcgccgcgctcggctgctcggtgctgatggagatcggcccgcaacctgtgctgaccggggccgcggtacaggtgtggcccgaacacatggctgccccgcgggccgtcgcctcgctgcgcaagggcgtcgccgatcgccgacagatcgccgatgcgctggcgtcggcctacgtcggcggccaccgccccgacttcgccgcggtgcaccggcagccacgccgcccggtcgagttgcccacctatccgttccagcgccgcaggttctggccgaaggcctcgagcatcgccgtcgacggtgggaccgcggcgtcgggaatcctgggcagcggcaaggatctggcgtccggcgacaccatctacacgagccggctgtccgtcaaatcgcagccgtggctggccgatcacgtcatctacggcaccgtcgtcgtccccggcgcgacgtatgcggcgatggcgctcgccgcggtcggcacaccggcacgcctgaaggacgtgttcttctacgagccgatcatcctgcccgagaaggcctctcgcgaggtccagctgacgttgcaccctgccgacggtggcagcgtgctcagcttccagatccacagccgcccgtacggtgaacgtggtgccgactggtcgctgaacgccgagggcacggtcgacacggccggcagcgacccggacgccgccgcagacgagcagtcggatccggtcgacgaggtgatcgaacgcctcgagcgcatgcgtccgcaggacctcttcgagaccttcgccgacctggaactggcgtggggtcccacatggtcgggctcgctgaaatcgttgtggctcggcgacggtgaggccgtcggcgacatcctcgtcggcgccgaactcgccgagcaactcggctccgagcccatgcacccggtgctgatggacctgtgcacgggcgtcgcgttccccgcgttcccggcgctgctcgcggccgaacagggcgtcaacgacctgttcctgccgctgcgctacgggcaggtgacgctcgcggagaagatgccgcggcggttctactgccgggcgaggtggcaccgtagcgcgctcgacagcgaaacgcaggtgttcgaactcgaattcgtcgacctcggtgggcgagcgctcggcggcatccgcgagttcaccgtcaaacgtgcgccgcgcgaggcactgctgcgcggcctcgggggtgacgcgacccgcctgctctacacgctcgggtggcacgaggtgccgccgccaccatcggccgacgacgcggtcggcacgtggctgatcgccgggttcgacgagctggccgcgaaggtgcccggctgtgtccccatcgaccgcaacaccgatctgcaggtcctcggtgaggtgctgtcgcaggcgcacgctcgcggcgcgggattctcgggcgtcgtgtggcgttgcgcgacgcctggccggaaggaatcgggttctgcctccgccgaatccgcccgtctggaggccgagatcaccaatctgctcagcgcggtgcacgccgcgcaggggccgagcgtgaaactgcccggcggactctggatcgtcaccgaacacggcgtggcatgtgaatccggcgagccggtggacccggtgcaggccgcgctgtggggcttcggccgaacgacgatcaacgaggaaccggcgctgcactgccggctcgtcgactgcgacggatcttccgaggccgtcgacctgctggccgctctgctggccgcaccgggcatccaggaaccggaaatcgccgtgcggcagggcaaactgctggcgtcacggttgttgccgtgggcccgcagtggtcatctcacgctgccgcgcgggggcgacttcgtccttgcgcccaccgagcgcggcgcgatcgacaacctgcgcctcacggagacggacgtgccggagccggacgagggctacgtgcaggttcgggtcgaggccgcgggcctcaacttccgcgacgtgctcaacgtgctcggcctgtaccccggtgatccgggaccgatcggcggcgacttcgcaggcaccgtgacccaactgggggcaggtgtgagtggactcgaaatcggccaacgcgtgtacgggtcgatgcagggtgccttcgcgagccgcttcaacgtgcccgcccagttcctggcgccgattcccgacggcatcggcgcggtcgaggccgccacgatcccggccgcggcgctgacggtccggctcgcgttcgactgggcgcaactcaaacccggcgacaaggtgctcatccacgccgccagcggtggcgtggggctcgcggccatccagatggcgcagcagtccggcgccgaggtcttcgccaccgcgagcaccttcaagcgttcgaccctgcgcaggctgggcgtgaagtacgtctacgactctcggacgacggacttcgccgaccagatcctcgcggacaccaacggtgccggcgtggacgtggtgctcaacagcctcactagtgcgggtttcgtcgaggcgacgctgaaggccaccgcgcagggcggacgcttcgccgagatcgccaagcgcgacatctggtcgcatgagcagatggccgaggcccgccccgacatcgcctacgagatcgtcgcgctggacaccgtgatgttcaccgagcccgatcgcatccgcgacctgctcaccgaggtgtccgacgggatggcgcgcggcgagtggacaccactacccgccgagatctacccgatcaccgaggcccggtcggcgttccggcgcatgcaacaggcacggcacatcggcaagattgtgctgcaggtccccaaaccccttcagccgcagcaggaccggagctacctgatcaccggcggactcggggcgatcggcctgcacacggcggcgtatctggcgcagctcggcgcgggcgacatcgtgctgaccagccggcgcgaccccgacgagtcggcccagcgggcgatcgacgagatcaccgagcgccacaagtgccgcatccacacctatgcggccgatgtgggtgacgaggcacaggtggccgatctgctggcgcggatccgggccgagttgcctccactggccggaatcgcccatctggcgggcgtgctcgacgacgcgctgctgtcggcgcagaaccccgagcgcttccggaccacgttggcgcccaaggcctatggcgcgagccacctggaccgcctgacgcgcgacgacgacctggacttcttcatcgtgtcctcgtcggtgtcgagcctgttcggatcaccggggcaggccaactacgcaaccgccaatgcgatgctcgacggtctggtcgccaagcgccgtgcgcagggcctggtggccacgggcatcaacttcgggccgtggggtcagggcggcatggcgtcgtcggaggccgcgaccgcgaacatcaccgcccaaggtctgatcccgttggatccgtcggccgcgttgcacgcgctggccgaggtcatcgcgaacggctccgggcaggcgacggtgctcaaggccaactggcagcgcgcggcgaaggttctcggctcggcgcgcccgccgatcctggacctcgtcctgccgagcgcggccggggaggccacgggtgacagcgagctgctcaagcagttgatggagatcccgataccgcagcgcgccgggttcgtcaccgagttcctgcaacgcgaagtgcagaacttcctgcgtctcgcgtcgccgcccgcggcgtcgagccggttcctggacctcggcacggattcgctgatggccatcgagctgcgcaaccggctgcacagccagttcgggggcaagttcaccatcaacgcgaccgcggtgttcgactacccgaccatcggcgggctcgcggagtacctcgtgggtcagctgcccgacgccactagtcagctcgacagcgggactcccgcccgggaagcgagcagcgctcttcgcgacggctaccggcaggcgggcgtgtcgggcagggtccggtcctacctcgacctgctggcggggctgtcggacttccgcgagcacttcgacggctccgacgggttctccctcgatctcgtggacatggccgacggtcccggagaggtcacggtgatctgctgcgcgggaacggcggcgatctccggtccgcacgagttcacccggctcgccggggcgctgcgcggaatcgctccggttcgggccgtgccccagcccggctacgaggagggcgaacctctgccgtcgtcgatggcggcggtggcggcggtgcaggccgatgcggtcatcaggacacagggggacaagccgttcgtggtggccggtcactccgcgggggcactgatggcctacgcgctggcgaccgaactgctcgatcgcgggcacccgccacgcggtgtcgtcctgatcgacgtctacccgcccggtcaccaggacgcgatgaacgcctggctggaggagctgaccgccacgctgttcgaccgcgagacggtgcggatggacgacaccaggctcaccgccctgggcgcctacgaccgcctcaccggtcagtggcgaccccgggaaaccgggctgccgacgctgctggtcagcgccggcgagccgatgggtccgtggcccgacgacagctggaagccgacgtggcccttcgagcacgacaccgtcgccgtccccggcgaccacttcacgatggtgcaggaacacgccgacgcgatcgcgcggcacatcgacgcctggctgggcggagggaattcatga

SEQ ID NO: 13 - protein, 3928 aa, synthetic construct
  REGION 1..3928: note = Depicts the amino acid sequence of PKS7TE protein
MVSAEHPIEPTPSFAIIGYAARFPGAASAEEYWALLRDGREAISDVPKDRWDIEEFFDPDPSTPGKVVTRRAGFVDDVTGFDAPFFGMSAREVRLMDPQHRILMETAWRAVEHSGIAPTSLANSNTGVFVGLATHDYLGMASDELTYPEIEAYMAIGTSNAAAAGRISYRLGLQGPAVAVDTACSSSLVAIHQACQALRLNECDLALAGGANVLLTPATMITFSNAHMLAPDGRCKTFDAAADGYVRGEGCGVVVVKRLEDAVRDGDRIRAVIRGSAINQDGASGGLTVPNGVAQQRVIADALKRAGLRPADVGYLEAHGTGTSLGDPIEAQAAGEVLGAGRAPEEPLLIGSAKTNIGHLEAAAGIAGVIKVVMALENETLPKHLNFETPSPHIPWERLPIEVVRETVPWQRNGKPRIAGVSSFGFAGTNAHVILEEAPAVGTPATTEPVEDPRRYSVLPLSARTPEALVRLAGEYRDWLRAHPQARLADVSHTAGTARAHLEQRAALVVNSRESAIELLGALADDRPAPGLVRGESHEAPKTAWLFTGQGSQYPGMARELFDTEPVFAETVRKCAEAVADILEKPLLDVIFDADELDGDTESPLRHTTYAQPALFAVELGLARLWQSWGFEPDVVVGHSVGQYSAACVAGVFDVEDGARLIAERGRLFGSLPAGGRMAAVFTAPERAERLTDEYPSLSVAAYNGANTVLSGPAKDLESAVATLVADGVRCDWLDTSHAFHSALLDPILDDFESFAQKLEYREPQRILIDNRTGSALGRSTRLDGAYWRRHARQPVEFAKSVRTLADMNCRVLLEIGPRPVLTATALAAWPDPATTPQVLASLRPTTADHRQITEAVAAAYAAGHLPDFRAFRQPDARKLDLPTYPFEHRQFWFSDNRAIDRDTQTASAGSVTPHRTQAVRLLEDGQIEELAALIDGETVDEQTLRVLNKLAARHNQQRSTHVDPDARYEIRWDAIASAAASTGAAADWILVGDDTAAVLEFADALTAGGHRHQIVALPGSDADEAQLVDTLRAASAGELYVVHIAAGDGAAAPMRDLLRVQHRTLGGLRRLFRAANDAELRSPIWLVTRDGQRVTDTDTVVPEQSCLWGFGRAAALELPHIWGGLADLSGADTEWPRLIARITSPRDGEDQIALRGDAVYAPRLVRRTGEPSDTPLPVREDRTYLVTGGLGAIGLEVAGYLASHGAGHVVLTSRREPGDDARRRLDGLREQYACDVRVITADVADAHDVARLMTTVAAELPPLAGIVHAAGEIGTTPLSGLDDAEVDRVFAGKVWGAWHLSEALTDLRTDLDFFVSTSSIASVWGGFGQTAYGAANAFLDGLAWRLRERGVTAVSVNFGPWAAGMADAESRARLEQRGVRTLSPAEALAGMADVVAGPVQGVVAKIDWSRFLPLYQQAGRRAFLSELESELPAAATGAAAPATVPGKPPLVEQLTKAPVQQRKSLITNYLRNAVAEVTRVDADEIRDEAGFFDLGMDSLMAVELRRRIEQGVGKDIPVTLVMDHPRLSDAADYLLVEVLGLGEQTNVRQASTVTARTDDPIAVVAVSCRFPGAPDPESFWELLAGGVDAIREVPEDRWDIDEFYDPDPDTPGKTYTRFGGFLDGIDGFDPEFFGISPREAVWIEPQQRLMLETVWEGLERAGYAPSDLRGSRTGVFTGVAANEYAHLLSAESIDKIEPHFITGNALNAISGRVAFALGFEGPAVAVDTACSSALVAVHQACQALQSGDCDLALAGGVNVLLSPVTVVAASRARMLSPVGRCKTFDASADGYVRSEGCGVLVLKRLSDAVRDGDRVCAVIPASSVNQDGASSGLTVPNGGAQQRLIEATLARAGLSGADVDYLEAHGTGTPLGDPIEVQAAAAAYGAGRDADRPLLMGSVKTNIGHTESASGAAGLIKVVLSLQHGVLPQSLHFENPSPHIPWDALPVRVIDEPVPWQTNGRVRRAGVSSFGFTGTNAHVLVEEAPPQLHTADAAPVEPAATAEAGTDDSPQVLALSARSPEALVELARRYDDWLSATPGLDIADVCHTAGVGRSHFEYRAALVVDSVERAREGLAALAEGRTPAGVVRGEHVHRPTTAWLFTGQGSQYPGMARELFDTQPVFAQTVRQCAEAVADMLPRPLLDVLFATDRETADLLQHTSFAQPALFAVEMGLARLWQSWAIEPDVVLGHSVGQYAAACVAGVFSLDDGARLMAERGRMFGSLPEGGRMVAVFADAEHVEQVAGEFPRVSVGAYNGPNTVLSGPGEDLEEIVERFEDEGIRCTWLATSHAFHSELLDPVLDEFEAYAAQFQFAAPTLPLVCNRTGAVLTGQTPLDAQYWRRHSRQPVQFAESVRTVAALGCSVLMEIGPQPVLTGAAVQVWPEHMAAPRAVASLRKGVADRRQIADALASAYVGGHRPDFAAVHRQPRRPVELPTYPFQRRRFWPKASSIAVDGGTAASGILGSGKDLASGDTIYTSRLSVKSQPWLADHVIYGTVVVPGATYAAMALAAVGTPARLKDVFFYEPIILPEKASREVQLTLHPADGGSVLSFQIHSRPYGERGADWSLNAEGTVDTAGSDPDAAADEQSDPVDEVIERLERMRPQDLFETFADLELAWGPTWSGSLKSLWLGDGEAVGDILVGAELAEQLGSEPMHPVLMDLCTGVAFPAFPALLAAEQGVNDLFLPLRYGQVTLAEKMPRRFYCRARWHRSALDSETQVFELEFVDLGGRALGGIREFTVKRAPREALLRGLGGDATRLLYTLGWHEVPPPPSADDAVGTWLIAGFDELAAKVPGCVPIDRNTDLQVLGEVLSQAHARGAGFSGVVWRCATPGRKESGSASAESARLEAEITNLLSAVHAAQGPSVKLPGGLWIVTEHGVACESGEPVDPVQAALWGFGRTTINEEPALHCRLVDCDGSSEAVDLLAALLAAPGIQEPEIAVRQGKLLASRLLPWARSGHLTLPRGGDFVLAPTERGAIDNLRLTETDVPEPDEGYVQVRVEAAGLNFRDVLNVLGLYPGDPGPIGGDFAGTVTQLGAGVSGLEIGQRVYGSMQGAFASRFNVPAQFLAPIPDGIGAVEAATIPAAALTVRLAFDWAQLKPGDKVLIHAASGGVGLAAIQMAQQSGAEVFATASTFKRSTLRRLGVKYVYDSRTTDFADQILADTNGAGVDVVLNSLTSAGFVEATLKATAQGGRFAEIAKRDIWSHEQMAEARPDIAYEIVALDTVMFTEPDRIRDLLTEVSDGMARGEWTPLPAEIYPITEARSAFRRMQQARHIGKIVLQVPKPLQPQQDRSYLITGGLGAIGLHTAAYLAQLGAGDIVLTSRRDPDESAQRAIDEITERHKCRIHTYAADVGDEAQVADLLARIRAELPPLAGIAHLAGVLDDALLSAQNPERFRTTLAPKAYGASHLDRLTRDDDLDFFIVSSSVSSLFGSPGQANYATANAMLDGLVAKRRAQGLVATGINFGPWGQGGMASSEAATANITAQGLIPLDPSAALHALAEVIANGSGQATVLKANWQRAAKVLGSARPPILDLVLPSAAGEATGDSELLKQLMEIPIPQRAGFVTEFLQREVQNFLRLASPPAASSRFLDLGTDSLMAIELRNRLHSQFGGKFTINATAVFDYPTIGGLAEYLVGQLPDATSQLDSGTPAREASSALRDGYRQAGVSGRVRSYLDLLAGLSDFREHFDGSDGFSLDLVDMADGPGEVTVICCAGTAAISGPHEFTRLAGALRGIAPVRAVPQPGYEEGEPLPSSMAAVAAVQADAVIRTQGDKPFVVAGHSAGALMAYALATELLDRGHPPRGVVLIDVYPPGHQDAMNAWLEELTATLFDRETVRMDDTRLTALGAYDRLTGQWRPRETGLPTLLVSAGEPMGPWPDDSWKPTWPFEHDTVAVPGDHFTMVQEHADAIARHIDAWLGGGNS

SEQ ID NO: 14 - DNA, 17169 bp, synthetic construct
  misc_feature 1..17169: note = Depicts the nucliec acid sequence of a non-working vector pSSRi14
  source 1..17169: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatggtctctgccgaacatccgatcgagccgaccccgagcttcgcaatcatcggctacgccgcacgttttcccggcgcggcgagcgccgaggaatactgggcgttgttgcgggacgggcgtgaagcgatatccgatgttccgaaagaccgctgggacatcgaggagttcttcgacccggacccgtcgacaccgggcaaggtcgtcacgcgccgcgcggggttcgtcgatgacgtaacgggtttcgacgcgccgttcttcggtatgtccgcccgcgaggtacgtctgatggacccgcagcaccggattctcatggaaaccgcgtggcgcgcggtcgaacactcgggaatcgcgccgacgtcgctggccaacagcaacacgggcgtcttcgtcggcctggccacccacgactacctcggtatggcctccgacgaactcacctaccccgagatcgaggcctacatggccatcggaacctccaatgccgctgcggccggacggatcagttaccgcctgggattgcagggaccggcggtggcggtcgacaccgcgtgcagttcgtcgctggtggcgattcaccaggcgtgtcaggcacttcgcctgaacgagtgcgatctcgcgttggccggcggggcgaacgtcctgctcaccccggccaccatgatcacgttctccaacgcgcacatgctggctccggacggccggtgcaagaccttcgacgcggcggccgacggctacgtgcgcggcgagggctgcggtgtcgtcgtggtcaagcgcctcgaggacgcggtgcgcgacggcgaccggatccgcgcggtgatccgcggaagcgcaatcaaccaggacggggcgtcgggcgggctgaccgtgccgaacggcgttgctcagcaacgggttatcgccgacgcgttgaagcgcgccggcctgcgaccggccgatgtcggttatctcgaggcgcacggcaccggaacctcacttggcgaccccatcgaggcccaggccgcgggcgaggtgctcggggccggacgcgcacctgaagaaccgctgctgatcggctcggcgaagaccaacatcggccatctggaagcggccgcgggcatcgccggtgtcatcaaggtggtcatggcgctcgagaacgagacgttgcccaagcacctcaatttcgagactccgtcgccccacatcccgtgggagcgtctacccatcgaggtggtgagagagaccgttccctggcagcgcaacggcaagccgcggatcgccggggtgagctcattcgggtttgccgggactaacgcgcacgtcatcctcgaagaggcgcccgccgtcggaacaccggcgacgacagagcctgtcgaggatccgaggcgctacagcgtccttccgctgtcggcacgcacgcccgaggcgttggtccgactcgccggtgagtaccgcgactggctgcgcgcccacccgcaggcccgcctggccgacgtgtcccacaccgcgggaaccgcacgtgcccacctggagcagcgtgccgcgttggtggtcaactcgcgggaatcggccatcgaactgctcggcgcgctcgccgacgaccgtccggcgcccggcctggtccgcggtgaatcccacgaggcacccaagaccgcgtggttgttcaccggccagggcagccagtaccccggaatggcgcgtgagttgttcgacaccgaaccggtgttcgccgaaaccgtgaggaaatgcgccgaggccgtcgcagatatcctcgaaaagcctttgctcgacgtcattttcgacgccgacgagttggatggtgacaccgaatcgccgctgcggcacacgacctatgcgcaacccgcgctgttcgccgtcgaattgggcctggcccggctctggcagtcctggggcttcgaacccgacgtggtggtcggacacagtgtcgggcagtactcggccgcctgtgtggcgggtgtgttcgacgtcgaggacggcgcacgactgatcgccgagcgcggccgattgttcggcagcctgcccgcgggtggacgcatggcggcggttttcaccgcccccgagcgtgcagagcgcctcaccgacgagtaccccagcctgtcggttgccgcgtacaacggtgccaacaccgtattgtccggtcccgcaaaggatctcgaatccgccgtggccacattggtggccgacggtgtgcggtgcgactggctggacaccagccacgccttccactcggcactgctcgacccgattctcgacgatttcgagtcgttcgcccagaagctcgaataccgtgaaccgcaacgtatcctgatcgacaaccgcaccgggtccgcgctcgggcggagcaccaggctcgacggtgcctactggcggcggcacgcacgccagcctgtcgagttcgccaagagcgtccgcaccctcgccgacatgaactgcagggtgctgctggagatagggcccaggcccgtgctcaccgccaccgcgctggcagcatggcccgacccggccaccacgccgcaggtgctcgcgtcgctgcggcccaccacggccgatcaccggcagatcaccgaggccgtcgccgccgcctatgccgcgggacatctgcccgatttccgcgcgttccggcaaccggacgcgcgcaaactcgacctgcccacatatcccttcgagcatcgccagttctggttctcggacaaccgtgcgatcgaccgtgacacccagaccgcgagcgccggttccgtcacgcctcaccgcacacaggccgtccgacttctcgaagacggccagatcgaggaactcgccgccctcatcgacggcgagaccgtcgacgagcagaccctgcgcgtgctgaacaagctcgcggcgcgccacaaccagcagcggtccacacacgtcgaccctgatgcgcggtacgagatccgttgggacgcgatcgcttcggccgccgcgagcacgggcgcggcagccgactggatcctggtcggcgacgacaccgctgccgtcctggagttcgccgatgcgctcaccgcaggcggtcatcgccaccagatcgtcgcgctgcccggctccgacgccgacgaggcccagctggtcgatacgctgcgcgctgcgtcggccggtgagctgtacgtcgtgcacatcgcggcaggagacggtgccgctgccccgatgcgggacctgctgcgcgtgcagcaccgaacccttggtgggctgcggcgcctgttccgcgccgcgaacgacgcggaactgcgcagccccatctggttggtgacacgtgacgggcagcgggtcaccgacaccgacaccgtggtaccggaacagagttgcctgtggggattcggccgcgcggcggccctggaactcccgcacatctggggtggcctcgccgacctctccggtgcggacaccgagtggccgcggctcatcgcgcggatcacgtcaccgcgcgacggtgaggaccagatcgcgctgcgcggggacgccgtgtacgcaccccgactggtccgccggaccggcgagccgagcgacacccccctgccggtgcgggaggaccgcacctatctggtgacgggcggtctcggggcgatcgggctcgaggtcgccggctacctggcctcccacggtgccggtcatgtggtgctcaccagccgacgggaaccgggcgacgacgcgcgccggcgcctcgacggtctccgcgagcagtacgcatgtgacgtccgggtgatcaccgccgatgtcgccgacgcgcacgacgtcgcgcgcctcatgaccaccgtcgccgccgaactcccgccgctggccggcatcgtgcacgccgcgggcgagatcggcaccacgccgctgagcggcctcgacgacgccgaggtggatcgcgtcttcgccgggaaggtctggggcgcctggcatctgagcgaggccctgacagatcttcgcaccgatctcgacttcttcgtcagcacatcgtcgatcgcgtcggtgtggggcggattcgggcagaccgcctacggcgcggcgaacgcgtttctcgacggtctggcgtggcgactgcgcgagcggggtgtcaccgctgtcagcgtgaacttcgggccgtgggccgcgggcatggccgacgccgaatcacgtgcccgcctcgagcagcgaggcgtgcggacgttgtcgcccgccgaggccctcgcaggcatggccgacgtcgtggccggccccgttcagggagtcgtcgccaagatcgactggtcacgtttcctgccgctgtaccagcaggcaggccggcgcgcgttcctgtcggaactcgaaagcgaattgcccgcagcggcaaccggcgctgctgcgcccgcgacggtgcccgggaaaccaccgctcgtcgagcagttgaccaaagcgcctgtgcagcaacgcaaaagcctcatcacgaactacctgcgcaacgcggtcgccgaggtcacacgggtcgacgccgacgagatccgcgatgaggccgggttcttcgacctcgggatggactccctgatggccgtcgaactgcggcggcgcatcgagcagggtgtcggcaaggacattcccgtcacgctcgtgatggatcatccgcggttgtccgacgcggccgactacctgctggtcgaggtactcgggttgggcgagcagaccaacgtgcggcaggcgtcgaccgtgaccgcgcgaaccgacgatcccatcgcggtcgtcgcggtgtcctgccgcttccccggcgcacccgacccggagtcgttctgggagctgctcgccggtggtgtcgatgccatccgagaggtccccgaggaccgctgggacatcgacgagttctacgaccccgatccggacaccccgggcaagacctacacgcggttcggcggatttctagacggcatcgacggattcgatcccgagttcttcggcatctcaccgcgtgaggccgtgtggatcgagccgcagcagcgcctcatgctcgagacggtgtgggagggtctcgagcgggccggctacgcaccttcggacctccggggcagccgcacgggtgtcttcaccggtgttgccgccaacgagtacgcgcatctgctgtcggccgagtcgatcgacaagatcgagccgcacttcatcacgggcaatgcgctcaacgcgatctccggtcgcgtggcgttcgcgctgggcttcgaaggtcctgccgtcgcggtcgacaccgcgtgcagctcggctctggtcgcggtccatcaggcatgtcaggcactgcagtccggggactgcgatctggccctcgcgggaggcgtcaacgtgctgctgagccccgtcaccgtggtcgcggcctcacgcgcgcggatgctgtcgccggtgggccggtgcaagacgttcgacgcgtccgccgacgggtatgtgcgcagtgagggctgcggcgtcctggtgctcaagcgcctcagcgacgccgtgcgtgacggcgaccgggtgtgcgcggtgatccctgcgagctcggtgaaccaggacggcgcctccagcgggttgaccgtccccaacggcggtgcgcagcaacggctcatcgaggcgacgctggcccgcgccggactgagcggcgccgatgtggactacctcgaggcacacggcacgggtacgcccctcggtgacccgatcgaggtgcaggccgccgcggccgcgtacggcgccgggcgtgacgccgaccggccgctgctgatgggatcggtgaagaccaacatcgggcacaccgaatccgcctcgggtgcagcgggtctgatcaaggtggtgctgtcactgcaacacggggtgctgccgcagagcctgcacttcgagaatccgtcaccgcacatcccgtgggacgcattgccggtgcgggtgatcgacgaaccggtgccgtggcagaccaacggccgggtgcgccgtgccggtgtcagttcgttcgggttcaccggcaccaacgcgcacgtgctcgtcgaggaggccccgccgcaactgcacaccgccgacgctgcgcccgtggaaccggccgccaccgcggaggccgggaccgacgactcgccgcaggtgctggcgctgtcggcgcggtcgcccgaggcgctcgtggagttggcgcggcgctacgacgactggttgagcgcgacgcccggcctcgacatcgccgacgtgtgccacaccgcgggcgtgggccgttcgcacttcgaataccgcgcagccctggtggtcgactcggtcgagcgggcacgcgagggtctcgccgcgctcgccgaggggcgcactcctgcgggcgtggtgcgcggcgagcatgtgcaccgtccgacgacggcatggctgttcaccggacagggcagccagtacccgggcatggcccgcgaattgttcgacacgcaaccggttttcgcccagaccgttcggcagtgcgccgaggcggtcgccgacatgctgccgcgaccactgctggacgtgctgttcgcgaccgaccgcgaaaccgcggacctgctgcagcacacgtcgttcgcgcagcccgcgctgttcgcggtcgagatggggctagcgcggctgtggcagtcatgggccatcgaacccgatgtggtgctggggcacagcgtcggccagtacgccgcggcctgcgtggcgggcgtgttcagccttgacgacggcgcacgactgatggccgagcgcggccgcatgttcggcagccttcccgaaggcggccggatggtggccgtgttcgccgacgccgaacacgtcgagcaggtggccggtgagttcccccgggtgtcggtcggcgcctacaacggccccaacaccgtgctctcgggtcccggcgaggacctcgaagagatcgtcgagaggttcgaggacgaggggatccgctgcacgtggctggcgaccagccacgcgttccactccgaactgctcgatcccgtgctcgacgagttcgaggcctacgcggcccagttccagttcgcagcgccgacactgcctttggtgtgcaaccggaccggggccgtgctcaccgggcagaccccgctcgacgcgcagtactggcgcaggcattcccgccagcccgtgcagttcgccgagagtgtgcgcaccgtcgccgcgctcggctgctcggtgctgatggagatcggcccgcaacctgtgctgaccggggccgcggtacaggtgtggcccgaacacatggctgccccgcgggccgtcgcctcgctgcgcaagggcgtcgccgatcgccgacagatcgccgatgcgctggcgtcggcctacgtcggcggccaccgccccgacttcgccgcggtgcaccggcagccacgccgcccggtcgagttgcccacctatccgttccagcgccgcaggttctggccgaaggcctcgagcatcgccgtcgacggtgggaccgcggcgtcgggaatcctgggcagcggcaaggatctggcgtccggcgacaccatctacacgagccggctgtccgtcaaatcgcagccgtggctggccgatcacgtcatctacggcaccgtcgtcgtccccggcgcgacgtatgcggcgatggcgctcgccgcggtcggcacaccggcacgcctgaaggacgtgttcttctacgagccgatcatcctgcccgagaaggcctctcgcgaggtccagctgacgttgcaccctgccgacggtggcagcgtgctcagcttccagatccacagccgcccgtacggtgaacgtggtgccgactggtcgctgaacgccgagggcacggtcgacacggccggcagcgacccggacgccgccgcagacgagcagtcggatccggtcgacgaggtgatcgaacgcctcgagcgcatgcgtccgcaggacctcttcgagaccttcgccgacctggaactggcgtggggtcccacatggtcgggctcgctgaaatcgttgtggctcggcgacggtgaggccgtcggcgacatcctcgtcggcgccgaactcgccgagcaactcggctccgagcccatgcacccggtgctgatggacctgtgcacgggcgtcgcgttccccgcgttcccggcgctgctcgcggccgaacagggcgtcaacgacctgttcctgccgctgcgctacgggcaggtgacgctcgcggagaagatgccgcggcggttctactgccgggcgaggtggcaccgtagcgcgctcgacagcgaaacgcaggtgttcgaactcgaattcgtcgacctcggtgggcgagcgctcggcggcatccgcgagttcaccgtcaaacgtgcgccgcgcgaggcactgctgcgcggcctcgggggtgacgcgacccgcctgctctacacgctcgggtggcacgaggtgccgccgccaccatcggccgacgacgcggtcggcacgtggctgatcgccgggttcgacgagctggccgcgaaggtgcccggctgtgtccccatcgaccgcaacaccgatctgcaggtcctcggtgaggtgctgtcgcaggcgcacgctcgcggcgcgggattctcgggcgtcgtgtggcgttgcgcgacgcctggccggaaggaatcgggttctgcctccgccgaatccgcccgtctggaggccgagatcaccaatctgctcagcgcggtgcacgccgcgcaggggccgagcgtgaaactgcccggcggactctggatcgtcaccgaacacggcgtggcatgtgaatccggcgagccggtggacccggtgcaggccgcgctgtggggcttcggccgaacgacgatcaacgaggaaccggcgctgcactgccggctcgtcgactgcgacggatcttccgaggccgtcgacctgctggccgctctgctggccgcaccgggcatccaggaaccggaaatcgccgtgcggcagggcaaactgctggcgtcacggttgttgccgtgggcccgcagtggtcatctcacgctgccgcgcgggggcgacttcgtccttgcgcccaccgagcgcggcgcgatcgacaacctgcgcctcacggagacggacgtgccggagccggacgagggctacgtgcaggttcgggtcgaggccgcgggcctcaacttccgcgacgtgctcaacgtgctcggcctgtaccccggtgatccgggaccgatcggcggcgacttcgcaggcaccgtgacccaactgggggcaggtgtgagtggactcgaaatcggccaacgcgtgtacgggtcgatgcagggtgccttcgcgagccgcttcaacgtgcccgcccagttcctggcgccgattcccgacggcatcggcgcggtcgaggccgccacgatcccggccgcggcgctgacggtccggctcgcgttcgactgggcgcaactcaaacccggcgacaaggtgctcatccacgccgccagcggtggcgtggggctcgcggccatccagatggcgcagcagtccggcgccgaggtcttcgccaccgcgagcaccttcaagcgttcgaccctgcgcaggctgggcgtgaagtacgtctacgactctcggacgacggacttcgccgaccagatcctcgcggacaccaacggtgccggcgtggacgtggtgctcaacagcctcactagtgcgggtttcgtcgaggcgacgctgaaggccaccgcgcagggcggacgcttcgccgagatcgccaagcgcgacatctggtcgcatgagcagatggccgaggcccgccccgacatcgcctacgagatcgtcgcgctggacaccgtgatgttcaccgagcccgatcgcatccgcgacctgctcaccgaggtgtccgacgggatggcgcgcggcgagtggacaccactacccgccgagatctacccgatcaccgaggcccggtcggcgttccggcgcatgcaacaggcacggcacatcggcaagattgtgctgcaggtccccaaaccccttcagccgcagcaggaccggagctacctgatcaccggcggactcggggcgatcggcctgcacacggcggcgtatctggcgcagctcggcgcgggcgacatcgtgctgaccagccggcgcgaccccgacgagtcggcccagcgggcgatcgacgagatcaccgagcgccacaagtgccgcatccacacctatgcggccgatgtgggtgacgaggcacaggtggccgatctgctggcgcggatccgggccgagttgcctccactggccggaatcgcccatctggcgggcgtgctcgacgacgcgctgctgtcggcgcagaaccccgagcgcttccggaccacgttggcgcccaaggcctatggcgcgagccacctggaccgcctgacgcgcgacgacgacctggacttcttcatcgtgtcctcgtcggtgtcgagcctgttcggatcaccggggcaggccaactacgcaaccgccaatgcgatgctcgacggtctggtcgccaagcgccgtgcgcagggcctggtggccacgggcatcaacttcgggccgtggggtcagggcggcatggcgtcgtcggaggccgcgaccgcgaacatcaccgcccaaggtctgatcccgttggatccgtcggccgcgttgcacgcgctggccgaggtcatcgcgaacggctccgggcaggcgacggtgctcaaggccaactggcagcgcgcggcgaaggttctcggctcggcgcgcccgccgatcctggacctcgtcctgccgagcgcggccggggaggccacgggtgacagcgagctgctcaagcagttgatggagatcccgataccgcagcgcgccgggttcgtcaccgagttcctgcaacgcgaagtgcagaacttcctgcgtctcgcgtcgccgcccgcggcgtcgagccggttcctggacctcggcacggattcgctgatggccatcgagctgcgcaaccggctgcacagccagttcgggggcaagttcaccatcaacgcgaccgcggtgttcgactacccgaccatcggcgggctcgcggagtacctcgtgggtcagctgcccgacgccactagtcagctcgacagcgggactcccgcccgggaagcgagcagcgctcttcgcgacggctaccggcaggcgggcgtgtcgggcagggtccggtcctacctcgacctgctggcggggctgtcggacttccgcgagcacttcgacggctccgacgggttctccctcgatctcgtggacatggccgacggtcccggagaggtcacggtgatctgctgcgcgggaacggcggcgatctccggtccgcacgagttcacccggctcgccggggcgctgcgcggaatcgctccggttcgggccgtgccccagcccggctacgaggagggcgaacctctgccgtcgtcgatggcggcggtggcggcggtgcaggccgatgcggtcatcaggacacagggggacaagccgttcgtggtggccggtcactccgcgggggcactgatggcctacgcgctggcgaccgaactgctcgatcgcgggcacccgccacgcggtgtcgtcctgatcgacgtctacccgcccggtcaccaggacgcgatgaacgcctggctggaggagctgaccgccacgctgttcgaccgcgagacggtgcggatggacgacaccaggctcaccgccctgggcgcctacgaccgcctcaccggtcagtggcgaccccgggaaaccgggctgccgacgctgctggtcagcgccggcgagccgatgggtccgtggcccgacgacagctggaagccgacgtggcccttcgagcacgacaccgtcgccgtccccggcgaccacttcacgatggtgcaggaacacgccgacgcgatcgcgcggcacatcgacgcctggctgggcggagggaattcatgaaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

(19) United States
(12) Patent Application Publication
(10) Pub. No.: US 2026/0167999 A1
(43) Pub. Date: Jun. 18, 2026

(54) RECOMBINANT DNA, RECOMBINANT VECTOR FOR PRODUCING DELTA-ACYL LACTONES AND ITS IMPLEMENTATION THEREOF

(71) Applicant: NATIONAL INSTITUTE OF IMMUNOLOGY, New Delhi (IN)

(72) Inventors: Rajesh S. GOKHALE, New Delhi (IN); Vinay NANDICOORI, New Delhi (IN); Sonali SRIVASTAVA, Delhi (IN)

(21) Appl. No.: 19/125,585

(22) PCT Filed: Oct. 31, 2023
(86) PCT No.: PCT/IN2023/050997

§ 371 Date: Apr. 29, 2025

(30) Foreign Application Priority Data
Oct. 31, 2022 (IN) 202211061834

(51) Int. Cl. C12P 17/06; B01D 15/38; C12N 1/20; C12N 9/10; C12N 15/52; C12N 15/62; C12N 15/70; C12P 21/02; C12R 1/19

(52) U.S. Cl. CPC C12P 17/06; B01D 15/3804; C12N 1/20; C12N 9/1029; C12N 15/52; C12N 15/62; C12N 15/70; C12P 21/02; C07K 2319/00; C12R 2001/19; C12Y 203/01094

(57) ABSTRACT

The present invention discloses a recombinant DNA encoding a protein having an amino acid sequence as set forth in SEQ ID NO: 2, or SEQ ID NO: 4. The present invention further discloses a recombinant vector comprising the recombinant DNA. Furthermore, a recombinant host cell comprising the recombinant vector is also described herein. The present invention further discloses a recombinant protein and a method for producing the said protein. There is also provided herein a method for producing the delta acyl lactone using the recombinant vector and recombinant host cell as described herein.

### FIELD OF INVENTION

The present invention relates to the field of recombinant DNA technology. Particularly, the present invention discloses a recombinant DNA. The present invention further discloses a recombinant vector comprising the said recombinant DNA. The present invention also discloses a method for producing delta-acyl lactones and its implementation thereof.

### BACKGROUND OF INVENTION

Delta acyl lactones such as δ-decalactone and δ-dodecalactone are aroma compounds of high commercial value. These lactones impart fruity and milky aroma and are widely used in food and perfume industry.

Currently, the commercial flavours are either produced chemically or by enzymatic or microbial conversion of hydroxy fatty acids (Gatfield, I. L., 1997. Biotechnological production of flavour-active lactones. In: Berger, R. G. (Ed.), Biotechnology of Aroma Compounds. Springer Berlin Heidelberg, pp. 221-238; Kang W R, et al. Production of δ-decalactone from linoleic acid via 13-hydroxy-9(Z)-octadecenoic acid intermediate by one-pot reaction using linoleate 13-hydratase and whole Yarrowia lipolytica cells. Biotechnol Lett. 2016 May; 38(5): 817-23.; and Marella E R, et al. A single-host fermentation process for the production of flavor lactones from non-hydroxylated fatty acids. Metab Eng. 2020 September; 61: 427-436; and van der Schaft, et. al. 1992). However, the extraction of aroma lactones from natural sources are not economically viable due to their low abundance. With the growing demand for natural flavours, bio-production of aroma lactones can be achieved using synthetic biology toolkit.

While various efforts have been made in the past to provide methods to produce the aroma lactones, however, there still exists a dire need in the art to provide a cost-effective method for producing the delta acyl lactones effectively.

### OBJECTS OF THE INVENTION

The principal object of the present invention is to provide a recombinant DNA.

Another object of the present invention is to provide a recombinant protein encoded by the recombinant DNA as described herein.

Yet another object of the present invention is to provide a recombinant vector comprising the recombinant DNA.

One another object of the present invention is to provide a recombinant host cell comprising the recombinant vector.

Alternate object of the present invention is to provide a method for producing a recombinant protein as described herein.

Yet another object of the present invention is to provide a method for producing delta acyl lactone.

### SUMMARY OF THE INVENTION

The present invention provides a recombinant DNA comprising a nucleic acid fragment operably linked to a heterologous promoter, wherein the nucleic acid fragment encodes a protein having an amino acid sequence as set forth in SEQ ID NO: 2, or SEQ ID NO: 4. The present invention also provides a recombinant vector comprising the said recombinant DNA. The recombinant host cell comprising the recombinant vector is also provided herein. The present invention further provides a recombinant protein and method for producing the said product. Moreover, there is also provided herein a method for producing the delta acyl lactone using the recombinant vector and the recombinant host cell as described herein. The method of the present invention is economical as it deploys a culture medium supplemented with glucose as a substrate material.

### DESCRIPTION OF ACCOMPANYING FIGURES

The accompanying drawings constitute a part of the description and are used to provide further understanding of the present invention. Such accompanying drawings illustrate the embodiments of the present invention, which are used to describe the principles of the present invention together with the description.

FIG. 1 depicts generation of overexpression clone of PKSGPL-TEDEBS. FIG. 1 A) Schematic representation for the cloning of PKSGPL-TEDEBS in pET21c vector. FIG. 1 B) depicts restriction digestion pattern for screening of positive clones. Lane 1, 2-pRSG34 NdeI-HindIII, lane 3, 4-pAV37 NdeI-HindIII, lane 5 to 16-screening of pSSRi10 clones: 5—transformant a—NdeI-speI, 6—transformant b—NdeI-speI, 7—transformant c—NdeI-speI, 8—transformant d—NdeI-speI, 9—transformant e—NdeI-speI, 10—transformant f—NdeI-speI, 11—transformant a—EcoRI, 12—transformant b—EcoRI, 13—transformant c—EcoRI, 14—transformant d—EcoRI, 15—transformant e—EcoRI, 16—transformant f—EcoRI. Expected restriction digestion pattern for pSSRi10 positive clones: NdeI-SpeI: −9535 bp and 6297 bp; coRI: −13567 bp and 2265 bp. Transformants a, c, d e and f are the positive clones and were labelled as pSSRi10a, b, c, d and e respectively. Lane 17, 18—PCR amplification of 1.4 kb fragment of PKSGPL with SpeI site at both 5′ and 3′ end; Lane 19 to 27—Screening for pSSRi11 clones: 19-transformant a NdeI-EcoRI, 20-transformant b NdeI-EcoRI, 21—transformant c NdeI-EcoRI, 22—transformant d NdeI-EcoRI, 23—transformant a SpeI, 24—transformant b SpeI, 25—transformant c SpeI, 26-transformant d SpeI, 27—transformant c BglII. Expected restriction digestion pattern for positive clones: pSSRi11 NdeI-EcoRI: −8170 bp, 5397 bp and 3689 bp; SpeI: −15832 bp and 1424 bp, BglII: −7340 bp, 5855 bp and 4022 bp. Transformants c is the only positive clone and was labelled as pSSRi11a. Lane 28 to 30 screening for pSSRi15 clone: 28-transformant a NdeI-ECORI, 29-transformant a SpeI, 30-transformant a BglII. Expected restriction digestion pattern for positive clones: pSSRi15 NdeI-EcoRI: −8170 bp, 5397 bp and 3689 bp; SpeI: −15832 bp and 1424 bp, BglII: −7340 bp, 5855 bp and 4022 bp. Transformant a was labelled as pSSRi15a, in accordance with an implementation of the present invention.

FIG. 2 depicts protein purification for the recombinant proteins PKSTE (SEQ ID NO: 2) and PKS14TE (SEQ ID NO: 4), in accordance with an implementation of the present invention.

FIG. 3 depicts biochemical assay for PKSTE and PKS14TE; A) Enzymatic assay with C14 labelled MCoA and dodecanoyl NAC as substrate; B) Enzymatic assay for PKS14TE with C14 labelled MCoA and octanoyl NAC as substrate. C) GC-MS chromatograms for PKS14TE assay with MCoA and octanoyl NAC as substrate. CON: no protein control; 1: PKSTE; 2: PKS14TE, in accordance with an embodiment of the present invention.

FIG. 4 depicts GC-MS chromatograms for fraction 3 of metabolites extracted and fractionated from culture filtrate of ΔfadAB BAP1 Mtb FAAL10 pSSRi15a strain, in accordance with an embodiment of the present invention.

FIG. 5 depicts GC-MS chromatograms for δ-dodecalactone standard, in accordance with an embodiment of the present invention.

FIG. 6 depicts intrinsic pathways (marked in blue) and the engineered pathway (marked in red), for producing δ-dodecalactone, in accordance with an embodiment of the pre-sent invention.

FIG. 7 depicts the thin-layer chromatography (TLC) data for the enzymatic function of the recombinant proteins, in accordance with an embodiment of the present invention.

FIG. 8 depicts the TLC data that shows the presence of delta dodecalactone only in the case in which pAV37 is subjected to alkali hydrolysis, in accordance with an embodiment of the present invention.

### DETAILED DESCRIPTION OF THE INVENTION

While the invention is susceptible to various modifications and alternative forms, specific embodiment thereof will be described in detail below. It should be understood, however that it is not intended to limit the invention to the particular forms disclosed, but on the contrary, the invention is to cover all modifications, equivalents, and alternative falling within the scope of the invention as defined by the appended claims.

Although one or more features and/or elements may be described herein in the context of only a single embodiment, or alternatively in the context of more than one embodiment, or further alternatively in the context of all embodiments, the features and/or elements may instead be provided separately or in any appropriate combination or not at all. Conversely, any features and/or elements described in the context of separate embodiments may alternatively be realized as existing together in the context of a single embodiment.

The terminology used herein is for the purpose of describing particular various embodiments only and is not intended to be limiting of various embodiments. As used herein, the singular forms “a,” “an” and “the” are intended to include the plural forms as well, unless the context clearly indicates otherwise.

For the purposes of the present document, the term “recombinant DNA” used herein refers to a DNA molecule formed by laboratory methods of genetic recombination that bring together genetic material from multiple sources, creating sequences that would not otherwise be found in the genome. The recombinant DNA comprises only the coding sequences, that are not found together in nature. The recombinant DNA is a result of human intervention. Such a recombinant DNA may be used in conjunction with a vector. The recombinant DNA encodes a recombinant protein.

As used herein, the term “vector” refers to a DNA molecule which is operably linked to a segment of recombinant DNA. This vector is used as vehicle to carry this segment of recombinant DNA into a host cell where it can be replicated or expressed. The expression and replication of the vector is regulated by promoter and ori region of replication, respectively. Plasmid is one of the examples of vector.

The term “recombinant host cell” refers to a cell that has been manipulated by any method to take up a DNA sequence (e.g., an expression cassette). In the present invention, the host cell that is used herein a prokaryotic cell.

The term “heterologous promoter” refers to a promoter which is obtained from a different source as compared to the gene which is getting regulated by the promoter.

The term “expression”, as used herein, refers to the production of a functional end-product (e.g., protein).

In the present invention, specific nomenclature has been assigned to the recombinant vectors, i.e., pSSRi11 and pSSRi15. In said nomenclature, P stands for plasmid, SSR stands for Sonali Srivastava, I stands for IML lab and the number indicates the number of clone generated.

### Sequences Used in the Present Invention

SEQ ID NO: 1 depicts the nucleic acid sequence encoding amino acid sequence
of PKSTE protein
ATGGTCTCTGCCGAACATCCGATCGAGCCGACCCCGAGCTTCGCAATCATCGGCT
ACGCCGCACGTTTTCCCGGCGCGGCGAGCGCCGAGGAATACTGGGCGTTGTTGC
GGGACGGGCGTGAAGCGATATCCGATGTTCCGAAAGACCGCTGGGACATCGAGG
AGTTCTTCGACCCGGACCCGTCGACACCGGGCAAGGTCGTCACGCGCCGCGCGG
GGTTCGTCGATGACGTAACGGGTTTCGACGCGCCGTTCTTCGGTATGTCCGCCCG
CGAGGTACGTCTGATGGACCCGCAGCACCGGATTCTCATGGAAACCGCGTGGCG
CGCGGTCGAACACTCGGGAATCGCGCCGACGTCGCTGGCCAACAGCAACACGGG
CGTCTTCGTCGGCCTGGCCACCCACGACTACCTCGGTATGGCCTCCGACGAACTC
ACCTACCCCGAGATCGAGGCCTACATGGCCATCGGAACCTCCAATGCCGCTGCG
GCCGGACGGATCAGTTACCGCCTGGGATTGCAGGGACCGGCGGTGGCGGTCGAC
ACCGCGTGCAGTTCGTCGCTGGTGGCGATTCACCAGGCGTGTCAGGCACTTCGCC
TGAACGAGTGCGATCTCGCGTTGGCCGGGGGGCGAACGTCCTGCTCACCCCGG
CCACCATGATCACGTTCTCCAACGCGCACATGCTGGCTCCGGACGGCCGGTGCAA
GACCTTCGACGCGGCGGCCGACGGCTACGTGCGCGGCGAGGGCTGCGGTGTCGT
CGTGGTCAAGCGCCTCGAGGACGCGGTGCGCGACGGCGACCGGATCCGCGCGGT
GATCCGCGGAAGCGCAATCAACCAGGACGGGGCGTCGGGCGGGCTGACCGTGCC
GAACGGCGTTGCTCAGCAACGGGTTATCGCCGACGCGTTGAAGCGCGCCGGCCT
GCGACCGGCCGATGTCGGTTATCTCGAGGCGCACGGCACCGGAACCTCACTTGG
CGACCCCATCGAGGCCCAGGCCGCGGGCGAGGTGCTCGGGGCCGGACGCGCACC
TGAAGAACCGCTGCTGATCGGCTCGGCGAAGACCAACATCGGCCATCTGGAAGC
GGCCGCGGGCATCGCCGGTGTCATCAAGGTGGTCATGGCGCTCGAGAACGAGAC
GTTGCCCAAGCACCTCAATTTCGAGACTCCGTCGCCCCACATCCCGTGGGAGCGT
CTACCCATCGAGGTGGTGAGAGAGACCGTTCCCTGGCAGCGCAACGGCAAGCCG
CGGATCGCCGGGGTGAGCTCATTCGGGTTTGCCGGGACTAACGCGCACGTCATCC
TCGAAGAGGCGCCCGCCGTCGGAACACCGGCGACGACAGAGCCTGTCGAGGATC
CGAGGCGCTACAGCGTCCTTCCGCTGTCGGCACGCACGCCCGAGGCGTTGGTCCG
ACTCGCCGGTGAGTACCGCGACTGGCTGCGCGCCCACCCGCAGGCCCGCCTGGC
CGACGTGTCCCACACCGCGGGAACCGCACGTGCCCACCTGGAGCAGCGTGCCGC
GTTGGTGGTCAACTCGCGGGAATCGGCCATCGAACTGCTCGGCGCGCTCGCCGAC
GACCGTCCGGCGCCCGGCCTGGTCCGCGGTGAATCCCACGAGGCACCCAAGACC
GCGTGGTTGTTCACCGGCCAGGGCAGCCAGTACCCCGGAATGGCGCGTGAGTTG
TTCGACACCGAACCGGTGTTCGCCGAAACCGTGAGGAAATGCGCCGAGGCCGTC
GCAGATATCCTCGAAAAGCCTTTGCTCGACGTCATTTTCGACGCCGACGAGTTGG
ATGGTGACACCGAATCGCCGCTGCGGCACACGACCTATGCGCAACCCGCGCTGTT
CGCCGTCGAATTGGGCCTGGCCCGGCTCTGGCAGTCCTGGGGCTTCGAACCCGAC
GTGGTGGTCGGACACAGTGTCGGGCAGTACTCGGCCGCCTGTGTGGCGGGTGTGT
TCGACGTCGAGGACGGCGCACGACTGATCGCCGAGCGCGGCCGATTGTTCGGCA
GCCTGCCCGCGGGTGGACGCATGGCGGCGGTTTTCACCGCCCCCGAGCGTGCAG
AGCGCCTCACCGACGAGTACCCCAGCCTGTCGGTTGCCGCGTACAACGGTGCCA
ACACCGTATTGTCCGGTCCCGCAAAGGATCTCGAATCCGCCGTGGCCACATTGGT
GGCCGACGGTGTGCGGTGCGACTGGCTGGACACCAGCCACGCCTTCCACTCGGC
ACTGCTCGACCCGATTCTCGACGATTTCGAGTCGTTCGCCCAGAAGCTCGAATAC
CGTGAACCGCAACGTATCCTGATCGACAACCGCACCGGGTCCGCGCTCGGGCGG
AGCACCAGGCTCGACGGTGCCTACTGGCGGCGGCACGCACGCCAGCCTGTCGAG
TTCGCCAAGAGCGTCCGCACCCTCGCCGACATGAACTGCAGGGTGCTGCTGGAG
ATAGGGCCCAGGCCCGTGCTCACCGCCACCGCGCTGGCAGCATGGCCCGACCCG
GCCACCACGCCGCAGGTGCTCGCGTCGCTGCGGCCCACCACGGCCGATCACCGG
CAGATCACCGAGGCCGTCGCCGCCGCCTATGCCGCGGGACATCTGCCCGATTTCC
GCGCGTTCCGGCAACCGGACGCGCGCAAACTCGACCTGCCCACATATCCCTTCGA
GCATCGCCAGTTCTGGTTCTCGGACAACCGTGCGATCGACCGTGACACCCAGACC
GCGAGCGCCGGTTCCGTCACGCCTCACCGCACACAGGCCGTCCGACTTCTCGAAG
ACGGCCAGATCGAGGAACTCGCCGCCCTCATCGACGGCGAGACCGTCGACGAGC
AGACCCTGCGCGTGCTGAACAAGCTCGCGGCGCGCCACAACCAGCAGCGGTCCA
CACACGTCGACCCTGATGCGCGGTACGAGATCCGTTGGGACGCGATCGCTTCGGC
CGCCGCGAGCACGGGCGCGGCAGCCGACTGGATCCTGGTCGGCGACGACACCGC
TGCCGTCCTGGAGTTCGCCGATGCGCTCACCGCAGGCGGTCATCGCCACCAGATC
GTCGCGCTGCCCGGCTCCGACGCCGACGAGGCCCAGCTGGTCGATACGCTGCGC
GCTGCGTCGGCCGGTGAGCTGTACGTCGTGCACATCGCGGCAGGAGACGGTGCC
GCTGCCCCGATGCGGGACCTGCTGCGCGTGCAGCACCGAACCCTTGGTGGGCTGC
GGCGCCTGTTCCGCGCCGCGAACGACGCGGAACTGCGCAGCCCCATCTGGTTGGT
GACACGTGACGGGCAGCGGGTCACCGACACCGACACCGTGGTACCGGAACAGAG
TTGCCTGTGGGGATTCGGCCGCGCGGCGGCCCTGGAACTCCCGCACATCTGGGGT
GGCCTCGCCGACCTCTCCGGTGCGGACACCGAGTGGCCGCGGCTCATCGCGCGG
ATCACGTCACCGCGCGACGGTGAGGACCAGATCGCGCTGCGCGGGGACGCCGTG
TACGCACCCCGACTGGTCCGCCGGACCGGCGAGCCGAGCGACACCCCCCTGCCG
GTGCGGGAGGACCGCACCTATCTGGTGACGGGCGGTCTCGGGGCGATCGGGCTC
GAGGTCGCCGGCTACCTGGCCTCCCACGGTGCCGGTCATGTGGTGCTCACCAGCC
GACGGGAACCGGGCGACGACGCGCGCCGGCGCCTCGACGGTCTCCGCGAGCAGT
ACGCATGTGACGTCCGGGTGATCACCGCCGATGTCGCCGACGCGCACGACGTCG
CGCGCCTCATGACCACCGTCGCCGCCGAACTCCCGCCGCTGGCCGGCATCGTGCA
CGCCGCGGGCGAGATCGGCACCACGCCGCTGAGCGGCCTCGACGACGCCGAGGT
GGATCGCGTCTTCGCCGGGAAGGTCTGGGGCGCCTGGCATCTGAGCGAGGCCCT
GACAGATCTTCGCACCGATCTCGACTTCTTCGTCAGCACATCGTCGATCGCGTCG
GTGTGGGGCGGATTCGGGCAGACCGCCTACGGCGCGGCGAACGCGTTTCTCGAC
GGTCTGGCGTGGCGACTGCGCGAGCGGGGTGTCACCGCTGTCAGCGTGAACTTC
GGGCCGTGGGCCGCGGGCATGGCCGACGCCGAATCACGTGCCCGCCTCGAGCAG
CGAGGCGTGCGGACGTTGTCGCCCGCCGAGGCCCTCGCAGGCATGGCCGACGTC
GTGGCCGGCCCCGTTCAGGGAGTCGTCGCCAAGATCGACTGGTCACGTTTCCTGC
CGCTGTACCAGCAGGCAGGCCGGCGCGCGTTCCTGTCGGAACTCGAAAGCGAAT
TGCCCGCAGCGGCAACCGGCGCTGCTGCGCCCGCGACGGTGCCCGGGAAACCAC
CGCTCGTCGAGCAGTTGACCAAAGCGCCTGTGCAGCAACGCAAAAGCCTCATCA
CGAACTACCTGCGCAACGCGGTCGCCGAGGTCACACGGGTCGACGCCGACGAGA
TCCGCGATGAGGCCGGGTTCTTCGACCTCGGGATGGACTCCCTGATGGCCGTCGA
ACTGCGGCGGCGCATCGAGCAGGGTGTCGGCAAGGACATTCCCGTCACGCTCGT
GATGGATCATCCGCGGTTGTCCGACGCGGCCGACTACCTGCTGGTCGAGGTACTC
GGGTTGGGCGAGCAGACCAACGTGCGGCAGGCGTCGACCGTGACCGCGCGAACC
GACGATCCCATCGCGGTCGTCGCGGTGTCCTGCCGCTTCCCCGGCGCACCCGACC
CGGAGTCGTTCTGGGAGCTGCTCGCCGGTGGTGTCGATGCCATCCGAGAGGTCCC
CGAGGACCGCTGGGACATCGACGAGTTCTACGACCCCGATCCGGACACCCCGGG
CAAGACCTACACGCGGTTCGGCGGATTTCTAGACGGCATCGACGGATTCGATCCC
GAGTTCTTCGGCATCTCACCGCGTGAGGCCGTGTGGATCGAGCCGCAGCAGCGCC
TCATGCTCGAGACGGTGTGGGAGGGTCTCGAGCGGGCCGGCTACGCACCTTCGG
ACCTCCGGGGCAGCCGCACGGGTGTCTTCACCGGTGTTGCCGCCAACGAGTACGC
GCATCTGCTGTCGGCCGAGTCGATCGACAAGATCGAGCCGCACTTCATCACGGGC
AATGCGCTCAACGCGATCTCCGGTCGCGTGGCGTTCGCGCTGGGCTTCGAAGGTC
CTGCCGTCGCGGTCGACACCGCGTGCAGCTCGGCTCTGGTCGCGGTCCATCAGGC
ATGTCAGGCACTGCAGTCCGGGGACTGCGATCTGGCCCTCGCGGGAGGCGTCAA
CGTGCTGCTGAGCCCCGTCACCGTGGTCGCGGCCTCACGCGCGCGGATGCTGTCG
CCGGTGGGCCGGTGCAAGACGTTCGACGCGTCCGCCGACGGGTATGTGCGCAGT
GAGGGCTGCGGCGTCCTGGTGCTCAAGCGCCTCAGCGACGCCGTGCGTGACGGC
GACCGGGTGTGCGCGGTGATCCCTGCGAGCTCGGTGAACCAGGACGGCGCCTCC
AGCGGGTTGACCGTCCCCAACGGCGGTGCGCAGCAACGGCTCATCGAGGCGACG
CTGGCCCGCGCCGGACTGAGCGGCGCCGATGTGGACTACCTCGAGGCACACGGC
ACGGGTACGCCCCTCGGTGACCCGATCGAGGTGCAGGCCGCCGCGGCCGCGTAC
GGCGCCGGGCGTGACGCCGACCGGCCGCTGCTGATGGGATCGGTGAAGACCAAC
ATCGGGCACACCGAATCCGCCTCGGGTGCAGCGGGTCTGATCAAGGTGGTGCTG
TCACTGCAACACGGGGTGCTGCCGCAGAGCCTGCACTTCGAGAATCCGTCACCGC
ACATCCCGTGGGACGCATTGCCGGTGCGGGTGATCGACGAACCGGTGCCGTGGC
AGACCAACGGCCGGGTGCGCCGTGCCGGTGTCAGTTCGTTCGGGTTCACCGGCAC
CAACGCGCACGTGCTCGTCGAGGAGGCCCCGCCGCAACTGCACACCGCCGACGC
TGCGCCCGTGGAACCGGCCGCCACCGCGGAGGCCGGGACCGACGACTCGCCGCA
GGTGCTGGCGCTGTCGGCGCGGTCGCCCGAGGCGCTCGTGGAGTTGGCGCGGCG
CTACGACGACTGGTTGAGCGCGACGCCCGGCCTCGACATCGCCGACGTGTGCCA
CACCGCGGGCGTGGGCCGTTCGCACTTCGAATACCGCGCAGCCCTGGTGGTCGAC
TCGGTCGAGCGGGCACGCGAGGGTCTCGCCGCGCTCGCCGAGGGGCGCACTCCT
GCGGGCGTGGTGCGCGGCGAGCATGTGCACCGTCCGACGACGGCATGGCTGTTC
ACCGGACAGGGCAGCCAGTACCCGGGCATGGCCCGCGAATTGTTCGACACGCAA
CCGGTTTTCGCCCAGACCGTTCGGCAGTGCGCCGAGGCGGTCGCCGACATGCTGC
CGCGACCACTGCTGGACGTGCTGTTCGCGACCGACCGCGAAACCGCGGACCTGC
TGCAGCACACGTCGTTCGCGCAGCCCGCGCTGTTCGCGGTCGAGATGGGGCTAGC
GCGGCTGTGGCAGTCATGGGCCATCGAACCCGATGTGGTGCTGGGGCACAGCGT
CGGCCAGTACGCCGCGGCCTGCGTGGCGGGCGTGTTCAGCCTTGACGACGGCGC
ACGACTGATGGCCGAGCGCGGCCGCATGTTCGGCAGCCTTCCCGAAGGCGGCCG
GATGGTGGCCGTGTTCGCCGACGCCGAACACGTCGAGCAGGTGGCCGGTGAGTT
CCCCCGGGTGTCGGTCGGCGCCTACAACGGCCCCAACACCGTGCTCTCGGGTCCC
GGCGAGGACCTCGAAGAGATCGTCGAGAGGTTCGAGGACGAGGGGATCCGCTGC
ACGTGGCTGGCGACCAGCCACGCGTTCCACTCCGAACTGCTCGATCCCGTGCTCG
ACGAGTTCGAGGCCTACGCGGCCCAGTTCCAGTTCGCAGCGCCGACACTGCCTTT
GGTGTGCAACCGGACCGGGGCCGTGCTCACCGGGCAGACCCCGCTCGACGCGCA
GTACTGGCGCAGGCATTCCCGCCAGCCCGTGCAGTTCGCCGAGAGTGTGCGCACC
GTCGCCGCGCTCGGCTGCTCGGTGCTGATGGAGATCGGCCCGCAACCTGTGCTGA
CCGGGGCCGCGGTACAGGTGTGGCCCGAACACATGGCTGCCCCGCGGGCCGTCG
CCTCGCTGCGCAAGGGCGTCGCCGATCGCCGACAGATCGCCGATGCGCTGGCGT
CGGCCTACGTCGGCGGCCACCGCCCCGACTTCGCCGCGGTGCACCGGCAGCCAC
GCCGCCCGGTCGAGTTGCCCACCTATCCGTTCCAGCGCCGCAGGTTCTGGCCGAA
GGCCTCGAGCATCGCCGTCGACGGTGGGACCGCGGCGTCGGGAATCCTGGGCAG
CGGCAAGGATCTGGCGTCCGGCGACACCATCTACACGAGCCGGCTGTCCGTCAA
ATCGCAGCCGTGGCTGGCCGATCACGTCATCTACGGCACCGTCGTCGTCCCCGGC
GCGACGTATGCGGCGATGGCGCTCGCCGCGGTCGGCACACCGGCACGCCTGAAG
GACGTGTTCTTCTACGAGCCGATCATCCTGCCCGAGAAGGCCTCTCGCGAGGTCC
AGCTGACGTTGCACCCTGCCGACGGTGGCAGCGTGCTCAGCTTCCAGATCCACAG
CCGCCCGTACGGTGAACGTGGTGCCGACTGGTCGCTGAACGCCGAGGGCACGGT
CGACACGGCCGGCAGCGACCCGGACGCCGCCGCAGACGAGCAGTCGGATCCGGT
CGACGAGGTGATCGAACGCCTCGAGCGCATGCGTCCGCAGGACCTCTTCGAGAC
CTTCGCCGACCTGGAACTGGCGTGGGGTCCCACATGGTCGGGCTCGCTGAAATCG
TTGTGGCTCGGCGACGGTGAGGCCGTCGGCGACATCCTCGTCGGCGCCGAACTCG
CCGAGCAACTCGGCTCCGAGCCCATGCACCCGGTGCTGATGGACCTGTGCACGG
GCGTCGCGTTCCCCGCGTTCCCGGCGCTGCTCGCGGCCGAACAGGGCGTCAACGA
CCTGTTCCTGCCGCTGCGCTACGGGCAGGTGACGCTCGCGGAGAAGATGCCGCG
GCGGTTCTACTGCCGGGCGAGGTGGCACCGTAGCGCGCTCGACAGCGAAACGCA
GGTGTTCGAACTCGAATTCGTCGACCTCGGTGGGCGAGCGCTCGGCGGCATCCGC
GAGTTCACCGTCAAACGTGCGCCGCGCGAGGCACTGCTGCGCGGCCTCGGGGGT
GACGCGACCCGCCTGCTCTACACGCTCGGGTGGCACGAGGTGCCGCCGCCACCA
TCGGCCGACGACGCGGTCGGCACGTGGCTGATCGCCGGGTTCGACGAGCTGGCC
GCGAAGGTGCCCGGCTGTGTCCCCATCGACCGCAACACCGATCTGCAGGTCCTCG
GTGAGGTGCTGTCGCAGGCGCACGCTCGCGGCGCGGGATTCTCGGGCGTCGTGT
GGCGTTGCGCGACGCCTGGCCGGAAGGAATCGGGTTCTGCCTCCGCCGAATCCG
CCCGTCTGGAGGCCGAGATCACCAATCTGCTCAGCGCGGTGCACGCCGCGCAGG
GGCCGAGCGTGAAACTGCCCGGCGGACTCTGGATCGTCACCGAACACGGCGTGG
CATGTGAATCCGGCGAGCCGGTGGACCCGGTGCAGGCCGCGCTGTGGGGCTTCG
GCCGAACGACGATCAACGAGGAACCGGCGCTGCACTGCCGGCTCGTCGACTGCG
ACGGATCTTCCGAGGCCGTCGACCTGCTGGCCGCTCTGCTGGCCGCACCGGGCAT
CCAGGAACCGGAAATCGCCGTGCGGCAGGGCAAACTGCTGGCGTCACGGTTGTT
GCCGTGGGCCCGCAGTGGTCATCTCACGCTGCCGCGCGGGGGCGACTTCGTCCTT
GCGCCCACCGAGCGCGGCGCGATCGACAACCTGCGCCTCACGGAGACGGACGTG
CCGGAGCCGGACGAGGGCTACGTGCAGGTTCGGGTCGAGGCCGCGGGCCTCAAC
TTCCGCGACGTGCTCAACGTGCTCGGCCTGTACCCCGGTGATCCGGGACCGATCG
GCGGCGACTTCGCAGGCACCGTGACCCAACTGGGGGCAGGTGTGAGTGGACTCG
AAATCGGCCAACGCGTGTACGGGTCGATGCAGGGTGCCTTCGCGAGCCGCTTCA
ACGTGCCCGCCCAGTTCCTGGCGCCGATTCCCGACGGCATCGGCGCGGTCGAGGC
CGCCACGATCCCGGCCGCGGCGCTGACGGTCCGGCTCGCGTTCGACTGGGCGCA
ACTCAAACCCGGCGACAAGGTGCTCATCCACGCCGCCAGCGGTGGCGTGGGGCT
CGCGGCCATCCAGATGGCGCAGCAGTCCGGCGCCGAGGTCTTCGCCACCGCGAG
CACCTTCAAGCGTTCGACCCTGCGCAGGCTGGGCGTGAAGTACGTCTACGACTCT
CGGACGACGGACTTCGCCGACCAGATCCTCGCGGACACCAACGGTGCCGGCGTG
GACGTGGTGCTCAACAGCCTCACTAGTGCGGGTTTCGTCGAGGCGACGCTGAAG
GCCACCGCGCAGGGCGGACGCTTCGCCGAGATCGCCAAGCGCGACATCTGGTCG
CATGAGCAGATGGCCGAGGCCCGCCCCGACATCGCCTACGAGATCGTCGCGCTG
GACACCGTGATGTTCACCGAGCCCGATCGCATCCGCGACCTGCTCACCGAGGTGT
CCGACGGGATGGCGCGCGGCGAGTGGACACCACTACCCGCCGAGATCTACCCGA
TCACCGAGGCCCGGTCGGCGTTCCGGCGCATGCAACAGGCACGGCACATCGGCA
AGATTGTGCTGCAGGTCCCCAAACCCCTTCAGCCGCAGCAGGACCGGAGCTACCT
GATCACCGGCGGACTCGGGGCGATCGGCCTGCACACGGCGGCGTATCTGGCGCA
GCTCGGCGCGGGCGACATCGTGCTGACCAGCCGGCGCGACCCCGACGAGTCGGC
CCAGCGGGCGATCGACGAGATCACCGAGCGCCACAAGTGCCGCATCCACACCTA
TGCGGCCGATGTGGGTGACGAGGCACAGGTGGCCGATCTGCTGGCGCGGATCCG
GGCCGAGTTGCCTCCACTGGCCGGAATCGCCCATCTGGCGGGCGTGCTCGACGAC
GCGCTGCTGTCGGCGCAGAACCCCGAGCGCTTCCGGACCACGTTGGCGCCCAAG
GCCTATGGCGCGAGCCACCTGGACCGCCTGACGCGCGACGACGACCTGGACTTC
TTCATCGTGTCCTCGTCGGTGTCGAGCCTGTTCGGATCACCGGGGCAGGCCAACT
ACGCAACCGCCAATGCGATGCTCGACGGTCTGGTCGCCAAGCGCCGTGCGCAGG
GCCTGGTGGCCACGGGCATCAACTTCGGGCCGTGGGGTCAGGGCGGCATGGCGT
CGTCGGAGGCCGCGACCGCGAACATCACCGCCCAAGGTCTGATCCCGTTGGATC
CGTCGGCCGCGTTGCACGCGCTGGCCGAGGTCATCGCGAACGGCTCCGGGCAGG
CGACGGTGCTCAAGGCCAACTGGCAGCGCGCGGCGAAGGTTCTCGGCTCGGCGC
GCCCGCCGATCCTGGACCTCGTCCTGCCGAGCGCGGCCGGGGAGGCCACGGGTG
ACAGCGAGCTGCTCAAGCAGTTGATGGAGATCCCGATACCGCAGCGCGCCGGGT
TCGTCACCGAGTTCCTGCAACGCGAAGTGCAGAACTTCCTGCGTCTCGCGTCGCC
GCCCGCGGCGTCGAGCCGGTTCCTGGACCTCGGCACGGATTCGCTGATGGCCATC
GAGCTGCGCAACCGGCTGCACAGCCAGTTCGGGGGCAAGTTCACCATCAACGCG
ACCGCGGTGTTCGACTACCCGACCATCGGCGGGCTCGCGGAGTACCTCACTAGTC
AGCTCGACAGCGGGACTCCCGCCCGGGAAGCGAGCAGCGCTCTTCGCGACGGCT
ACCGGCAGGCGGGCGTGTCGGGCAGGGTCCGGTCCTACCTCGACCTGCTGGCGG
GGCTGTCGGACTTCCGCGAGCACTTCGACGGCTCCGACGGGTTCTCCCTCGATCT
CGTGGACATGGCCGACGGTCCCGGAGAGGTCACGGTGATCTGCTGCGCGGGAAC
GGCGGCGATCTCCGGTCCGCACGAGTTCACCCGGCTCGCCGGGGCGCTGCGCGG
AATCGCTCCGGTTCGGGCCGTGCCCCAGCCCGGCTACGAGGAGGGCGAACCTCT
GCCGTCGTCGATGGCGGCGGTGGCGGCGGTGCAGGCCGATGCGGTCATCAGGAC
ACAGGGGGACAAGCCGTTCGTGGTGGCCGGTCACTCCGCGGGGGCACTGATGGC
CTACGCGCTGGCGACCGAACTGCTCGATCGCGGGCACCCGCCACGCGGTGTCGTC
CTGATCGACGTCTACCCGCCCGGTCACCAGGACGCGATGAACGCCTGGCTGGAG
GAGCTGACCGCCACGCTGTTCGACCGCGAGACGGTGCGGATGGACGACACCAGG
CTCACCGCCCTGGGCGCCTACGACCGCCTCACCGGTCAGTGGCGACCCCGGGAA
ACCGGGCTGCCGACGCTGCTGGTCAGCGCCGGCGAGCCGATGGGTCCGTGGCCC
GACGACAGCTGGAAGCCGACGTGGCCCTTCGAGCACGACACCGTCGCCGTCCCC
GGCGACCACTTCACGATGGTGCAGGAACACGCCGACGCGATCGCGCGGCACATC
GACGCCTGGCTGGGCGGAGGGAATTCATGA
SEQ ID NO: 2 depicts the amino acid sequence of PKSTE protein
MVSAEHPIEPTPSFAIIGYAARFPGAASAEEYWALLRDGREAISDVPKDRWDIEEFFDP
DPSTPGKVVTRRAGFVDDVTGFDAPFFGMSAREVRLMDPQHRILMETAWRAVEHSG
IAPTSLANSNTGVFVGLATHDYLGMASDELTYPEIEAYMAIGTSNAAAAGRISYRLG
LQGPAVAVDTACSSSLVAIHQACQALRLNECDLALAGGANVLLTPATMITFSNAHM
LAPDGRCKTFDAAADGYVRGEGCGVVVVKRLEDAVRDGDRIRAVIRGSAINQDGAS
GGLTVPNGVAQQRVIADALKRAGLRPADVGYLEAHGTGTSLGDPIEAQAAGEVLGA
GRAPEEPLLIGSAKTNIGHLEAAAGIAGVIKVVMALENETLPKHLNFETPSPHIPWERL
PIEVVRETVPWQRNGKPRIAGVSSFGFAGTNAHVILEEAPAVGTPATTEPVEDPRRYS
VLPLSARTPEALVRLAGEYRDWLRAHPQARLADVSHTAGTARAHLEQRAALVVNS
RESAIELLGALADDRPAPGLVRGESHEAPKTAWLFTGQGSQYPGMARELFDTEPVFA
ETVRKCAEAVADILEKPLLDVIFDADELDGDTESPLRHTTYAQPALFAVELGLARLW
QSWGFEPDVVVGHSVGQYSAACVAGVFDVEDGARLIAERGRLFGSLPAGGRMAAV
FTAPERAERLTDEYPSLSVAAYNGANTVLSGPAKDLESAVATLVADGVRCDWLDTS
HAFHSALLDPILDDFESFAQKLEYREPQRILIDNRTGSALGRSTRLDGAYWRRHARQP
VEFAKSVRTLADMNCRVLLEIGPRPVLTATALAAWPDPATTPQVLASLRPTTADHRQ
ITEAVAAAYAAGHLPDFRAFRQPDARKLDLPTYPFEHRQFWFSDNRAIDRDTQTASA
GSVTPHRTQAVRLLEDGQIEELAALIDGETVDEQTLRVLNKLAARHNQQRSTHVDPD
ARYEIRWDAIASAAASTGAAADWILVGDDTAAVLEFADALTAGGHRHQIVALPGSD
ADEAQLVDTLRAASAGELYVVHIAAGDGAAAPMRDLLRVQHRTLGGLRRLFRAAN
DAELRSPIWLVTRDGQRVTDTDTVVPEQSCLWGFGRAAALELPHIWGGLADLSGAD
TEWPRLIARITSPRDGEDQIALRGDAVYAPRLVRRTGEPSDTPLPVREDRTYLVTGGL
GAIGLEVAGYLASHGAGHVVLTSRREPGDDARRRLDGLREQYACDVRVITADVADA
HDVARLMTTVAAELPPLAGIVHAAGEIGTTPLSGLDDAEVDRVFAGKVWGAWHLSE
ALTDLRTDLDFFVSTSSIASVWGGFGQTAYGAANAFLDGLAWRLRERGVTAVSVNF
GPWAAGMADAESRARLEQRGVRTLSPAEALAGMADVVAGPVQGVVAKIDWSRFLP
LYQQAGRRAFLSELESELPAAATGAAAPATVPGKPPLVEQLTKAPVQQRKSLITNYL
RNAVAEVTRVDADEIRDEAGFFDLGMDSLMAVELRRRIEQGVGKDIPVTLVMDHPR
LSDAADYLLVEVLGLGEQTNVRQASTVTARTDDPIAVVAVSCRFPGAPDPESFWELL
AGGVDAIREVPEDRWDIDEFYDPDPDTPGKTYTRFGGFLDGIDGFDPEFFGISPREAV
WIEPQQRLMLETVWEGLERAGYAPSDLRGSRTGVFTGVAANEYAHLLSAESIDKIEP
HFITGNALNAISGRVAFALGFEGPAVAVDTACSSALVAVHQACQALQSGDCDLALA
GGVNVLLSPVTVVAASRARMLSPVGRCKTFDASADGYVRSEGCGVLVLKRLSDAV
RDGDRVCAVIPASSVNQDGASSGLTVPNGGAQQRLIEATLARAGLSGADVDYLEAH
GTGTPLGDPIEVQAAAAAYGAGRDADRPLLMGSVKTNIGHTESASGAAGLIKVVLSL
QHGVLPQSLHFENPSPHIPWDALPVRVIDEPVPWQTNGRVRRAGVSSFGFTGTNAHV
LVEEAPPQLHTADAAPVEPAATAEAGTDDSPQVLALSARSPEALVELARRYDDWLS
ATPGLDIADVCHTAGVGRSHFEYRAALVVDSVERAREGLAALAEGRTPAGVVRGEH
VHRPTTAWLFTGQGSQYPGMARELFDTQPVFAQTVRQCAEAVADMLPRPLLDVLFA
TDRETADLLQHTSFAQPALFAVEMGLARLWQSWAIEPDVVLGHSVGQYAAACVAG
VFSLDDGARLMAERGRMFGSLPEGGRMVAVFADAEHVEQVAGEFPRVSVGAYNGP
NTVLSGPGEDLEEIVERFEDEGIRCTWLATSHAFHSELLDPVLDEFEAYAAQFQFAAP
TLPLVCNRTGAVLTGQTPLDAQYWRRHSRQPVQFAESVRTVAALGCSVLMEIGPQP
VLTGAAVQVWPEHMAAPRAVASLRKGVADRRQIADALASAYVGGHRPDFAAVHR
QPRRPVELPTYPFQRRRFWPKASSIAVDGGTAASGILGSGKDLASGDTIYTSRLSVKS
QPWLADHVIYGTVVVPGATYAAMALAAVGTPARLKDVFFYEPIILPEKASREVQLTL
HPADGGSVLSFQIHSRPYGERGADWSLNAEGTVDTAGSDPDAAADEQSDPVDEVIER
LERMRPQDLFETFADLELAWGPTWSGSLKSLWLGDGEAVGDILVGAELAEQLGSEP
MHPVLMDLCTGVAFPAFPALLAAEQGVNDLFLPLRYGQVTLAEKMPRRFYCRARW
HRSALDSETQVFELEFVDLGGRALGGIREFTVKRAPREALLRGLGGDATRLLYTLGW
HEVPPPPSADDAVGTWLIAGFDELAAKVPGCVPIDRNTDLQVLGEVLSQAHARGAG
FSGVVWRCATPGRKESGSASAESARLEAEITNLLSAVHAAQGPSVKLPGGLWIVTEH
GVACESGEPVDPVQAALWGFGRTTINEEPALHCRLVDCDGSSEAVDLLAALLAAPGI
QEPEIAVRQGKLLASRLLPWARSGHLTLPRGGDFVLAPTERGAIDNLRLTETDVPEPD
EGYVQVRVEAAGLNFRDVLNVLGLYPGDPGPIGGDFAGTVTQLGAGVSGLEIGQRV
YGSMQGAFASRFNVPAQFLAPIPDGIGAVEAATIPAAALTVRLAFDWAQLKPGDKVL
IHAASGGVGLAAIQMAQQSGAEVFATASTFKRSTLRRLGVKYVYDSRTTDFADQILA
DTNGAGVDVVLNSLTSAGFVEATLKATAQGGRFAEIAKRDIWSHEQMAEARPDIAY
EIVALDTVMFTEPDRIRDLLTEVSDGMARGEWTPLPAEIYPITEARSAFRRMQQARHI
GKIVLQVPKPLQPQQDRSYLITGGLGAIGLHTAAYLAQLGAGDIVLTSRRDPDESAQR
AIDEITERHKCRIHTYAADVGDEAQVADLLARIRAELPPLAGIAHLAGVLDDALLSAQ
NPERFRTTLAPKAYGASHLDRLTRDDDLDFFIVSSSVSSLFGSPGQANYATANAMLD
GLVAKRRAQGLVATGINFGPWGQGGMASSEAATANITAQGLIPLDPSAALHALAEVI
ANGSGQATVLKANWQRAAKVLGSARPPILDLVLPSAAGEATGDSELLKQLMEIPIPQ
RAGFVTEFLQREVQNFLRLASPPAASSRFLDLGTDSLMAIELRNRLHSQFGGKFTINA
TAVFDYPTIGGLAEYLTSQLDSGTPAREASSALRDGYRQAGVSGRVRSYLDLLAGLS
DFREHFDGSDGFSLDLVDMADGPGEVTVICCAGTAAISGPHEFTRLAGALRGIAPVR
AVPQPGYEEGEPLPSSMAAVAAVQADAVIRTQGDKPFVVAGHSAGALMAYALATE
LLDRGHPPRGVVLIDVYPPGHQDAMNAWLEELTATLFDRETVRMDDTRLTALGAY
DRLTGQWRPRETGLPTLLVSAGEPMGPWPDDSWKPTWPFEHDTVAVPGDHFTMVQ
EHADAIARHIDAWLGGGNS
SEQ ID NO: 3 depicts the nucleic acid sequence encoding PKS14TE protein
ATGGTCTCTGCCGAACATCCGATCGAGCCGACCCCGAGCTTCGCAATCATCGGCT
ACGCCGCACGTTTTCCCGGCGCGGCGAGCGCCGAGGAATACTGGGCGTTGTTGC
GGGACGGGCGTGAAGCGATATCCGATGTTCCGAAAGACCGCTGGGACATCGAGG
AGTTCTTCGACCCGGACCCGTCGACACCGGGCAAGGTCGTCACGCGCCGCGCGG
GGTTCGTCGATGACGTAACGGGTTTCGACGCGCCGTTCTTCGGTATGTCCGCCCG
CGAGGTACGTCTGATGGACCCGCAGCACCGGATTCTCATGGAAACCGCGTGGCG
CGCGGTCGAACACTCGGGAATCGCGCCGACGTCGCTGGCCAACAGCAACACGGG
CGTCTTCGTCGGCCTGGCCACCCACGACTACCTCGGTATGGCCTCCGACGAACTC
ACCTACCCCGAGATCGAGGCCTACATGGCCATCGGAACCTCCAATGCCGCTGCG
GCCGGACGGATCAGTTACCGCCTGGGATTGCAGGGACCGGCGGTGGCGGTCGAC
ACCGCGTGCAGTTCGTCGCTGGTGGCGATTCACCAGGCGTGTCAGGCACTTCGCC
TGAACGAGTGCGATCTCGCGTTGGCCGGCGGGGCGAACGTCCTGCTCACCCCGG
CCACCATGATCACGTTCTCCAACGCGCACATGCTGGCTCCGGACGGCCGGTGCAA
GACCTTCGACGCGGCGGCCGACGGCTACGTGCGCGGCGAGGGCTGCGGTGTCGT
CGTGGTCAAGCGCCTCGAGGACGCGGTGCGCGACGGCGACCGGATCCGCGCGGT
GATCCGCGGAAGCGCAATCAACCAGGACGGGGCGTCGGGCGGGCTGACCGTGCC
GAACGGCGTTGCTCAGCAACGGGTTATCGCCGACGCGTTGAAGCGCGCCGGCCT
GCGACCGGCCGATGTCGGTTATCTCGAGGCGCACGGCACCGGAACCTCACTTGG
CGACCCCATCGAGGCCCAGGCCGCGGGCGAGGTGCTCGGGGCCGGACGCGCACC
TGAAGAACCGCTGCTGATCGGCTCGGCGAAGACCAACATCGGCCATCTGGAAGC
GGCCGCGGGCATCGCCGGTGTCATCAAGGTGGTCATGGCGCTCGAGAACGAGAC
GTTGCCCAAGCACCTCAATTTCGAGACTCCGTCGCCCCACATCCCGTGGGAGCGT
CTACCCATCGAGGTGGTGAGAGAGACCGTTCCCTGGCAGCGCAACGGCAAGCCG
CGGATCGCCGGGGTGAGCTCATTCGGGTTTGCCGGGACTAACGCGCACGTCATCC
TCGAAGAGGCGCCCGCCGTCGGAACACCGGCGACGACAGAGCCTGTCGAGGATC
CGAGGCGCTACAGCGTCCTTCCGCTGTCGGCACGCACGCCCGAGGCGTTGGTCCG
ACTCGCCGGTGAGTACCGCGACTGGCTGCGCGCCCACCCGCAGGCCCGCCTGGC
CGACGTGTCCCACACCGCGGGAACCGCACGTGCCCACCTGGAGCAGCGTGCCGC
GTTGGTGGTCAACTCGCGGGAATCGGCCATCGAACTGCTCGGCGCGCTCGCCGAC
GACCGTCCGGCGCCCGGCCTGGTCCGCGGTGAATCCCACGAGGCACCCAAGACC
GCGTGGTTGTTCACCGGCCAGGGCAGCCAGTACCCCGGAATGGCGCGTGAGTTG
TTCGACACCGAACCGGTGTTCGCCGAAACCGTGAGGAAATGCGCCGAGGCCGTC
GCAGATATCCTCGAAAAGCCTTTGCTCGACGTCATTTTCGACGCCGACGAGTTGG
ATGGTGACACCGAATCGCCGCTGCGGCACACGACCTATGCGCAACCCGCGCTGTT
CGCCGTCGAATTGGGCCTGGCCCGGCTCTGGCAGTCCTGGGGCTTCGAACCCGAC
GTGGTGGTCGGACACAGTGTCGGGCAGTACTCGGCCGCCTGTGTGGCGGGTGTGT
TCGACGTCGAGGACGGCGCACGACTGATCGCCGAGCGCGGCCGATTGTTCGGCA
GCCTGCCCGCGGGTGGACGCATGGCGGCGGTTTTCACCGCCCCCGAGCGTGCAG
AGCGCCTCACCGACGAGTACCCCAGCCTGTCGGTTGCCGCGTACAACGGTGCCA
ACACCGTATTGTCCGGTCCCGCAAAGGATCTCGAATCCGCCGTGGCCACATTGGT
GGCCGACGGTGTGCGGTGCGACTGGCTGGACACCAGCCACGCCTTCCACTCGGC
ACTGCTCGACCCGATTCTCGACGATTTCGAGTCGTTCGCCCAGAAGCTCGAATAC
CGTGAACCGCAACGTATCCTGATCGACAACCGCACCGGGTCCGCGCTCGGGCGG
AGCACCAGGCTCGACGGTGCCTACTGGCGGCGGCACGCACGCCAGCCTGTCGAG
TTCGCCAAGAGCGTCCGCACCCTCGCCGACATGAACTGCAGGGTGCTGCTGGAG
ATAGGGCCCAGGCCCGTGCTCACCGCCACCGCGCTGGCAGCATGGCCCGACCCG
GCCACCACGCCGCAGGTGCTCGCGTCGCTGCGGCCCACCACGGCCGATCACCGG
CAGATCACCGAGGCCGTCGCCGCCGCCTATGCCGCGGGACATCTGCCCGATTTCC
GCGCGTTCCGGCAACCGGACGCGCGCAAACTCGACCTGCCCACATATCCCTTCGA
GCATCGCCAGTTCTGGTTCTCGGACAACCGTGCGATCGACCGTGACACCCAGACC
GCGAGCGCCGGTTCCGTCACGCCTCACCGCACACAGGCCGTCCGACTTCTCGAAG
ACGGCCAGATCGAGGAACTCGCCGCCCTCATCGACGGCGAGACCGTCGACGAGC
AGACCCTGCGCGTGCTGAACAAGCTCGCGGCGCGCCACAACCAGCAGCGGTCCA
CACACGTCGACCCTGATGCGCGGTACGAGATCCGTTGGGACGCGATCGCTTCGGC
CGCCGCGAGCACGGGCGCGGCAGCCGACTGGATCCTGGTCGGCGACGACACCGC
TGCCGTCCTGGAGTTCGCCGATGCGCTCACCGCAGGCGGTCATCGCCACCAGATC
GTCGCGCTGCCCGGCTCCGACGCCGACGAGGCCCAGCTGGTCGATACGCTGCGC
GCTGCGTCGGCCGGTGAGCTGTACGTCGTGCACATCGCGGCAGGAGACGGTGCC
GCTGCCCCGATGCGGGACCTGCTGCGCGTGCAGCACCGAACCCTTGGTGGGCTGC
GGCGCCTGTTCCGCGCCGCGAACGACGCGGAACTGCGCAGCCCCATCTGGTTGGT
GACACGTGACGGGCAGCGGGTCACCGACACCGACACCGTGGTACCGGAACAGAG
TTGCCTGTGGGGATTCGGCCGCGCGGCGGCCCTGGAACTCCCGCACATCTGGGGT
GGCCTCGCCGACCTCTCCGGTGCGGACACCGAGTGGCCGCGGCTCATCGCGCGG
ATCACGTCACCGCGCGACGGTGAGGACCAGATCGCGCTGCGCGGGGACGCCGTG
TACGCACCCCGACTGGTCCGCCGGACCGGCGAGCCGAGCGACACCCCCCTGCCG
GTGCGGGAGGACCGCACCTATCTGGTGACGGGCGGTCTCGGGGCGATCGGGCTC
GAGGTCGCCGGCTACCTGGCCTCCCACGGTGCCGGTCATGTGGTGCTCACCAGCC
GACGGGAACCGGGCGACGACGCGCGCCGGCGCCTCGACGGTCTCCGCGAGCAGT
ACGCATGTGACGTCCGGGTGATCACCGCCGATGTCGCCGACGCGCACGACGTCG
CGCGCCTCATGACCACCGTCGCCGCCGAACTCCCGCCGCTGGCCGGCATCGTGCA
CGCCGCGGGCGAGATCGGCACCACGCCGCTGAGCGGCCTCGACGACGCCGAGGT
GGATCGCGTCTTCGCCGGGAAGGTCTGGGGCGCCTGGCATCTGAGCGAGGCCCT
GACAGATCTTCGCACCGATCTCGACTTCTTCGTCAGCACATCGTCGATCGCGTCG
GTGTGGGGCGGATTCGGGCAGACCGCCTACGGCGCGGCGAACGCGTTTCTCGAC
GGTCTGGCGTGGCGACTGCGCGAGCGGGGTGTCACCGCTGTCAGCGTGAACTTC
GGGCCGTGGGCCGCGGGCATGGCCGACGCCGAATCACGTGCCCGCCTCGAGCAG
CGAGGCGTGCGGACGTTGTCGCCCGCCGAGGCCCTCGCAGGCATGGCCGACGTC
GTGGCCGGCCCCGTTCAGGGAGTCGTCGCCAAGATCGACTGGTCACGTTTCCTGC
CGCTGTACCAGCAGGCAGGCCGGCGCGCGTTCCTGTCGGAACTCGAAAGCGAAT
TGCCCGCAGCGGCAACCGGCGCTGCTGCGCCCGCGACGGTGCCCGGGAAACCAC
CGCTCGTCGAGCAGTTGACCAAAGCGCCTGTGCAGCAACGCAAAAGCCTCATCA
CGAACTACCTGCGCAACGCGGTCGCCGAGGTCACACGGGTCGACGCCGACGAGA
TCCGCGATGAGGCCGGGTTCTTCGACCTCGGGATGGACTCCCTGATGGCCGTCGA
ACTGCGGCGGCGCATCGAGCAGGGTGTCGGCAAGGACATTCCCGTCACGCTCGT
GATGGATCATCCGCGGTTGTCCGACGCGGCCGACTACCTGCTGGTCGAGGTACTC
GGGTTGGGCGAGCAGACCAACGTGCGGCAGGCGTCGACCGTGACCGCGCGAACC
GACGATCCCATCGCGGTCGTCGCGGTGTCCTGCCGCTTCCCCGGCGCACCCGACC
CGGAGTCGTTCTGGGAGCTGCTCGCCGGTGGTGTCGATGCCATCCGAGAGGTCCC
CGAGGACCGCTGGGACATCGACGAGTTCTACGACCCCGATCCGGACACCCCGGG
CAAGACCTACACGCGGTTCGGCGGATTTCTAGACGGCATCGACGGATTCGATCCC
GAGTTCTTCGGCATCTCACCGCGTGAGGCCGTGTGGATCGAGCCGCAGCAGCGCC
TCATGCTCGAGACGGTGTGGGAGGGTCTCGAGCGGGCCGGCTACGCACCTTCGG
ACCTCCGGGGCAGCCGCACGGGTGTCTTCACCGGTGTTGCCGCCAACGAGTACGC
GCATCTGCTGTCGGCCGAGTCGATCGACAAGATCGAGCCGCACTTCATCACGGGC
AATGCGCTCAACGCGATCTCCGGTCGCGTGGCGTTCGCGCTGGGCTTCGAAGGTC
CTGCCGTCGCGGTCGACACCGCGTGCAGCTCGGCTCTGGTCGCGGTCCATCAGGC
ATGTCAGGCACTGCAGTCCGGGGACTGCGATCTGGCCCTCGCGGGAGGCGTCAA
CGTGCTGCTGAGCCCCGTCACCGTGGTCGCGGCCTCACGCGCGCGGATGCTGTCG
CCGGTGGGCCGGTGCAAGACGTTCGACGCGTCCGCCGACGGGTATGTGCGCAGT
GAGGGCTGCGGCGTCCTGGTGCTCAAGCGCCTCAGCGACGCCGTGCGTGACGGC
GACCGGGTGTGCGCGGTGATCCCTGCGAGCTCGGTGAACCAGGACGGCGCCTCC
AGCGGGTTGACCGTCCCCAACGGCGGTGCGCAGCAACGGCTCATCGAGGCGACG
CTGGCCCGCGCCGGACTGAGCGGCGCCGATGTGGACTACCTCGAGGCACACGGC
ACGGGTACGCCCCTCGGTGACCCGATCGAGGTGCAGGCCGCCGCGGCCGCGTAC
GGCGCCGGGCGTGACGCCGACCGGCCGCTGCTGATGGGATCGGTGAAGACCAAC
ATCGGGCACACCGAATCCGCCTCGGGTGCAGCGGGTCTGATCAAGGTGGTGCTG
TCACTGCAACACGGGGTGCTGCCGCAGAGCCTGCACTTCGAGAATCCGTCACCGC
ACATCCCGTGGGACGCATTGCCGGTGCGGGTGATCGACGAACCGGTGCCGTGGC
AGACCAACGGCCGGGTGCGCCGTGCCGGTGTCAGTTCGTTCGGGTTCACCGGCAC
CAACGCGCACGTGCTCGTCGAGGAGGCCCCGCCGCAACTGCACACCGCCGACGC
TGCGCCCGTGGAACCGGCCGCCACCGCGGAGGCCGGGACCGACGACTCGCCGCA
GGTGCTGGCGCTGTCGGCGCGGTCGCCCGAGGCGCTCGTGGAGTTGGCGCGGCG
CTACGACGACTGGTTGAGCGCGACGCCCGGCCTCGACATCGCCGACGTGTGCCA
CACCGCGGGCGTGGGCCGTTCGCACTTCGAATACCGCGCAGCCCTGGTGGTCGAC
TCGGTCGAGCGGGCACGCGAGGGTCTCGCCGCGCTCGCCGAGGGGCGCACTCCT
GCGGGCGTGGTGCGCGGCGAGCATGTGCACCGTCCGACGACGGCATGGCTGTTC
ACCGGACAGGGCAGCCAGTACCCGGGCATGGCCCGCGAATTGTTCGACACGCAA
CCGGTTTTCGCCCAGACCGTTCGGCAGTGCGCCGAGGCGGTCGCCGACATGCTGC
CGCGACCACTGCTGGACGTGCTGTTCGCGACCGACCGCGAAACCGCGGACCTGC
TGCAGCACACGTCGTTCGCGCAGCCCGCGCTGTTCGCGGTCGAGATGGGGCTAGC
GCGGCTGTGGCAGTCATGGGCCATCGAACCCGATGTGGTGCTGGGGCACAGCGT
CGGCCAGTACGCCGCGGCCTGCGTGGCGGGCGTGTTCAGCCTTGACGACGGCGC
ACGACTGATGGCCGAGCGCGGCCGCATGTTCGGCAGCCTTCCCGAAGGCGGCCG
GATGGTGGCCGTGTTCGCCGACGCCGAACACGTCGAGCAGGTGGCCGGTGAGTT
CCCCCGGGTGTCGGTCGGCGCCTACAACGGCCCCAACACCGTGCTCTCGGGTCCC
GGCGAGGACCTCGAAGAGATCGTCGAGAGGTTCGAGGACGAGGGGATCCGCTGC
ACGTGGCTGGCGACCAGCCACGCGTTCCACTCCGAACTGCTCGATCCCGTGCTCG
ACGAGTTCGAGGCCTACGCGGCCCAGTTCCAGTTCGCAGCGCCGACACTGCCTTT
GGTGTGCAACCGGACCGGGGCCGTGCTCACCGGGCAGACCCCGCTCGACGCGCA
GTACTGGCGCAGGCATTCCCGCCAGCCCGTGCAGTTCGCCGAGAGTGTGCGCACC
GTCGCCGCGCTCGGCTGCTCGGTGCTGATGGAGATCGGCCCGCAACCTGTGCTGA
CCGGGGCCGCGGTACAGGTGTGGCCCGAACACATGGCTGCCCCGCGGGCCGTCG
CCTCGCTGCGCAAGGGCGTCGCCGATCGCCGACAGATCGCCGATGCGCTGGCGT
CGGCCTACGTCGGCGGCCACCGCCCCGACTTCGCCGCGGTGCACCGGCAGCCAC
GCCGCCCGGTCGAGTTGCCCACCTATCCGTTCCAGCGCCGCAGGTTCTGGCCGAA
GGCCTCGAGCATCGCCGTCGACGGTGGGACCGCGGCGTCGGGAATCCTGGGCAG
CGGCAAGGATCTGGCGTCCGGCGACACCATCTACACGAGCCGGCTGTCCGTCAA
ATCGCAGCCGTGGCTGGCCGATCACGTCATCTACGGCACCGTCGTCGTCCCCGGC
GCGACGTATGCGGCGATGGCGCTCGCCGCGGTCGGCACACCGGCACGCCTGAAG
GACGTGTTCTTCTACGAGCCGATCATCCTGCCCGAGAAGGCCTCTCGCGAGGTCC
AGCTGACGTTGCACCCTGCCGACGGTGGCAGCGTGCTCAGCTTCCAGATCCACAG
CCGCCCGTACGGTGAACGTGGTGCCGACTGGTCGCTGAACGCCGAGGGCACGGT
CGACACGGCCGGCAGCGACCCGGACGCCGCCGCAGACGAGCAGTCGGATCCGGT
CGACGAGGTGATCGAACGCCTCGAGCGCATGCGTCCGCAGGACCTCTTCGAGAC
CTTCGCCGACCTGGAACTGGCGTGGGGTCCCACATGGTCGGGCTCGCTGAAATCG
TTGTGGCTCGGCGACGGTGAGGCCGTCGGCGACATCCTCGTCGGCGCCGAACTCG
CCGAGCAACTCGGCTCCGAGCCCATGCACCCGGTGCTGATGGACCTGTGCACGG
GCGTCGCGTTCCCCGCGTTCCCGGCGCTGCTCGCGGCCGAACAGGGCGTCAACGA
CCTGTTCCTGCCGCTGCGCTACGGGCAGGTGACGCTCGCGGAGAAGATGCCGCG
GCGGTTCTACTGCCGGGCGAGGTGGCACCGTAGCGCGCTCGACAGCGAAACGCA
GGTGTTCGAACTCGAATTCGTCGACCTCGGTGGGCGAGCGCTCGGCGGCATCCGC
GAGTTCACCGTCAAACGTGCGCCGCGCGAGGCACTGCTGCGCGGCCTCGGGGGT
GACGCGACCCGCCTGCTCTACACGCTCGGGTGGCACGAGGTGCCGCCGCCACCA
TCGGCCGACGACGCGGTCGGCACGTGGCTGATCGCCGGGTTCGACGAGCTGGCC
GCGAAGGTGCCCGGCTGTGTCCCCATCGACCGCAACACCGATCTGCAGGTCCTCG
GTGAGGTGCTGTCGCAGGCGCACGCTCGCGGCGCGGGATTCTCGGGCGTCGTGT
GGCGTTGCGCGACGCCTGGCCGGAAGGAATCGGGTTCTGCCTCCGCCGAATCCG
CCCGTCTGGAGGCCGAGATCACCAATCTGCTCAGCGCGGTGCACGCCGCGCAGG
GGCCGAGCGTGAAACTGCCCGGCGGACTCTGGATCGTCACCGAACACGGCGTGG
CATGTGAATCCGGCGAGCCGGTGGACCCGGTGCAGGCCGCGCTGTGGGGCTTCG
GCCGAACGACGATCAACGAGGAACCGGCGCTGCACTGCCGGCTCGTCGACTGCG
ACGGATCTTCCGAGGCCGTCGACCTGCTGGCCGCTCTGCTGGCCGCACCGGGCAT
CCAGGAACCGGAAATCGCCGTGCGGCAGGGCAAACTGCTGGCGTCACGGTTGTT
GCCGTGGGCCCGCAGTGGTCATCTCACGCTGCCGCGCGGGGGCGACTTCGTCCTT
GCGCCCACCGAGCGCGGCGCGATCGACAACCTGCGCCTCACGGAGACGGACGTG
CCGGAGCCGGACGAGGGCTACGTGCAGGTTCGGGTCGAGGCCGCGGGCCTCAAC
TTCCGCGACGTGCTCAACGTGCTCGGCCTGTACCCCGGTGATCCGGGACCGATCG
GCGGCGACTTCGCAGGCACCGTGACCCAACTGGGGGCAGGTGTGAGTGGACTCG
AAATCGGCCAACGCGTGTACGGGTCGATGCAGGGTGCCTTCGCGAGCCGCTTCA
ACGTGCCCGCCCAGTTCCTGGCGCCGATTCCCGACGGCATCGGCGCGGTCGAGGC
CGCCACGATCCCGGCCGCGGCGCTGACGGTCCGGCTCGCGTTCGACTGGGCGCA
ACTCAAACCCGGCGACAAGGTGCTCATCCACGCCGCCAGCGGTGGCGTGGGGCT
CGCGGCCATCCAGATGGCGCAGCAGTCCGGCGCCGAGGTCTTCGCCACCGCGAG
CACCTTCAAGCGTTCGACCCTGCGCAGGCTGGGCGTGAAGTACGTCTACGACTCT
CGGACGACGGACTTCGCCGACCAGATCCTCGCGGACACCAACGGTGCCGGCGTG
GACGTGGTGCTCAACAGCCTCACTAGTGCGGGTTTCGTCGAGGCGACGCTGAAG
GCCACCGCGCAGGGCGGACGCTTCGCCGAGATCGCCAAGCGCGACATCTGGTCG
CATGAGCAGATGGCCGAGGCCCGCCCCGACATCGCCTACGAGATCGTCGCGCTG
GACACCGTGATGTTCACCGAGCCCGATCGCATCCGCGACCTGCTCACCGAGGTGT
CCGACGGGATGGCGCGCGGCGAGTGGACACCACTACCCGCCGAGATCTACCCGA
TCACCGAGGCCCGGTCGGCGTTCCGGCGCATGCAACAGGCACGGCACATCGGCA
AGATTGTGCTGCAGGTCCCCAAACCCCTTCAGCCGCAGCAGGACCGGAGCTACCT
GATCACCGGCGGACTCGGGGCGATCGGCCTGCACACGGCGGCGTATCTGGCGCA
GCTCGGCGCGGGCGACATCGTGCTGACCAGCCGGCGCGACCCCGACGAGTCGGC
CCAGCGGGCGATCGACGAGATCACCGAGCGCCACAAGTGCCGCATCCACACCTA
TGCGGCCGATGTGGGTGACGAGGCACAGGTGGCCGATCTGCTGGCGCGGATCCG
GGCCGAGTTGCCTCCACTGGCCGGAATCGCCCATCTGGCGGGCGTGCTCGACGAC
GCGCTGCTGTCGGCGCAGAACCCCGAGCGCTTCCGGACCACGTTGGCGCCCAAG
GCCTATGGCGCGAGCCACCTGGACCGCCTGACGCGCGACGACGACCTGGACTTC
TTCATCGTGTCCTCGTCGGTGTCGAGCCTGTTCGGATCACCGGGGCAGGCCAACT
ACGCAACCGCCAATGCGATGCTCGACGGTCTGGTCGCCAAGCGCCGTGCGCAGG
GCCTGGTGGCCACGGGCATCAACTTCGGGCCGTGGGGTCAGGGCGGCATGGCGT
CGTCGGAGGCCGCGACCGCGAACATCACCGCCCAAGGTCTGATCCCGTTGGATC
CGTCGGCCGCGTTGCACGCGCTGGCCGAGGTCATCGCGAACGGCTCCGGGCAGG
CGACGGTGCTCAAGGCCAACTGGCAGCGCGCGGCGAAGGTTCTCGGCTCGGCGC
GCCCGCCGATCCTGGACCTCGTCCTGCCGAGCGCGGCCGGGGAGGCCACGGGTG
ACAGCGAGCTGCTCAAGCAGTTGATGGAGATCCCGATACCGCAGCGCGCCGGGT
TCGTCACCGAGTTCCTGCAACGCGAAGTGCAGAACTTCCTGCGTCTCGCGTCGCC
GCCCGCGGCGTCGAGCCGGTTCCTGGACCTCGGCACGGATTCGCTGATGGCCATC
GAGCTGCGCAACCGGCTGCACAGCCAGTTCGGGGGCAAGTTCACCATCAACGCG
ACCGCGGTGTTCGACTACCCGACCATCGGCGGGCTCGCGGAGTACCTCGTGGGTC
AGCTGCCCGACGCCGAGGCGCCCGAAACGGCCACTAGTCAGCTCGACAGCGGGA
CTCCCGCCCGGGAAGCGAGCAGCGCTCTTCGCGACGGCTACCGGCAGGCGGGCG
TGTCGGGCAGGGTCCGGTCCTACCTCGACCTGCTGGCGGGGCTGTCGGACTTCCG
CGAGCACTTCGACGGCTCCGACGGGTTCTCCCTCGATCTCGTGGACATGGCCGAC
GGTCCCGGAGAGGTCACGGTGATCTGCTGCGCGGGAACGGCGGCGATCTCCGGT
CCGCACGAGTTCACCCGGCTCGCCGGGGCGCTGCGCGGAATCGCTCCGGTTCGG
GCCGTGCCCCAGCCCGGCTACGAGGAGGGCGAACCTCTGCCGTCGTCGATGGCG
GCGGTGGCGGCGGTGCAGGCCGATGCGGTCATCAGGACACAGGGGGACAAGCC
GTTCGTGGTGGCCGGTCACTCCGCGGGGGCACTGATGGCCTACGCGCTGGCGACC
GAACTGCTCGATCGCGGGCACCCGCCACGCGGTGTCGTCCTGATCGACGTCTACC
CGCCCGGTCACCAGGACGCGATGAACGCCTGGCTGGAGGAGCTGACCGCCACGC
TGTTCGACCGCGAGACGGTGCGGATGGACGACACCAGGCTCACCGCCCTGGGCG
CCTACGACCGCCTCACCGGTCAGTGGCGACCCCGGGAAACCGGGCTGCCGACGC
TGCTGGTCAGCGCCGGCGAGCCGATGGGTCCGTGGCCCGACGACAGCTGGAAGC
CGACGTGGCCCTTCGAGCACGACACCGTCGCCGTCCCCGGCGACCACTTCACGAT
GGTGCAGGAACACGCCGACGCGATCGCGCGGCACATCGACGCCTGGCTGGGCGG
AGGGAATTCATGA
SEQ ID NO: 4 depicts the amino acid sequence encoding PKS14TE protein
MVSAEHPIEPTPSFAIIGYAARFPGAASAEEYWALLRDGREAISDVPKDRWDIEEFFDP
DPSTPGKVVTRRAGFVDDVTGFDAPFFGMSAREVRLMDPQHRILMETAWRAVEHSG
IAPTSLANSNTGVFVGLATHDYLGMASDELTYPEIEAYMAIGTSNAAAAGRISYRLG
LQGPAVAVDTACSSSLVAIHQACQALRLNECDLALAGGANVLLTPATMITFSNAHM
LAPDGRCKTFDAAADGYVRGEGCGVVVVKRLEDAVRDGDRIRAVIRGSAINQDGAS
GGLTVPNGVAQQRVIADALKRAGLRPADVGYLEAHGTGTSLGDPIEAQAAGEVLGA
GRAPEEPLLIGSAKTNIGHLEAAAGIAGVIKVVMALENETLPKHLNFETPSPHIPWERL
PIEVVRETVPWQRNGKPRIAGVSSFGFAGTNAHVILEEAPAVGTPATTEPVEDPRRYS
VLPLSARTPEALVRLAGEYRDWLRAHPQARLADVSHTAGTARAHLEQRAALVVNS
RESAIELLGALADDRPAPGLVRGESHEAPKTAWLFTGQGSQYPGMARELFDTEPVFA
ETVRKCAEAVADILEKPLLDVIFDADELDGDTESPLRHTTYAQPALFAVELGLARLW
QSWGFEPDVVVGHSVGQYSAACVAGVFDVEDGARLIAERGRLFGSLPAGGRMAAV
FTAPERAERLTDEYPSLSVAAYNGANTVLSGPAKDLESAVATLVADGVRCDWLDTS
HAFHSALLDPILDDFESFAQKLEYREPQRILIDNRTGSALGRSTRLDGAYWRRHARQP
VEFAKSVRTLADMNCRVLLEIGPRPVLTATALAAWPDPATTPQVLASLRPTTADHRQ
ITEAVAAAYAAGHLPDFRAFRQPDARKLDLPTYPFEHRQFWFSDNRAIDRDTQTASA
GSVTPHRTQAVRLLEDGQIEELAALIDGETVDEQTLRVLNKLAARHNQQRSTHVDPD
ARYEIRWDAIASAAASTGAAADWILVGDDTAAVLEFADALTAGGHRHQIVALPGSD
ADEAQLVDTLRAASAGELYVVHIAAGDGAAAPMRDLLRVQHRTLGGLRRLFRAAN
DAELRSPIWLVTRDGQRVTDTDTVVPEQSCLWGFGRAAALELPHIWGGLADLSGAD
TEWPRLIARITSPRDGEDQIALRGDAVYAPRLVRRTGEPSDTPLPVREDRTYLVTGGL
GAIGLEVAGYLASHGAGHVVLTSRREPGDDARRRLDGLREQYACDVRVITADVADA
HDVARLMTTVAAELPPLAGIVHAAGEIGTTPLSGLDDAEVDRVFAGKVWGAWHLSE
ALTDLRTDLDFFVSTSSIASVWGGFGQTAYGAANAFLDGLAWRLRERGVTAVSVNF
GPWAAGMADAESRARLEQRGVRTLSPAEALAGMADVVAGPVQGVVAKIDWSRFLP
LYQQAGRRAFLSELESELPAAATGAAAPATVPGKPPLVEQLTKAPVQQRKSLITNYL
RNAVAEVTRVDADEIRDEAGFFDLGMDSLMAVELRRRIEQGVGKDIPVTLVMDHPR
LSDAADYLLVEVLGLGEQTNVRQASTVTARTDDPIAVVAVSCRFPGAPDPESFWELL
AGGVDAIREVPEDRWDIDEFYDPDPDTPGKTYTRFGGFLDGIDGFDPEFFGISPREAV
WIEPQQRLMLETVWEGLERAGYAPSDLRGSRTGVFTGVAANEYAHLLSAESIDKIEP
HFITGNALNAISGRVAFALGFEGPAVAVDTACSSALVAVHQACQALQSGDCDLALA
GGVNVLLSPVTVVAASRARMLSPVGRCKTFDASADGYVRSEGCGVLVLKRLSDAV
RDGDRVCAVIPASSVNQDGASSGLTVPNGGAQQRLIEATLARAGLSGADVDYLEAH
GTGTPLGDPIEVQAAAAAYGAGRDADRPLLMGSVKTNIGHTESASGAAGLIKVVLSL
QHGVLPQSLHFENPSPHIPWDALPVRVIDEPVPWQTNGRVRRAGVSSFGFTGTNAHV
LVEEAPPQLHTADAAPVEPAATAEAGTDDSPQVLALSARSPEALVELARRYDDWLS
ATPGLDIADVCHTAGVGRSHFEYRAALVVDSVERAREGLAALAEGRTPAGVVRGEH
VHRPTTAWLFTGQGSQYPGMARELFDTQPVFAQTVRQCAEAVADMLPRPLLDVLFA
TDRETADLLQHTSFAQPALFAVEMGLARLWQSWAIEPDVVLGHSVGQYAAACVAG
VFSLDDGARLMAERGRMFGSLPEGGRMVAVFADAEHVEQVAGEFPRVSVGAYNGP
NTVLSGPGEDLEEIVERFEDEGIRCTWLATSHAFHSELLDPVLDEFEAYAAQFQFAAP
TLPLVCNRTGAVLTGQTPLDAQYWRRHSRQPVQFAESVRTVAALGCSVLMEIGPQP
VLTGAAVQVWPEHMAAPRAVASLRKGVADRRQIADALASAYVGGHRPDFAAVHR
QPRRPVELPTYPFQRRRFWPKASSIAVDGGTAASGILGSGKDLASGDTIYTSRLSVKS
QPWLADHVIYGTVVVPGATYAAMALAAVGTPARLKDVFFYEPIILPEKASREVQLTL
HPADGGSVLSFQIHSRPYGERGADWSLNAEGTVDTAGSDPDAAADEQSDPVDEVIER
LERMRPQDLFETFADLELAWGPTWSGSLKSLWLGDGEAVGDILVGAELAEQLGSEP
MHPVLMDLCTGVAFPAFPALLAAEQGVNDLFLPLRYGQVTLAEKMPRRFYCRARW
HRSALDSETQVFELEFVDLGGRALGGIREFTVKRAPREALLRGLGGDATRLLYTLGW
HEVPPPPSADDAVGTWLIAGFDELAAKVPGCVPIDRNTDLQVLGEVLSQAHARGAG
FSGVVWRCATPGRKESGSASAESARLEAEITNLLSAVHAAQGPSVKLPGGLWIVTEH
GVACESGEPVDPVQAALWGFGRTTINEEPALHCRLVDCDGSSEAVDLLAALLAAPGI
QEPEIAVRQGKLLASRLLPWARSGHLTLPRGGDFVLAPTERGAIDNLRLTETDVPEPD
EGYVQVRVEAAGLNFRDVLNVLGLYPGDPGPIGGDFAGTVTQLGAGVSGLEIGQRV
YGSMQGAFASRFNVPAQFLAPIPDGIGAVEAATIPAAALTVRLAFDWAQLKPGDKVL
IHAASGGVGLAAIQMAQQSGAEVFATASTFKRSTLRRLGVKYVYDSRTTDFADQILA
DTNGAGVDVVLNSLTSAGFVEATLKATAQGGRFAEIAKRDIWSHEQMAEARPDIAY
EIVALDTVMFTEPDRIRDLLTEVSDGMARGEWTPLPAEIYPITEARSAFRRMQQARHI
GKIVLQVPKPLQPQQDRSYLITGGLGAIGLHTAAYLAQLGAGDIVLTSRRDPDESAQR
AIDEITERHKCRIHTYAADVGDEAQVADLLARIRAELPPLAGIAHLAGVLDDALLSAQ
NPERFRTTLAPKAYGASHLDRLTRDDDLDFFIVSSSVSSLFGSPGQANYATANAMLD
GLVAKRRAQGLVATGINFGPWGQGGMASSEAATANITAQGLIPLDPSAALHALAEVI
ANGSGQATVLKANWQRAAKVLGSARPPILDLVLPSAAGEATGDSELLKQLMEIPIPQ
RAGFVTEFLQREVQNFLRLASPPAASSRFLDLGTDSLMAIELRNRLHSQFGGKFTINA
TAVFDYPTIGGLAEYLVGQLPDAEAPETATSQLDSGTPAREASSALRDGYRQAGVSG
RVRSYLDLLAGLSDFREHFDGSDGFSLDLVDMADGPGEVTVICCAGTAAISGPHEFT
RLAGALRGIAPVRAVPQPGYEEGEPLPSSMAAVAAVQADAVIRTQGDKPFVVAGHS
AGALMAYALATELLDRGHPPRGVVLIDVYPPGHQDAMNAWLEELTATLFDRETVR
MDDTRLTALGAYDRLTGQWRPRETGLPTLLVSAGEPMGPWPDDSWKPTWPFEHDT
VAVPGDHFTMVQEHADAIARHIDAWLGGGNS
SEQ ID NO: 5 depicts the nucleic acid sequence of recombinant vector pSSRi11
TGGCGAATGGGACGCGCCCTGTAGCGGCGCATTAAGCGCGGCGGGTGTGGTGGT
TACGCGCAGCGTGACCGCTACACTTGCCAGCGCCCTAGCGCCCGCTCCTTTCGCT
TTCTTCCCTTCCTTTCTCGCCACGTTCGCCGGCTTTCCCCGTCAAGCTCTAAATCG
GGGGCTCCCTTTAGGGTTCCGATTTAGTGCTTTACGGCACCTCGACCCCAAAAAA
CTTGATTAGGGTGATGGTTCACGTAGTGGGCCATCGCCCTGATAGACGGTTTTTC
GCCCTTTGACGTTGGAGTCCACGTTCTTTAATAGTGGACTCTTGTTCCAAACTGGA
ACAACACTCAACCCTATCTCGGTCTATTCTTTTGATTTATAAGGGATTTTGCCGAT
TTCGGCCTATTGGTTAAAAAATGAGCTGATTTAACAAAAATTTAACGCGAATTTT
AACAAAATATTAACGTTTACAATTTCAGGTGGCACTTTTCGGGGAAATGTGCGCG
GAACCCCTATTTGTTTATTTTTCTAAATACATTCAAATATGTATCCGCTCATGAGA
CAATAACCCTGATAAATGCTTCAATAATATTGAAAAAGGAAGAGTATGAGTATT
CAACATTTCCGTGTCGCCCTTATTCCCTTTTTTGCGGCATTTTGCCTTCCTGTTTTT
GCTCACCCAGAAACGCTGGTGAAAGTAAAAGATGCTGAAGATCAGTTGGGTGCA
CGAGTGGGTTACATCGAACTGGATCTCAACAGCGGTAAGATCCTTGAGAGTTTTC
GCCCCGAAGAACGTTTTCCAATGATGAGCACTTTTAAAGTTCTGCTATGTGGCGC
GGTATTATCCCGTATTGACGCCGGGCAAGAGCAACTCGGTCGCCGCATACACTAT
TCTCAGAATGACTTGGTTGAGTACTCACCAGTCACAGAAAAGCATCTTACGGATG
GCATGACAGTAAGAGAATTATGCAGTGCTGCCATAACCATGAGTGATAACACTG
CGGCCAACTTACTTCTGACAACGATCGGAGGACCGAAGGAGCTAACCGCTTTTTT
GCACAACATGGGGGATCATGTAACTCGCCTTGATCGTTGGGAACCGGAGCTGAA
TGAAGCCATACCAAACGACGAGCGTGACACCACGATGCCTGCAGCAATGGCAAC
AACGTTGCGCAAACTATTAACTGGCGAACTACTTACTCTAGCTTCCCGGCAACAA
TTAATAGACTGGATGGAGGCGGATAAAGTTGCAGGACCACTTCTGCGCTCGGCC
CTTCCGGCTGGCTGGTTTATTGCTGATAAATCTGGAGCCGGTGAGCGTGGGTCTC
GCGGTATCATTGCAGCACTGGGGCCAGATGGTAAGCCCTCCCGTATCGTAGTTAT
CTACACGACGGGGAGTCAGGCAACTATGGATGAACGAAATAGACAGATCGCTGA
GATAGGTGCCTCACTGATTAAGCATTGGTAACTGTCAGACCAAGTTTACTCATAT
ATACTTTAGATTGATTTAAAACTTCATTTTTAATTTAAAAGGATCTAGGTGAAGA
TCCTTTTTGATAATCTCATGACCAAAATCCCTTAACGTGAGTTTTCGTTCCACTGA
GCGTCAGACCCCGTAGAAAAGATCAAAGGATCTTCTTGAGATCCTTTTTTTCTGC
GCGTAATCTGCTGCTTGCAAACAAAAAAACCACCGCTACCAGCGGTGGTTTGTTT
GCCGGATCAAGAGCTACCAACTCTTTTTCCGAAGGTAACTGGCTTCAGCAGAGCG
CAGATACCAAATACTGTCCTTCTAGTGTAGCCGTAGTTAGGCCACCACTTCAAGA
ACTCTGTAGCACCGCCTACATACCTCGCTCTGCTAATCCTGTTACCAGTGGCTGCT
GCCAGTGGCGATAAGTCGTGTCTTACCGGGTTGGACTCAAGACGATAGTTACCGG
ATAAGGCGCAGCGGTCGGGCTGAACGGGGGGTTCGTGCACACAGCCCAGCTTGG
AGCGAACGACCTACACCGAACTGAGATACCTACAGCGTGAGCTATGAGAAAGCG
CCACGCTTCCCGAAGGGAGAAAGGCGGACAGGTATCCGGTAAGCGGCAGGGTCG
GAACAGGAGAGCGCACGAGGGAGCTTCCAGGGGGAAACGCCTGGTATCTTTATA
GTCCTGTCGGGTTTCGCCACCTCTGACTTGAGCGTCGATTTTTGTGATGCTCGTCA
GGGGGGCGGAGCCTATGGAAAAACGCCAGCAACGCGGCCTTTTTACGGTTCCTG
GCCTTTTGCTGGCCTTTTGCTCACATGTTCTTTCCTGCGTTATCCCCTGATTCTGTG
GATAACCGTATTACCGCCTTTGAGTGAGCTGATACCGCTCGCCGCAGCCGAACGA
CCGAGCGCAGCGAGTCAGTGAGCGAGGAAGCGGAAGAGCGCCTGATGCGGTATT
TTCTCCTTACGCATCTGTGCGGTATTTCACACCGCATATATGGTGCACTCTCAGTA
CAATCTGCTCTGATGCCGCATAGTTAAGCCAGTATACACTCCGCTATCGCTACGT
GACTGGGTCATGGCTGCGCCCCGACACCCGCCAACACCCGCTGACGCGCCCTGA
CGGGCTTGTCTGCTCCCGGCATCCGCTTACAGACAAGCTGTGACCGTCTCCGGGA
GCTGCATGTGTCAGAGGTTTTCACCGTCATCACCGAAACGCGCGAGGCAGCTGCG
GTAAAGCTCATCAGCGTGGTCGTGAAGCGATTCACAGATGTCTGCCTGTTCATCC
GCGTCCAGCTCGTTGAGTTTCTCCAGAAGCGTTAATGTCTGGCTTCTGATAAAGC
GGGCCATGTTAAGGGCGGTTTTTTCCTGTTTGGTCACTGATGCCTCCGTGTAAGG
GGGATTTCTGTTCATGGGGGTAATGATACCGATGAAACGAGAGAGGATGCTCAC
GATACGGGTTACTGATGATGAACATGCCCGGTTACTGGAACGTTGTGAGGGTAA
ACAACTGGCGGTATGGATGCGGCGGGACCAGAGAAAAATCACTCAGGGTCAATG
CCAGCGCTTCGTTAATACAGATGTAGGTGTTCCACAGGGTAGCCAGCAGCATCCT
GCGATGCAGATCCGGAACATAATGGTGCAGGGCGCTGACTTCCGCGTTTCCAGA
CTTTACGAAACACGGAAACCGAAGACCATTCATGTTGTTGCTCAGGTCGCAGACG
TTTTGCAGCAGCAGTCGCTTCACGTTCGCTCGCGTATCGGTGATTCATTCTGCTAA
CCAGTAAGGCAACCCCGCCAGCCTAGCCGGGTCCTCAACGACAGGAGCACGATC
ATGCGCACCCGTGGGGCCGCCATGCCGGCGATAATGGCCTGCTTCTCGCCGAAAC
GTTTGGTGGCGGGACCAGTGACGAAGGCTTGAGCGAGGGCGTGCAAGATTCCGA
ATACCGCAAGCGACAGGCCGATCATCGTCGCGCTCCAGCGAAAGCGGTCCTCGC
CGAAAATGACCCAGAGCGCTGCCGGCACCTGTCCTACGAGTTGCATGATAAAGA
AGACAGTCATAAGTGCGGCGACGATAGTCATGCCCCGCGCCCACCGGAAGGAGC
TGACTGGGTTGAAGGCTCTCAAGGGCATCGGTCGAGATCCCGGTGCCTAATGAGT
GAGCTAACTTACATTAATTGCGTTGCGCTCACTGCCCGCTTTCCAGTCGGGAAAC
CTGTCGTGCCAGCTGCATTAATGAATCGGCCAACGCGCGGGGAGAGGCGGTTTG
CGTATTGGGCGCCAGGGTGGTTTTTCTTTTCACCAGTGAGACGGGCAACAGCTGA
TTGCCCTTCACCGCCTGGCCCTGAGAGAGTTGCAGCAAGCGGTCCACGCTGGTTT
GCCCCAGCAGGCGAAAATCCTGTTTGATGGTGGTTAACGGCGGGATATAACATG
AGCTGTCTTCGGTATCGTCGTATCCCACTACCGAGATATCCGCACCAACGCGCAG
CCCGGACTCGGTAATGGCGCGCATTGCGCCCAGCGCCATCTGATCGTTGGCAACC
AGCATCGCAGTGGGAACGATGCCCTCATTCAGCATTTGCATGGTTTGTTGAAAAC
CGGACATGGCACTCCAGTCGCCTTCCCGTTCCGCTATCGGCTGAATTTGATTGCG
AGTGAGATATTTATGCCAGCCAGCCAGACGCAGACGCGCCGAGACAGAACTTAA
TGGGCCCGCTAACAGCGCGATTTGCTGGTGACCCAATGCGACCAGATGCTCCACG
CCCAGTCGCGTACCGTCTTCATGGGAGAAAATAATACTGTTGATGGGTGTCTGGT
CAGAGACATCAAGAAATAACGCCGGAACATTAGTGCAGGCAGCTTCCACAGCAA
TGGCATCCTGGTCATCCAGCGGATAGTTAATGATCAGCCCACTGACGCGTTGCGC
GAGAAGATTGTGCACCGCCGCTTTACAGGCTTCGACGCCGCTTCGTTCTACCATC
GACACCACCACGCTGGCACCCAGTTGATCGGCGCGAGATTTAATCGCCGCGACA
ATTTGCGACGGCGCGTGCAGGGCCAGACTGGAGGTGGCAACGCCAATCAGCAAC
GACTGTTTGCCCGCCAGTTGTTGTGCCACGCGGTTGGGAATGTAATTCAGCTCCG
CCATCGCCGCTTCCACTTTTTCCCGCGTTTTCGCAGAAACGTGGCTGGCCTGGTTC
ACCACGCGGGAAACGGTCTGATAAGAGACACCGGCATACTCTGCGACATCGTAT
AACGTTACTGGTTTCACATTCACCACCCTGAATTGACTCTCTTCCGGGCGCTATCA
TGCCATACCGCGAAAGGTTTTGCGCCATTCGATGGTGTCCGGGATCTCGACGCTC
TCCCTTATGCGACTCCTGCATTAGGAAGCAGCCCAGTAGTAGGTTGAGGCCGTTG
AGCACCGCCGCCGCAAGGAATGGTGCATGCAAGGAGATGGCGCCCAACAGTCCC
CCGGCCACGGGGCCTGCCACCATACCCACGCCGAAACAAGCGCTCATGAGCCCG
AAGTGGCGAGCCCGATCTTCCCCATCGGTGATGTCGGCGATATAGGCGCCAGCA
ACCGCACCTGTGGCGCCGGTGATGCCGGCCACGATGCGTCCGGCGTAGAGGATC
GAGATCTCGATCCCGCGAAATTAATACGACTCACTATAGGGGAATTGTGAGCGG
ATAACAATTCCCCTCTAGAAATAATTTTGTTTAACTTTAAGAAGGAGATATACAT
ATGGTCTCTGCCGAACATCCGATCGAGCCGACCCCGAGCTTCGCAATCATCGGCT
ACGCCGCACGTTTTCCCGGCGCGGCGAGCGCCGAGGAATACTGGGCGTTGTTGC
GGGACGGGCGTGAAGCGATATCCGATGTTCCGAAAGACCGCTGGGACATCGAGG
AGTTCTTCGACCCGGACCCGTCGACACCGGGCAAGGTCGTCACGCGCCGCGCGG
GGTTCGTCGATGACGTAACGGGTTTCGACGCGCCGTTCTTCGGTATGTCCGCCCG
CGAGGTACGTCTGATGGACCCGCAGCACCGGATTCTCATGGAAACCGCGTGGCG
CGCGGTCGAACACTCGGGAATCGCGCCGACGTCGCTGGCCAACAGCAACACGGG
CGTCTTCGTCGGCCTGGCCACCCACGACTACCTCGGTATGGCCTCCGACGAACTC
ACCTACCCCGAGATCGAGGCCTACATGGCCATCGGAACCTCCAATGCCGCTGCG
GCCGGACGGATCAGTTACCGCCTGGGATTGCAGGGACCGGCGGTGGCGGTCGAC
ACCGCGTGCAGTTCGTCGCTGGTGGCGATTCACCAGGCGTGTCAGGCACTTCGCC
TGAACGAGTGCGATCTCGCGTTGGCCGGCGGGGCGAACGTCCTGCTCACCCCGG
CCACCATGATCACGTTCTCCAACGCGCACATGCTGGCTCCGGACGGCCGGTGCAA
GACCTTCGACGCGGCGGCCGACGGCTACGTGCGCGGCGAGGGCTGCGGTGTCGT
CGTGGTCAAGCGCCTCGAGGACGCGGTGCGCGACGGCGACCGGATCCGCGCGGT
GATCCGCGGAAGCGCAATCAACCAGGACGGGGCGTCGGGCGGGCTGACCGTGCC
GAACGGCGTTGCTCAGCAACGGGTTATCGCCGACGCGTTGAAGCGCGCCGGCCT
GCGACCGGCCGATGTCGGTTATCTCGAGGCGCACGGCACCGGAACCTCACTTGG
CGACCCCATCGAGGCCCAGGCCGCGGGCGAGGTGCTCGGGGCCGGACGCGCACC
TGAAGAACCGCTGCTGATCGGCTCGGCGAAGACCAACATCGGCCATCTGGAAGC
GGCCGCGGGCATCGCCGGTGTCATCAAGGTGGTCATGGCGCTCGAGAACGAGAC
GTTGCCCAAGCACCTCAATTTCGAGACTCCGTCGCCCCACATCCCGTGGGAGCGT
CTACCCATCGAGGTGGTGAGAGAGACCGTTCCCTGGCAGCGCAACGGCAAGCCG
CGGATCGCCGGGGTGAGCTCATTCGGGTTTGCCGGGACTAACGCGCACGTCATCC
TCGAAGAGGCGCCCGCCGTCGGAACACCGGCGACGACAGAGCCTGTCGAGGATC
CGAGGCGCTACAGCGTCCTTCCGCTGTCGGCACGCACGCCCGAGGCGTTGGTCCG
ACTCGCCGGTGAGTACCGCGACTGGCTGCGCGCCCACCCGCAGGCCCGCCTGGC
CGACGTGTCCCACACCGCGGGAACCGCACGTGCCCACCTGGAGCAGCGTGCCGC
GTTGGTGGTCAACTCGCGGGAATCGGCCATCGAACTGCTCGGCGCGCTCGCCGAC
GACCGTCCGGCGCCCGGCCTGGTCCGCGGTGAATCCCACGAGGCACCCAAGACC
GCGTGGTTGTTCACCGGCCAGGGCAGCCAGTACCCCGGAATGGCGCGTGAGTTG
TTCGACACCGAACCGGTGTTCGCCGAAACCGTGAGGAAATGCGCCGAGGCCGTC
GCAGATATCCTCGAAAAGCCTTTGCTCGACGTCATTTTCGACGCCGACGAGTTGG
ATGGTGACACCGAATCGCCGCTGCGGCACACGACCTATGCGCAACCCGCGCTGTT
CGCCGTCGAATTGGGCCTGGCCCGGCTCTGGCAGTCCTGGGGCTTCGAACCCGAC
GTGGTGGTCGGACACAGTGTCGGGCAGTACTCGGCCGCCTGTGTGGCGGGTGTGT
TCGACGTCGAGGACGGCGCACGACTGATCGCCGAGCGCGGCCGATTGTTCGGCA
GCCTGCCCGCGGGTGGACGCATGGCGGCGGTTTTCACCGCCCCCGAGCGTGCAG
AGCGCCTCACCGACGAGTACCCCAGCCTGTCGGTTGCCGCGTACAACGGTGCCA
ACACCGTATTGTCCGGTCCCGCAAAGGATCTCGAATCCGCCGTGGCCACATTGGT
GGCCGACGGTGTGCGGTGCGACTGGCTGGACACCAGCCACGCCTTCCACTCGGC
ACTGCTCGACCCGATTCTCGACGATTTCGAGTCGTTCGCCCAGAAGCTCGAATAC
CGTGAACCGCAACGTATCCTGATCGACAACCGCACCGGGTCCGCGCTCGGGCGG
AGCACCAGGCTCGACGGTGCCTACTGGCGGCGGCACGCACGCCAGCCTGTCGAG
TTCGCCAAGAGCGTCCGCACCCTCGCCGACATGAACTGCAGGGTGCTGCTGGAG
ATAGGGCCCAGGCCCGTGCTCACCGCCACCGCGCTGGCAGCATGGCCCGACCCG
GCCACCACGCCGCAGGTGCTCGCGTCGCTGCGGCCCACCACGGCCGATCACCGG
CAGATCACCGAGGCCGTCGCCGCCGCCTATGCCGCGGGACATCTGCCCGATTTCC
GCGCGTTCCGGCAACCGGACGCGCGCAAACTCGACCTGCCCACATATCCCTTCGA
GCATCGCCAGTTCTGGTTCTCGGACAACCGTGCGATCGACCGTGACACCCAGACC
GCGAGCGCCGGTTCCGTCACGCCTCACCGCACACAGGCCGTCCGACTTCTCGAAG
ACGGCCAGATCGAGGAACTCGCCGCCCTCATCGACGGCGAGACCGTCGACGAGC
AGACCCTGCGCGTGCTGAACAAGCTCGCGGCGCGCCACAACCAGCAGCGGTCCA
CACACGTCGACCCTGATGCGCGGTACGAGATCCGTTGGGACGCGATCGCTTCGGC
CGCCGCGAGCACGGGCGCGGCAGCCGACTGGATCCTGGTCGGCGACGACACCGC
TGCCGTCCTGGAGTTCGCCGATGCGCTCACCGCAGGCGGTCATCGCCACCAGATC
GTCGCGCTGCCCGGCTCCGACGCCGACGAGGCCCAGCTGGTCGATACGCTGCGC
GCTGCGTCGGCCGGTGAGCTGTACGTCGTGCACATCGCGGCAGGAGACGGTGCC
GCTGCCCCGATGCGGGACCTGCTGCGCGTGCAGCACCGAACCCTTGGTGGGCTGC
GGCGCCTGTTCCGCGCCGCGAACGACGCGGAACTGCGCAGCCCCATCTGGTTGGT
GACACGTGACGGGCAGCGGGTCACCGACACCGACACCGTGGTACCGGAACAGAG
TTGCCTGTGGGGATTCGGCCGCGCGGCGGCCCTGGAACTCCCGCACATCTGGGGT
GGCCTCGCCGACCTCTCCGGTGCGGACACCGAGTGGCCGCGGCTCATCGCGCGG
ATCACGTCACCGCGCGACGGTGAGGACCAGATCGCGCTGCGCGGGGACGCCGTG
TACGCACCCCGACTGGTCCGCCGGACCGGCGAGCCGAGCGACACCCCCCTGCCG
GTGCGGGAGGACCGCACCTATCTGGTGACGGGCGGTCTCGGGGCGATCGGGCTC
GAGGTCGCCGGCTACCTGGCCTCCCACGGTGCCGGTCATGTGGTGCTCACCAGCC
GACGGGAACCGGGCGACGACGCGCGCCGGCGCCTCGACGGTCTCCGCGAGCAGT
ACGCATGTGACGTCCGGGTGATCACCGCCGATGTCGCCGACGCGCACGACGTCG
CGCGCCTCATGACCACCGTCGCCGCCGAACTCCCGCCGCTGGCCGGCATCGTGCA
CGCCGCGGGCGAGATCGGCACCACGCCGCTGAGCGGCCTCGACGACGCCGAGGT
GGATCGCGTCTTCGCCGGGAAGGTCTGGGGCGCCTGGCATCTGAGCGAGGCCCT
GACAGATCTTCGCACCGATCTCGACTTCTTCGTCAGCACATCGTCGATCGCGTCG
GTGTGGGGCGGATTCGGGCAGACCGCCTACGGCGCGGCGAACGCGTTTCTCGAC
GGTCTGGCGTGGCGACTGCGCGAGCGGGGTGTCACCGCTGTCAGCGTGAACTTC
GGGCCGTGGGCCGCGGGCATGGCCGACGCCGAATCACGTGCCCGCCTCGAGCAG
CGAGGCGTGCGGACGTTGTCGCCCGCCGAGGCCCTCGCAGGCATGGCCGACGTC
GTGGCCGGCCCCGTTCAGGGAGTCGTCGCCAAGATCGACTGGTCACGTTTCCTGC
CGCTGTACCAGCAGGCAGGCCGGCGCGCGTTCCTGTCGGAACTCGAAAGCGAAT
TGCCCGCAGCGGCAACCGGCGCTGCTGCGCCCGCGACGGTGCCCGGGAAACCAC
CGCTCGTCGAGCAGTTGACCAAAGCGCCTGTGCAGCAACGCAAAAGCCTCATCA
CGAACTACCTGCGCAACGCGGTCGCCGAGGTCACACGGGTCGACGCCGACGAGA
TCCGCGATGAGGCCGGGTTCTTCGACCTCGGGATGGACTCCCTGATGGCCGTCGA
ACTGCGGCGGCGCATCGAGCAGGGTGTCGGCAAGGACATTCCCGTCACGCTCGT
GATGGATCATCCGCGGTTGTCCGACGCGGCCGACTACCTGCTGGTCGAGGTACTC
GGGTTGGGCGAGCAGACCAACGTGCGGCAGGCGTCGACCGTGACCGCGCGAACC
GACGATCCCATCGCGGTCGTCGCGGTGTCCTGCCGCTTCCCCGGCGCACCCGACC
CGGAGTCGTTCTGGGAGCTGCTCGCCGGTGGTGTCGATGCCATCCGAGAGGTCCC
CGAGGACCGCTGGGACATCGACGAGTTCTACGACCCCGATCCGGACACCCCGGG
CAAGACCTACACGCGGTTCGGCGGATTTCTAGACGGCATCGACGGATTCGATCCC
GAGTTCTTCGGCATCTCACCGCGTGAGGCCGTGTGGATCGAGCCGCAGCAGCGCC
TCATGCTCGAGACGGTGTGGGAGGGTCTCGAGCGGGCCGGCTACGCACCTTCGG
ACCTCCGGGGCAGCCGCACGGGTGTCTTCACCGGTGTTGCCGCCAACGAGTACGC
GCATCTGCTGTCGGCCGAGTCGATCGACAAGATCGAGCCGCACTTCATCACGGGC
AATGCGCTCAACGCGATCTCCGGTCGCGTGGCGTTCGCGCTGGGCTTCGAAGGTC
CTGCCGTCGCGGTCGACACCGCGTGCAGCTCGGCTCTGGTCGCGGTCCATCAGGC
ATGTCAGGCACTGCAGTCCGGGGACTGCGATCTGGCCCTCGCGGGAGGCGTCAA
CGTGCTGCTGAGCCCCGTCACCGTGGTCGCGGCCTCACGCGCGCGGATGCTGTCG
CCGGTGGGCCGGTGCAAGACGTTCGACGCGTCCGCCGACGGGTATGTGCGCAGT
GAGGGCTGCGGCGTCCTGGTGCTCAAGCGCCTCAGCGACGCCGTGCGTGACGGC
GACCGGGTGTGCGCGGTGATCCCTGCGAGCTCGGTGAACCAGGACGGCGCCTCC
AGCGGGTTGACCGTCCCCAACGGCGGTGCGCAGCAACGGCTCATCGAGGCGACG
CTGGCCCGCGCCGGACTGAGCGGCGCCGATGTGGACTACCTCGAGGCACACGGC
ACGGGTACGCCCCTCGGTGACCCGATCGAGGTGCAGGCCGCCGCGGCCGCGTAC
GGCGCCGGGCGTGACGCCGACCGGCCGCTGCTGATGGGATCGGTGAAGACCAAC
ATCGGGCACACCGAATCCGCCTCGGGTGCAGCGGGTCTGATCAAGGTGGTGCTG
TCACTGCAACACGGGGTGCTGCCGCAGAGCCTGCACTTCGAGAATCCGTCACCGC
ACATCCCGTGGGACGCATTGCCGGTGCGGGTGATCGACGAACCGGTGCCGTGGC
AGACCAACGGCCGGGTGCGCCGTGCCGGTGTCAGTTCGTTCGGGTTCACCGGCAC
CAACGCGCACGTGCTCGTCGAGGAGGCCCCGCCGCAACTGCACACCGCCGACGC
TGCGCCCGTGGAACCGGCCGCCACCGCGGAGGCCGGGACCGACGACTCGCCGCA
GGTGCTGGCGCTGTCGGCGCGGTCGCCCGAGGCGCTCGTGGAGTTGGCGCGGCG
CTACGACGACTGGTTGAGCGCGACGCCCGGCCTCGACATCGCCGACGTGTGCCA
CACCGCGGGCGTGGGCCGTTCGCACTTCGAATACCGCGCAGCCCTGGTGGTCGAC
TCGGTCGAGCGGGCACGCGAGGGTCTCGCCGCGCTCGCCGAGGGGCGCACTCCT
GCGGGCGTGGTGCGCGGCGAGCATGTGCACCGTCCGACGACGGCATGGCTGTTC
ACCGGACAGGGCAGCCAGTACCCGGGCATGGCCCGCGAATTGTTCGACACGCAA
CCGGTTTTCGCCCAGACCGTTCGGCAGTGCGCCGAGGCGGTCGCCGACATGCTGC
CGCGACCACTGCTGGACGTGCTGTTCGCGACCGACCGCGAAACCGCGGACCTGC
TGCAGCACACGTCGTTCGCGCAGCCCGCGCTGTTCGCGGTCGAGATGGGGCTAGC
GCGGCTGTGGCAGTCATGGGCCATCGAACCCGATGTGGTGCTGGGGCACAGCGT
CGGCCAGTACGCCGCGGCCTGCGTGGCGGGCGTGTTCAGCCTTGACGACGGCGC
ACGACTGATGGCCGAGCGCGGCCGCATGTTCGGCAGCCTTCCCGAAGGCGGCCG
GATGGTGGCCGTGTTCGCCGACGCCGAACACGTCGAGCAGGTGGCCGGTGAGTT
CCCCCGGGTGTCGGTCGGCGCCTACAACGGCCCCAACACCGTGCTCTCGGGTCCC
GGCGAGGACCTCGAAGAGATCGTCGAGAGGTTCGAGGACGAGGGGATCCGCTGC
ACGTGGCTGGCGACCAGCCACGCGTTCCACTCCGAACTGCTCGATCCCGTGCTCG
ACGAGTTCGAGGCCTACGCGGCCCAGTTCCAGTTCGCAGCGCCGACACTGCCTTT
GGTGTGCAACCGGACCGGGGCCGTGCTCACCGGGCAGACCCCGCTCGACGCGCA
GTACTGGCGCAGGCATTCCCGCCAGCCCGTGCAGTTCGCCGAGAGTGTGCGCACC
GTCGCCGCGCTCGGCTGCTCGGTGCTGATGGAGATCGGCCCGCAACCTGTGCTGA
CCGGGGCCGCGGTACAGGTGTGGCCCGAACACATGGCTGCCCCGCGGGCCGTCG
CCTCGCTGCGCAAGGGCGTCGCCGATCGCCGACAGATCGCCGATGCGCTGGCGT
CGGCCTACGTCGGCGGCCACCGCCCCGACTTCGCCGCGGTGCACCGGCAGCCAC
GCCGCCCGGTCGAGTTGCCCACCTATCCGTTCCAGCGCCGCAGGTTCTGGCCGAA
GGCCTCGAGCATCGCCGTCGACGGTGGGACCGCGGCGTCGGGAATCCTGGGCAG
CGGCAAGGATCTGGCGTCCGGCGACACCATCTACACGAGCCGGCTGTCCGTCAA
ATCGCAGCCGTGGCTGGCCGATCACGTCATCTACGGCACCGTCGTCGTCCCCGGC
GCGACGTATGCGGCGATGGCGCTCGCCGCGGTCGGCACACCGGCACGCCTGAAG
GACGTGTTCTTCTACGAGCCGATCATCCTGCCCGAGAAGGCCTCTCGCGAGGTCC
AGCTGACGTTGCACCCTGCCGACGGTGGCAGCGTGCTCAGCTTCCAGATCCACAG
CCGCCCGTACGGTGAACGTGGTGCCGACTGGTCGCTGAACGCCGAGGGCACGGT
CGACACGGCCGGCAGCGACCCGGACGCCGCCGCAGACGAGCAGTCGGATCCGGT
CGACGAGGTGATCGAACGCCTCGAGCGCATGCGTCCGCAGGACCTCTTCGAGAC
CTTCGCCGACCTGGAACTGGCGTGGGGTCCCACATGGTCGGGCTCGCTGAAATCG
TTGTGGCTCGGCGACGGTGAGGCCGTCGGCGACATCCTCGTCGGCGCCGAACTCG
CCGAGCAACTCGGCTCCGAGCCCATGCACCCGGTGCTGATGGACCTGTGCACGG
GCGTCGCGTTCCCCGCGTTCCCGGCGCTGCTCGCGGCCGAACAGGGCGTCAACGA
CCTGTTCCTGCCGCTGCGCTACGGGCAGGTGACGCTCGCGGAGAAGATGCCGCG
GCGGTTCTACTGCCGGGCGAGGTGGCACCGTAGCGCGCTCGACAGCGAAACGCA
GGTGTTCGAACTCGAATTCGTCGACCTCGGTGGGCGAGCGCTCGGCGGCATCCGC
GAGTTCACCGTCAAACGTGCGCCGCGCGAGGCACTGCTGCGCGGCCTCGGGGGT
GACGCGACCCGCCTGCTCTACACGCTCGGGTGGCACGAGGTGCCGCCGCCACCA
TCGGCCGACGACGCGGTCGGCACGTGGCTGATCGCCGGGTTCGACGAGCTGGCC
GCGAAGGTGCCCGGCTGTGTCCCCATCGACCGCAACACCGATCTGCAGGTCCTCG
GTGAGGTGCTGTCGCAGGCGCACGCTCGCGGCGCGGGATTCTCGGGCGTCGTGT
GGCGTTGCGCGACGCCTGGCCGGAAGGAATCGGGTTCTGCCTCCGCCGAATCCG
CCCGTCTGGAGGCCGAGATCACCAATCTGCTCAGCGCGGTGCACGCCGCGCAGG
GGCCGAGCGTGAAACTGCCCGGCGGACTCTGGATCGTCACCGAACACGGCGTGG
CATGTGAATCCGGCGAGCCGGTGGACCCGGTGCAGGCCGCGCTGTGGGGCTTCG
GCCGAACGACGATCAACGAGGAACCGGCGCTGCACTGCCGGCTCGTCGACTGCG
ACGGATCTTCCGAGGCCGTCGACCTGCTGGCCGCTCTGCTGGCCGCACCGGGCAT
CCAGGAACCGGAAATCGCCGTGCGGCAGGGCAAACTGCTGGCGTCACGGTTGTT
GCCGTGGGCCCGCAGTGGTCATCTCACGCTGCCGCGCGGGGGCGACTTCGTCCTT
GCGCCCACCGAGCGCGGCGCGATCGACAACCTGCGCCTCACGGAGACGGACGTG
CCGGAGCCGGACGAGGGCTACGTGCAGGTTCGGGTCGAGGCCGCGGGCCTCAAC
TTCCGCGACGTGCTCAACGTGCTCGGCCTGTACCCCGGTGATCCGGGACCGATCG
GCGGCGACTTCGCAGGCACCGTGACCCAACTGGGGGCAGGTGTGAGTGGACTCG
AAATCGGCCAACGCGTGTACGGGTCGATGCAGGGTGCCTTCGCGAGCCGCTTCA
ACGTGCCCGCCCAGTTCCTGGCGCCGATTCCCGACGGCATCGGCGCGGTCGAGGC
CGCCACGATCCCGGCCGCGGCGCTGACGGTCCGGCTCGCGTTCGACTGGGCGCA
ACTCAAACCCGGCGACAAGGTGCTCATCCACGCCGCCAGCGGTGGCGTGGGGCT
CGCGGCCATCCAGATGGCGCAGCAGTCCGGCGCCGAGGTCTTCGCCACCGCGAG
CACCTTCAAGCGTTCGACCCTGCGCAGGCTGGGCGTGAAGTACGTCTACGACTCT
CGGACGACGGACTTCGCCGACCAGATCCTCGCGGACACCAACGGTGCCGGCGTG
GACGTGGTGCTCAACAGCCTCACTAGTGCGGGTTTCGTCGAGGCGACGCTGAAG
GCCACCGCGCAGGGCGGACGCTTCGCCGAGATCGCCAAGCGCGACATCTGGTCG
CATGAGCAGATGGCCGAGGCCCGCCCCGACATCGCCTACGAGATCGTCGCGCTG
GACACCGTGATGTTCACCGAGCCCGATCGCATCCGCGACCTGCTCACCGAGGTGT
CCGACGGGATGGCGCGCGGCGAGTGGACACCACTACCCGCCGAGATCTACCCGA
TCACCGAGGCCCGGTCGGCGTTCCGGCGCATGCAACAGGCACGGCACATCGGCA
AGATTGTGCTGCAGGTCCCCAAACCCCTTCAGCCGCAGCAGGACCGGAGCTACCT
GATCACCGGCGGACTCGGGGCGATCGGCCTGCACACGGCGGCGTATCTGGCGCA
GCTCGGCGCGGGCGACATCGTGCTGACCAGCCGGCGCGACCCCGACGAGTCGGC
CCAGCGGGCGATCGACGAGATCACCGAGCGCCACAAGTGCCGCATCCACACCTA
TGCGGCCGATGTGGGTGACGAGGCACAGGTGGCCGATCTGCTGGCGCGGATCCG
GGCCGAGTTGCCTCCACTGGCCGGAATCGCCCATCTGGCGGGCGTGCTCGACGAC
GCGCTGCTGTCGGCGCAGAACCCCGAGCGCTTCCGGACCACGTTGGCGCCCAAG
GCCTATGGCGCGAGCCACCTGGACCGCCTGACGCGCGACGACGACCTGGACTTC
TTCATCGTGTCCTCGTCGGTGTCGAGCCTGTTCGGATCACCGGGGCAGGCCAACT
ACGCAACCGCCAATGCGATGCTCGACGGTCTGGTCGCCAAGCGCCGTGCGCAGG
GCCTGGTGGCCACGGGCATCAACTTCGGGCCGTGGGGTCAGGGCGGCATGGCGT
CGTCGGAGGCCGCGACCGCGAACATCACCGCCCAAGGTCTGATCCCGTTGGATC
CGTCGGCCGCGTTGCACGCGCTGGCCGAGGTCATCGCGAACGGCTCCGGGCAGG
CGACGGTGCTCAAGGCCAACTGGCAGCGCGCGGCGAAGGTTCTCGGCTCGGCGC
GCCCGCCGATCCTGGACCTCGTCCTGCCGAGCGCGGCCGGGGAGGCCACGGGTG
ACAGCGAGCTGCTCAAGCAGTTGATGGAGATCCCGATACCGCAGCGCGCCGGGT
TCGTCACCGAGTTCCTGCAACGCGAAGTGCAGAACTTCCTGCGTCTCGCGTCGCC
GCCCGCGGCGTCGAGCCGGTTCCTGGACCTCGGCACGGATTCGCTGATGGCCATC
GAGCTGCGCAACCGGCTGCACAGCCAGTTCGGGGGCAAGTTCACCATCAACGCG
ACCGCGGTGTTCGACTACCCGACCATCGGCGGGCTCGCGGAGTACCTCACTAGTC
AGCTCGACAGCGGGACTCCCGCCCGGGAAGCGAGCAGCGCTCTTCGCGACGGCT
ACCGGCAGGCGGGCGTGTCGGGCAGGGTCCGGTCCTACCTCGACCTGCTGGCGG
GGCTGTCGGACTTCCGCGAGCACTTCGACGGCTCCGACGGGTTCTCCCTCGATCT
CGTGGACATGGCCGACGGTCCCGGAGAGGTCACGGTGATCTGCTGCGCGGGAAC
GGCGGCGATCTCCGGTCCGCACGAGTTCACCCGGCTCGCCGGGGCGCTGCGCGG
AATCGCTCCGGTTCGGGCCGTGCCCCAGCCCGGCTACGAGGAGGGCGAACCTCT
GCCGTCGTCGATGGCGGCGGTGGCGGCGGTGCAGGCCGATGCGGTCATCAGGAC
ACAGGGGGACAAGCCGTTCGTGGTGGCCGGTCACTCCGCGGGGGCACTGATGGC
CTACGCGCTGGCGACCGAACTGCTCGATCGCGGGCACCCGCCACGCGGTGTCGTC
CTGATCGACGTCTACCCGCCCGGTCACCAGGACGCGATGAACGCCTGGCTGGAG
GAGCTGACCGCCACGCTGTTCGACCGCGAGACGGTGCGGATGGACGACACCAGG
CTCACCGCCCTGGGCGCCTACGACCGCCTCACCGGTCAGTGGCGACCCCGGGAA
ACCGGGCTGCCGACGCTGCTGGTCAGCGCCGGCGAGCCGATGGGTCCGTGGCCC
GACGACAGCTGGAAGCCGACGTGGCCCTTCGAGCACGACACCGTCGCCGTCCCC
GGCGACCACTTCACGATGGTGCAGGAACACGCCGACGCGATCGCGCGGCACATC
GACGCCTGGCTGGGCGGAGGGAATTCATGAAAGCTTGCGGCCGCACTCGAGCAC
CACCACCACCACCACTGAGATCCGGCTGCTAACAAAGCCCGAAAGGAAGCTGAG
TTGGCTGCTGCCACCGCTGAGCAATAACTAGCATAACCCCTTGGGGCCTCTAAAC
GGGTCTTGAGGGGTTTTTTGCTGAAAGGAGGAACTATATCCGGAT
SEQ ID NO: 6 depicts the nucleic acid sequence of recombinant vector pSSRi15
TGGCGAATGGGACGCGCCCTGTAGCGGCGCATTAAGCGCGGCGGGTGTGGTGGT
TACGCGCAGCGTGACCGCTACACTTGCCAGCGCCCTAGCGCCCGCTCCTTTCGCT
TTCTTCCCTTCCTTTCTCGCCACGTTCGCCGGCTTTCCCCGTCAAGCTCTAAATCG
GGGGCTCCCTTTAGGGTTCCGATTTAGTGCTTTACGGCACCTCGACCCCAAAAAA
CTTGATTAGGGTGATGGTTCACGTAGTGGGCCATCGCCCTGATAGACGGTTTTTC
GCCCTTTGACGTTGGAGTCCACGTTCTTTAATAGTGGACTCTTGTTCCAAACTGGA
ACAACACTCAACCCTATCTCGGTCTATTCTTTTGATTTATAAGGGATTTTGCCGAT
TTCGGCCTATTGGTTAAAAAATGAGCTGATTTAACAAAAATTTAACGCGAATTTT
AACAAAATATTAACGTTTACAATTTCAGGTGGCACTTTTCGGGGAAATGTGCGCG
GAACCCCTATTTGTTTATTTTTCTAAATACATTCAAATATGTATCCGCTCATGAGA
CAATAACCCTGATAAATGCTTCAATAATATTGAAAAAGGAAGAGTATGAGTATT
CAACATTTCCGTGTCGCCCTTATTCCCTTTTTTGCGGCATTTTGCCTTCCTGTTTTT
GCTCACCCAGAAACGCTGGTGAAAGTAAAAGATGCTGAAGATCAGTTGGGTGCA
CGAGTGGGTTACATCGAACTGGATCTCAACAGCGGTAAGATCCTTGAGAGTTTTC
GCCCCGAAGAACGTTTTCCAATGATGAGCACTTTTAAAGTTCTGCTATGTGGCGC
GGTATTATCCCGTATTGACGCCGGGCAAGAGCAACTCGGTCGCCGCATACACTAT
TCTCAGAATGACTTGGTTGAGTACTCACCAGTCACAGAAAAGCATCTTACGGATG
GCATGACAGTAAGAGAATTATGCAGTGCTGCCATAACCATGAGTGATAACACTG
CGGCCAACTTACTTCTGACAACGATCGGAGGACCGAAGGAGCTAACCGCTTTTTT
GCACAACATGGGGGATCATGTAACTCGCCTTGATCGTTGGGAACCGGAGCTGAA
TGAAGCCATACCAAACGACGAGCGTGACACCACGATGCCTGCAGCAATGGCAAC
AACGTTGCGCAAACTATTAACTGGCGAACTACTTACTCTAGCTTCCCGGCAACAA
TTAATAGACTGGATGGAGGCGGATAAAGTTGCAGGACCACTTCTGCGCTCGGCC
CTTCCGGCTGGCTGGTTTATTGCTGATAAATCTGGAGCCGGTGAGCGTGGGTCTC
GCGGTATCATTGCAGCACTGGGGCCAGATGGTAAGCCCTCCCGTATCGTAGTTAT
CTACACGACGGGGAGTCAGGCAACTATGGATGAACGAAATAGACAGATCGCTGA
GATAGGTGCCTCACTGATTAAGCATTGGTAACTGTCAGACCAAGTTTACTCATAT
ATACTTTAGATTGATTTAAAACTTCATTTTTAATTTAAAAGGATCTAGGTGAAGA
TCCTTTTTGATAATCTCATGACCAAAATCCCTTAACGTGAGTTTTCGTTCCACTGA
GCGTCAGACCCCGTAGAAAAGATCAAAGGATCTTCTTGAGATCCTTTTTTTCTGC
GCGTAATCTGCTGCTTGCAAACAAAAAAACCACCGCTACCAGCGGTGGTTTGTTT
GCCGGATCAAGAGCTACCAACTCTTTTTCCGAAGGTAACTGGCTTCAGCAGAGCG
CAGATACCAAATACTGTCCTTCTAGTGTAGCCGTAGTTAGGCCACCACTTCAAGA
ACTCTGTAGCACCGCCTACATACCTCGCTCTGCTAATCCTGTTACCAGTGGCTGCT
GCCAGTGGCGATAAGTCGTGTCTTACCGGGTTGGACTCAAGACGATAGTTACCGG
ATAAGGCGCAGCGGTCGGGCTGAACGGGGGGTTCGTGCACACAGCCCAGCTTGG
AGCGAACGACCTACACCGAACTGAGATACCTACAGCGTGAGCTATGAGAAAGCG
CCACGCTTCCCGAAGGGAGAAAGGCGGACAGGTATCCGGTAAGCGGCAGGGTCG
GAACAGGAGAGCGCACGAGGGAGCTTCCAGGGGGAAACGCCTGGTATCTTTATA
GTCCTGTCGGGTTTCGCCACCTCTGACTTGAGCGTCGATTTTTGTGATGCTCGTCA
GGGGGGCGGAGCCTATGGAAAAACGCCAGCAACGCGGCCTTTTTACGGTTCCTG
GCCTTTTGCTGGCCTTTTGCTCACATGTTCTTTCCTGCGTTATCCCCTGATTCTGTG
GATAACCGTATTACCGCCTTTGAGTGAGCTGATACCGCTCGCCGCAGCCGAACGA
CCGAGCGCAGCGAGTCAGTGAGCGAGGAAGCGGAAGAGCGCCTGATGCGGTATT
TTCTCCTTACGCATCTGTGCGGTATTTCACACCGCATATATGGTGCACTCTCAGTA
CAATCTGCTCTGATGCCGCATAGTTAAGCCAGTATACACTCCGCTATCGCTACGT
GACTGGGTCATGGCTGCGCCCCGACACCCGCCAACACCCGCTGACGCGCCCTGA
CGGGCTTGTCTGCTCCCGGCATCCGCTTACAGACAAGCTGTGACCGTCTCCGGGA
GCTGCATGTGTCAGAGGTTTTCACCGTCATCACCGAAACGCGCGAGGCAGCTGCG
GTAAAGCTCATCAGCGTGGTCGTGAAGCGATTCACAGATGTCTGCCTGTTCATCC
GCGTCCAGCTCGTTGAGTTTCTCCAGAAGCGTTAATGTCTGGCTTCTGATAAAGC
GGGCCATGTTAAGGGCGGTTTTTTCCTGTTTGGTCACTGATGCCTCCGTGTAAGG
GGGATTTCTGTTCATGGGGGTAATGATACCGATGAAACGAGAGAGGATGCTCAC
GATACGGGTTACTGATGATGAACATGCCCGGTTACTGGAACGTTGTGAGGGTAA
ACAACTGGCGGTATGGATGCGGCGGGACCAGAGAAAAATCACTCAGGGTCAATG
CCAGCGCTTCGTTAATACAGATGTAGGTGTTCCACAGGGTAGCCAGCAGCATCCT
GCGATGCAGATCCGGAACATAATGGTGCAGGGCGCTGACTTCCGCGTTTCCAGA
CTTTACGAAACACGGAAACCGAAGACCATTCATGTTGTTGCTCAGGTCGCAGACG
TTTTGCAGCAGCAGTCGCTTCACGTTCGCTCGCGTATCGGTGATTCATTCTGCTAA
CCAGTAAGGCAACCCCGCCAGCCTAGCCGGGTCCTCAACGACAGGAGCACGATC
ATGCGCACCCGTGGGGCCGCCATGCCGGCGATAATGGCCTGCTTCTCGCCGAAAC
GTTTGGTGGCGGGACCAGTGACGAAGGCTTGAGCGAGGGCGTGCAAGATTCCGA
ATACCGCAAGCGACAGGCCGATCATCGTCGCGCTCCAGCGAAAGCGGTCCTCGC
CGAAAATGACCCAGAGCGCTGCCGGCACCTGTCCTACGAGTTGCATGATAAAGA
AGACAGTCATAAGTGCGGCGACGATAGTCATGCCCCGCGCCCACCGGAAGGAGC
TGACTGGGTTGAAGGCTCTCAAGGGCATCGGTCGAGATCCCGGTGCCTAATGAGT
GAGCTAACTTACATTAATTGCGTTGCGCTCACTGCCCGCTTTCCAGTCGGGAAAC
CTGTCGTGCCAGCTGCATTAATGAATCGGCCAACGCGCGGGGAGAGGCGGTTTG
CGTATTGGGCGCCAGGGTGGTTTTTCTTTTCACCAGTGAGACGGGCAACAGCTGA
TTGCCCTTCACCGCCTGGCCCTGAGAGAGTTGCAGCAAGCGGTCCACGCTGGTTT
GCCCCAGCAGGCGAAAATCCTGTTTGATGGTGGTTAACGGCGGGATATAACATG
AGCTGTCTTCGGTATCGTCGTATCCCACTACCGAGATATCCGCACCAACGCGCAG
CCCGGACTCGGTAATGGCGCGCATTGCGCCCAGCGCCATCTGATCGTTGGCAACC
AGCATCGCAGTGGGAACGATGCCCTCATTCAGCATTTGCATGGTTTGTTGAAAAC
CGGACATGGCACTCCAGTCGCCTTCCCGTTCCGCTATCGGCTGAATTTGATTGCG
AGTGAGATATTTATGCCAGCCAGCCAGACGCAGACGCGCCGAGACAGAACTTAA
TGGGCCCGCTAACAGCGCGATTTGCTGGTGACCCAATGCGACCAGATGCTCCACG
CCCAGTCGCGTACCGTCTTCATGGGAGAAAATAATACTGTTGATGGGTGTCTGGT
CAGAGACATCAAGAAATAACGCCGGAACATTAGTGCAGGCAGCTTCCACAGCAA
TGGCATCCTGGTCATCCAGCGGATAGTTAATGATCAGCCCACTGACGCGTTGCGC
GAGAAGATTGTGCACCGCCGCTTTACAGGCTTCGACGCCGCTTCGTTCTACCATC
GACACCACCACGCTGGCACCCAGTTGATCGGCGCGAGATTTAATCGCCGCGACA
ATTTGCGACGGCGCGTGCAGGGCCAGACTGGAGGTGGCAACGCCAATCAGCAAC
GACTGTTTGCCCGCCAGTTGTTGTGCCACGCGGTTGGGAATGTAATTCAGCTCCG
CCATCGCCGCTTCCACTTTTTCCCGCGTTTTCGCAGAAACGTGGCTGGCCTGGTTC
ACCACGCGGGAAACGGTCTGATAAGAGACACCGGCATACTCTGCGACATCGTAT
AACGTTACTGGTTTCACATTCACCACCCTGAATTGACTCTCTTCCGGGCGCTATCA
TGCCATACCGCGAAAGGTTTTGCGCCATTCGATGGTGTCCGGGATCTCGACGCTC
TCCCTTATGCGACTCCTGCATTAGGAAGCAGCCCAGTAGTAGGTTGAGGCCGTTG
AGCACCGCCGCCGCAAGGAATGGTGCATGCAAGGAGATGGCGCCCAACAGTCCC
CCGGCCACGGGGCCTGCCACCATACCCACGCCGAAACAAGCGCTCATGAGCCCG
AAGTGGCGAGCCCGATCTTCCCCATCGGTGATGTCGGCGATATAGGCGCCAGCA
ACCGCACCTGTGGCGCCGGTGATGCCGGCCACGATGCGTCCGGCGTAGAGGATC
GAGATCTCGATCCCGCGAAATTAATACGACTCACTATAGGGGAATTGTGAGCGG
ATAACAATTCCCCTCTAGAAATAATTTTGTTTAACTTTAAGAAGGAGATATACAT
ATGGTCTCTGCCGAACATCCGATCGAGCCGACCCCGAGCTTCGCAATCATCGGCT
ACGCCGCACGTTTTCCCGGCGCGGCGAGCGCCGAGGAATACTGGGCGTTGTTGC
GGGACGGGCGTGAAGCGATATCCGATGTTCCGAAAGACCGCTGGGACATCGAGG
AGTTCTTCGACCCGGACCCGTCGACACCGGGCAAGGTCGTCACGCGCCGCGCGG
GGTTCGTCGATGACGTAACGGGTTTCGACGCGCCGTTCTTCGGTATGTCCGCCCG
CGAGGTACGTCTGATGGACCCGCAGCACCGGATTCTCATGGAAACCGCGTGGCG
CGCGGTCGAACACTCGGGAATCGCGCCGACGTCGCTGGCCAACAGCAACACGGG
CGTCTTCGTCGGCCTGGCCACCCACGACTACCTCGGTATGGCCTCCGACGAACTC
ACCTACCCCGAGATCGAGGCCTACATGGCCATCGGAACCTCCAATGCCGCTGCG
GCCGGACGGATCAGTTACCGCCTGGGATTGCAGGGACCGGCGGTGGCGGTCGAC
ACCGCGTGCAGTTCGTCGCTGGTGGCGATTCACCAGGCGTGTCAGGCACTTCGCC
TGAACGAGTGCGATCTCGCGTTGGCCGGCGGGGCGAACGTCCTGCTCACCCCGG
CCACCATGATCACGTTCTCCAACGCGCACATGCTGGCTCCGGACGGCCGGTGCAA
GACCTTCGACGCGGCGGCCGACGGCTACGTGCGCGGCGAGGGCTGCGGTGTCGT
CGTGGTCAAGCGCCTCGAGGACGCGGTGCGCGACGGCGACCGGATCCGCGCGGT
GATCCGCGGAAGCGCAATCAACCAGGACGGGGCGTCGGGCGGGCTGACCGTGCC
GAACGGCGTTGCTCAGCAACGGGTTATCGCCGACGCGTTGAAGCGCGCCGGCCT
GCGACCGGCCGATGTCGGTTATCTCGAGGCGCACGGCACCGGAACCTCACTTGG
CGACCCCATCGAGGCCCAGGCCGCGGGCGAGGTGCTCGGGGCCGGACGCGCACC
TGAAGAACCGCTGCTGATCGGCTCGGCGAAGACCAACATCGGCCATCTGGAAGC
GGCCGCGGGCATCGCCGGTGTCATCAAGGTGGTCATGGCGCTCGAGAACGAGAC
GTTGCCCAAGCACCTCAATTTCGAGACTCCGTCGCCCCACATCCCGTGGGAGCGT
CTACCCATCGAGGTGGTGAGAGAGACCGTTCCCTGGCAGCGCAACGGCAAGCCG
CGGATCGCCGGGGTGAGCTCATTCGGGTTTGCCGGGACTAACGCGCACGTCATCC
TCGAAGAGGCGCCCGCCGTCGGAACACCGGCGACGACAGAGCCTGTCGAGGATC
CGAGGCGCTACAGCGTCCTTCCGCTGTCGGCACGCACGCCCGAGGCGTTGGTCCG
ACTCGCCGGTGAGTACCGCGACTGGCTGCGCGCCCACCCGCAGGCCCGCCTGGC
CGACGTGTCCCACACCGCGGGAACCGCACGTGCCCACCTGGAGCAGCGTGCCGC
GTTGGTGGTCAACTCGCGGGAATCGGCCATCGAACTGCTCGGCGCGCTCGCCGAC
GACCGTCCGGCGCCCGGCCTGGTCCGCGGTGAATCCCACGAGGCACCCAAGACC
GCGTGGTTGTTCACCGGCCAGGGCAGCCAGTACCCCGGAATGGCGCGTGAGTTG
TTCGACACCGAACCGGTGTTCGCCGAAACCGTGAGGAAATGCGCCGAGGCCGTC
GCAGATATCCTCGAAAAGCCTTTGCTCGACGTCATTTTCGACGCCGACGAGTTGG
ATGGTGACACCGAATCGCCGCTGCGGCACACGACCTATGCGCAACCCGCGCTGTT
CGCCGTCGAATTGGGCCTGGCCCGGCTCTGGCAGTCCTGGGGCTTCGAACCCGAC
GTGGTGGTCGGACACAGTGTCGGGCAGTACTCGGCCGCCTGTGTGGCGGGTGTGT
TCGACGTCGAGGACGGCGCACGACTGATCGCCGAGCGCGGCCGATTGTTCGGCA
GCCTGCCCGCGGGTGGACGCATGGCGGCGGTTTTCACCGCCCCCGAGCGTGCAG
AGCGCCTCACCGACGAGTACCCCAGCCTGTCGGTTGCCGCGTACAACGGTGCCA
ACACCGTATTGTCCGGTCCCGCAAAGGATCTCGAATCCGCCGTGGCCACATTGGT
GGCCGACGGTGTGCGGTGCGACTGGCTGGACACCAGCCACGCCTTCCACTCGGC
ACTGCTCGACCCGATTCTCGACGATTTCGAGTCGTTCGCCCAGAAGCTCGAATAC
CGTGAACCGCAACGTATCCTGATCGACAACCGCACCGGGTCCGCGCTCGGGCGG
AGCACCAGGCTCGACGGTGCCTACTGGCGGCGGCACGCACGCCAGCCTGTCGAG
TTCGCCAAGAGCGTCCGCACCCTCGCCGACATGAACTGCAGGGTGCTGCTGGAG
ATAGGGCCCAGGCCCGTGCTCACCGCCACCGCGCTGGCAGCATGGCCCGACCCG
GCCACCACGCCGCAGGTGCTCGCGTCGCTGCGGCCCACCACGGCCGATCACCGG
CAGATCACCGAGGCCGTCGCCGCCGCCTATGCCGCGGGACATCTGCCCGATTTCC
GCGCGTTCCGGCAACCGGACGCGCGCAAACTCGACCTGCCCACATATCCCTTCGA
GCATCGCCAGTTCTGGTTCTCGGACAACCGTGCGATCGACCGTGACACCCAGACC
GCGAGCGCCGGTTCCGTCACGCCTCACCGCACACAGGCCGTCCGACTTCTCGAAG
ACGGCCAGATCGAGGAACTCGCCGCCCTCATCGACGGCGAGACCGTCGACGAGC
AGACCCTGCGCGTGCTGAACAAGCTCGCGGCGCGCCACAACCAGCAGCGGTCCA
CACACGTCGACCCTGATGCGCGGTACGAGATCCGTTGGGACGCGATCGCTTCGGC
CGCCGCGAGCACGGGCGCGGCAGCCGACTGGATCCTGGTCGGCGACGACACCGC
TGCCGTCCTGGAGTTCGCCGATGCGCTCACCGCAGGCGGTCATCGCCACCAGATC
GTCGCGCTGCCCGGCTCCGACGCCGACGAGGCCCAGCTGGTCGATACGCTGCGC
GCTGCGTCGGCCGGTGAGCTGTACGTCGTGCACATCGCGGCAGGAGACGGTGCC
GCTGCCCCGATGCGGGACCTGCTGCGCGTGCAGCACCGAACCCTTGGTGGGCTGC
GGCGCCTGTTCCGCGCCGCGAACGACGCGGAACTGCGCAGCCCCATCTGGTTGGT
GACACGTGACGGGCAGCGGGTCACCGACACCGACACCGTGGTACCGGAACAGAG
TTGCCTGTGGGGATTCGGCCGCGCGGCGGCCCTGGAACTCCCGCACATCTGGGGT
GGCCTCGCCGACCTCTCCGGTGCGGACACCGAGTGGCCGCGGCTCATCGCGCGG
ATCACGTCACCGCGCGACGGTGAGGACCAGATCGCGCTGCGCGGGGACGCCGTG
TACGCACCCCGACTGGTCCGCCGGACCGGCGAGCCGAGCGACACCCCCCTGCCG
GTGCGGGAGGACCGCACCTATCTGGTGACGGGCGGTCTCGGGGCGATCGGGCTC
GAGGTCGCCGGCTACCTGGCCTCCCACGGTGCCGGTCATGTGGTGCTCACCAGCC
GACGGGAACCGGGCGACGACGCGCGCCGGCGCCTCGACGGTCTCCGCGAGCAGT
ACGCATGTGACGTCCGGGTGATCACCGCCGATGTCGCCGACGCGCACGACGTCG
CGCGCCTCATGACCACCGTCGCCGCCGAACTCCCGCCGCTGGCCGGCATCGTGCA
CGCCGCGGGCGAGATCGGCACCACGCCGCTGAGCGGCCTCGACGACGCCGAGGT
GGATCGCGTCTTCGCCGGGAAGGTCTGGGGCGCCTGGCATCTGAGCGAGGCCCT
GACAGATCTTCGCACCGATCTCGACTTCTTCGTCAGCACATCGTCGATCGCGTCG
GTGTGGGGCGGATTCGGGCAGACCGCCTACGGCGCGGCGAACGCGTTTCTCGAC
GGTCTGGCGTGGCGACTGCGCGAGCGGGGTGTCACCGCTGTCAGCGTGAACTTC
GGGCCGTGGGCCGCGGGCATGGCCGACGCCGAATCACGTGCCCGCCTCGAGCAG
CGAGGCGTGCGGACGTTGTCGCCCGCCGAGGCCCTCGCAGGCATGGCCGACGTC
GTGGCCGGCCCCGTTCAGGGAGTCGTCGCCAAGATCGACTGGTCACGTTTCCTGC
CGCTGTACCAGCAGGCAGGCCGGCGCGCGTTCCTGTCGGAACTCGAAAGCGAAT
TGCCCGCAGCGGCAACCGGCGCTGCTGCGCCCGCGACGGTGCCCGGGAAACCAC
CGCTCGTCGAGCAGTTGACCAAAGCGCCTGTGCAGCAACGCAAAAGCCTCATCA
CGAACTACCTGCGCAACGCGGTCGCCGAGGTCACACGGGTCGACGCCGACGAGA
TCCGCGATGAGGCCGGGTTCTTCGACCTCGGGATGGACTCCCTGATGGCCGTCGA
ACTGCGGCGGCGCATCGAGCAGGGTGTCGGCAAGGACATTCCCGTCACGCTCGT
GATGGATCATCCGCGGTTGTCCGACGCGGCCGACTACCTGCTGGTCGAGGTACTC
GGGTTGGGCGAGCAGACCAACGTGCGGCAGGCGTCGACCGTGACCGCGCGAACC
GACGATCCCATCGCGGTCGTCGCGGTGTCCTGCCGCTTCCCCGGCGCACCCGACC
CGGAGTCGTTCTGGGAGCTGCTCGCCGGTGGTGTCGATGCCATCCGAGAGGTCCC
CGAGGACCGCTGGGACATCGACGAGTTCTACGACCCCGATCCGGACACCCCGGG
CAAGACCTACACGCGGTTCGGCGGATTTCTAGACGGCATCGACGGATTCGATCCC
GAGTTCTTCGGCATCTCACCGCGTGAGGCCGTGTGGATCGAGCCGCAGCAGCGCC
TCATGCTCGAGACGGTGTGGGAGGGTCTCGAGCGGGCCGGCTACGCACCTTCGG
ACCTCCGGGGCAGCCGCACGGGTGTCTTCACCGGTGTTGCCGCCAACGAGTACGC
GCATCTGCTGTCGGCCGAGTCGATCGACAAGATCGAGCCGCACTTCATCACGGGC
AATGCGCTCAACGCGATCTCCGGTCGCGTGGCGTTCGCGCTGGGCTTCGAAGGTC
CTGCCGTCGCGGTCGACACCGCGTGCAGCTCGGCTCTGGTCGCGGTCCATCAGGC
ATGTCAGGCACTGCAGTCCGGGGACTGCGATCTGGCCCTCGCGGGAGGCGTCAA
CGTGCTGCTGAGCCCCGTCACCGTGGTCGCGGCCTCACGCGCGCGGATGCTGTCG
CCGGTGGGCCGGTGCAAGACGTTCGACGCGTCCGCCGACGGGTATGTGCGCAGT
GAGGGCTGCGGCGTCCTGGTGCTCAAGCGCCTCAGCGACGCCGTGCGTGACGGC
GACCGGGTGTGCGCGGTGATCCCTGCGAGCTCGGTGAACCAGGACGGCGCCTCC
AGCGGGTTGACCGTCCCCAACGGCGGTGCGCAGCAACGGCTCATCGAGGCGACG
CTGGCCCGCGCCGGACTGAGCGGCGCCGATGTGGACTACCTCGAGGCACACGGC
ACGGGTACGCCCCTCGGTGACCCGATCGAGGTGCAGGCCGCCGCGGCCGCGTAC
GGCGCCGGGCGTGACGCCGACCGGCCGCTGCTGATGGGATCGGTGAAGACCAAC
ATCGGGCACACCGAATCCGCCTCGGGTGCAGCGGGTCTGATCAAGGTGGTGCTG
TCACTGCAACACGGGGTGCTGCCGCAGAGCCTGCACTTCGAGAATCCGTCACCGC
ACATCCCGTGGGACGCATTGCCGGTGCGGGTGATCGACGAACCGGTGCCGTGGC
AGACCAACGGCCGGGTGCGCCGTGCCGGTGTCAGTTCGTTCGGGTTCACCGGCAC
CAACGCGCACGTGCTCGTCGAGGAGGCCCCGCCGCAACTGCACACCGCCGACGC
TGCGCCCGTGGAACCGGCCGCCACCGCGGAGGCCGGGACCGACGACTCGCCGCA
GGTGCTGGCGCTGTCGGCGCGGTCGCCCGAGGCGCTCGTGGAGTTGGCGCGGCG
CTACGACGACTGGTTGAGCGCGACGCCCGGCCTCGACATCGCCGACGTGTGCCA
CACCGCGGGCGTGGGCCGTTCGCACTTCGAATACCGCGCAGCCCTGGTGGTCGAC
TCGGTCGAGCGGGCACGCGAGGGTCTCGCCGCGCTCGCCGAGGGGCGCACTCCT
GCGGGCGTGGTGCGCGGCGAGCATGTGCACCGTCCGACGACGGCATGGCTGTTC
ACCGGACAGGGCAGCCAGTACCCGGGCATGGCCCGCGAATTGTTCGACACGCAA
CCGGTTTTCGCCCAGACCGTTCGGCAGTGCGCCGAGGCGGTCGCCGACATGCTGC
CGCGACCACTGCTGGACGTGCTGTTCGCGACCGACCGCGAAACCGCGGACCTGC
TGCAGCACACGTCGTTCGCGCAGCCCGCGCTGTTCGCGGTCGAGATGGGGCTAGC
GCGGCTGTGGCAGTCATGGGCCATCGAACCCGATGTGGTGCTGGGGCACAGCGT
CGGCCAGTACGCCGCGGCCTGCGTGGCGGGCGTGTTCAGCCTTGACGACGGCGC
ACGACTGATGGCCGAGCGCGGCCGCATGTTCGGCAGCCTTCCCGAAGGCGGCCG
GATGGTGGCCGTGTTCGCCGACGCCGAACACGTCGAGCAGGTGGCCGGTGAGTT
CCCCCGGGTGTCGGTCGGCGCCTACAACGGCCCCAACACCGTGCTCTCGGGTCCC
GGCGAGGACCTCGAAGAGATCGTCGAGAGGTTCGAGGACGAGGGGATCCGCTGC
ACGTGGCTGGCGACCAGCCACGCGTTCCACTCCGAACTGCTCGATCCCGTGCTCG
ACGAGTTCGAGGCCTACGCGGCCCAGTTCCAGTTCGCAGCGCCGACACTGCCTTT
GGTGTGCAACCGGACCGGGGCCGTGCTCACCGGGCAGACCCCGCTCGACGCGCA
GTACTGGCGCAGGCATTCCCGCCAGCCCGTGCAGTTCGCCGAGAGTGTGCGCACC
GTCGCCGCGCTCGGCTGCTCGGTGCTGATGGAGATCGGCCCGCAACCTGTGCTGA
CCGGGGCCGCGGTACAGGTGTGGCCCGAACACATGGCTGCCCCGCGGGCCGTCG
CCTCGCTGCGCAAGGGCGTCGCCGATCGCCGACAGATCGCCGATGCGCTGGCGT
CGGCCTACGTCGGCGGCCACCGCCCCGACTTCGCCGCGGTGCACCGGCAGCCAC
GCCGCCCGGTCGAGTTGCCCACCTATCCGTTCCAGCGCCGCAGGTTCTGGCCGAA
GGCCTCGAGCATCGCCGTCGACGGTGGGACCGCGGCGTCGGGAATCCTGGGCAG
CGGCAAGGATCTGGCGTCCGGCGACACCATCTACACGAGCCGGCTGTCCGTCAA
ATCGCAGCCGTGGCTGGCCGATCACGTCATCTACGGCACCGTCGTCGTCCCCGGC
GCGACGTATGCGGCGATGGCGCTCGCCGCGGTCGGCACACCGGCACGCCTGAAG
GACGTGTTCTTCTACGAGCCGATCATCCTGCCCGAGAAGGCCTCTCGCGAGGTCC
AGCTGACGTTGCACCCTGCCGACGGTGGCAGCGTGCTCAGCTTCCAGATCCACAG
CCGCCCGTACGGTGAACGTGGTGCCGACTGGTCGCTGAACGCCGAGGGCACGGT
CGACACGGCCGGCAGCGACCCGGACGCCGCCGCAGACGAGCAGTCGGATCCGGT
CGACGAGGTGATCGAACGCCTCGAGCGCATGCGTCCGCAGGACCTCTTCGAGAC
CTTCGCCGACCTGGAACTGGCGTGGGGTCCCACATGGTCGGGCTCGCTGAAATCG
TTGTGGCTCGGCGACGGTGAGGCCGTCGGCGACATCCTCGTCGGCGCCGAACTCG
CCGAGCAACTCGGCTCCGAGCCCATGCACCCGGTGCTGATGGACCTGTGCACGG
GCGTCGCGTTCCCCGCGTTCCCGGCGCTGCTCGCGGCCGAACAGGGCGTCAACGA
CCTGTTCCTGCCGCTGCGCTACGGGCAGGTGACGCTCGCGGAGAAGATGCCGCG
GCGGTTCTACTGCCGGGCGAGGTGGCACCGTAGCGCGCTCGACAGCGAAACGCA
GGTGTTCGAACTCGAATTCGTCGACCTCGGTGGGCGAGCGCTCGGCGGCATCCGC
GAGTTCACCGTCAAACGTGCGCCGCGCGAGGCACTGCTGCGCGGCCTCGGGGGT
GACGCGACCCGCCTGCTCTACACGCTCGGGTGGCACGAGGTGCCGCCGCCACCA
TCGGCCGACGACGCGGTCGGCACGTGGCTGATCGCCGGGTTCGACGAGCTGGCC
GCGAAGGTGCCCGGCTGTGTCCCCATCGACCGCAACACCGATCTGCAGGTCCTCG
GTGAGGTGCTGTCGCAGGCGCACGCTCGCGGCGCGGGATTCTCGGGCGTCGTGT
GGCGTTGCGCGACGCCTGGCCGGAAGGAATCGGGTTCTGCCTCCGCCGAATCCG
CCCGTCTGGAGGCCGAGATCACCAATCTGCTCAGCGCGGTGCACGCCGCGCAGG
GGCCGAGCGTGAAACTGCCCGGCGGACTCTGGATCGTCACCGAACACGGCGTGG
CATGTGAATCCGGCGAGCCGGTGGACCCGGTGCAGGCCGCGCTGTGGGGCTTCG
GCCGAACGACGATCAACGAGGAACCGGCGCTGCACTGCCGGCTCGTCGACTGCG
ACGGATCTTCCGAGGCCGTCGACCTGCTGGCCGCTCTGCTGGCCGCACCGGGCAT
CCAGGAACCGGAAATCGCCGTGCGGCAGGGCAAACTGCTGGCGTCACGGTTGTT
GCCGTGGGCCCGCAGTGGTCATCTCACGCTGCCGCGCGGGGGCGACTTCGTCCTT
GCGCCCACCGAGCGCGGCGCGATCGACAACCTGCGCCTCACGGAGACGGACGTG
CCGGAGCCGGACGAGGGCTACGTGCAGGTTCGGGTCGAGGCCGCGGGCCTCAAC
TTCCGCGACGTGCTCAACGTGCTCGGCCTGTACCCCGGTGATCCGGGACCGATCG
GCGGCGACTTCGCAGGCACCGTGACCCAACTGGGGGCAGGTGTGAGTGGACTCG
AAATCGGCCAACGCGTGTACGGGTCGATGCAGGGTGCCTTCGCGAGCCGCTTCA
ACGTGCCCGCCCAGTTCCTGGCGCCGATTCCCGACGGCATCGGCGCGGTCGAGGC
CGCCACGATCCCGGCCGCGGCGCTGACGGTCCGGCTCGCGTTCGACTGGGCGCA
ACTCAAACCCGGCGACAAGGTGCTCATCCACGCCGCCAGCGGTGGCGTGGGGCT
CGCGGCCATCCAGATGGCGCAGCAGTCCGGCGCCGAGGTCTTCGCCACCGCGAG
CACCTTCAAGCGTTCGACCCTGCGCAGGCTGGGCGTGAAGTACGTCTACGACTCT
CGGACGACGGACTTCGCCGACCAGATCCTCGCGGACACCAACGGTGCCGGCGTG
GACGTGGTGCTCAACAGCCTCACTAGTGCGGGTTTCGTCGAGGCGACGCTGAAG
GCCACCGCGCAGGGCGGACGCTTCGCCGAGATCGCCAAGCGCGACATCTGGTCG
CATGAGCAGATGGCCGAGGCCCGCCCCGACATCGCCTACGAGATCGTCGCGCTG
GACACCGTGATGTTCACCGAGCCCGATCGCATCCGCGACCTGCTCACCGAGGTGT
CCGACGGGATGGCGCGCGGCGAGTGGACACCACTACCCGCCGAGATCTACCCGA
TCACCGAGGCCCGGTCGGCGTTCCGGCGCATGCAACAGGCACGGCACATCGGCA
AGATTGTGCTGCAGGTCCCCAAACCCCTTCAGCCGCAGCAGGACCGGAGCTACCT
GATCACCGGCGGACTCGGGGCGATCGGCCTGCACACGGCGGCGTATCTGGCGCA
GCTCGGCGCGGGCGACATCGTGCTGACCAGCCGGCGCGACCCCGACGAGTCGGC
CCAGCGGGCGATCGACGAGATCACCGAGCGCCACAAGTGCCGCATCCACACCTA
TGCGGCCGATGTGGGTGACGAGGCACAGGTGGCCGATCTGCTGGCGCGGATCCG
GGCCGAGTTGCCTCCACTGGCCGGAATCGCCCATCTGGCGGGCGTGCTCGACGAC
GCGCTGCTGTCGGCGCAGAACCCCGAGCGCTTCCGGACCACGTTGGCGCCCAAG
GCCTATGGCGCGAGCCACCTGGACCGCCTGACGCGCGACGACGACCTGGACTTC
TTCATCGTGTCCTCGTCGGTGTCGAGCCTGTTCGGATCACCGGGGCAGGCCAACT
ACGCAACCGCCAATGCGATGCTCGACGGTCTGGTCGCCAAGCGCCGTGCGCAGG
GCCTGGTGGCCACGGGCATCAACTTCGGGCCGTGGGGTCAGGGCGGCATGGCGT
CGTCGGAGGCCGCGACCGCGAACATCACCGCCCAAGGTCTGATCCCGTTGGATC
CGTCGGCCGCGTTGCACGCGCTGGCCGAGGTCATCGCGAACGGCTCCGGGCAGG
CGACGGTGCTCAAGGCCAACTGGCAGCGCGCGGCGAAGGTTCTCGGCTCGGCGC
GCCCGCCGATCCTGGACCTCGTCCTGCCGAGCGCGGCCGGGGAGGCCACGGGTG
ACAGCGAGCTGCTCAAGCAGTTGATGGAGATCCCGATACCGCAGCGCGCCGGGT
TCGTCACCGAGTTCCTGCAACGCGAAGTGCAGAACTTCCTGCGTCTCGCGTCGCC
GCCCGCGGCGTCGAGCCGGTTCCTGGACCTCGGCACGGATTCGCTGATGGCCATC
GAGCTGCGCAACCGGCTGCACAGCCAGTTCGGGGGCAAGTTCACCATCAACGCG
ACCGCGGTGTTCGACTACCCGACCATCGGCGGGCTCGCGGAGTACCTCGTGGGTC
AGCTGCCCGACGCCGAGGCGCCCGAAACGGCCACTAGTCAGCTCGACAGCGGGA
CTCCCGCCCGGGAAGCGAGCAGCGCTCTTCGCGACGGCTACCGGCAGGCGGGCG
TGTCGGGCAGGGTCCGGTCCTACCTCGACCTGCTGGCGGGGCTGTCGGACTTCCG
CGAGCACTTCGACGGCTCCGACGGGTTCTCCCTCGATCTCGTGGACATGGCCGAC
GGTCCCGGAGAGGTCACGGTGATCTGCTGCGCGGGAACGGCGGCGATCTCCGGT
CCGCACGAGTTCACCCGGCTCGCCGGGGCGCTGCGCGGAATCGCTCCGGTTCGG
GCCGTGCCCCAGCCCGGCTACGAGGAGGGCGAACCTCTGCCGTCGTCGATGGCG
GCGGTGGCGGCGGTGCAGGCCGATGCGGTCATCAGGACACAGGGGGACAAGCC
GTTCGTGGTGGCCGGTCACTCCGCGGGGGCACTGATGGCCTACGCGCTGGCGACC
GAACTGCTCGATCGCGGGCACCCGCCACGCGGTGTCGTCCTGATCGACGTCTACC
CGCCCGGTCACCAGGACGCGATGAACGCCTGGCTGGAGGAGCTGACCGCCACGC
TGTTCGACCGCGAGACGGTGCGGATGGACGACACCAGGCTCACCGCCCTGGGCG
CCTACGACCGCCTCACCGGTCAGTGGCGACCCCGGGAAACCGGGCTGCCGACGC
TGCTGGTCAGCGCCGGCGAGCCGATGGGTCCGTGGCCCGACGACAGCTGGAAGC
CGACGTGGCCCTTCGAGCACGACACCGTCGCCGTCCCCGGCGACCACTTCACGAT
GGTGCAGGAACACGCCGACGCGATCGCGCGGCACATCGACGCCTGGCTGGGCGG
AGGGAATTCATGAAAGCTTGCGGCCGCACTCGAGCACCACCACCACCACCACTG
AGATCCGGCTGCTAACAAAGCCCGAAAGGAAGCTGAGTTGGCTGCTGCCACCGC
TGAGCAATAACTAGCATAACCCCTTGGGGCCTCTAAACGGGTCTTGAGGGGTTTT
TTGCTGAAAGGAGGAACTATATCCGGAT
SEQ ID NO: 7 depicts the sequence of the recombinant vector pSSRi11
confirmed by sanger Sequencing
CGCGAAGTGCAGAACTTCCTGCGTCTCGCGTCGCCGCCCGCGGCGTCGAGCCGGT
TCCTGGACCTCGGCACGGATTCGCTGATGGCCATCGAGCTGCGCAACCGGCTGCA
CAGCCAGTTCGGGGGCAAGTTCACCATCAACGCGACCGCGGTGTTCGACTACCC
GACCATCGGCGGGCTCGCGGAGTACCTCACTAGTCAGCTCGACAGCGGGACTCC
CGCCCGGGAAGCGAGCAGCGCTCTTCGCGACGGCTACCGGCAGGCGGGCGTGTC
GGGCAGGGTCCGGTCCTACCTCGACCTGCTGGCGGGGCTGTCGGACTTCCGCGAG
CACTTCGACGGCTCCGAC
SEQ ID NO: 8 depicts the sequence of the recombinant vector pSSRi15
confirmed by sanger Sequencing
TCGCTGATGGCCATCGAGCTGCGCAACCGGCTGCACAGCCAGTTCGGGGGCAAG
TTCACCATCAACGCGACCGCGGTGTTCGACTACCCGACCATCGGCGGGCTCGCGG
AGTACCTCGTGGGTCAGCTGCCCGACGCCGAGGCGCCCGAAACGGCCACTAGTC
AGCTCGACAGCGGGACTCCCGCCCGGGAAGCGAGCAGCGCTCTTCGCGACGGCT
ACCGGCAGGCGGGCGTGTCGGGCAGGGTCCGGTCCTACCTCGACCTGCTGGCGG
GGCTGTCGGACTTCCGCGAGCACTTCGACGGCTCCGACGGGTTCTCCCTCGATCT
CGTGGACATGGCCGACGGTCCCGGAGAGGTCACGGTGATCTGCTGCGCG
SEQ ID NO: 9 depicts the nucleotide sequence of forward primer IML578
CTCAACAGCCTCACTAGTGCGGGTTTC
SEQ ID NO: 10 depicts the nucleotide sequence of reverse primer IML579
CTGCTGACTAGTGAGGTACTCCGCGAGCCC
SEQ ID NO: 11 depicts the nucleotide sequence of IML808
CTGCTGACTAGTGGCCGTTTCGGGCGC
SEQ ID NO: 12 depicts the nucleic acid sequence encoding PKS7TE protein
ATGGTCTCTGCCGAACATCCGATCGAGCCGACCCCGAGCTTCGCAATCATCGGCT
ACGCCGCACGTTTTCCCGGCGCGGCGAGCGCCGAGGAATACTGGGCGTTGTTGC
GGGACGGGCGTGAAGCGATATCCGATGTTCCGAAAGACCGCTGGGACATCGAGG
AGTTCTTCGACCCGGACCCGTCGACACCGGGCAAGGTCGTCACGCGCCGCGCGG
GGTTCGTCGATGACGTAACGGGTTTCGACGCGCCGTTCTTCGGTATGTCCGCCCG
CGAGGTACGTCTGATGGACCCGCAGCACCGGATTCTCATGGAAACCGCGTGGCG
CGCGGTCGAACACTCGGGAATCGCGCCGACGTCGCTGGCCAACAGCAACACGGG
CGTCTTCGTCGGCCTGGCCACCCACGACTACCTCGGTATGGCCTCCGACGAACTC
ACCTACCCCGAGATCGAGGCCTACATGGCCATCGGAACCTCCAATGCCGCTGCG
GCCGGACGGATCAGTTACCGCCTGGGATTGCAGGGACCGGCGGTGGCGGTCGAC
ACCGCGTGCAGTTCGTCGCTGGTGGCGATTCACCAGGCGTGTCAGGCACTTCGCC
TGAACGAGTGCGATCTCGCGTTGGCCGGCGGGGCGAACGTCCTGCTCACCCCGG
CCACCATGATCACGTTCTCCAACGCGCACATGCTGGCTCCGGACGGCCGGTGCAA
GACCTTCGACGCGGCGGCCGACGGCTACGTGCGCGGCGAGGGCTGCGGTGTCGT
CGTGGTCAAGCGCCTCGAGGACGCGGTGCGCGACGGCGACCGGATCCGCGCGGT
GATCCGCGGAAGCGCAATCAACCAGGACGGGGCGTCGGGCGGGCTGACCGTGCC
GAACGGCGTTGCTCAGCAACGGGTTATCGCCGACGCGTTGAAGCGCGCCGGCCT
GCGACCGGCCGATGTCGGTTATCTCGAGGCGCACGGCACCGGAACCTCACTTGG
CGACCCCATCGAGGCCCAGGCCGCGGGCGAGGTGCTCGGGGCCGGACGCGCACC
TGAAGAACCGCTGCTGATCGGCTCGGCGAAGACCAACATCGGCCATCTGGAAGC
GGCCGCGGGCATCGCCGGTGTCATCAAGGTGGTCATGGCGCTCGAGAACGAGAC
GTTGCCCAAGCACCTCAATTTCGAGACTCCGTCGCCCCACATCCCGTGGGAGCGT
CTACCCATCGAGGTGGTGAGAGAGACCGTTCCCTGGCAGCGCAACGGCAAGCCG
CGGATCGCCGGGGTGAGCTCATTCGGGTTTGCCGGGACTAACGCGCACGTCATCC
TCGAAGAGGCGCCCGCCGTCGGAACACCGGCGACGACAGAGCCTGTCGAGGATC
CGAGGCGCTACAGCGTCCTTCCGCTGTCGGCACGCACGCCCGAGGCGTTGGTCCG
ACTCGCCGGTGAGTACCGCGACTGGCTGCGCGCCCACCCGCAGGCCCGCCTGGC
CGACGTGTCCCACACCGCGGGAACCGCACGTGCCCACCTGGAGCAGCGTGCCGC
GTTGGTGGTCAACTCGCGGGAATCGGCCATCGAACTGCTCGGCGCGCTCGCCGAC
GACCGTCCGGCGCCCGGCCTGGTCCGCGGTGAATCCCACGAGGCACCCAAGACC
GCGTGGTTGTTCACCGGCCAGGGCAGCCAGTACCCCGGAATGGCGCGTGAGTTG
TTCGACACCGAACCGGTGTTCGCCGAAACCGTGAGGAAATGCGCCGAGGCCGTC
GCAGATATCCTCGAAAAGCCTTTGCTCGACGTCATTTTCGACGCCGACGAGTTGG
ATGGTGACACCGAATCGCCGCTGCGGCACACGACCTATGCGCAACCCGCGCTGTT
CGCCGTCGAATTGGGCCTGGCCCGGCTCTGGCAGTCCTGGGGCTTCGAACCCGAC
GTGGTGGTCGGACACAGTGTCGGGCAGTACTCGGCCGCCTGTGTGGCGGGTGTGT
TCGACGTCGAGGACGGCGCACGACTGATCGCCGAGCGCGGCCGATTGTTCGGCA
GCCTGCCCGCGGGTGGACGCATGGCGGCGGTTTTCACCGCCCCCGAGCGTGCAG
AGCGCCTCACCGACGAGTACCCCAGCCTGTCGGTTGCCGCGTACAACGGTGCCA
ACACCGTATTGTCCGGTCCCGCAAAGGATCTCGAATCCGCCGTGGCCACATTGGT
GGCCGACGGTGTGCGGTGCGACTGGCTGGACACCAGCCACGCCTTCCACTCGGC
ACTGCTCGACCCGATTCTCGACGATTTCGAGTCGTTCGCCCAGAAGCTCGAATAC
CGTGAACCGCAACGTATCCTGATCGACAACCGCACCGGGTCCGCGCTCGGGCGG
AGCACCAGGCTCGACGGTGCCTACTGGCGGCGGCACGCACGCCAGCCTGTCGAG
TTCGCCAAGAGCGTCCGCACCCTCGCCGACATGAACTGCAGGGTGCTGCTGGAG
ATAGGGCCCAGGCCCGTGCTCACCGCCACCGCGCTGGCAGCATGGCCCGACCCG
GCCACCACGCCGCAGGTGCTCGCGTCGCTGCGGCCCACCACGGCCGATCACCGG
CAGATCACCGAGGCCGTCGCCGCCGCCTATGCCGCGGGACATCTGCCCGATTTCC
GCGCGTTCCGGCAACCGGACGCGCGCAAACTCGACCTGCCCACATATCCCTTCGA
GCATCGCCAGTTCTGGTTCTCGGACAACCGTGCGATCGACCGTGACACCCAGACC
GCGAGCGCCGGTTCCGTCACGCCTCACCGCACACAGGCCGTCCGACTTCTCGAAG
ACGGCCAGATCGAGGAACTCGCCGCCCTCATCGACGGCGAGACCGTCGACGAGC
AGACCCTGCGCGTGCTGAACAAGCTCGCGGCGCGCCACAACCAGCAGCGGTCCA
CACACGTCGACCCTGATGCGCGGTACGAGATCCGTTGGGACGCGATCGCTTCGGC
CGCCGCGAGCACGGGCGCGGCAGCCGACTGGATCCTGGTCGGCGACGACACCGC
TGCCGTCCTGGAGTTCGCCGATGCGCTCACCGCAGGCGGTCATCGCCACCAGATC
GTCGCGCTGCCCGGCTCCGACGCCGACGAGGCCCAGCTGGTCGATACGCTGCGC
GCTGCGTCGGCCGGTGAGCTGTACGTCGTGCACATCGCGGCAGGAGACGGTGCC
GCTGCCCCGATGCGGGACCTGCTGCGCGTGCAGCACCGAACCCTTGGTGGGCTGC
GGCGCCTGTTCCGCGCCGCGAACGACGCGGAACTGCGCAGCCCCATCTGGTTGGT
GACACGTGACGGGCAGCGGGTCACCGACACCGACACCGTGGTACCGGAACAGAG
TTGCCTGTGGGGATTCGGCCGCGCGGCGGCCCTGGAACTCCCGCACATCTGGGGT
GGCCTCGCCGACCTCTCCGGTGCGGACACCGAGTGGCCGCGGCTCATCGCGCGG
ATCACGTCACCGCGCGACGGTGAGGACCAGATCGCGCTGCGCGGGGACGCCGTG
TACGCACCCCGACTGGTCCGCCGGACCGGCGAGCCGAGCGACACCCCCCTGCCG
GTGCGGGAGGACCGCACCTATCTGGTGACGGGCGGTCTCGGGGCGATCGGGCTC
GAGGTCGCCGGCTACCTGGCCTCCCACGGTGCCGGTCATGTGGTGCTCACCAGCC
GACGGGAACCGGGCGACGACGCGCGCCGGCGCCTCGACGGTCTCCGCGAGCAGT
ACGCATGTGACGTCCGGGTGATCACCGCCGATGTCGCCGACGCGCACGACGTCG
CGCGCCTCATGACCACCGTCGCCGCCGAACTCCCGCCGCTGGCCGGCATCGTGCA
CGCCGCGGGCGAGATCGGCACCACGCCGCTGAGCGGCCTCGACGACGCCGAGGT
GGATCGCGTCTTCGCCGGGAAGGTCTGGGGCGCCTGGCATCTGAGCGAGGCCCT
GACAGATCTTCGCACCGATCTCGACTTCTTCGTCAGCACATCGTCGATCGCGTCG
GTGTGGGGCGGATTCGGGCAGACCGCCTACGGCGCGGCGAACGCGTTTCTCGAC
GGTCTGGCGTGGCGACTGCGCGAGCGGGGTGTCACCGCTGTCAGCGTGAACTTC
GGGCCGTGGGCCGCGGGCATGGCCGACGCCGAATCACGTGCCCGCCTCGAGCAG
CGAGGCGTGCGGACGTTGTCGCCCGCCGAGGCCCTCGCAGGCATGGCCGACGTC
GTGGCCGGCCCCGTTCAGGGAGTCGTCGCCAAGATCGACTGGTCACGTTTCCTGC
CGCTGTACCAGCAGGCAGGCCGGCGCGCGTTCCTGTCGGAACTCGAAAGCGAAT
TGCCCGCAGCGGCAACCGGCGCTGCTGCGCCCGCGACGGTGCCCGGGAAACCAC
CGCTCGTCGAGCAGTTGACCAAAGCGCCTGTGCAGCAACGCAAAAGCCTCATCA
CGAACTACCTGCGCAACGCGGTCGCCGAGGTCACACGGGTCGACGCCGACGAGA
TCCGCGATGAGGCCGGGTTCTTCGACCTCGGGATGGACTCCCTGATGGCCGTCGA
ACTGCGGCGGCGCATCGAGCAGGGTGTCGGCAAGGACATTCCCGTCACGCTCGT
GATGGATCATCCGCGGTTGTCCGACGCGGCCGACTACCTGCTGGTCGAGGTACTC
GGGTTGGGCGAGCAGACCAACGTGCGGCAGGCGTCGACCGTGACCGCGCGAACC
GACGATCCCATCGCGGTCGTCGCGGTGTCCTGCCGCTTCCCCGGCGCACCCGACC
CGGAGTCGTTCTGGGAGCTGCTCGCCGGTGGTGTCGATGCCATCCGAGAGGTCCC
CGAGGACCGCTGGGACATCGACGAGTTCTACGACCCCGATCCGGACACCCCGGG
CAAGACCTACACGCGGTTCGGCGGATTTCTAGACGGCATCGACGGATTCGATCCC
GAGTTCTTCGGCATCTCACCGCGTGAGGCCGTGTGGATCGAGCCGCAGCAGCGCC
TCATGCTCGAGACGGTGTGGGAGGGTCTCGAGCGGGCCGGCTACGCACCTTCGG
ACCTCCGGGGCAGCCGCACGGGTGTCTTCACCGGTGTTGCCGCCAACGAGTACGC
GCATCTGCTGTCGGCCGAGTCGATCGACAAGATCGAGCCGCACTTCATCACGGGC
AATGCGCTCAACGCGATCTCCGGTCGCGTGGCGTTCGCGCTGGGCTTCGAAGGTC
CTGCCGTCGCGGTCGACACCGCGTGCAGCTCGGCTCTGGTCGCGGTCCATCAGGC
ATGTCAGGCACTGCAGTCCGGGGACTGCGATCTGGCCCTCGCGGGAGGCGTCAA
CGTGCTGCTGAGCCCCGTCACCGTGGTCGCGGCCTCACGCGCGCGGATGCTGTCG
CCGGTGGGCCGGTGCAAGACGTTCGACGCGTCCGCCGACGGGTATGTGCGCAGT
GAGGGCTGCGGCGTCCTGGTGCTCAAGCGCCTCAGCGACGCCGTGCGTGACGGC
GACCGGGTGTGCGCGGTGATCCCTGCGAGCTCGGTGAACCAGGACGGCGCCTCC
AGCGGGTTGACCGTCCCCAACGGCGGTGCGCAGCAACGGCTCATCGAGGCGACG
CTGGCCCGCGCCGGACTGAGCGGCGCCGATGTGGACTACCTCGAGGCACACGGC
ACGGGTACGCCCCTCGGTGACCCGATCGAGGTGCAGGCCGCCGCGGCCGCGTAC
GGCGCCGGGCGTGACGCCGACCGGCCGCTGCTGATGGGATCGGTGAAGACCAAC
ATCGGGCACACCGAATCCGCCTCGGGTGCAGCGGGTCTGATCAAGGTGGTGCTG
TCACTGCAACACGGGGTGCTGCCGCAGAGCCTGCACTTCGAGAATCCGTCACCGC
ACATCCCGTGGGACGCATTGCCGGTGCGGGTGATCGACGAACCGGTGCCGTGGC
AGACCAACGGCCGGGTGCGCCGTGCCGGTGTCAGTTCGTTCGGGTTCACCGGCAC
CAACGCGCACGTGCTCGTCGAGGAGGCCCCGCCGCAACTGCACACCGCCGACGC
TGCGCCCGTGGAACCGGCCGCCACCGCGGAGGCCGGGACCGACGACTCGCCGCA
GGTGCTGGCGCTGTCGGCGCGGTCGCCCGAGGCGCTCGTGGAGTTGGCGCGGCG
CTACGACGACTGGTTGAGCGCGACGCCCGGCCTCGACATCGCCGACGTGTGCCA
CACCGCGGGCGTGGGCCGTTCGCACTTCGAATACCGCGCAGCCCTGGTGGTCGAC
TCGGTCGAGCGGGCACGCGAGGGTCTCGCCGCGCTCGCCGAGGGGCGCACTCCT
GCGGGCGTGGTGCGCGGCGAGCATGTGCACCGTCCGACGACGGCATGGCTGTTC
ACCGGACAGGGCAGCCAGTACCCGGGCATGGCCCGCGAATTGTTCGACACGCAA
CCGGTTTTCGCCCAGACCGTTCGGCAGTGCGCCGAGGCGGTCGCCGACATGCTGC
CGCGACCACTGCTGGACGTGCTGTTCGCGACCGACCGCGAAACCGCGGACCTGC
TGCAGCACACGTCGTTCGCGCAGCCCGCGCTGTTCGCGGTCGAGATGGGGCTAGC
GCGGCTGTGGCAGTCATGGGCCATCGAACCCGATGTGGTGCTGGGGCACAGCGT
CGGCCAGTACGCCGCGGCCTGCGTGGCGGGCGTGTTCAGCCTTGACGACGGCGC
ACGACTGATGGCCGAGCGCGGCCGCATGTTCGGCAGCCTTCCCGAAGGCGGCCG
GATGGTGGCCGTGTTCGCCGACGCCGAACACGTCGAGCAGGTGGCCGGTGAGTT
CCCCCGGGTGTCGGTCGGCGCCTACAACGGCCCCAACACCGTGCTCTCGGGTCCC
GGCGAGGACCTCGAAGAGATCGTCGAGAGGTTCGAGGACGAGGGGATCCGCTGC
ACGTGGCTGGCGACCAGCCACGCGTTCCACTCCGAACTGCTCGATCCCGTGCTCG
ACGAGTTCGAGGCCTACGCGGCCCAGTTCCAGTTCGCAGCGCCGACACTGCCTTT
GGTGTGCAACCGGACCGGGGCCGTGCTCACCGGGCAGACCCCGCTCGACGCGCA
GTACTGGCGCAGGCATTCCCGCCAGCCCGTGCAGTTCGCCGAGAGTGTGCGCACC
GTCGCCGCGCTCGGCTGCTCGGTGCTGATGGAGATCGGCCCGCAACCTGTGCTGA
CCGGGGCCGCGGTACAGGTGTGGCCCGAACACATGGCTGCCCCGCGGGCCGTCG
CCTCGCTGCGCAAGGGCGTCGCCGATCGCCGACAGATCGCCGATGCGCTGGCGT
CGGCCTACGTCGGCGGCCACCGCCCCGACTTCGCCGCGGTGCACCGGCAGCCAC
GCCGCCCGGTCGAGTTGCCCACCTATCCGTTCCAGCGCCGCAGGTTCTGGCCGAA
GGCCTCGAGCATCGCCGTCGACGGTGGGACCGCGGCGTCGGGAATCCTGGGCAG
CGGCAAGGATCTGGCGTCCGGCGACACCATCTACACGAGCCGGCTGTCCGTCAA
ATCGCAGCCGTGGCTGGCCGATCACGTCATCTACGGCACCGTCGTCGTCCCCGGC
GCGACGTATGCGGCGATGGCGCTCGCCGCGGTCGGCACACCGGCACGCCTGAAG
GACGTGTTCTTCTACGAGCCGATCATCCTGCCCGAGAAGGCCTCTCGCGAGGTCC
AGCTGACGTTGCACCCTGCCGACGGTGGCAGCGTGCTCAGCTTCCAGATCCACAG
CCGCCCGTACGGTGAACGTGGTGCCGACTGGTCGCTGAACGCCGAGGGCACGGT
CGACACGGCCGGCAGCGACCCGGACGCCGCCGCAGACGAGCAGTCGGATCCGGT
CGACGAGGTGATCGAACGCCTCGAGCGCATGCGTCCGCAGGACCTCTTCGAGAC
CTTCGCCGACCTGGAACTGGCGTGGGGTCCCACATGGTCGGGCTCGCTGAAATCG
TTGTGGCTCGGCGACGGTGAGGCCGTCGGCGACATCCTCGTCGGCGCCGAACTCG
CCGAGCAACTCGGCTCCGAGCCCATGCACCCGGTGCTGATGGACCTGTGCACGG
GCGTCGCGTTCCCCGCGTTCCCGGCGCTGCTCGCGGCCGAACAGGGCGTCAACGA
CCTGTTCCTGCCGCTGCGCTACGGGCAGGTGACGCTCGCGGAGAAGATGCCGCG
GCGGTTCTACTGCCGGGCGAGGTGGCACCGTAGCGCGCTCGACAGCGAAACGCA
GGTGTTCGAACTCGAATTCGTCGACCTCGGTGGGCGAGCGCTCGGCGGCATCCGC
GAGTTCACCGTCAAACGTGCGCCGCGCGAGGCACTGCTGCGCGGCCTCGGGGGT
GACGCGACCCGCCTGCTCTACACGCTCGGGTGGCACGAGGTGCCGCCGCCACCA
TCGGCCGACGACGCGGTCGGCACGTGGCTGATCGCCGGGTTCGACGAGCTGGCC
GCGAAGGTGCCCGGCTGTGTCCCCATCGACCGCAACACCGATCTGCAGGTCCTCG
GTGAGGTGCTGTCGCAGGCGCACGCTCGCGGCGCGGGATTCTCGGGCGTCGTGT
GGCGTTGCGCGACGCCTGGCCGGAAGGAATCGGGTTCTGCCTCCGCCGAATCCG
CCCGTCTGGAGGCCGAGATCACCAATCTGCTCAGCGCGGTGCACGCCGCGCAGG
GGCCGAGCGTGAAACTGCCCGGCGGACTCTGGATCGTCACCGAACACGGCGTGG
CATGTGAATCCGGCGAGCCGGTGGACCCGGTGCAGGCCGCGCTGTGGGGCTTCG
GCCGAACGACGATCAACGAGGAACCGGCGCTGCACTGCCGGCTCGTCGACTGCG
ACGGATCTTCCGAGGCCGTCGACCTGCTGGCCGCTCTGCTGGCCGCACCGGGCAT
CCAGGAACCGGAAATCGCCGTGCGGCAGGGCAAACTGCTGGCGTCACGGTTGTT
GCCGTGGGCCCGCAGTGGTCATCTCACGCTGCCGCGCGGGGGCGACTTCGTCCTT
GCGCCCACCGAGCGCGGCGCGATCGACAACCTGCGCCTCACGGAGACGGACGTG
CCGGAGCCGGACGAGGGCTACGTGCAGGTTCGGGTCGAGGCCGCGGGCCTCAAC
TTCCGCGACGTGCTCAACGTGCTCGGCCTGTACCCCGGTGATCCGGGACCGATCG
GCGGCGACTTCGCAGGCACCGTGACCCAACTGGGGGCAGGTGTGAGTGGACTCG
AAATCGGCCAACGCGTGTACGGGTCGATGCAGGGTGCCTTCGCGAGCCGCTTCA
ACGTGCCCGCCCAGTTCCTGGCGCCGATTCCCGACGGCATCGGCGCGGTCGAGGC
CGCCACGATCCCGGCCGCGGCGCTGACGGTCCGGCTCGCGTTCGACTGGGCGCA
ACTCAAACCCGGCGACAAGGTGCTCATCCACGCCGCCAGCGGTGGCGTGGGGCT
CGCGGCCATCCAGATGGCGCAGCAGTCCGGCGCCGAGGTCTTCGCCACCGCGAG
CACCTTCAAGCGTTCGACCCTGCGCAGGCTGGGCGTGAAGTACGTCTACGACTCT
CGGACGACGGACTTCGCCGACCAGATCCTCGCGGACACCAACGGTGCCGGCGTG
GACGTGGTGCTCAACAGCCTCACTAGTGCGGGTTTCGTCGAGGCGACGCTGAAG
GCCACCGCGCAGGGCGGACGCTTCGCCGAGATCGCCAAGCGCGACATCTGGTCG
CATGAGCAGATGGCCGAGGCCCGCCCCGACATCGCCTACGAGATCGTCGCGCTG
GACACCGTGATGTTCACCGAGCCCGATCGCATCCGCGACCTGCTCACCGAGGTGT
CCGACGGGATGGCGCGCGGCGAGTGGACACCACTACCCGCCGAGATCTACCCGA
TCACCGAGGCCCGGTCGGCGTTCCGGCGCATGCAACAGGCACGGCACATCGGCA
AGATTGTGCTGCAGGTCCCCAAACCCCTTCAGCCGCAGCAGGACCGGAGCTACCT
GATCACCGGCGGACTCGGGGCGATCGGCCTGCACACGGCGGCGTATCTGGCGCA
GCTCGGCGCGGGCGACATCGTGCTGACCAGCCGGCGCGACCCCGACGAGTCGGC
CCAGCGGGCGATCGACGAGATCACCGAGCGCCACAAGTGCCGCATCCACACCTA
TGCGGCCGATGTGGGTGACGAGGCACAGGTGGCCGATCTGCTGGCGCGGATCCG
GGCCGAGTTGCCTCCACTGGCCGGAATCGCCCATCTGGCGGGCGTGCTCGACGAC
GCGCTGCTGTCGGCGCAGAACCCCGAGCGCTTCCGGACCACGTTGGCGCCCAAG
GCCTATGGCGCGAGCCACCTGGACCGCCTGACGCGCGACGACGACCTGGACTTC
TTCATCGTGTCCTCGTCGGTGTCGAGCCTGTTCGGATCACCGGGGCAGGCCAACT
ACGCAACCGCCAATGCGATGCTCGACGGTCTGGTCGCCAAGCGCCGTGCGCAGG
GCCTGGTGGCCACGGGCATCAACTTCGGGCCGTGGGGTCAGGGCGGCATGGCGT
CGTCGGAGGCCGCGACCGCGAACATCACCGCCCAAGGTCTGATCCCGTTGGATC
CGTCGGCCGCGTTGCACGCGCTGGCCGAGGTCATCGCGAACGGCTCCGGGCAGG
CGACGGTGCTCAAGGCCAACTGGCAGCGCGCGGCGAAGGTTCTCGGCTCGGCGC
GCCCGCCGATCCTGGACCTCGTCCTGCCGAGCGCGGCCGGGGAGGCCACGGGTG
ACAGCGAGCTGCTCAAGCAGTTGATGGAGATCCCGATACCGCAGCGCGCCGGGT
TCGTCACCGAGTTCCTGCAACGCGAAGTGCAGAACTTCCTGCGTCTCGCGTCGCC
GCCCGCGGCGTCGAGCCGGTTCCTGGACCTCGGCACGGATTCGCTGATGGCCATC
GAGCTGCGCAACCGGCTGCACAGCCAGTTCGGGGGCAAGTTCACCATCAACGCG
ACCGCGGTGTTCGACTACCCGACCATCGGCGGGCTCGCGGAGTACCTCGTGGGTC
AGCTGCCCGACGCCACTAGTCAGCTCGACAGCGGGACTCCCGCCCGGGAAGCGA
GCAGCGCTCTTCGCGACGGCTACCGGCAGGCGGGCGTGTCGGGCAGGGTCCGGT
CCTACCTCGACCTGCTGGCGGGGCTGTCGGACTTCCGCGAGCACTTCGACGGCTC
CGACGGGTTCTCCCTCGATCTCGTGGACATGGCCGACGGTCCCGGAGAGGTCACG
GTGATCTGCTGCGCGGGAACGGCGGCGATCTCCGGTCCGCACGAGTTCACCCGG
CTCGCCGGGGCGCTGCGCGGAATCGCTCCGGTTCGGGCCGTGCCCCAGCCCGGCT
ACGAGGAGGGCGAACCTCTGCCGTCGTCGATGGCGGCGGTGGCGGCGGTGCAGG
CCGATGCGGTCATCAGGACACAGGGGGACAAGCCGTTCGTGGTGGCCGGTCACT
CCGCGGGGGCACTGATGGCCTACGCGCTGGCGACCGAACTGCTCGATCGCGGGC
ACCCGCCACGCGGTGTCGTCCTGATCGACGTCTACCCGCCCGGTCACCAGGACGC
GATGAACGCCTGGCTGGAGGAGCTGACCGCCACGCTGTTCGACCGCGAGACGGT
GCGGATGGACGACACCAGGCTCACCGCCCTGGGCGCCTACGACCGCCTCACCGG
TCAGTGGCGACCCCGGGAAACCGGGCTGCCGACGCTGCTGGTCAGCGCCGGCGA
GCCGATGGGTCCGTGGCCCGACGACAGCTGGAAGCCGACGTGGCCCTTCGAGCA
CGACACCGTCGCCGTCCCCGGCGACCACTTCACGATGGTGCAGGAACACGCCGA
CGCGATCGCGCGGCACATCGACGCCTGGCTGGGCGGAGGGAATTCATGA
SEQ ID NO: 13 depicts the amino acid sequence of PKS7TE
MVSAEHPIEPTPSFAIIGYAARFPGAASAEEYWALLRDGREAISDVPKDRWDIEEFFDP
DPSTPGKVVTRRAGFVDDVTGFDAPFFGMSAREVRLMDPQHRILMETAWRAVEHSG
IAPTSLANSNTGVFVGLATHDYLGMASDELTYPEIEAYMAIGTSNAAAAGRISYRLG
LQGPAVAVDTACSSSLVAIHQACQALRLNECDLALAGGANVLLTPATMITFSNAHM
LAPDGRCKTFDAAADGYVRGEGCGVVVVKRLEDAVRDGDRIRAVIRGSAINQDGAS
GGLTVPNGVAQQRVIADALKRAGLRPADVGYLEAHGTGTSLGDPIEAQAAGEVLGA
GRAPEEPLLIGSAKTNIGHLEAAAGIAGVIKVVMALENETLPKHLNFETPSPHIPWERL
PIEVVRETVPWQRNGKPRIAGVSSFGFAGTNAHVILEEAPAVGTPATTEPVEDPRRYS
VLPLSARTPEALVRLAGEYRDWLRAHPQARLADVSHTAGTARAHLEQRAALVVNS
RESAIELLGALADDRPAPGLVRGESHEAPKTAWLFTGQGSQYPGMARELFDTEPVFA
ETVRKCAEAVADILEKPLLDVIFDADELDGDTESPLRHTTYAQPALFAVELGLARLW
QSWGFEPDVVVGHSVGQYSAACVAGVFDVEDGARLIAERGRLFGSLPAGGRMAAV
FTAPERAERLTDEYPSLSVAAYNGANTVLSGPAKDLESAVATLVADGVRCDWLDTS
HAFHSALLDPILDDFESFAQKLEYREPQRILIDNRTGSALGRSTRLDGAYWRRHARQP
VEFAKSVRTLADMNCRVLLEIGPRPVLTATALAAWPDPATTPQVLASLRPTTADHRQ
ITEAVAAAYAAGHLPDFRAFRQPDARKLDLPTYPFEHRQFWFSDNRAIDRDTQTASA
GSVTPHRTQAVRLLEDGQIEELAALIDGETVDEQTLRVLNKLAARHNQQRSTHVDPD
ARYEIRWDAIASAAASTGAAADWILVGDDTAAVLEFADALTAGGHRHQIVALPGSD
ADEAQLVDTLRAASAGELYVVHIAAGDGAAAPMRDLLRVQHRTLGGLRRLFRAAN
DAELRSPIWLVTRDGQRVTDTDTVVPEQSCLWGFGRAAALELPHIWGGLADLSGAD
TEWPRLIARITSPRDGEDQIALRGDAVYAPRLVRRTGEPSDTPLPVREDRTYLVTGGL
GAIGLEVAGYLASHGAGHVVLTSRREPGDDARRRLDGLREQYACDVRVITADVADA
HDVARLMTTVAAELPPLAGIVHAAGEIGTTPLSGLDDAEVDRVFAGKVWGAWHLSE
ALTDLRTDLDFFVSTSSIASVWGGFGQTAYGAANAFLDGLAWRLRERGVTAVSVNF
GPWAAGMADAESRARLEQRGVRTLSPAEALAGMADVVAGPVQGVVAKIDWSRFLP
LYQQAGRRAFLSELESELPAAATGAAAPATVPGKPPLVEQLTKAPVQQRKSLITNYL
RNAVAEVTRVDADEIRDEAGFFDLGMDSLMAVELRRRIEQGVGKDIPVTLVMDHPR
LSDAADYLLVEVLGLGEQTNVRQASTVTARTDDPIAVVAVSCRFPGAPDPESFWELL
AGGVDAIREVPEDRWDIDEFYDPDPDTPGKTYTRFGGFLDGIDGFDPEFFGISPREAV
WIEPQQRLMLETVWEGLERAGYAPSDLRGSRTGVFTGVAANEYAHLLSAESIDKIEP
HFITGNALNAISGRVAFALGFEGPAVAVDTACSSALVAVHQACQALQSGDCDLALA
GGVNVLLSPVTVVAASRARMLSPVGRCKTFDASADGYVRSEGCGVLVLKRLSDAV
RDGDRVCAVIPASSVNQDGASSGLTVPNGGAQQRLIEATLARAGLSGADVDYLEAH
GTGTPLGDPIEVQAAAAAYGAGRDADRPLLMGSVKTNIGHTESASGAAGLIKVVLSL
QHGVLPQSLHFENPSPHIPWDALPVRVIDEPVPWQTNGRVRRAGVSSFGFTGTNAHV
LVEEAPPQLHTADAAPVEPAATAEAGTDDSPQVLALSARSPEALVELARRYDDWLS
ATPGLDIADVCHTAGVGRSHFEYRAALVVDSVERAREGLAALAEGRTPAGVVRGEH
VHRPTTAWLFTGQGSQYPGMARELFDTQPVFAQTVRQCAEAVADMLPRPLLDVLFA
TDRETADLLQHTSFAQPALFAVEMGLARLWQSWAIEPDVVLGHSVGQYAAACVAG
VFSLDDGARLMAERGRMFGSLPEGGRMVAVFADAEHVEQVAGEFPRVSVGAYNGP
NTVLSGPGEDLEEIVERFEDEGIRCTWLATSHAFHSELLDPVLDEFEAYAAQFQFAAP
TLPLVCNRTGAVLTGQTPLDAQYWRRHSRQPVQFAESVRTVAALGCSVLMEIGPQP
VLTGAAVQVWPEHMAAPRAVASLRKGVADRRQIADALASAYVGGHRPDFAAVHR
QPRRPVELPTYPFQRRRFWPKASSIAVDGGTAASGILGSGKDLASGDTIYTSRLSVKS
QPWLADHVIYGTVVVPGATYAAMALAAVGTPARLKDVFFYEPIILPEKASREVQLTL
HPADGGSVLSFQIHSRPYGERGADWSLNAEGTVDTAGSDPDAAADEQSDPVDEVIER
LERMRPQDLFETFADLELAWGPTWSGSLKSLWLGDGEAVGDILVGAELAEQLGSEP
MHPVLMDLCTGVAFPAFPALLAAEQGVNDLFLPLRYGQVTLAEKMPRRFYCRARW
HRSALDSETQVFELEFVDLGGRALGGIREFTVKRAPREALLRGLGGDATRLLYTLGW
HEVPPPPSADDAVGTWLIAGFDELAAKVPGCVPIDRNTDLQVLGEVLSQAHARGAG
FSGVVWRCATPGRKESGSASAESARLEAEITNLLSAVHAAQGPSVKLPGGLWIVTEH
GVACESGEPVDPVQAALWGFGRTTINEEPALHCRLVDCDGSSEAVDLLAALLAAPGI
QEPEIAVRQGKLLASRLLPWARSGHLTLPRGGDFVLAPTERGAIDNLRLTETDVPEPD
EGYVQVRVEAAGLNFRDVLNVLGLYPGDPGPIGGDFAGTVTQLGAGVSGLEIGQRV
YGSMQGAFASRFNVPAQFLAPIPDGIGAVEAATIPAAALTVRLAFDWAQLKPGDKVL
IHAASGGVGLAAIQMAQQSGAEVFATASTFKRSTLRRLGVKYVYDSRTTDFADQILA
DTNGAGVDVVLNSLTSAGFVEATLKATAQGGRFAEIAKRDIWSHEQMAEARPDIAY
EIVALDTVMFTEPDRIRDLLTEVSDGMARGEWTPLPAEIYPITEARSAFRRMQQARHI
GKIVLQVPKPLQPQQDRSYLITGGLGAIGLHTAAYLAQLGAGDIVLTSRRDPDESAQR
AIDEITERHKCRIHTYAADVGDEAQVADLLARIRAELPPLAGIAHLAGVLDDALLSAQ
NPERFRTTLAPKAYGASHLDRLTRDDDLDFFIVSSSVSSLFGSPGQANYATANAMLD
GLVAKRRAQGLVATGINFGPWGQGGMASSEAATANITAQGLIPLDPSAALHALAEVI
ANGSGQATVLKANWQRAAKVLGSARPPILDLVLPSAAGEATGDSELLKQLMEIPIPQ
RAGFVTEFLQREVQNFLRLASPPAASSRFLDLGTDSLMAIELRNRLHSQFGGKFTINA
TAVFDYPTIGGLAEYLVGQLPDATSQLDSGTPAREASSALRDGYRQAGVSGRVRSYL
DLLAGLSDFREHFDGSDGFSLDLVDMADGPGEVTVICCAGTAAISGPHEFTRLAGAL
RGIAPVRAVPQPGYEEGEPLPSSMAAVAAVQADAVIRTQGDKPFVVAGHSAGALMA
YALATELLDRGHPPRGVVLIDVYPPGHQDAMNAWLEELTATLFDRETVRMDDTRLT
ALGAYDRLTGQWRPRETGLPTLLVSAGEPMGPWPDDSWKPTWPFEHDTVAVPGDH
FTMVQEHADAIARHIDAWLGGGNS
SEQ ID NO: 14 depicts the nucleic acid sequence of a non-working vector
pSSRi14
TGGCGAATGGGACGCGCCCTGTAGCGGCGCATTAAGCGCGGCGGGTGTGGTGGT
TACGCGCAGCGTGACCGCTACACTTGCCAGCGCCCTAGCGCCCGCTCCTTTCGCT
TTCTTCCCTTCCTTTCTCGCCACGTTCGCCGGCTTTCCCCGTCAAGCTCTAAATCG
GGGGCTCCCTTTAGGGTTCCGATTTAGTGCTTTACGGCACCTCGACCCCAAAAAA
CTTGATTAGGGTGATGGTTCACGTAGTGGGCCATCGCCCTGATAGACGGTTTTTC
GCCCTTTGACGTTGGAGTCCACGTTCTTTAATAGTGGACTCTTGTTCCAAACTGGA
ACAACACTCAACCCTATCTCGGTCTATTCTTTTGATTTATAAGGGATTTTGCCGAT
TTCGGCCTATTGGTTAAAAAATGAGCTGATTTAACAAAAATTTAACGCGAATTTT
AACAAAATATTAACGTTTACAATTTCAGGTGGCACTTTTCGGGGAAATGTGCGCG
GAACCCCTATTTGTTTATTTTTCTAAATACATTCAAATATGTATCCGCTCATGAGA
CAATAACCCTGATAAATGCTTCAATAATATTGAAAAAGGAAGAGTATGAGTATT
CAACATTTCCGTGTCGCCCTTATTCCCTTTTTTGCGGCATTTTGCCTTCCTGTTTTT
GCTCACCCAGAAACGCTGGTGAAAGTAAAAGATGCTGAAGATCAGTTGGGTGCA
CGAGTGGGTTACATCGAACTGGATCTCAACAGCGGTAAGATCCTTGAGAGTTTTC
GCCCCGAAGAACGTTTTCCAATGATGAGCACTTTTAAAGTTCTGCTATGTGGCGC
GGTATTATCCCGTATTGACGCCGGGCAAGAGCAACTCGGTCGCCGCATACACTAT
TCTCAGAATGACTTGGTTGAGTACTCACCAGTCACAGAAAAGCATCTTACGGATG
GCATGACAGTAAGAGAATTATGCAGTGCTGCCATAACCATGAGTGATAACACTG
CGGCCAACTTACTTCTGACAACGATCGGAGGACCGAAGGAGCTAACCGCTTTTTT
GCACAACATGGGGGATCATGTAACTCGCCTTGATCGTTGGGAACCGGAGCTGAA
TGAAGCCATACCAAACGACGAGCGTGACACCACGATGCCTGCAGCAATGGCAAC
AACGTTGCGCAAACTATTAACTGGCGAACTACTTACTCTAGCTTCCCGGCAACAA
TTAATAGACTGGATGGAGGCGGATAAAGTTGCAGGACCACTTCTGCGCTCGGCC
CTTCCGGCTGGCTGGTTTATTGCTGATAAATCTGGAGCCGGTGAGCGTGGGTCTC
GCGGTATCATTGCAGCACTGGGGCCAGATGGTAAGCCCTCCCGTATCGTAGTTAT
CTACACGACGGGGAGTCAGGCAACTATGGATGAACGAAATAGACAGATCGCTGA
GATAGGTGCCTCACTGATTAAGCATTGGTAACTGTCAGACCAAGTTTACTCATAT
ATACTTTAGATTGATTTAAAACTTCATTTTTAATTTAAAAGGATCTAGGTGAAGA
TCCTTTTTGATAATCTCATGACCAAAATCCCTTAACGTGAGTTTTCGTTCCACTGA
GCGTCAGACCCCGTAGAAAAGATCAAAGGATCTTCTTGAGATCCTTTTTTTCTGC
GCGTAATCTGCTGCTTGCAAACAAAAAAACCACCGCTACCAGCGGTGGTTTGTTT
GCCGGATCAAGAGCTACCAACTCTTTTTCCGAAGGTAACTGGCTTCAGCAGAGCG
CAGATACCAAATACTGTCCTTCTAGTGTAGCCGTAGTTAGGCCACCACTTCAAGA
ACTCTGTAGCACCGCCTACATACCTCGCTCTGCTAATCCTGTTACCAGTGGCTGCT
GCCAGTGGCGATAAGTCGTGTCTTACCGGGTTGGACTCAAGACGATAGTTACCGG
ATAAGGCGCAGCGGTCGGGCTGAACGGGGGGTTCGTGCACACAGCCCAGCTTGG
AGCGAACGACCTACACCGAACTGAGATACCTACAGCGTGAGCTATGAGAAAGCG
CCACGCTTCCCGAAGGGAGAAAGGCGGACAGGTATCCGGTAAGCGGCAGGGTCG
GAACAGGAGAGCGCACGAGGGAGCTTCCAGGGGGAAACGCCTGGTATCTTTATA
GTCCTGTCGGGTTTCGCCACCTCTGACTTGAGCGTCGATTTTTGTGATGCTCGTCA
GGGGGGCGGAGCCTATGGAAAAACGCCAGCAACGCGGCCTTTTTACGGTTCCTG
GCCTTTTGCTGGCCTTTTGCTCACATGTTCTTTCCTGCGTTATCCCCTGATTCTGTG
GATAACCGTATTACCGCCTTTGAGTGAGCTGATACCGCTCGCCGCAGCCGAACGA
CCGAGCGCAGCGAGTCAGTGAGCGAGGAAGCGGAAGAGCGCCTGATGCGGTATT
TTCTCCTTACGCATCTGTGCGGTATTTCACACCGCATATATGGTGCACTCTCAGTA
CAATCTGCTCTGATGCCGCATAGTTAAGCCAGTATACACTCCGCTATCGCTACGT
GACTGGGTCATGGCTGCGCCCCGACACCCGCCAACACCCGCTGACGCGCCCTGA
CGGGCTTGTCTGCTCCCGGCATCCGCTTACAGACAAGCTGTGACCGTCTCCGGGA
GCTGCATGTGTCAGAGGTTTTCACCGTCATCACCGAAACGCGCGAGGCAGCTGCG
GTAAAGCTCATCAGCGTGGTCGTGAAGCGATTCACAGATGTCTGCCTGTTCATCC
GCGTCCAGCTCGTTGAGTTTCTCCAGAAGCGTTAATGTCTGGCTTCTGATAAAGC
GGGCCATGTTAAGGGCGGTTTTTTCCTGTTTGGTCACTGATGCCTCCGTGTAAGG
GGGATTTCTGTTCATGGGGGTAATGATACCGATGAAACGAGAGAGGATGCTCAC
GATACGGGTTACTGATGATGAACATGCCCGGTTACTGGAACGTTGTGAGGGTAA
ACAACTGGCGGTATGGATGCGGCGGGACCAGAGAAAAATCACTCAGGGTCAATG
CCAGCGCTTCGTTAATACAGATGTAGGTGTTCCACAGGGTAGCCAGCAGCATCCT
GCGATGCAGATCCGGAACATAATGGTGCAGGGCGCTGACTTCCGCGTTTCCAGA
CTTTACGAAACACGGAAACCGAAGACCATTCATGTTGTTGCTCAGGTCGCAGACG
TTTTGCAGCAGCAGTCGCTTCACGTTCGCTCGCGTATCGGTGATTCATTCTGCTAA
CCAGTAAGGCAACCCCGCCAGCCTAGCCGGGTCCTCAACGACAGGAGCACGATC
ATGCGCACCCGTGGGGCCGCCATGCCGGCGATAATGGCCTGCTTCTCGCCGAAAC
GTTTGGTGGCGGGACCAGTGACGAAGGCTTGAGCGAGGGCGTGCAAGATTCCGA
ATACCGCAAGCGACAGGCCGATCATCGTCGCGCTCCAGCGAAAGCGGTCCTCGC
CGAAAATGACCCAGAGCGCTGCCGGCACCTGTCCTACGAGTTGCATGATAAAGA
AGACAGTCATAAGTGCGGCGACGATAGTCATGCCCCGCGCCCACCGGAAGGAGC
TGACTGGGTTGAAGGCTCTCAAGGGCATCGGTCGAGATCCCGGTGCCTAATGAGT
GAGCTAACTTACATTAATTGCGTTGCGCTCACTGCCCGCTTTCCAGTCGGGAAAC
CTGTCGTGCCAGCTGCATTAATGAATCGGCCAACGCGCGGGGAGAGGCGGTTTG
CGTATTGGGCGCCAGGGTGGTTTTTCTTTTCACCAGTGAGACGGGCAACAGCTGA
TTGCCCTTCACCGCCTGGCCCTGAGAGAGTTGCAGCAAGCGGTCCACGCTGGTTT
GCCCCAGCAGGCGAAAATCCTGTTTGATGGTGGTTAACGGCGGGATATAACATG
AGCTGTCTTCGGTATCGTCGTATCCCACTACCGAGATATCCGCACCAACGCGCAG
CCCGGACTCGGTAATGGCGCGCATTGCGCCCAGCGCCATCTGATCGTTGGCAACC
AGCATCGCAGTGGGAACGATGCCCTCATTCAGCATTTGCATGGTTTGTTGAAAAC
CGGACATGGCACTCCAGTCGCCTTCCCGTTCCGCTATCGGCTGAATTTGATTGCG
AGTGAGATATTTATGCCAGCCAGCCAGACGCAGACGCGCCGAGACAGAACTTAA
TGGGCCCGCTAACAGCGCGATTTGCTGGTGACCCAATGCGACCAGATGCTCCACG
CCCAGTCGCGTACCGTCTTCATGGGAGAAAATAATACTGTTGATGGGTGTCTGGT
CAGAGACATCAAGAAATAACGCCGGAACATTAGTGCAGGCAGCTTCCACAGCAA
TGGCATCCTGGTCATCCAGCGGATAGTTAATGATCAGCCCACTGACGCGTTGCGC
GAGAAGATTGTGCACCGCCGCTTTACAGGCTTCGACGCCGCTTCGTTCTACCATC
GACACCACCACGCTGGCACCCAGTTGATCGGCGCGAGATTTAATCGCCGCGACA
ATTTGCGACGGCGCGTGCAGGGCCAGACTGGAGGTGGCAACGCCAATCAGCAAC
GACTGTTTGCCCGCCAGTTGTTGTGCCACGCGGTTGGGAATGTAATTCAGCTCCG
CCATCGCCGCTTCCACTTTTTCCCGCGTTTTCGCAGAAACGTGGCTGGCCTGGTTC
ACCACGCGGGAAACGGTCTGATAAGAGACACCGGCATACTCTGCGACATCGTAT
AACGTTACTGGTTTCACATTCACCACCCTGAATTGACTCTCTTCCGGGCGCTATCA
TGCCATACCGCGAAAGGTTTTGCGCCATTCGATGGTGTCCGGGATCTCGACGCTC
TCCCTTATGCGACTCCTGCATTAGGAAGCAGCCCAGTAGTAGGTTGAGGCCGTTG
AGCACCGCCGCCGCAAGGAATGGTGCATGCAAGGAGATGGCGCCCAACAGTCCC
CCGGCCACGGGGCCTGCCACCATACCCACGCCGAAACAAGCGCTCATGAGCCCG
AAGTGGCGAGCCCGATCTTCCCCATCGGTGATGTCGGCGATATAGGCGCCAGCA
ACCGCACCTGTGGCGCCGGTGATGCCGGCCACGATGCGTCCGGCGTAGAGGATC
GAGATCTCGATCCCGCGAAATTAATACGACTCACTATAGGGGAATTGTGAGCGG
ATAACAATTCCCCTCTAGAAATAATTTTGTTTAACTTTAAGAAGGAGATATACAT
ATGGTCTCTGCCGAACATCCGATCGAGCCGACCCCGAGCTTCGCAATCATCGGCT
ACGCCGCACGTTTTCCCGGCGCGGCGAGCGCCGAGGAATACTGGGCGTTGTTGC
GGGACGGGCGTGAAGCGATATCCGATGTTCCGAAAGACCGCTGGGACATCGAGG
AGTTCTTCGACCCGGACCCGTCGACACCGGGCAAGGTCGTCACGCGCCGCGCGG
GGTTCGTCGATGACGTAACGGGTTTCGACGCGCCGTTCTTCGGTATGTCCGCCCG
CGAGGTACGTCTGATGGACCCGCAGCACCGGATTCTCATGGAAACCGCGTGGCG
CGCGGTCGAACACTCGGGAATCGCGCCGACGTCGCTGGCCAACAGCAACACGGG
CGTCTTCGTCGGCCTGGCCACCCACGACTACCTCGGTATGGCCTCCGACGAACTC
ACCTACCCCGAGATCGAGGCCTACATGGCCATCGGAACCTCCAATGCCGCTGCG
GCCGGACGGATCAGTTACCGCCTGGGATTGCAGGGACCGGCGGTGGCGGTCGAC
ACCGCGTGCAGTTCGTCGCTGGTGGCGATTCACCAGGCGTGTCAGGCACTTCGCC
TGAACGAGTGCGATCTCGCGTTGGCCGGCGGGGCGAACGTCCTGCTCACCCCGG
CCACCATGATCACGTTCTCCAACGCGCACATGCTGGCTCCGGACGGCCGGTGCAA
GACCTTCGACGCGGCGGCCGACGGCTACGTGCGCGGCGAGGGCTGCGGTGTCGT
CGTGGTCAAGCGCCTCGAGGACGCGGTGCGCGACGGCGACCGGATCCGCGCGGT
GATCCGCGGAAGCGCAATCAACCAGGACGGGGCGTCGGGCGGGCTGACCGTGCC
GAACGGCGTTGCTCAGCAACGGGTTATCGCCGACGCGTTGAAGCGCGCCGGCCT
GCGACCGGCCGATGTCGGTTATCTCGAGGCGCACGGCACCGGAACCTCACTTGG
CGACCCCATCGAGGCCCAGGCCGCGGGCGAGGTGCTCGGGGCCGGACGCGCACC
TGAAGAACCGCTGCTGATCGGCTCGGCGAAGACCAACATCGGCCATCTGGAAGC
GGCCGCGGGCATCGCCGGTGTCATCAAGGTGGTCATGGCGCTCGAGAACGAGAC
GTTGCCCAAGCACCTCAATTTCGAGACTCCGTCGCCCCACATCCCGTGGGAGCGT
CTACCCATCGAGGTGGTGAGAGAGACCGTTCCCTGGCAGCGCAACGGCAAGCCG
CGGATCGCCGGGGTGAGCTCATTCGGGTTTGCCGGGACTAACGCGCACGTCATCC
TCGAAGAGGCGCCCGCCGTCGGAACACCGGCGACGACAGAGCCTGTCGAGGATC
CGAGGCGCTACAGCGTCCTTCCGCTGTCGGCACGCACGCCCGAGGCGTTGGTCCG
ACTCGCCGGTGAGTACCGCGACTGGCTGCGCGCCCACCCGCAGGCCCGCCTGGC
CGACGTGTCCCACACCGCGGGAACCGCACGTGCCCACCTGGAGCAGCGTGCCGC
GTTGGTGGTCAACTCGCGGGAATCGGCCATCGAACTGCTCGGCGCGCTCGCCGAC
GACCGTCCGGCGCCCGGCCTGGTCCGCGGTGAATCCCACGAGGCACCCAAGACC
GCGTGGTTGTTCACCGGCCAGGGCAGCCAGTACCCCGGAATGGCGCGTGAGTTG
TTCGACACCGAACCGGTGTTCGCCGAAACCGTGAGGAAATGCGCCGAGGCCGTC
GCAGATATCCTCGAAAAGCCTTTGCTCGACGTCATTTTCGACGCCGACGAGTTGG
ATGGTGACACCGAATCGCCGCTGCGGCACACGACCTATGCGCAACCCGCGCTGTT
CGCCGTCGAATTGGGCCTGGCCCGGCTCTGGCAGTCCTGGGGCTTCGAACCCGAC
GTGGTGGTCGGACACAGTGTCGGGCAGTACTCGGCCGCCTGTGTGGCGGGTGTGT
TCGACGTCGAGGACGGCGCACGACTGATCGCCGAGCGCGGCCGATTGTTCGGCA
GCCTGCCCGCGGGTGGACGCATGGCGGCGGTTTTCACCGCCCCCGAGCGTGCAG
AGCGCCTCACCGACGAGTACCCCAGCCTGTCGGTTGCCGCGTACAACGGTGCCA
ACACCGTATTGTCCGGTCCCGCAAAGGATCTCGAATCCGCCGTGGCCACATTGGT
GGCCGACGGTGTGCGGTGCGACTGGCTGGACACCAGCCACGCCTTCCACTCGGC
ACTGCTCGACCCGATTCTCGACGATTTCGAGTCGTTCGCCCAGAAGCTCGAATAC
CGTGAACCGCAACGTATCCTGATCGACAACCGCACCGGGTCCGCGCTCGGGCGG
AGCACCAGGCTCGACGGTGCCTACTGGCGGCGGCACGCACGCCAGCCTGTCGAG
TTCGCCAAGAGCGTCCGCACCCTCGCCGACATGAACTGCAGGGTGCTGCTGGAG
ATAGGGCCCAGGCCCGTGCTCACCGCCACCGCGCTGGCAGCATGGCCCGACCCG
GCCACCACGCCGCAGGTGCTCGCGTCGCTGCGGCCCACCACGGCCGATCACCGG
CAGATCACCGAGGCCGTCGCCGCCGCCTATGCCGCGGGACATCTGCCCGATTTCC
GCGCGTTCCGGCAACCGGACGCGCGCAAACTCGACCTGCCCACATATCCCTTCGA
GCATCGCCAGTTCTGGTTCTCGGACAACCGTGCGATCGACCGTGACACCCAGACC
GCGAGCGCCGGTTCCGTCACGCCTCACCGCACACAGGCCGTCCGACTTCTCGAAG
ACGGCCAGATCGAGGAACTCGCCGCCCTCATCGACGGCGAGACCGTCGACGAGC
AGACCCTGCGCGTGCTGAACAAGCTCGCGGCGCGCCACAACCAGCAGCGGTCCA
CACACGTCGACCCTGATGCGCGGTACGAGATCCGTTGGGACGCGATCGCTTCGGC
CGCCGCGAGCACGGGCGCGGCAGCCGACTGGATCCTGGTCGGCGACGACACCGC
TGCCGTCCTGGAGTTCGCCGATGCGCTCACCGCAGGCGGTCATCGCCACCAGATC
GTCGCGCTGCCCGGCTCCGACGCCGACGAGGCCCAGCTGGTCGATACGCTGCGC
GCTGCGTCGGCCGGTGAGCTGTACGTCGTGCACATCGCGGCAGGAGACGGTGCC
GCTGCCCCGATGCGGGACCTGCTGCGCGTGCAGCACCGAACCCTTGGTGGGCTGC
GGCGCCTGTTCCGCGCCGCGAACGACGCGGAACTGCGCAGCCCCATCTGGTTGGT
GACACGTGACGGGCAGCGGGTCACCGACACCGACACCGTGGTACCGGAACAGAG
TTGCCTGTGGGGATTCGGCCGCGCGGCGGCCCTGGAACTCCCGCACATCTGGGGT
GGCCTCGCCGACCTCTCCGGTGCGGACACCGAGTGGCCGCGGCTCATCGCGCGG
ATCACGTCACCGCGCGACGGTGAGGACCAGATCGCGCTGCGCGGGGACGCCGTG
TACGCACCCCGACTGGTCCGCCGGACCGGCGAGCCGAGCGACACCCCCCTGCCG
GTGCGGGAGGACCGCACCTATCTGGTGACGGGCGGTCTCGGGGCGATCGGGCTC
GAGGTCGCCGGCTACCTGGCCTCCCACGGTGCCGGTCATGTGGTGCTCACCAGCC
GACGGGAACCGGGCGACGACGCGCGCCGGCGCCTCGACGGTCTCCGCGAGCAGT
ACGCATGTGACGTCCGGGTGATCACCGCCGATGTCGCCGACGCGCACGACGTCG
CGCGCCTCATGACCACCGTCGCCGCCGAACTCCCGCCGCTGGCCGGCATCGTGCA
CGCCGCGGGCGAGATCGGCACCACGCCGCTGAGCGGCCTCGACGACGCCGAGGT
GGATCGCGTCTTCGCCGGGAAGGTCTGGGGCGCCTGGCATCTGAGCGAGGCCCT
GACAGATCTTCGCACCGATCTCGACTTCTTCGTCAGCACATCGTCGATCGCGTCG
GTGTGGGGCGGATTCGGGCAGACCGCCTACGGCGCGGCGAACGCGTTTCTCGAC
GGTCTGGCGTGGCGACTGCGCGAGCGGGGTGTCACCGCTGTCAGCGTGAACTTC
GGGCCGTGGGCCGCGGGCATGGCCGACGCCGAATCACGTGCCCGCCTCGAGCAG
CGAGGCGTGCGGACGTTGTCGCCCGCCGAGGCCCTCGCAGGCATGGCCGACGTC
GTGGCCGGCCCCGTTCAGGGAGTCGTCGCCAAGATCGACTGGTCACGTTTCCTGC
CGCTGTACCAGCAGGCAGGCCGGCGCGCGTTCCTGTCGGAACTCGAAAGCGAAT
TGCCCGCAGCGGCAACCGGCGCTGCTGCGCCCGCGACGGTGCCCGGGAAACCAC
CGCTCGTCGAGCAGTTGACCAAAGCGCCTGTGCAGCAACGCAAAAGCCTCATCA
CGAACTACCTGCGCAACGCGGTCGCCGAGGTCACACGGGTCGACGCCGACGAGA
TCCGCGATGAGGCCGGGTTCTTCGACCTCGGGATGGACTCCCTGATGGCCGTCGA
ACTGCGGCGGCGCATCGAGCAGGGTGTCGGCAAGGACATTCCCGTCACGCTCGT
GATGGATCATCCGCGGTTGTCCGACGCGGCCGACTACCTGCTGGTCGAGGTACTC
GGGTTGGGCGAGCAGACCAACGTGCGGCAGGCGTCGACCGTGACCGCGCGAACC
GACGATCCCATCGCGGTCGTCGCGGTGTCCTGCCGCTTCCCCGGCGCACCCGACC
CGGAGTCGTTCTGGGAGCTGCTCGCCGGTGGTGTCGATGCCATCCGAGAGGTCCC
CGAGGACCGCTGGGACATCGACGAGTTCTACGACCCCGATCCGGACACCCCGGG
CAAGACCTACACGCGGTTCGGCGGATTTCTAGACGGCATCGACGGATTCGATCCC
GAGTTCTTCGGCATCTCACCGCGTGAGGCCGTGTGGATCGAGCCGCAGCAGCGCC
TCATGCTCGAGACGGTGTGGGAGGGTCTCGAGCGGGCCGGCTACGCACCTTCGG
ACCTCCGGGGCAGCCGCACGGGTGTCTTCACCGGTGTTGCCGCCAACGAGTACGC
GCATCTGCTGTCGGCCGAGTCGATCGACAAGATCGAGCCGCACTTCATCACGGGC
AATGCGCTCAACGCGATCTCCGGTCGCGTGGCGTTCGCGCTGGGCTTCGAAGGTC
CTGCCGTCGCGGTCGACACCGCGTGCAGCTCGGCTCTGGTCGCGGTCCATCAGGC
ATGTCAGGCACTGCAGTCCGGGGACTGCGATCTGGCCCTCGCGGGAGGCGTCAA
CGTGCTGCTGAGCCCCGTCACCGTGGTCGCGGCCTCACGCGCGCGGATGCTGTCG
CCGGTGGGCCGGTGCAAGACGTTCGACGCGTCCGCCGACGGGTATGTGCGCAGT
GAGGGCTGCGGCGTCCTGGTGCTCAAGCGCCTCAGCGACGCCGTGCGTGACGGC
GACCGGGTGTGCGCGGTGATCCCTGCGAGCTCGGTGAACCAGGACGGCGCCTCC
AGCGGGTTGACCGTCCCCAACGGCGGTGCGCAGCAACGGCTCATCGAGGCGACG
CTGGCCCGCGCCGGACTGAGCGGCGCCGATGTGGACTACCTCGAGGCACACGGC
ACGGGTACGCCCCTCGGTGACCCGATCGAGGTGCAGGCCGCCGCGGCCGCGTAC
GGCGCCGGGCGTGACGCCGACCGGCCGCTGCTGATGGGATCGGTGAAGACCAAC
ATCGGGCACACCGAATCCGCCTCGGGTGCAGCGGGTCTGATCAAGGTGGTGCTG
TCACTGCAACACGGGGTGCTGCCGCAGAGCCTGCACTTCGAGAATCCGTCACCGC
ACATCCCGTGGGACGCATTGCCGGTGCGGGTGATCGACGAACCGGTGCCGTGGC
AGACCAACGGCCGGGTGCGCCGTGCCGGTGTCAGTTCGTTCGGGTTCACCGGCAC
CAACGCGCACGTGCTCGTCGAGGAGGCCCCGCCGCAACTGCACACCGCCGACGC
TGCGCCCGTGGAACCGGCCGCCACCGCGGAGGCCGGGACCGACGACTCGCCGCA
GGTGCTGGCGCTGTCGGCGCGGTCGCCCGAGGCGCTCGTGGAGTTGGCGCGGCG
CTACGACGACTGGTTGAGCGCGACGCCCGGCCTCGACATCGCCGACGTGTGCCA
CACCGCGGGCGTGGGCCGTTCGCACTTCGAATACCGCGCAGCCCTGGTGGTCGAC
TCGGTCGAGCGGGCACGCGAGGGTCTCGCCGCGCTCGCCGAGGGGCGCACTCCT
GCGGGCGTGGTGCGCGGCGAGCATGTGCACCGTCCGACGACGGCATGGCTGTTC
ACCGGACAGGGCAGCCAGTACCCGGGCATGGCCCGCGAATTGTTCGACACGCAA
CCGGTTTTCGCCCAGACCGTTCGGCAGTGCGCCGAGGCGGTCGCCGACATGCTGC
CGCGACCACTGCTGGACGTGCTGTTCGCGACCGACCGCGAAACCGCGGACCTGC
TGCAGCACACGTCGTTCGCGCAGCCCGCGCTGTTCGCGGTCGAGATGGGGCTAGC
GCGGCTGTGGCAGTCATGGGCCATCGAACCCGATGTGGTGCTGGGGCACAGCGT
CGGCCAGTACGCCGCGGCCTGCGTGGCGGGCGTGTTCAGCCTTGACGACGGCGC
ACGACTGATGGCCGAGCGCGGCCGCATGTTCGGCAGCCTTCCCGAAGGCGGCCG
GATGGTGGCCGTGTTCGCCGACGCCGAACACGTCGAGCAGGTGGCCGGTGAGTT
CCCCCGGGTGTCGGTCGGCGCCTACAACGGCCCCAACACCGTGCTCTCGGGTCCC
GGCGAGGACCTCGAAGAGATCGTCGAGAGGTTCGAGGACGAGGGGATCCGCTGC
ACGTGGCTGGCGACCAGCCACGCGTTCCACTCCGAACTGCTCGATCCCGTGCTCG
ACGAGTTCGAGGCCTACGCGGCCCAGTTCCAGTTCGCAGCGCCGACACTGCCTTT
GGTGTGCAACCGGACCGGGGCCGTGCTCACCGGGCAGACCCCGCTCGACGCGCA
GTACTGGCGCAGGCATTCCCGCCAGCCCGTGCAGTTCGCCGAGAGTGTGCGCACC
GTCGCCGCGCTCGGCTGCTCGGTGCTGATGGAGATCGGCCCGCAACCTGTGCTGA
CCGGGGCCGCGGTACAGGTGTGGCCCGAACACATGGCTGCCCCGCGGGCCGTCG
CCTCGCTGCGCAAGGGCGTCGCCGATCGCCGACAGATCGCCGATGCGCTGGCGT
CGGCCTACGTCGGCGGCCACCGCCCCGACTTCGCCGCGGTGCACCGGCAGCCAC
GCCGCCCGGTCGAGTTGCCCACCTATCCGTTCCAGCGCCGCAGGTTCTGGCCGAA
GGCCTCGAGCATCGCCGTCGACGGTGGGACCGCGGCGTCGGGAATCCTGGGCAG
CGGCAAGGATCTGGCGTCCGGCGACACCATCTACACGAGCCGGCTGTCCGTCAA
ATCGCAGCCGTGGCTGGCCGATCACGTCATCTACGGCACCGTCGTCGTCCCCGGC
GCGACGTATGCGGCGATGGCGCTCGCCGCGGTCGGCACACCGGCACGCCTGAAG
GACGTGTTCTTCTACGAGCCGATCATCCTGCCCGAGAAGGCCTCTCGCGAGGTCC
AGCTGACGTTGCACCCTGCCGACGGTGGCAGCGTGCTCAGCTTCCAGATCCACAG
CCGCCCGTACGGTGAACGTGGTGCCGACTGGTCGCTGAACGCCGAGGGCACGGT
CGACACGGCCGGCAGCGACCCGGACGCCGCCGCAGACGAGCAGTCGGATCCGGT
CGACGAGGTGATCGAACGCCTCGAGCGCATGCGTCCGCAGGACCTCTTCGAGAC
CTTCGCCGACCTGGAACTGGCGTGGGGTCCCACATGGTCGGGCTCGCTGAAATCG
TTGTGGCTCGGCGACGGTGAGGCCGTCGGCGACATCCTCGTCGGCGCCGAACTCG
CCGAGCAACTCGGCTCCGAGCCCATGCACCCGGTGCTGATGGACCTGTGCACGG
GCGTCGCGTTCCCCGCGTTCCCGGCGCTGCTCGCGGCCGAACAGGGCGTCAACGA
CCTGTTCCTGCCGCTGCGCTACGGGCAGGTGACGCTCGCGGAGAAGATGCCGCG
GCGGTTCTACTGCCGGGCGAGGTGGCACCGTAGCGCGCTCGACAGCGAAACGCA
GGTGTTCGAACTCGAATTCGTCGACCTCGGTGGGCGAGCGCTCGGCGGCATCCGC
GAGTTCACCGTCAAACGTGCGCCGCGCGAGGCACTGCTGCGCGGCCTCGGGGGT
GACGCGACCCGCCTGCTCTACACGCTCGGGTGGCACGAGGTGCCGCCGCCACCA
TCGGCCGACGACGCGGTCGGCACGTGGCTGATCGCCGGGTTCGACGAGCTGGCC
GCGAAGGTGCCCGGCTGTGTCCCCATCGACCGCAACACCGATCTGCAGGTCCTCG
GTGAGGTGCTGTCGCAGGCGCACGCTCGCGGCGCGGGATTCTCGGGCGTCGTGT
GGCGTTGCGCGACGCCTGGCCGGAAGGAATCGGGTTCTGCCTCCGCCGAATCCG
CCCGTCTGGAGGCCGAGATCACCAATCTGCTCAGCGCGGTGCACGCCGCGCAGG
GGCCGAGCGTGAAACTGCCCGGCGGACTCTGGATCGTCACCGAACACGGCGTGG
CATGTGAATCCGGCGAGCCGGTGGACCCGGTGCAGGCCGCGCTGTGGGGCTTCG
GCCGAACGACGATCAACGAGGAACCGGCGCTGCACTGCCGGCTCGTCGACTGCG
ACGGATCTTCCGAGGCCGTCGACCTGCTGGCCGCTCTGCTGGCCGCACCGGGCAT
CCAGGAACCGGAAATCGCCGTGCGGCAGGGCAAACTGCTGGCGTCACGGTTGTT
GCCGTGGGCCCGCAGTGGTCATCTCACGCTGCCGCGCGGGGGCGACTTCGTCCTT
GCGCCCACCGAGCGCGGCGCGATCGACAACCTGCGCCTCACGGAGACGGACGTG
CCGGAGCCGGACGAGGGCTACGTGCAGGTTCGGGTCGAGGCCGCGGGCCTCAAC
TTCCGCGACGTGCTCAACGTGCTCGGCCTGTACCCCGGTGATCCGGGACCGATCG
GCGGCGACTTCGCAGGCACCGTGACCCAACTGGGGGCAGGTGTGAGTGGACTCG
AAATCGGCCAACGCGTGTACGGGTCGATGCAGGGTGCCTTCGCGAGCCGCTTCA
ACGTGCCCGCCCAGTTCCTGGCGCCGATTCCCGACGGCATCGGCGCGGTCGAGGC
CGCCACGATCCCGGCCGCGGCGCTGACGGTCCGGCTCGCGTTCGACTGGGCGCA
ACTCAAACCCGGCGACAAGGTGCTCATCCACGCCGCCAGCGGTGGCGTGGGGCT
CGCGGCCATCCAGATGGCGCAGCAGTCCGGCGCCGAGGTCTTCGCCACCGCGAG
CACCTTCAAGCGTTCGACCCTGCGCAGGCTGGGCGTGAAGTACGTCTACGACTCT
CGGACGACGGACTTCGCCGACCAGATCCTCGCGGACACCAACGGTGCCGGCGTG
GACGTGGTGCTCAACAGCCTCACTAGTGCGGGTTTCGTCGAGGCGACGCTGAAG
GCCACCGCGCAGGGCGGACGCTTCGCCGAGATCGCCAAGCGCGACATCTGGTCG
CATGAGCAGATGGCCGAGGCCCGCCCCGACATCGCCTACGAGATCGTCGCGCTG
GACACCGTGATGTTCACCGAGCCCGATCGCATCCGCGACCTGCTCACCGAGGTGT
CCGACGGGATGGCGCGCGGCGAGTGGACACCACTACCCGCCGAGATCTACCCGA
TCACCGAGGCCCGGTCGGCGTTCCGGCGCATGCAACAGGCACGGCACATCGGCA
AGATTGTGCTGCAGGTCCCCAAACCCCTTCAGCCGCAGCAGGACCGGAGCTACCT
GATCACCGGCGGACTCGGGGCGATCGGCCTGCACACGGCGGCGTATCTGGCGCA
GCTCGGCGCGGGCGACATCGTGCTGACCAGCCGGCGCGACCCCGACGAGTCGGC
CCAGCGGGCGATCGACGAGATCACCGAGCGCCACAAGTGCCGCATCCACACCTA
TGCGGCCGATGTGGGTGACGAGGCACAGGTGGCCGATCTGCTGGCGCGGATCCG
GGCCGAGTTGCCTCCACTGGCCGGAATCGCCCATCTGGCGGGCGTGCTCGACGAC
GCGCTGCTGTCGGCGCAGAACCCCGAGCGCTTCCGGACCACGTTGGCGCCCAAG
GCCTATGGCGCGAGCCACCTGGACCGCCTGACGCGCGACGACGACCTGGACTTC
TTCATCGTGTCCTCGTCGGTGTCGAGCCTGTTCGGATCACCGGGGCAGGCCAACT
ACGCAACCGCCAATGCGATGCTCGACGGTCTGGTCGCCAAGCGCCGTGCGCAGG
GCCTGGTGGCCACGGGCATCAACTTCGGGCCGTGGGGTCAGGGCGGCATGGCGT
CGTCGGAGGCCGCGACCGCGAACATCACCGCCCAAGGTCTGATCCCGTTGGATC
CGTCGGCCGCGTTGCACGCGCTGGCCGAGGTCATCGCGAACGGCTCCGGGCAGG
CGACGGTGCTCAAGGCCAACTGGCAGCGCGCGGCGAAGGTTCTCGGCTCGGCGC
GCCCGCCGATCCTGGACCTCGTCCTGCCGAGCGCGGCCGGGGAGGCCACGGGTG
ACAGCGAGCTGCTCAAGCAGTTGATGGAGATCCCGATACCGCAGCGCGCCGGGT
TCGTCACCGAGTTCCTGCAACGCGAAGTGCAGAACTTCCTGCGTCTCGCGTCGCC
GCCCGCGGCGTCGAGCCGGTTCCTGGACCTCGGCACGGATTCGCTGATGGCCATC
GAGCTGCGCAACCGGCTGCACAGCCAGTTCGGGGGCAAGTTCACCATCAACGCG
ACCGCGGTGTTCGACTACCCGACCATCGGCGGGCTCGCGGAGTACCTCGTGGGTC
AGCTGCCCGACGCCACTAGTCAGCTCGACAGCGGGACTCCCGCCCGGGAAGCGA
GCAGCGCTCTTCGCGACGGCTACCGGCAGGCGGGCGTGTCGGGCAGGGTCCGGT
CCTACCTCGACCTGCTGGCGGGGCTGTCGGACTTCCGCGAGCACTTCGACGGCTC
CGACGGGTTCTCCCTCGATCTCGTGGACATGGCCGACGGTCCCGGAGAGGTCACG
GTGATCTGCTGCGCGGGAACGGCGGCGATCTCCGGTCCGCACGAGTTCACCCGG
CTCGCCGGGGCGCTGCGCGGAATCGCTCCGGTTCGGGCCGTGCCCCAGCCCGGCT
ACGAGGAGGGCGAACCTCTGCCGTCGTCGATGGCGGCGGTGGCGGCGGTGCAGG
CCGATGCGGTCATCAGGACACAGGGGGACAAGCCGTTCGTGGTGGCCGGTCACT
CCGCGGGGGCACTGATGGCCTACGCGCTGGCGACCGAACTGCTCGATCGCGGGC
ACCCGCCACGCGGTGTCGTCCTGATCGACGTCTACCCGCCCGGTCACCAGGACGC
GATGAACGCCTGGCTGGAGGAGCTGACCGCCACGCTGTTCGACCGCGAGACGGT
GCGGATGGACGACACCAGGCTCACCGCCCTGGGCGCCTACGACCGCCTCACCGG
TCAGTGGCGACCCCGGGAAACCGGGCTGCCGACGCTGCTGGTCAGCGCCGGCGA
GCCGATGGGTCCGTGGCCCGACGACAGCTGGAAGCCGACGTGGCCCTTCGAGCA
CGACACCGTCGCCGTCCCCGGCGACCACTTCACGATGGTGCAGGAACACGCCGA
CGCGATCGCGCGGCACATCGACGCCTGGCTGGGCGGAGGGAATTCATGAAAGCT
TGCGGCCGCACTCGAGCACCACCACCACCACCACTGAGATCCGGCTGCTAACAA
AGCCCGAAAGGAAGCTGAGTTGGCTGCTGCCACCGCTGAGCAATAACTAGCATA
ACCCCTTGGGGCCTCTAAACGGGTCTTGAGGGGTTTTTTGCTGAAAGGAGGAACT
ATATCCGGAT

As discussed in the background section of the present invention, the extraction of aroma lactones from natural sources are not economically viable due to their low abundance. Further, delta acyl lactones produced chemically or by enzymatic or microbial conversion of hydroxy fatty acids are not very effective and cost-effective.

To circumvent the problems existing in the art, the present invention provides a recombinant DNA comprising a nucleotide sequence encoding a protein having an amino acid sequence as set forth in SEQ ID NO: 2 or SEQ ID NO: 4. The recombinant DNA has a nucleotide sequence as set forth in SEQ ID NO: 1 or SEQ ID NO:3. The present invention also discloses a recombinant vector comprising the said recombinant DNA. The present invention further discloses a method for producing δ-acyl lactones using the recombinant vector comprising said recombinant DNA. In the said method, the recombinant vector is transformed in a host cell (such as E. coli cells) such that recombinant proteins are expressed and δ-acyl lactones are produced. The recombinant host cells are grown in a culture medium comprising the glucose or supplemented with glucose. The method as described herein utilizes channelizing of intrinsic fatty acyl chains as substrate towards the recombinant protein PKSTE or PKS14TE to produce delta hydroxy acyl chain release as well as cyclization to produce delta acyl lactones. Overall, it can be inferred that 8-dodecalactone was detected in the recombinant host cells when transformed with the recombinant vector (SEQ ID NO: 5 or SEQ ID NO: 6), as compared to the host cells when transformed with a non-working vector.

Since no supplementation of fatty acid precursors are essential for production of delta acyl lactones, this method provides economic benefits over the methods existing in the art. The present invention thus enables 8-hydroxy acyl chain release as well as cyclization to produce δ-acyl lactone.

In an embodiment of the present invention, there is provided a recombinant DNA encoding a protein having an amino acid sequence as set forth in SEQ ID NO: 2, or SEQ ID NO: 4.

In an embodiment of the present invention, there is provided a recombinant DNA encoding a protein having an amino acid sequence as set forth in SEQ ID NO: 2, has a nucleotide sequence as set forth in SEQ ID NO: 1.

In an embodiment of the present invention, there is provided a recombinant DNA encoding a protein having an amino acid sequence as set forth in SEQ ID NO: 2, has a nucleotide sequence as set forth in SEQ ID NO: 3.

In an embodiment of the present invention, there is provided a recombinant vector comprising the recombinant DNA operably linked to a heterologous promoter, wherein the recombinant DNA encodes a protein having an amino acid sequence as set forth in SEQ ID NO: 2, or SEQ ID NO: 4, and wherein the recombinant vector has a nucleic acid sequence as set forth in SEQ ID NO: 5, or SEQ ID NO: 6.

In an embodiment of the present invention, there is provided a recombinant vector comprising the recombinant DNA operably linked to a heterologous promoter, wherein the recombinant DNA having a nucleotide sequence as set forth in SEQ ID NO: 1 encodes a protein having an amino acid sequence as set forth in SEQ ID NO: 2, and wherein the recombinant vector has a nucleic acid sequence as set forth in SEQ ID NO: 5.

In an embodiment of the present invention, there is provided a recombinant vector comprising the recombinant DNA operably linked to a heterologous promoter, wherein the recombinant DNA having a nucleotide sequence as set forth in SEQ ID NO: 3 encodes a protein having an amino acid sequence as set forth in SEQ ID NO: 4, and wherein the recombinant vector has a nucleic acid sequence as set forth in SEQ ID NO: 6.

In an embodiment of the present invention, there is provided a recombinant vector as described herein, wherein the heterologous promoter is a T7 promoter.

In an embodiment of the present invention, there is provided a recombinant vector as described herein, wherein the vector is selected from pET21c.

In an embodiment of the present invention, there is provided a recombinant host cell comprising the recombinant vector as described herein.

In an embodiment of the present invention, there is provided a recombinant host cell as described herein, wherein the host cell is a prokaryotic cell.

In an embodiment of the present invention, there is provided a recombinant host cell as described herein, wherein the host cell is an E-coli cell.

In an embodiment of the present invention, there is provided a recombinant protein having an amino acid sequence as set forth in SEQ ID NO: 2, or SEQ ID NO: 4.

In an embodiment of the present invention, there is provided a recombinant protein as described herein, wherein the recombinant protein having an amino acid sequence as set forth in SEQ ID NO: 2, is encoded by a nucleotide sequence as set forth in SEQ ID NO: 1.

In an embodiment of the present invention, there is provided a recombinant protein as described herein, wherein the recombinant protein having an amino acid sequence as set forth in SEQ ID NO: 4, is encoded by a nucleotide sequence as set forth in SEQ ID NO: 3.

In an embodiment of the present invention, there is provided a method for producing a recombinant protein as described herein, said method comprising the steps of: (a) obtaining a recombinant vector as described herein; (b) transforming a host cell with the recombinant vector of step (a) to obtain a recombinant host cell; (c) culturing the recombinant host cell of step (b) in a culture medium comprising an inducer, to obtain cultured cells expressing recombinant protein having an amino acid sequence as set forth in SEQ ID NO: 2, or SEQ ID NO: 4; and (d) subjecting the cultured cells of step (c) to purification to obtain the recombinant protein.

In an embodiment of the present invention, there is provided a method for producing a recombinant protein as described herein, wherein the culture medium is any culture medium comprising the glucose or a culture medium supplemented with glucose. In an example, the culture medium is Luria Bertani (LB) medium.

In an embodiment of the present invention, there is provided a method for producing a recombinant protein as described herein, wherein the inducer is Isopropyl β-d-1-thiogalactopyranoside (IPTG).

In an embodiment of the present invention, there is provided a method for producing a recombinant protein as described herein, wherein culturing comprising the steps of growing the recombinant host cell at a temperature of 30° C., followed by inducing the recombinant host cell at a temperature in the range of 16 to 25° C. for a time period of 16-20 hours. In another embodiment of the present invention, inducing the recombinant host cell is done at a temperature of 16° C. for a time period of 16 hours.

In an embodiment of the present invention, there is provided a method for producing a recombinant protein as described herein, wherein the purification is done by Ni-NTA based affinity chromatography.

In an embodiment of the present invention, there is provided a method for producing a recombinant protein as described herein, said method comprising the steps of: (a) obtaining a recombinant vector as described herein; (b) transforming a host cell with the recombinant vector of step (a) to obtain a recombinant host cell; (c) culturing the recombinant host cell of step (b) in culture medium comprising an inducer, wherein the culture medium is a Luria Bertani (LB) medium or a medium supplemented with glucose, and wherein the inducer is Isopropyl β-d-1-thiogalactopyranoside (IPTG), to obtain cultured cells expressing recombinant protein having an amino acid sequence as set forth in SEQ ID NO: 2, or SEQ ID NO: 4, wherein culturing comprising the steps of growing the recombinant host cell at a temperature of 30° C. till the optical density of 0.6 is attained, followed by inducing the recombinant host cell at a temperature of 22° C. for a time period of 16 hours; and (d) subjecting the cultured cells of step (c) to purification done by Ni-NTA based affinity chromatography, to obtain the recombinant protein.

In an embodiment of the present invention, there is provided a method for producing delta acyl lactone, said method comprising the steps of: (a) obtaining a recombinant vector as described herein; (b) transforming a host cell with the recombinant vector of step (a) to obtain a recombinant host cell; and (c) culturing the recombinant host cell of step (b) in a culture medium comprising an inducer, to obtain delta acyl lactone, wherein the recombinant host cell expresses the recombinant proteins having an amino acid sequence as set forth in SEQ ID NO: 2, or SEQ ID NO: 4.

In an embodiment of the present invention, there is provided a method for producing delta acyl lactone, said method comprising the steps of: (a) obtaining a recombinant vector as described herein; (b) transforming a host cell with the recombinant vector of step (a) to obtain a recombinant host cell; and (c) culturing the recombinant host cell of step (b) in a culture medium comprising an inducer, to obtain cultured cells expressing the recombinant proteins having an amino acid sequence as set forth in SEQ ID NO: 2, or SEQ ID NO: 4; (d) subjecting the cultured cells of step (c) to purification to obtain a recombinant protein; and (e) setting up enzymatic biochemical reaction with at least one purified substrate and the recombinant protein, to produce delta acyl lactone.

In an embodiment of the present invention, there is provided a method as described herein, wherein the purified substrate is selected from the group consisting of malonyl Coenzyme A (MCoA), synthetic dodecanoyl NAC, or octanoyl NAC.

In an embodiment of the present invention, there is provided a method for producing delta acyl lactone as described herein, wherein the culture medium is a medium comprising glucose or a medium supplemented with glucose. In another embodiment of the present invention, the culture medium is a Luria Bertani (LB) medium.

In an embodiment of the present invention, there is provided a method for producing delta acyl lactone as described herein, wherein the inducer is Isopropyl β-d-1-thiogalactopyranoside (IPTG).

In an embodiment of the present invention, there is provided a method for producing delta acyl lactone as described herein, wherein culturing is done at a time period in the range of 90 to 130 hours. In another embodiment of the present invention, culturing is done at a time period in the range of 100 to 120 hours. In yet another embodiment of the present invention, culturing is done at a time period of 120 hours.

In an embodiment of the present invention, there is provided a method for producing delta acyl lactone, said method comprising the steps of: (a) obtaining a recombinant vector as described herein; (b) transforming a host cell with the recombinant vector of step (a) to obtain a recombinant host cell; and (c) culturing the recombinant host cell of step (b) in a culture medium comprising an inducer, to obtain delta acyl lactone, wherein the culture medium is a medium comprising glucose or a medium supplemented with glucose, and wherein the inducer is Isopropyl β-d-1-thiogalactopyranoside (IPTG), and wherein culturing is done for a time period in the range of 90 to 130 hours, and wherein the recombinant host cell expresses the recombinant proteins having an amino acid sequence as set forth in SEQ ID NO: 2, or SEQ ID NO: 4.

In an embodiment of the present invention, there is provided a method for producing delta acyl lactone, said method comprising the steps of: (a) obtaining a recombinant vector as described herein; (b) transforming a host cell with the recombinant vector of step (a) to obtain a recombinant host cell; and (c) culturing the recombinant host cell of step (b) in a culture medium comprising an inducer, to obtain cultured cells expressing the recombinant proteins having an amino acid sequence as set forth in SEQ ID NO: 2, or SEQ ID NO: 4, wherein the culture medium is a medium comprising glucose or a medium supplemented with glucose, and wherein the inducer is Isopropyl β-d-1-thiogalactopyranoside (IPTG), and wherein culturing is done for a time period in the range of 90 to 130 hours; (d) subjecting the cultured cells of step (c) to purification to obtain a recombinant protein; and (e) setting up enzymatic biochemical reaction with at least one purified substrate and the recombinant protein, to produce delta acyl lactone.

The present invention is illustrated hereunder in greater detail in relation to non-limiting exemplary embodiments as per the following examples:

### EXAMPLES

The following examples are put forth so as to provide those of ordinary skill in the art with a complete disclosure and the description of how to make and use the present invention, and are not intended to limit the scope of what the inventors regard as their invention nor are they intended to represent that the experiments below are all and only experiments performed. The methodology of preparing few of the preferred embodiments shall become clearer with working examples provided below.

### Example 1: Cloning of the Recombinant or Engineered DNA

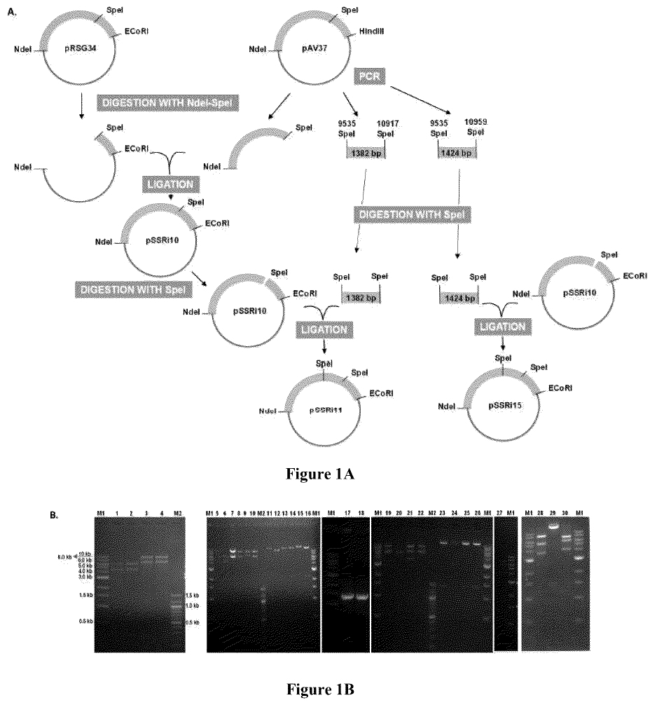

DEBS1 thioesterase (TE_{DEBS}) gene (Kao C M, et al. Manipulation of macrolide ring size by directed mutagenesis of a modular polyketide synthase. Journal of the American Chemical, Society. 1995 September; 117 (35): 9105-6.) was cloned in frame at the C-terminus of the Msmeg type I pAV37 vector (PKS_{GPL}). Two different recombinant vectors were made in the present invention. In the first recombinant vector, the TE_{DEBS} gene was fused right after the Acyl carrier protein (ACP) domain of PKS_{GPL} while, in the second recombinant vector, the TE_{DEBS} domain was placed at the end of the PKS_{GPL}. The cloning strategy for the same is depicted in FIG. 1A.

Recombinant vector 1 pSSRi11 (SEQ ID NO: 5): Clone pRSG34 (TE_{DEBS}) and pAV37 (PKS_{GPL}) (Vats, Singh et al., Mukherjee R, Chopra T, Ravindran M S, Mohanty D, Chatterji D, Reyrat J M, Gokhale R S. Retrobiosynthetic approach delineates the biosynthetic pathway and the structure of the acyl chain of mycobacterial glycopeptidolipids. J Biol Chem. 2012 Aug. 31; 287(36): 30677-87) were digested with NdeI-SpeI restriction endonucleases. The 9.5 kb DNA from pAV37 digestion was ligated with the ˜6.3 bp vector backbone from pRSG34 digestion. The ligation mixture was transformed in XL-1 blue competent cells. The transformants were screened for positive clone pSSRi10 by restriction digestion. The remaining 1382 bp DNA (9535 bp-10917 bp) of PKS_{GPL} was PCR amplified using forward primer IML578 (SEQ ID NO: 9) and reverse primer IML579 (SEQ ID NO: 10; Table 1). Both the forward and the reserve primers were engineered to contain SpeI restriction endonuclease enzyme site. This 1382 bp DNA fragment was cloned in pSSRi10 to generate a recombinant vector pSSRi11 (SEQ ID NO: 5) comprising a recombinant DNA having a nucleotide sequence as set forth in SEQ ID NO: 1, wherein the recombinant DNA is operably linked to T7 promoter in the vector. The positive clone was screened by restriction digestion (FIG. 1B).

Recombinant Vector 2 pSSRi15 (SEQ ID NO: 6): The strategy for obtaining the recombinant vector 2 was similar to the one described above. The 1424 bp DNA (953510959 bp) of PKS_{GPL} was PCR amplified using forward primer IML578 (SEQ ID NO: 9) and reverse primer IML579 (SEQ ID NO: 10; Table 1). Both the forward and the reserve primers were engineered to contain SpeI restriction endonuclease enzyme site. The ˜1.4 kb DNA fragment was cloned in pSSRi10 to generate a recombinant vector pSSRi15 (SEQ ID NO: 6) comprising a recombinant DNA having a nucleotide sequence as set forth in SEQ ID NO: 3, wherein the said nucleic acid fragment is operably linked to T7 promoter. The positive clone was screened by restriction digestion (FIG. 1B).

TABLE 1
List of primer sequences used in the present invention:

| Primer Name | Sequence (5′-3′) | SEQ ID NO |
|---|---|---|
| IML578 (Forward | CTCAACAGCCTCACTAGTGCGGGTTTC | SEQ ID NO: 9 |
| primer) | | |
| IML579 (Reverse | CTGCTGACTAGTGAGGTACTCCGCGAGCCC | SEQ ID NO: 10 |
| primer) | | |
| IML808 | CTGCTGACTAGTGGCCGTTTCGGGCGC | SEQ ID NO: 11 |

The recombinant vectors of the present invention (SEQ ID NO: 5 or SEQ ID NO: 6) were further used to transform the host cells, such as E. coli cells.

### Example 2: Expression and Purification of Recombinant Protein

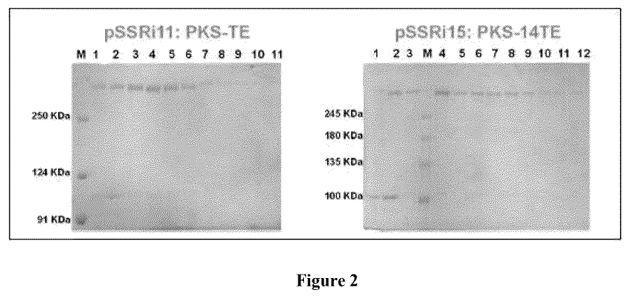

The recombinant vectors pSSRi11 (SEQ ID NO: 5) and pSSRi15 (SEQ ID NO: 6) were individually transformed in BAP1 E. coli competent cells, to obtain recombinant E. coli cells (host cells). The E. coli cells when transformed with the recombinant vectors pSSRi11 (SEQ ID NO: 5), expresses recombinant proteins, i.e., PKSTE (SEQ ID NO: 2), whereas, when the E. coli cells when transformed with the recombinant vector pSSRi15 (SEQ ID NO: 6), expresses recombinant proteins PKS14TE (SEQ ID NO: 4). The recombinant protein expression was induced with 0.5 mM IPTG at 22° C. for 16 hours. About ˜430 kda protein was purified to homogeneity (FIG. 2) using Ni-NTA based affinity chromatography. The protein was concentrated to ¼ volume using protein concentrator (Millipore) of 100 kda cut off.

### Example 3: In-Vitro Biochemical Assay

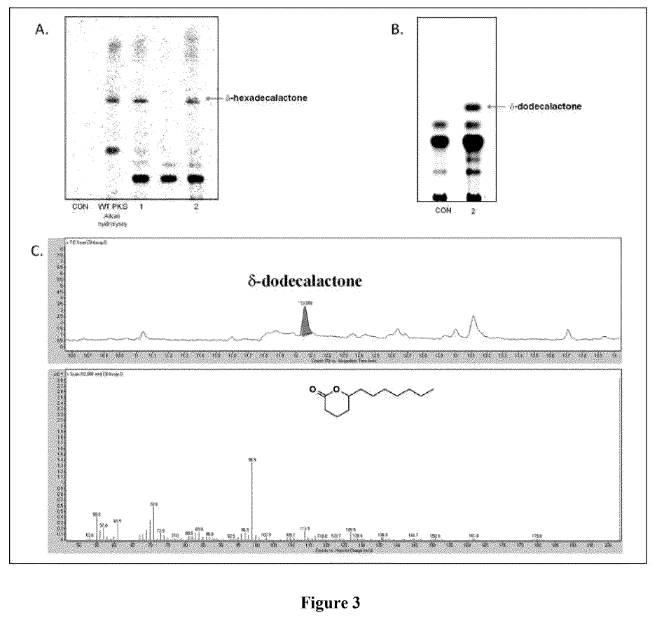

The biochemical assay for PKS_{GPL}-TE_{DEBS} protein was set up as described in Vats, Singh et al. 2012). Radiolabelled C14 malonyl Coenzyme A (MCoA) and synthetic dodecanoyl NAC or octanoyl NAC were used as the substrates. The reaction was set up for 16 hours. The reaction with wild type (WT) PKS was acid hydrolysed to release the product as described in Vats, Singh et al. 2012 while the PKS_{GPL}-TE_{DEBS} reactions were not. The metabolite was extracted with 1:2 volume of ethyl acetate for 3 times. The extract was dried using speed vacuum. The dried extract was resuspended in 20 μl of ethyl acetate and resolved on thin-layer chromatography (TLC) (FIG. 3A). 8-hexadecalactone was observed in lanes of PKSTE and PKS14TE at a Rf ˜0.54. FIG. 3B shows assay for PKS14TE with octanoyl SNAC as substrate. Product band at Rf ˜4.7 similar to that of δ-dodecanoyl lactone was observed. Further, cell free enzymatic assay for PKS14TE protein was set up with MCoA and octanoyl NAC as substrate and the reaction mixture was analysed using GC-MS. A peak for δ-dodecanoyl was detected (FIG. 3C). Successful protein engineering was achieved with PKSTE (SEQ ID NO: 2) and PKS14TE (SEQ ID NO: 4) recombinant vectors as developed in the present invention.

### Example 4: Metabolite Production by E. coli

The recombinant vectors/clone pSSRi15 (SEQ ID NO: 6) was co-transformed in ΔfadAB BAP1 (Unsaturated Lipid Assimilation by Mycobacteria Requires Auxiliary cis-trans Enoyl CoA

Isomerase. Chem Biol. 2015 Dec. 17; 22(12): 1577-87), and optionally along with Mtb FAAL10 (Chhabra A, et al. Nonprocessive [2+2]e-off-loading reductase domains from mycobacterial nonribosomal peptide synthetases. Proc Natl Acad Sci USA. 2012 Apr. 10; 109(15):5681-6), to obtain a recombinant E. coli cell.

The step of co-transforming the said recombinant vector of the present invention with FAAL10 protein (Fatty acyl AMP ligase 10) is an optional step because FAAL10 activates free fatty acids to fatty acyl-AMP which is the precursor for the PKS protein. E. coli also possess its own fatty acyl AMP ligase protein. The co-transformation of FAAL10 overexpresses FAAL10 and enables channelizing of the intrinsic precursors to the engineered protein. Therefore, in principle this co-transformation is an optional step.

This recombinant E. coli strain (ΔfadAB BAP1 Mtb FAAL10 pSSRi15a) was grown in Luria Bertani (LB) broth with 1 mM Isopropyl β-d-1-thiogalactopyranoside (IPTG) (inducer) for 120 hours, to obtain cultured cells. The cultured cells and the culture filtrate were separated by centrifugation at 5000 rpm for 15 min. The culture filtrate was acidified to pH 2 and then the metabolite was extracted with 1:1 volume of ethyl acetate for overnight. The organic layer was collected and dried using rotavap. The metabolites were dissolved in minimum volume of ethyl acetate and then adsorbed in silica. The metabolites were then fractionated based on their polarity using in-house packed silica column. The fractions were dried and then resuspended in 300 μl of ethyl acetate.

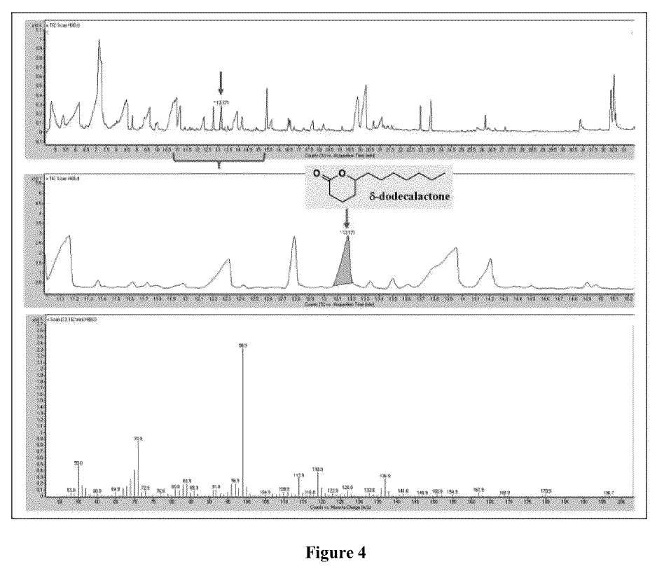

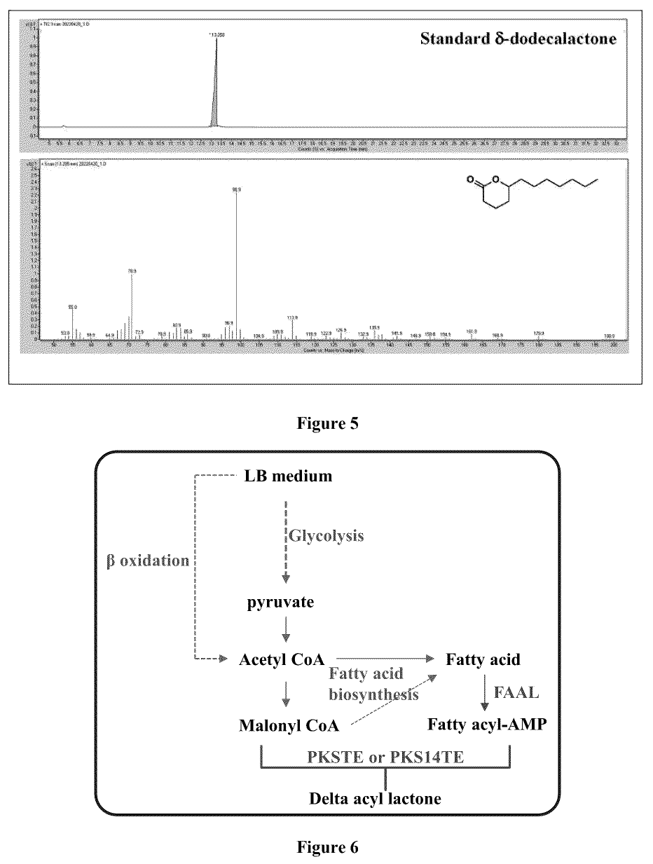

While the metabolite extraction from cells was performed with 1:10 volume of ethyl acetate for overnight. The organic layer was collected by centrifugation in glass tubes at 1800 rpm for 10 min. The organic layer was dried and then fractionated using silica column. The fractions were dried and then resuspended in 300 μl of ethyl acetate. Each fraction (Culture filtrate extract and cell extract) were then analysed on Gas chromatography-mass spectrometry (GC-MS). δ-dodecalactone was detected from both culture filtrate as well as cells. FIG. 4 depicts the GC-MS chromatogram for fraction 3 of culture filtrate that shows the presence of δ-dodecalactone. —The metabolite identity was confirmed by comparing with that of the δ-dodecalactone synthetic standard as well as library search based on the MS profile (FIG. 5). FIG. 6 summarizes the pathway for producing δ-dodecalactone using the recombinant vectors (SEQ ID NO: 5 or SEQ ID NO: 6).

### Example 5: Non-Working Example

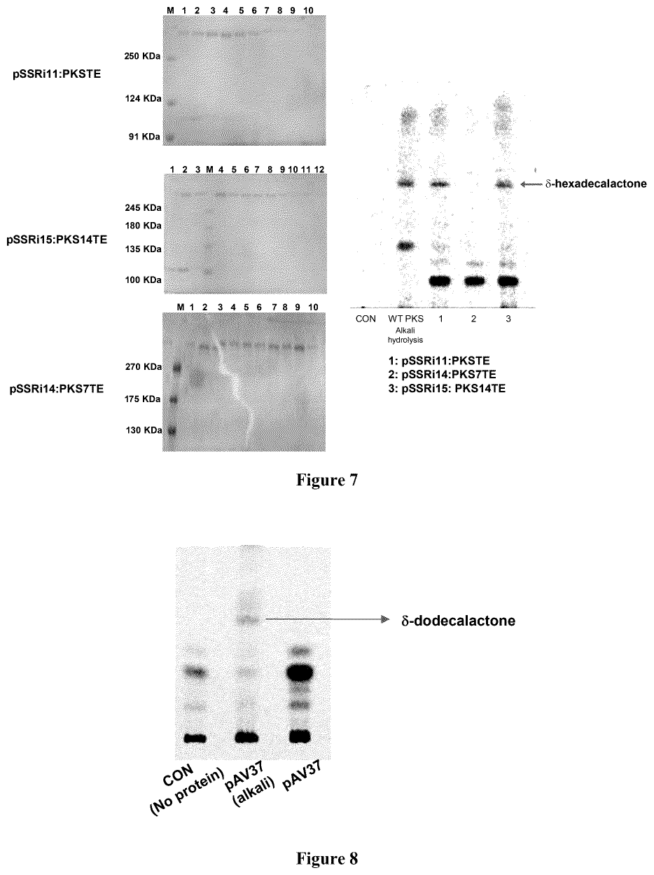

For the purpose of the present invention, three vectors were constructed: —pSSRi11 (SEQ ID NO: 5), pSSRi14 (SEQ ID NO: 14), and pSSRi15 (SEQ ID NO: 6). All the three were expressed in E. coli and the recombinant proteins were purified as described in Example 2. pSSRi14 is a non-working vector that comprises the DNA having a nucleotide sequence as set forth in SEQ ID NO: 12, which encodes a protein having an amino acid sequence as set forth in SEQ ID NO: 13. Out of three vectors, only recombinant proteins (SEQ ID NO: 2 and SEQ ID NO: 4) from the recombinant vectors-pSSRi11 (SEQ ID NO: 5) and pSSRi15 (SEQ ID NO: 6) gave the positive enzymatic activity. The TLC data for the enzymatic function of the recombinant proteins (SEQ ID NO: 2 and SEQ ID NO: 4) is depicted in FIG. 7.

From FIG. 7, it can be clearly deduced that 8-dodecalactone was detected in the cultured cells when transformed with the recombinant vectors (SEQ ID NO: 5 and 6) of the present invention, thus, indicating the presence of 8-dodecalactone. In contrast, the proteins produced from the cultured cells upon transforming with the non-working vector (pSSRi14; SEQ ID NO: 14) did not yield delta acyl lactone from the enzymatic activity.

Hence, it can be inferred that the recombinant vectors (SEQ ID NO: 5 and 6) of the present invention is technically advanced over the non-working vector (pSSRi14; SEQ ID NO: 14) in terms of the production of 8-dodecalactone.

Further, in the present invention, it was observed that the presence of delta dodecalactone was observed only in the case in which pAV37 was subjected to alkali hydrolysis. As known in the art, PKS_{GPL} encoded by pAV37 produces delta-hydroxy fatty acyl chains that are channelized to the downstream NRPS protein for further processing to make surface lipid in GPL in M. smegmatis. In order to release the delta-hydroxy fatty acyl chains from the PKS protein, alkali hydrolysis was done. The TLC data as depicted in FIG. 8 shows the presence of delta dodecalactone only in the case in which pAV37 was subjected to alkali hydrolysis.

### Example 6: Comparative Data

The method for producing the δ-acylactone by transforming the E. coli cells using the recombinant vectors (SEQ ID NO: 5 or 6) in a LB culture medium that uses glucose as a substrate is highly effective and cost-effective for producing the δ-acylactone, as compared to the conventional methods that uses fatty acids as a substrate.

Table 2 summarizes the methods known in the art for producing
δ-acylactone using fatty acids a starting material:

| Substrate | Organism | Methodology | Reference |
|---|---|---|---|
| Hydroxylated fatty | Sporobolomyces | oxidation of the secondary | J Org Chem |
| acid [13-hydroxy- | odorus | hydroxy group followed by | (1991) 57: |
| 9,11(Z,E)- | | a subsequent enantioselective | 1954-1956 |
| octadecadienoic | | reduction | |
| acid] and 13-hydroxy- | | | |
| 9(Z)-octadecenoic | | | |
| acid | | | |
| Massoic lactone | S. cerevisiae | Hydrogenation of α,β- | Appl Microbiol |
| | | unsaturated lactone | Biotechnol |
| | | | (1992) 36: |
| | | | 712-716 |
| Non-hydroxylated | Y. lipolytica | Linoleic acid to 13 hydroxy- | Biotechnol Lett |
| fatty acid | With | 9-octadecenoic acid by | (2016) 38: |
| (linoleic acid) | supplementation | linoleate 13 hydratase | 817-823 |
| | of linoleate 13 | followed by β-oxidation and | |
| | hydratase from | lactonization by Y. lipolytica | |
| | Lactobacillus | | |
| | acidophilus | | |
| Non-hydroxylated | Y. lipolytica | Engineered Y. lipolytica for | Metabolic |
| fatty acid | | controlled b-oxidation and | Engineering 61 |
| (linoleic acid) | | over-expression of linoleate | (2020) 427-436 |
| | | 13 hydratase from | |
| | | L. acidophilus | |

Overall, it can be inferred that the method of the present invention is a cost-effective method as it deploys the glucose as a substrate material for producing δ-acylactone, as compared to conventional methods that deploys the fatty acids a starting material.

### Advantages of the Present Invention:

The present invention discloses a recombinant DNA comprising a nucleotide sequence encoding a protein having an amino acid sequence as set forth in SEQ ID NO: 2, or SEQ ID NO: 4. The present invention also discloses a recombinant vector comprising the recombinant DNA and recombinant host cell comprising the recombinant vector. The present invention further discloses a recombinant protein and a method for producing the said product. Moreover, there is also provided herein a method for producing the delta acyl lactone using the recombinant vectors and recombinant host cell as described herein.

The advantages of the present invention are as follows:
(a) Cost-effective method: The method of the present invention deploys a recombinant vector that is used to transform the host cells (such as E. coli) in a culture medium comprising glucose or supplemented with glucose as a substrate. Thus, the method using glucose as a substrate is an economical viable method over the existing methods that use fatty acid precursor as a substrate.
(b) Effective and efficient: The presence of the recombinant vectors of the present invention (SEQ ID NO: 5 and 6) is important for producing the delta acyl lactone effectively.
(c) High commercial value: Delta acyl lactones such as δ-decalactone and δ-dodecalactone produced by the method of the present invention are aroma compounds of high commercial value. These lactones impart fruity and milky aroma and can be used in food and perfume industry.

1. A recombinant DNA encoding a protein having an amino acid sequence as set forth in SEQ ID NO: 2, or SEQ ID NO: 4.

2. The recombinant DNA as claimed in claim 1, wherein the recombinant DNA encoding a protein having an amino acid sequence as set forth in SEQ ID NO: 2, has a nucleotide sequence as set forth in SEQ ID NO: 1.

3. The recombinant DNA as claimed in claim 1, wherein the recombinant DNA encoding a protein having an amino acid sequence as set forth in SEQ ID NO: 4, has a nucleotide sequence as set forth in SEQ ID NO: 3.

4. A recombinant protein having an amino acid sequence as set forth in SEQ ID NO: 2, or SEQ ID NO: 4.

5. The recombinant protein as claimed in claim 4, wherein the recombinant protein having an amino acid sequence as set forth in SEQ ID NO: 2, is encoded by a nucleotide sequence as set forth in SEQ ID NO: 1.

6. The recombinant protein as claimed in claim 4, wherein the recombinant protein having an amino acid sequence as set forth in SEQ ID NO: 4, is encoded by a nucleotide sequence as set forth in SEQ ID NO: 3.

7. A recombinant vector comprising the recombinant DNA as claimed in claim 1, and a heterologous promoter, wherein the recombinant vector has a nucleic acid sequence as set forth in SEQ ID NO: 5, or SEQ ID NO: 6.

8. The recombinant vector as claimed in claim 7, wherein the recombinant DNA encoding a protein having an amino acid sequence as set forth in SEQ ID NO: 2, has a nucleotide sequence as set forth in SEQ ID NO: 1, and wherein the recombinant vector has a nucleic acid sequence as set forth in SEQ ID NO: 5.

9. The recombinant vector as claimed in claim 7, wherein the recombinant DNA encoding a protein having an amino acid sequence as set forth in SEQ ID NO: 4, has a nucleic acid sequence as set forth in SEQ ID NO: 3, and wherein the recombinant vector has a nucleic acid sequence as set forth in SEQ ID NO: 6.

10. The recombinant vector as claimed in claim 7, wherein the heterologous promoter is a T7 promoter.

11. The recombinant vector as claimed in claim 7, wherein the vector is selected from pET21c.

12. A recombinant host cell comprising the recombinant vector as claimed in claim 7.

13. The recombinant host cell as claimed in claim 12, wherein the host cell is a prokaryotic cell.

14. The recombinant host cell as claimed in claim 12, wherein the host cell is an E-coli cell.

15. A method for producing a recombinant protein as claimed in claim 4, said method comprising the steps of:
(a) obtaining a recombinant vector;
(b) transforming a host cell with the recombinant vector of step (a) to obtain a recombinant host cell;
(c) culturing the recombinant host cell of step (b) in a culture medium comprising an inducer, to obtain cultured cells expressing recombinant protein having an amino acid sequence as set forth in SEQ ID NO: 2, or SEQ ID NO:4; and
(d) subjecting the cultured cells of step (c) to purification to obtain the recombinant protein.

16. A method for producing delta acyl lactone, said method comprising the steps of:
(a) obtaining a recombinant vector as claimed in claim 7;
(b) transforming a host cell with the recombinant vector of step (a) to obtain a recombinant host cell;
(c) culturing the recombinant host cell of step (b) in a culture medium comprising an inducer, to obtain cultured cells expressing recombinant protein having an amino acid sequence as set forth in SEQ ID NO: 2, or SEQ ID NO:4;
(d) subjecting the cultured cells of step (c) to purification to obtain the recombinant protein; and
(e) setting up enzymatic biochemical reaction with the recombinant protein to produce the delta acyl lactone.

17. A method for producing delta acyl lactone, said method comprising the steps of:
(a) obtaining a recombinant vector as claimed in claim 7;
(b) transforming a host cell with the recombinant vector of step (a) to obtain a recombinant host cell; and
(c) culturing the recombinant host cell of step (b) in a culture medium comprising an inducer, to obtain delta acyl lactone, wherein the recombinant host cell expresses the recombinant proteins having an amino acid sequence as set forth in SEQ ID NO: 2, or SEQ ID NO: 4.

18. The method as claimed in claim 15, wherein the inducer is Isopropyl β-d-1-thiogalactopyranoside (IPTG).

19. The method as claimed in claim 15, wherein culturing comprises the steps of growing the recombinant host cell at a temperature of 30° C., followed by inducing the recombinant host cell at a temperature in the range of 16 to 25° C. for a time period in the range of 16 to 20 hours.

20. The method as claimed in claim 16, wherein culturing is done for a time period in the range of 90 to 130 hours, and at a temperature in the range of 37° C.

21. The method as claimed in claim 15, wherein the purification is done by Ni-NTA based affinity chromatography.
